SEQUENCE LISTING (323 sequences)

SEQ ID NO: 1 - DNA, 69 bp, Mus musculus
cccgaacccgaacccgaacccgaacccgaacccgaacccgaacccgaacccgaacccgaacccgaaccc

SEQ ID NO: 2 - DNA, 69 bp, Mus musculus
cgcgcgcgcgcgcgcgcgcgcgcgcgcgcgcgcgcgcgcgcgcgcgcgcgcgcgcgcgcgcgcgcgcgc

SEQ ID NO: 3 - DNA, 69 bp, Mus musculus
cgaacgaacgaacgaacgaacgaacgaacgaacgaacgaacgaacgaacgaacgaacgaacgaacgaac

SEQ ID NO: 4 - DNA, 69 bp, Mus musculus
cggacggacggacggacggacggacggacggacggacggacggacggacggacggacggacggacggac

SEQ ID NO: 5 - DNA, 69 bp, Mus musculus
gccgccgccgccgccgccgccgccgccgccgccgccgccgccgccgccgccgccgccgccgccgccgcc

SEQ ID NO: 6 - DNA, 69 bp, Mus musculus
gcccgcccgcccgcccgcccgcccgcccgcccgcccgcccgcccgcccgcccgcccgcccgcccgcccg

SEQ ID NO: 7 - DNA, 69 bp, Mus musculus
gccccgccccgccccgccccgccccgccccgccccgccccgccccgccccgccccgccccgccccgccc

SEQ ID NO: 8 - DNA, 69 bp, Mus musculus
gcccccgcccccgcccccgcccccgcccccgcccccgcccccgcccccgcccccgcccccgcccccgcc

SEQ ID NO: 9 - DNA, 69 bp, Mus musculus
gcgcagcgcagcgcagcgcagcgcagcgcagcgcagcgcagcgcagcgcagcgcagcgcagcgcagcgc

SEQ ID NO: 10 - DNA, 2524 bp, Mus musculus
  misc_feature (2408)..(2408): n is a, c, g, or t
cagtgtttctcaaagtgtggtccgcggaccactggcggtcccccgcggttctatcaagtggtccgcaggcggtttggcggtttcagaggaaaaagcgatgaaacaattttgttcacatacatttcacaaatttgaaatgtaagattaattatgattttcacagaaatcccgttacgttcttaataatcgttacgttcttaaaggttgcgcatgtgctacaaggactgcgttggtcagttcgtctcggctaacattcagttaacagggtgcagttcgtctcggctaacgtattttcacgtcatttgcatgttattgtttacgtttgttaaatttgcatttttcgttgttactattgtgttgtattatatccccaattcacaaaaatggatcaatggctcaaaagtggttcattgaagcgtaaaagtagtgatgaaaatagtaacgtaaatactacaactcagaataacgtcataaacgtaaatagtgaacaggactccagtgcgaatatagaatgtgaatctgtatgtgctgggacaagtgaatctgcgagtgtgatgatttcgcacaagcagccgaaaaagaaaagtgcgaataggaagtacgacgatgaatatctgaaaattggattttattggaccggcgatccatttgcccctagtccccagtgcgttgtctgttatgaaactttgtcaaatagtggcatgaagccatcgaagctttcgcgtcattttcaaacaaagcacagtgacctctctggtaaaccaatcgagtttttccagaacaagcgcaaaataatgctttccagtacgaaattgatgaattttgtcgctaaaggcagagaagagaccaaaactacagaggcatcattcaaagttgcactccttatagcaaaaacaggtacaagtcacgctattgccgagaagcttgtaaaaccagccgcaaagttaatgacaaatattatgctcggagagaaagcagaacgagctattggcaaaattcctttatcaaatgacactgttggacgtcgcataatatcaatggcatacaatgtggaagagcaattactatcacgtgtgcgtgctagcagatatttcgctttacagttggacgaaaccactgatgtgcagagtatgaatcagctcttggcatatgtgcggtacatatatgagggagaagtgctcgacgactttttgttctgtttatcactgaaaacccatgctacaggagaagattttttttatttagttaacgattattttgtgagccgtgatgtagactggaaaaggtgcgttgggatcagtactgacggagcaccagctatgtgtgcagcaaggaagggcgttgctacgcgaataaaagaggttgcacctgaatgccaatccacacactgctttattcacagagaacagttggcggtcaacaatatgcctcctgatcttgattcagtgttgaaggaaatagtgaaaattgtgaatacgatcaaatcgcggccactgagtgtacgtcttttcagcgtgctgtgcgaagaaatgggcagcgagtacaagactctgcttttccacactgaagtacgctggctgtcgaggggaaaggtgctcacacgagtttttgaaatgagggatgagataaaaacatttcttcatgacactgataatgccagtaaagaccatttctacgatttcaagtggcttgctcaagtggtatatctcagcgatatattcagtatcttgaatagcctgaacctatcacttcagggccgaaatatcacgatttttaatgttgaggataagatatcaggatttcttaagaagaccgaactgtggtgcaaacggctcgatcgccgagagtttgactctttcccaacacttgatgattttcttcactcgtcggagaaagaaatcgatgacgtattattgggcatatttaaaaaccacatccaaatgctgcaacagaacatgaagaaatactttccagagccgaatgcaaccaaagagtggattaggaatccattcgccgctatctcccaagttgaaacattcaaccttccagcttttgagtgtgatgtgctcgtcgacttagcgtccgacggagcgctgaaagtagtcttcagtgagaaatctctccataatttctgggtccatgttcgatccgaatatccagaactatctgacagagccaccaaacacttgctgccattcccgacgacctataattgtgagttaggattttctaatttagtagaaataaagagtaagaaaagaaaccgaccggatgtggaacctgacctacggctcaagctatccgtcatcgagccggatatagataccttggtgaaagattgcaaacaatatcatccctctcactgatatgaaacaattattattattattattatttttataatttanattttatttttaaaattgtgatgaatatttttaaaatttacctaaaattggtggtctgcgtgtgcgccgaacgcccattaagtggtctgcggatgccgaaagtttgagaaacactg

SEQ ID NO: 11 - DNA, 325 bp, Mus musculus
cagtgatgagcaacccgcggcccgcccggcttcgcgatacggcccgcgatctaatttcaggatgaaagattagaaagctgcctccgtgttgctacttctcaaatatgcccagatattaacattttagtggctaaaaagcagtgccaattttctcattgacattcttctattcaataaagtaggtaatctaagttgtaagaatatacattttcccccgtgggactcaataaagctattttcattttgaatgaaaaaaaaatgcggcccgtaaacatttttatttttcctgaattggcccttatgcaaaaaaagttgctcaccactg

SEQ ID NO: 12 - DNA, 1237 bp, Mus musculus
cagatatttttatttctcctaacggacaatttttgattaaagctagaaagtcatagtcgcaagacaagcaataatccaaaaagtttaagcgttaattaggcaatataatgaggattatctcaaatttggattcgtgtgctccgacaattatcccttttctgcctaaatgtcttatttgcatggaaaagttatcaagtgaagcaatggcgccaagcgaccgcaccgcctcgccacgcaatatcatcttaccagaaaaaaaagatttgaactttttaagcgtctgcaagcgcaaaataagaaacaaagttcttttatgagatcggttacaacagtatcagatcgagctcaagaagctagttacaaggtcgcgcaattaatagctaaagccaaaatgcctcacgcaattgcagaatcgctcattttaccagcctgcgtggaaattgtcgacaccgtgtttgggaccaatgaagcaaaggaaatagaaaaggtgccactttcgaataatactattagtagacgcattgacgacatgtcagatgacaagacgacactaatccagaagattattaaatcaaaaaagttttcattgcagattgatgaatctacaattagtacattactaattgctcagttaatagcactagttagaatccctgaagagaaatgcttggaagaacattatttgttctgcaaagaagtaccaaaacaaactactgggaatgaaatattcaaagtggtaaatgaatacttcgaaacaaatagatatgacggaagcctgctgtgttgcgcacgatgtggctgcaatgacggaaggcgtaaaggctttacatcaagagttcgttctgaaaaccccgagattcaagtaatcgtcgttttattcacagcctctcgtatgaaagtttgcctgtagatctgaattccacattaaatgacgttatcaaatgagtgaatctaataaaatccaagccgctacagtcccgtctgtttcagctttatgtgaagaaatgggatcagaacaccgtctccgctgtttcataccgaagtgcgttggctgtccaaagaagagatttgtcaagagtttatgagctaaaagaagaaattggaacatgtgaacgattctcacttgcagattcgttactcggcttaaaaaatggtgtacttaactgacgtttttgagcacctgaatgaacttaatcgaaaattgttagtttgcactgtgtgaaactgacaatatctg

SEQ ID NO: 13 - DNA, 357 bp, Mus musculus
  misc_feature (355)..(355): n is a, c, g, or t
cagcaggccggattcatcaaaaggataacgggtagatattttccttttgtagaattttaacgaataaacggcattcctattcgttatttatctactttcgaattttaacgaatagttctagtgataattaccgaatttctatatttatagaaaaccggcacttcataaatatcgaattgtgctattatctacatatgtgccggttttctataaatatagaaattcggtaattatcactagaactattcgttaaaattcgaaagtagataaataacgaataggaatgccatttattcgttaaaattctacaaaagaaaaatatctacccgttatccctttgatgaatccggcccgntg

SEQ ID NO: 14 - DNA, 277 bp, Mus musculus
  misc_feature (52)..(52): n is a, c, g, or t
  misc_feature (63)..(63): n is a, c, g, or t
  misc_feature (87)..(87): n is a, c, g, or t
  misc_feature (123)..(123): n is a, c, g, or t
  misc_feature (198)..(198): n is a, c, g, or t
  misc_feature (204)..(204): n is a, c, g, or t
  misc_feature (208)..(208): n is a, c, g, or t
  misc_feature (214)..(214): n is a, c, g, or t
  misc_feature (271)..(271): n is a, c, g, or t
ataaaccacggggcggattcgcgaaggagagtcggtaatcgtcgattccgtntattttgcttntatgttttcttctgattcatgaancgcttttcgaaattcgaaaagcggttcatgaatcgntcgggagccggcaaaaattaatagtaatgagctcatttccatagaaatgggctcattaccatgccggctgccganaatttncgangccggnttcgcgccggcaaacggggtcctgcaggcggtgtccttccgcctgcccgcgggaaanaatccg

SEQ ID NO: 15 - DNA, 375 bp, Mus musculus
  misc_feature (21)..(21): n is a, c, g, or t
  misc_feature (24)..(24): n is a, c, g, or t
  misc_feature (46)..(46): n is a, c, g, or t
  misc_feature (334)..(334): n is a, c, g, or t
  misc_feature (351)..(351): n is a, c, g, or t
  misc_feature (354)..(354): n is a, c, g, or t
gcacgccgcaagaaaataaanaanacttcgaaaaggtcttgaaccngcgtccttacgcgctttataccgcggcccaacgcgcagccgctagaccgccccgccgcaggtaagaaatgagaaatttcgagggctattgaacactgcgaattttcacagcggatcagcacaaagttatttagcacaggtgtttctgtaattgtgatacattgggaaaattcacagtgttcaatggccctcaaactcacgcttccacctgcgtggtgcggtggtctagtgttagtacactgggccgtaatataaaacatgcgggaacgccggccggctcgagacccgntgaagaggttttcgttntancgtccgtgcttccttcgttcg

SEQ ID NO: 16 - DNA, 184 bp, Mus musculus
  misc_feature (20)..(20): n is a, c, g, or t
  misc_feature (37)..(37): n is a, c, g, or t
  misc_feature (165)..(165): n is a, c, g, or t
cattgcataaaaaataacgnatagccaactgtgaatnacgaggctgtaattccatctcggggttccggtgacgttaataaaccgctcgagcttcgctctcgtggtttacgacgtcaccagaacccctcgatggaattacagcctcgtaattcgcagttggctatncgttattctgtatgcaatg

SEQ ID NO: 17 - DNA, 168 bp, Mus musculus
  misc_feature (62)..(62): n is a, c, g, or t
taacagataccagggagtgagtgattcaaggctgtaatctaatctcggggttcagatgacgnttataaaccgctcgagcttcgctctcgcggtttatgacgtcatctgaacccctcgattagattactgccttgtaatcactcccaggtatccattattcttacgtaa

SEQ ID NO: 18 - DNA, 273 bp, Mus musculus
  misc_feature (244)..(244): n is a, c, g, or t
taattaagagataatgtcaatggaatagaacgttgtcacaggataatggtctcccgctgctagataaatgccgaggcgsaagccgagacgtttattttcaaagcaggagacattgatcctgtgacaacgttctattacaatgactttatttctattataccaaatgattgatgtagatttaatcattttgtctgatggatgttggtgcagtagagtgacagttgctcgccgtaccgttattganctgccgcgttccgatcggcttagagaaca

SEQ ID NO: 19 - DNA, 169 bp, Mus musculus
  misc_feature (3)..(3): n is a, c, g, or t
  misc_feature (60)..(60): n is a, c, g, or t
  misc_feature (165)..(165): n is a, c, g, or t
tanttaagggataatgttcatggcggaggagtatacgaagcaataaacggcttttgcggntgattaaacgccgaagcgaagctgaggcgtttgatcaaccgcaaaagccgtttattgcgagtatactccaacgccgtgaacattattcctattatacgacaaganaaaa

SEQ ID NO: 20 - DNA, 424 bp, Mus musculus
  misc_feature (42)..(42): n is a, c, g, or t
  misc_feature (55)..(55): n is a, c, g, or t
  misc_feature (234)..(234): n is a, c, g, or t
ttcctttcattcgtttaatcattttttcggttcaattttcantttttttagatgntacatttttaaatcagttcaatatgtctcgaaccgctacgctagaatgctgcttgactcacttccaaattgaagcgcttataaaaaaaaatttgaagcgctccaataattttaaatcgctctgcgctgcgcgtagcgatttaaaattattggagcgcttcaaatttttataagcgcttnaatttggaagtgatcggggttctgggcatgcgcagtgcagagcgatttaaaattattagagcgcttcaaattttttataagcgcttcagtttgaaagtgatcggggttctgggcttgtgcagcgtaaagcgatttaaaattaccggagtgcttcaaatcgttctcaacgtttgagtttgggaatttggag

SEQ ID NO: 21 - DNA, 316 bp, Mus musculus
  misc_feature (82)..(83): n is a, c, g, or t
  misc_feature (171)..(171): n is a, c, g, or t
cttaattaagcaataacgatcgaggcgcagggcatttcctggggattaatgaccggctgggaggagttgatggcccgaggcnnagccgagggccattaaccccagccggtcattaatccccaggaaatgccctgcgccgaggtcgttattgctattataagctgaaaacgnagaaacgaacaggcgtatggattttttttatgggcgatgcagtttcaattggtatgtacagggcatttctagagaattaatgccctgtatattagccaatcagatcgctcgaatcatctctcaacattccattcggcttataatt

SEQ ID NO: 22 - DNA, 192 bp, Mus musculus
  misc_feature (166)..(166): n is a, c, g, or t
tatttaagcaataatccccgagaaatcggtcgttaccagcagttaacgaccggtttgttagttaacggcccgaggcgaagccgagggcggttaacgctctaacaaaccggtcgttaactgcggtaacgaccgatttcgaggggattattgctattataaaccgtantcaacggtttataacagcaataatgt

SEQ ID NO: 23 - DNA, 248 bp, Mus musculus
  misc_feature (44)..(44): n is a, c, g, or t
  misc_feature (186)..(186): n is a, c, g, or t
ttaattaagcaataagacacgacagggcgtgaattatggcgtantaattcacgcctagtgcgttgttaggcacgaggccgaaggccgagtgccgtcaacgcaactaggcgtgaattattacgccgtaattcacgccctggagtgtcttattgcgattataaaattttattattaaaggttattttnaaaaaatatttatatatgttaattaagcgatggggctcataaattccgagcagtgaattatg

SEQ ID NO: 24 - DNA, 208 bp, Mus musculus
  misc_feature (37)..(37): n is a, c, g, or t
  misc_feature (83)..(83): n is a, c, g, or t
  misc_feature (182)..(182): n is a, c, g, or t
  misc_feature (187)..(187): n is a, c, g, or t
  misc_feature (194)..(194): n is a, c, g, or t
ttaatatagcattaagacacgacagggcgtgttttantggtccattaatacacgcctcgggtgcgttgcgaggcacaaggctnaaggccaagtacttcgaccacccgaggcgtgtattaatggaccaataaaacacgccccggagtgtcttaatgctattataatacggctctttaattttnaattnaattttnaagaattcttttca

SEQ ID NO: 25 - DNA, 302 bp, Mus musculus
  misc_feature (264)..(264): n is a, c, g, or t
  misc_feature (270)..(270): n is a, c, g, or t
  misc_feature (281)..(281): n is a, c, g, or t
taattaagcaataagacacgacaggcagtgcatttctgggcgattatagcacgcctcgggtggcgttataaggcacgaggccgaaggccgagtgactttaaccacccgagaagtgcaataatcgccccgaaatgcactgcctggagtgtcttattgctattatgaaatggaatttatacataaaaataaggaaaacagtcagacccgcgcatttaccgggcattattgacgtgggcgtgacatcaccgacagccaatcagaaanctccgnttgcgtccggngttctaaagccgtttcataat

SEQ ID NO: 26 - DNA, 292 bp, Mus musculus
  misc_feature (41)..(41): n is a, c, g, or t
  misc_feature (199)..(199): n is a, c, g, or t
  misc_feature (215)..(215): n is a, c, g, or t
  misc_feature (221)..(221): n is a, c, g, or t
  misc_feature (236)..(236): n is a, c, g, or t
  misc_feature (270)..(270): n is a, c, g, or t
taattaagcaataagacacgacaggcggtgcgtttctgggngattattgcacgcctcgggtgcgttgcgaggcacgaggccgaaggccgagtgacttcaaccacccgagaagtgcaataatccctcagaaacgcaccgcctggagtgtcttattgctattatgaaatggaaattatgaaaacgaaagagggagaaaggnctgacctgtgcatttnctgggnattactgacacgagnatgacatcgccgacagttctacgtgtgtccaganagttcgaaagtcactgcataat

SEQ ID NO: 27 - DNA, 562 bp, Mus musculus
  misc_feature (7)..(7): n is a, c, g, or t
  misc_feature (209)..(209): n is a, c, g, or t
  misc_feature (279)..(279): n is a, c, g, or t
  misc_feature (504)..(504): n is a, c, g, or t
cagcgtncgaggaggaccacgagattacgatcttaagattgtaacgagaatgggttaagtttgtaccatttcccgttctcgttacaatcatcgtcacgagaatggattcatgtcgtgccgttttctgttctcgttacaatctttgtcgcgagaacgaggtatcagaattttaatgcctaatacgttctgcgaaatacggcagcgtgctntactgctttgaccttttcaatattctgcattttgattggctggccattccgcctcttcctcacaggttancgaggctgtaaacaggggagaacgggaatggccagccaatcaaattacagaatattgaaaaggtcaaagtagtacagcatactgctataattcacagaatatatgaggcattaaaattccgatacctcattctcgtgacaaagattgtaacgagaacagaaaatggcacgacatgaatccgttctcgtgacaatgattgtaacgagaacgggaaatggtacaaanttaacccattctcgttacaatcttaagattgtaatctcgcggtcctcctcggatgctg

SEQ ID NO: 28 - DNA, 301 bp, Mus musculus
  misc_feature (155)..(155): n is a, c, g, or t
  misc_feature (299)..(299): n is a, c, g, or t
caaaggaaagtaaaatgcaaaaaatcctacgttaatgcaacgttacggttgagattttaaacgcacaaaagtcaggaaattcaaagttacggttcccacagcaaccgtaactcggccacattgcacatactgatattaaggcataaatttaacancatatacagtaatacaaaatacttctatgcgcaagggggccgagttacggttgccgtgggaaccgtaactttgaatttcctgacttttgtgcgtttaaattctcaaccataatgttgcattaacgtagttttttgcatttaatnta

SEQ ID NO: 29 - DNA, 240 bp, Mus musculus
  misc_feature (2)..(2): n is a, c, g, or t
  misc_feature (22)..(22): n is a, c, g, or t
  misc_feature (96)..(96): n is a, c, g, or t
  misc_feature (180)..(181): n is a, c, g, or t
  misc_feature (232)..(232): n is a, c, g, or t
  misc_feature (235)..(235): n is a, c, g, or t
  misc_feature (237)..(237): n is a, c, g, or t
cnaaatgtgatggcaacattanggttgagattttaaacgcacaaaatgtcaggaaattcaaaattatggttcccacggcaaccgtaactcggccanattgcacatatatattaaggcattaaagttaacactatgcgcagtcacgtaataaactatatatgcataaggggatggagttanngttgctatgggaaccataacttcgaatttcctgactttcgtgcgtttgaanttncntac

SEQ ID NO: 30 - DNA, 270 bp, Mus musculus
  misc_feature (30)..(30): n is a, c, g, or t
  misc_feature (68)..(68): n is a, c, g, or t
  misc_feature (130)..(130): n is a, c, g, or t
  misc_feature (137)..(137): n is a, c, g, or t
  misc_feature (142)..(142): n is a, c, g, or t
  misc_feature (144)..(144): n is a, c, g, or t
tatgaatattaaatacaaaaaactacgttnaaataacgttaagggtctgacttaaacccacaaaagcnaggaaattcagagttaaggctgacactccgtccttaactcaccccgtcgtgcccgggtatcncttaatnttctntnaaataggcacaacggcgtgagttaaggacggagtgtcagccttaactttgaatttcctggcttttgtcggtttgtgtcacaaccttaacgttatttaaacgtagttttttgtatttaatattcata

SEQ ID NO: 31 - DNA, 471 bp, Mus musculus
gacctgtaatatggcgagaaaacagaaaatcacggaaaatgagaaatacacactttaggacgtgaaatatggcgaggaaaactgaaaaaggtggaaaatttagaaatgtccactgtaggacgtggaatatggcaagaaaactgaaaatcatggaaaatgataaacatccacttgacgacttgaaaaatgacgaaatcactaaaagacgtcaaaaatgagaaatgcacactgaaagacctggaatatggcgagaaaacttaaaatcacggaacatgagaaatacacactttaggacgtgaaatatggcaaggaaaactgaaaaaggtggaaaatttagaaatgtccactgtaggacgtggaatatggcaagaaaactgaaaatcatggaaaatgagaaaaatccacttgacgacttgaaaaatgacgaaatcactaaaagacgtgaaaaatgtgaaatgcacactgaaggac

SEQ ID NO: 32 - DNA, 356 bp, Mus musculus
tgtggagagccgtgccgcgagcaatcgcgtgcgtgccgtgagcaatcgccattataagatggcgctggcttccactgcgcctaactagtaaacaagccttatgcgcaagtgcaagagtgaactcacgcctagtcactgcccatctcgcggcgtagtaatggggtgatgggcgagcaacgaatcaggagctgtcacgccacatcaggtgctgaaacgtcacgctgcgggctatataagcagcgccattttcccggttcggggtcttccctcctgataagtaagcaataaaagctttgccgcagaagattccggttgtcctgagtgtgttcttgccggcggggacaaaagctcgggat

SEQ ID NO: 33 - DNA, 385 bp, Mus musculus
  misc_feature (103)..(103): n is a, c, g, or t
tgtggagagccggtacgtgccgcgagcaatcgcgtgcgtgccgccagtaatctctgtggagagccgtgtgtgccgcgagcaatcgccattataagatggcgcnggcctccgctgtgcctaactagtaaacaagccttgtacgcaggtgcgagagtgaactcactcctagtcactcccattctcggggtgtaatagtggggtgatgggcaagcaacgaatcgggagctgtcacgccatatcaggtgctgaaacgtcacgctgcgggctataaaagcggcgccattttcccggttcggggtcttcctgaagaagcaagcaataaagcttttgccgcggaagattccggtttgttgcgtctttcttgccggtcgagcgggacgcaata

SEQ ID NO: 34 - DNA, 970 bp, Mus musculus
ccgtatttcctcgattctaagacgcacgttttttcacattttaacgtttctgaaatcgggatgcgtcttacaatcgatgtcaaaagaaacttgccagccgccaggcagaggagtaagttgtgacgtagttgtcattgcctgcgcatgtgcgaacttagccgtgcatagaaggtatctgttcatccgattgtcacctcagttgagttatttgcattggtagcaccacacgcggttgaattttaacttaaatttggatccctaattgtcgcttaaaatgtcttcaaaaagattacactatgatgcagcattgaaacgaaaagttattgtgtacgcagaagattgcctgtcacacgccaggcaatgcaattaaaggcagtagaaattgccaaatctctcggaatagatcatagaattttcaaagctaggagaggttggtgtgaccgattcatgcgtcgtgaaggactatcactcaggcgccgaacatctatctgtcaaaagcttccggctgactttcaagagaagctgtttaacttccagcgatacgtaattcaattaaggaaaaaacgaaactacgagtttaaccaaataggaaatgcagacgaaaccccggtattcttcgatatgcctcgaaattatactgtcaatcctaaaggtgctaaagaggtcaagatcacgagcacgggttatgaaaagcagcgtgtcaccgtgatgctatgcataactgccgatggccaaaagtgattcagaagaatctttagactctgaatgtgaagaaggcttagactcaaactttgattgtgatactgaagaagaaagtggtatgtaattgtatggataaatgtatgctattgtcggttagttaaaaaacataatgtacatttaatgtagtgttttttctcttccgaaaagctgttattaaatcgatggtgcatcttacaatcgatggcgtcttagaatcgaggaaatatgg

SEQ ID NO: 35 - DNA, 3509 bp, Mus musculus
ctcaacctgaactcagtcgtgattacccgctgaacttaagcatatcatttagcggaggaaaagaaactaaaaaggattcccttagtaacggcgagtgaaacgggaagagcccagcgccgaatcgatcagtctttggctgcttcgaaatgtggcgtataggtgtaagtttccagcagtgtcgtatgtccgaagtccttacgattgaggccataaaccagagagggtgcgagccccgttctggatagcggcactgttggttcgcttgctccttggagtcgggttgcttgaaagtgcagcctaaagtgggtgataaacttcatctaaggctaaatatcgactcgattgcgatagcgaacaagtaccgtgagggaaagttgcaaaggactttgaagagagagttcaagagaacgtgaaatcgctggagtggaaccggagacagttgatgttgcttggagacaagcttggtgactggtcgcttagttgtgatcgttgccgggtgtcgtttcctatgctacgccgacggcgttggctgctcgttctagcccgacagtgttgcccatctcgcaagagaaggtgtcttgctggcggtagtgggttcgtggcggctagcgtttagttacgctagtgtgtgtgacgtcggtgtgaaagtcgacgacgtttccgacccgtcttgaaacacggattgcggagtgcttgtctactgcgagtcaaagggtgttaaaaccttgcggcgaaatgaaagtaaaggtcagtctcgaattggccgacgtgggatctgtgttcttcggagtgcagcgcaccacggccctgtgcgtgtcacttgtgactgtgcagaggttgagcagttggcaaacgacccgaaagatggtgaactatgcctgagcaggatgaagccagaggaaactctggtggaagtccgtatcggttctgacgtgcaaatcgatcgatagacttgggtataggggcgaaagactaatcgaaccatctagtagctggttccttccgaagtttccctcaggatagctggatctcaggcagttatattcggtaaagctaatgattagaggccttggggacgtaatgtcctcaacctattctcaaactttcaatggatatgaagttgcagtttctttagtgaactgtcaacgtgaatgcgaggtccaagtgggccatttttggtaagcagaactggcgctgtgggatgaaccaaacgtggagttaaggtgcctaacttctcgctcatgagaccccataaaaggtgttggttgatattgacagcaggacggtggccatggaagtcggtatccgctaaggagtgtgtaacaactcacctgccgaatcaactagccctgaaaatggatggcgcttaagcgagagacctatactccgccgttgcgacatgtgcgttgtctagcgccaggtcgtaacgagtaggaaggtcgtggcggttgcgttgaaggctatgagcgtaggctcggctggagcttccgtcagtgcagatcgtaatggtagtagcaaatattcaagttcgatccttgaagactgaagtggagaagggttccacgtgaacagtagttggatgtgggtcagtcgatcctaaggtactggcgaacgccttgtatcatcggtggcgaaaagcttgcttttagtccccgcttgtcgaaagggaatagggttaatattccctaactgagatgcaaagattgtgttcttcggagcacaagcgcggtaacgcattcgaacttggttagtcgctcaaagaccgagctagagttttcttctctagttaaggaacggactccctggaattggttcagccagagatggggacgttgtttccgaaaagcaccgcggtttctgtggtgtctcgtgctctttgaacggcccttaaaacaccaagggaggctattaatttgcactcaatcgtaccgatatccgcattaggtctccaaggtgaacagcctctagtcgatagaataatgtaggtaagggaagtcggcaaactagatccgtaacttcgggaaaaggattggctccagtggttggaacggttggccagttggttgatgcttgtccggcgcagttctgtctgcttgatactttcgggttgatggcggactagtgattgtgcttgcttgcggacgctttctggtgtgtgcttggacctcggttctagtatcctgatcgctcatctaaacaaccgtactggaaccggtacggactcagggaatccgactgtctaattaaaacagaggtgacagatggtccttgcggacgttgactgtcactgatttctgcccagtgctctgaatgttaaatcgtagtaattcgagtaagcgcgggtaaacggcgggagtaactatgactctcttaaggtagccaaatgcctcgtcatttaattgttgacgcgcatgaatggattaacgagattcctactgtccctaactactttctagcgaaaccacagccaagggaacgggcttggcaaaaatagcggggaaagaagaccctgttgagcttgactctagtttgacattgtgaagagtcatgagaggtgtagcataggtgggagtcttcggacgacagtgaaataccaccactttcatcgactctttacttattcggttaaaagagaattggcttcacggccttttttcgaagcattaagcggagccattttatggcaccgtgactctcctcgaagacagtgtcaagcggggagtttgactggggcggtacatctatcaaatcgtaacgtaggtgtcctaaggcgagctcagagaggacggaaacctctcgtagagcaaaagggcaaaagcttgcttgatcttgactttcagtacgagtacagaccgcgaaagcgtggcctatcgatccttttaatcctgattgtttcaggtaagaggtgtcagaaaagttaccacagggataactggcttgtggcagccaagcgtccatagcgacgttgctttttgatccttcgatgtcggctcttcctatcattgcgaagcagaattcgccaagcgttggattgttcacccactaatagggaacgtgagctgggtttagaccgtcgtgagacaggttagttttaccctactgttgacttgttattgcgaaagtaatcctgcttagtacgagaggaacagcgggttcaaacatttggttcataaacttgatcgacagatcaatggtctgaagctaccatttgagagattataactgaacgcctctaagttagaatctcgccttgtcaaggcgaaaatttcttgcttcccggtgtcgggaggcatctctatctcgtggcaacacgagagcttatgccctatgtatggccttggcgtcgtagtgaattctgcgacgcttgccaacgccagatcactctggttcaatgtcggggcgctaaatcacttgcatacgacttggtctcttggtcaaggtgttgtattcagtagagcagtccttttatactgcgatctgttgagactatcctttgattgagttttttg

SEQ ID NO: 36 - DNA, 5035 bp, Mus musculus
cgcgacctcagatcagacgtggcgacccgctgaatttaagcatattagtcagcggaggaaaagaaactaaccaggattccctcagtaacggcgagtgaacagggaagagcccagcgccgaatccccgccccgcggggcgcgggacatgtggcgtacggaagacccgctccccggcgccgctcgtggggggcccaagtccttctgatcgaggcccagcccgtggacggtgtgaggccggtagcggccggcgcgcgcccgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgggtccggccgtgtcggcggcccggcggatctttcccgccccccgttcctcccgacccctccacccgccctcccttcccccgccgcccctcctcctcctccccggagggggcgggctccggcgggtgcgggggtgggcgggcggggccgggggtggggtcggcgggggaccgtcccccgaccggcgaccggccgccgccgggcgcatttccaccgcggcggtgcgccgcgaccggctccgggacggctgggaaggcccggcggggaaggtggctcggggggccccgtccgtccgtccgtcctcctcctcccccgtctccgccccccggccccgcgtcctccctcgggagggcgcgcgggtcggggcggcggcggcggcggcggtggcggcggcggcgggggcggcgggaccgaaaccccccccgagtgttacagcccccccggcagcagcactcgccgaatcccggggccgagggagcgagacccgtcgccgcgctctcccccctcccggcgcccacccccgcggggaatcccccgcgaggggggtctcccccgcgggggcgcgccggcgtctcctcgtgggggggccgggccacccctcccacggcgcgaccgctctcccacccctcctccccgcgcccccgccccggcgacggggggggtgccgcgcgcgggtcggggggcggggcggactgtccccagtgcgccccgggcgggtcgcgccgtcgggcccgggggaggttctctcggggccacgcgcgcgtcccccgaagagggggacggcggagcgagcgcacggggtcggcggcgacgtcggctacccacccgacccgtcttgaaacacggaccaaggagtctaacacgtgcgcgagtcgggggctcgcacgaaagccgccgtggcgcaatgaaggtgaaggccggcgcgctcgccggccgaggtgggatcccgaggcctctccagtccgccgagggcgcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcgagagtggacgggagcggcgggggcggcgcgcgcgcgcgcgcgtgtggtgtgcgtcggagggcggcggcggcggcggcggcgggggtgtggggtccttcccccgcccccccccccacgcctcctcccctcctcccgcccacgccccgctccccgcccccggagccccgcggacgctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagcgccgttccgaagggacgggcgatggcctccgttgccctcggccgatcgaaagggagtcgggttcagatccccgaatccggagtggcggagatgggcgccgcgaggcgtccagtgcggtaacgcgaccgatcccggagaagccggcgggagccccggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgcgcgccccccccacgcccggggcacccccctcgcggccctcccccgccccacccgcgcgcgccgctcgctccctccccaccccgcgccctctctctctctctctcccccgctccccgtcctcccccctccccgggggagcgccgcgtgggggcgcggcggggggagaagggtcggggcggcaggggccgcgcggcggccgccggggcggccggcgggggcaggtccccgcgaggggggccccggggacccggggggccggcggcggcgcggactctggacgcgagccgggcccttcccgtggatcgccccagctgcggcgggcgtcgcggccgcccccggggagcccggcggcggcgcggcgcgccccccacccccaccccacgtctcggtcgcgcgcgcgtccgctgggggcgggagcggtcgggcggcggcggtcggcgggcggcggggcggggcggttcgtccccccgccctacccccccggccccgtccgccccccgttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgcggccctgcgggccgccggtgaaataccactactctgatcgttttttcactgacccggtgaggcgggggggcgagcccgaggggctctcgcttctggcgccaagcgcccgcccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcgccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcgaacgatacggcagcgccgcggagcctcggttggcctcggatagccggtcccccgcctgtccccgccggcgggccgcccccccctccacgcgccccgccgcgggagggcgcgtgccccgccgcgcgccgggaccggggtccggtgcggagtgcccttcgtcctgggaaacggggcgcggccggaaaggcggccgccccctcgcccgtcacgcaccgcacgttcgtggggaacctggcgctaaaccattcgtagacgacctgcttctgggtcggggtttcgtacgtagcagagcagctccctcgctgcgatctattgaaagtcagccctcgacacaagggtttgtc

SEQ ID NO: 37 - DNA, 123 bp, Mus musculus
caggggtgatattcaaaatatttaacaaccggtacggcacgggcaccgaccaatcagaacggacgccggccgtaaacaaccggtacggccataccggtgcgtaccggctgaatatcagccctg

SEQ ID NO: 38 - DNA, 180 bp, Mus musculus
ccgtatttcatcgattctaagatgcacattttttcacattttaacatctctgaaatcgggatgcatcttacaatcgatggcatgtcatagtttaattggcagcattttttctttcttagtggtacataaaataatggtgcatcttacaatcgatggcatcttagattcgatgaaatatgg

SEQ ID NO: 39 - DNA, 729 bp, Mus musculus
  misc_feature (471)..(471): n is a, c, g, or t
  misc_feature (540)..(540): n is a, c, g, or t
  misc_feature (547)..(547): n is a, c, g, or t
  misc_feature (577)..(577): n is a, c, g, or t
ccgtatttcatcgattctaagatgcacattttttcacattttaacatctctgaaatcgggatgcatcttacaatcgatggcatcttacaatcgctgtcagccaggcggcagtcgtgacgtagttgtcattgcctgcacgtgtgcgaacttggtcatagctgttcatattgtcatcacttcaattgagttatgtgcattgttggtactacacgtgttgagtttaattgccatttaaaatgtcttcaaaaagattacactatgattcagcattgaaatgaaaagttattgtgtacacagaaaggcacggaaacagagcagcggggcgtaaatttgatattagtgaagcaaatattcgtcgttggaggaatgaccgcaattccatattttcttgcaaagcaacaaccaagtgctttatgggacctaagaaaggaagatacccacaagtagatgaagctgtgttacgttttgttnctgagatacgtgcaaaaggattgcctatcacacgccaagcaatgcaactgaaggcaggagaaattgccnaatcccncggaatagatgaaagaaatttcaaagcaanaagaggctggtgtgaccgattcatgcgtcgtgcaggactatcgttaaggcatcgtgtcatagtttaattggcagcgttttttctttcttagtggtacataaaataatggtgcgtcttacaatcgatggcatcttagattcgatgaaatacgg

SEQ ID NO: 40 - DNA, 399 bp, Mus musculus
tagggatgggcgaaccggccgcgttttgggttcgtcgaacatctcaaactattttcaaacgttttgggttcggcaaaacccaaaacgcatttttgccaagcacttttccccttaatttttaaacccatgtgtatttcaagggaaatttaatccatatgtttctgattcatttacacttaactcatcaaaatgttgttttgtaagagctatttgatgtccaagaagccttttgagccttttaatagcttttctaaacctttttccccttagaaacaggaagtcgcattttgccaagagtaaacgaactcgaacccaaaaggttcgagttcggttcgaaactcgaacccaggagttcaagtgggttctaaacttggcaaaaccattctctcccatccctam

SEQ ID NO: 41 - DNA, 210 bp, Mus musculus
agggcccgattttaattcgcgtaatattcccgttaataacaacgtctaattaagacatccgttaaaagtccgtaacgttaatttaacggagaaaatctaatagagttctattggaatttttccattaaattaacgttacggacttttaacggatgtcttaattagacatcgttattaacgggaatattacacaaattaaaatcgggccct

SEQ ID NO: 42 - DNA, 176 bp, Mus musculus
  misc_feature (24)..(24): n is a, c, g, or t
  misc_feature (111)..(111): n is a, c, g, or t
  misc_feature (122)..(122): n is a, c, g, or t
tcggcaacgctttataataagtgnctaatcattattaattcctttggtattcattgtaataacattaatcatgatgaactcatttggtattaatgtggatgtcatacgtanttccatagganttccactgtaatttagcattaattaactggaccattattttaaagtgttaccga

SEQ ID NO: 43 - DNA, 364 bp, Mus musculus
  misc_feature (164)..(164): n is a, c, g, or t
  misc_feature (261)..(261): n is a, c, g, or t
  misc_feature (286)..(286): n is a, c, g, or t
  misc_feature (361)..(361): n is a, c, g, or t
cagcagaacctcgctaattctcgcttcgctaatccgcgaacccgataattctcaccaaaacccggcggtctcaccccacttctcagcaaagatttaatagcagagagctgtagcgaggtctcatattactaagacttcattacttttacaaaatatactacagnacatttactagtgtactatgaagtattatcataaataattaaaactaaactacacttgtcaaaataaatgaacaaagtacattttgtgatgcagtanccttgatttttatcgtgtttgtttncttactcgctaattcgcaaaattcggtaatccgcaatgggtctccccgtcattagtgcgaattagcgaggttctnctg

SEQ ID NO: 44 - DNA, 469 bp, Mus musculus
  misc_feature (50)..(50): n is a, c, g, or t
  misc_feature (71)..(71): n is a, c, g, or t
  misc_feature (356)..(356): n is a, c, g, or t
tggaatcccgttataaggatcgatttgggcaacccccgtttcgatcgctncgtccgaatgatcgctacatncagatccatgaaacagcgagcttcccaaatcagacacgcgcggagaagcaaaatctccgttttgcgaggacggagcgagttctactaggcattttagtgccacggcaggtcagtcaagttataattggctctaattagcactcccacaagctgtaacattctttacctgcagccgagtggcactcaaaaaggtgagaaattctttcctacctttgaaaacatcaaagaaaatcaaagaaatcgcttccaatctgatccttacaaccgaatgccccgctgatcggnataagcgaggggcgaacgcatcagcaccaatgggaaatggctttcggaaagtaagatttgatccatatagccgaacgatcgctacgagcagtgatcagcgaaaccgaattcca

SEQ ID NO: 45 - DNA, 475 bp, Mus musculus
  misc_feature (76)..(76): n is a, c, g, or t
  misc_feature (108)..(108): n is a, c, g, or t
  misc_feature (122)..(122): n is a, c, g, or t
  misc_feature (176)..(176): n is a, c, g, or t
  misc_feature (213)..(213): n is a, c, g, or t
  misc_feature (269)..(269): n is a, c, g, or t
  misc_feature (303)..(303): n is a, c, g, or t
  misc_feature (327)..(327): n is a, c, g, or t
  misc_feature (422)..(422): n is a, c, g, or t
  misc_feature (467)..(467): n is a, c, g, or t
tctgcttttggcacgtaagcgtcaacaggtgtgatcaagcgtaaagaggcgcgcggcgccagcgcttcggcgctgncacgggagaagggcctcccgcggaagagatgncacttgcagcgttntgcaggctgcccgtctaaacccatcgttgcttggcacctatgccctagggcaanggtccgaccaacttgtgagcgggcaccgtgccatccnaacagatgggcacgagcgtaggcagccaagagaccatgtatgtgcatcaagtgtgnttgctgagggcaggattcccagccgggaacgtcnaaacggctgtccgtcctgagcttncgcgcctacggttaaggggacgtgccatcgctaatccagctctgagccggattaactttcaaaaataaaaaatagcttccgcggccgcgtgaggngagtttttggcccgcttgaatcgggcggagcggatcgggcgggcnggatgaag

SEQ ID NO: 46 - DNA, 174 bp, Mus musculus
tattatagcggcgccgttcgcgccgctatagttaaggttgtgtcagcgtttccattataaacccctattttcaggggtttataactcggccgtaaaaattcgctccgggctgaaacttggcatacaaggtctcagcccgggagcgaaattttttttataaattgaaaaaaaaaa

SEQ ID NO: 47 - DNA, 106 bp, Mus musculus
  misc_feature (3)..(3): n is a, c, g, or t
  misc_feature (96)..(96): n is a, c, g, or t
tantaaggggtctattctcctctcgatgtgcgcgcgtaactcccattaacgttaatgggagttacgcgcgtgcatcgagaggagaatagacccctnagtgtgcaca

SEQ ID NO: 48 - DNA, 140 bp, Mus musculus
  misc_feature (3)..(3): n is a, c, g, or t
  misc_feature (14)..(14): n is a, c, g, or t
  misc_feature (120)..(120): n is a, c, g, or t
  misc_feature (122)..(122): n is a, c, g, or t
ttnattaaagacantgggccaaattctgccctcggatacgcgcgcgcaactcccattgaagtcaatgggagttgcgcgtgcgtatctgagggcagaatttggccctctgtatttgaaatncnaagagagaagagcattcc

SEQ ID NO: 49 - DNA, 213 bp, Mus musculus
atgcaataataagcagatattgacttctgttgaggtgaacatcaagatttattgacccgagaggtaaatattgaccgaggcgaagccgaggtcaatatttacctcgagggacaataaatcttgatgttcaccgaaacacgaagtcaatatgtgtattgttacatacattccgaatgtcttcatcagaaatatctggaaatctctccgttacgg

SEQ ID NO: 50 - DNA, 344 bp, Mus musculus
cagtcgtccctcggtatccgtgggggattggttccaggaccccccgcggataccaaaatccacggatgctcaagtccctgatataaaatggcgtagtatttgcatataacctacgcacatcctcccgtatactttaaatcatctctagattacttataatacctaatacaatgtaaatgctatgtaaatagttgttatactgtattgtttagggaataatgacaaggaaaaaagtctgtacatgttcagtacagacgcaaccatccattttttttctgaatattttcgatccgcggttggttgaatccacggatgcggaacccacggatacggagggccgactg

SEQ ID NO: 51 - DNA, 705 bp, Mus musculus
cagtagtccccccttatccgcggtttcgctttccgcggtttcagttacccgcggtcaaccgcggtccgaaaatattaaatggaaaattccagaaataaacaattcataagttttaaattgcgcgccgttctgagtagcgtgatgaaatctcgcgccgtcccgctccgtcccgcccgggacgtgaatcatccctttgtccagcgtatccacgctgtatacgctacccgcccgttagtcatcgacatcgtctgctcctgacatccaaccatcgacatcgtcatggctcgatgatccaggatcacccgaagcagatgatcctccttctgacgtatcgtcagaaggtcaatagtagcctaacgctacgtcacaatgcctacgtcattcacctcacttcatctcatcacgtaggcattttatcatctcacatcatcacaagaagaagggtgagtacagtacaataagatattttgagagagagaccacattcacataacttttattacagtatattgttataattgttctattttattattagttattgttgttaatctcttactgtgcctaatttataaattaaactttatcataggtatgtatgtataggaaaaaacatagtatatatagggttcggtactatccgcggtttcaggcatccactgggggtcttggaacgtatcccccgcggataaggggggactactg

SEQ ID NO: 52 - DNA, 418 bp, Mus musculus
cagatgctcctcgacttacgatggggttacgtcccgataaacccatcgtaagttgaaaatatcgtaagtcgaaaatgcatttaatacacctaacctaccgaacatcatagcttagcctagcctaccttaaacgtgctcagaacacttacattagcctacagttgggcaaaatcatctggcaacacagtacactgtagagtatcggttgtttaccctcgtgatcgcgtggctgactgggagctgcggctcgctgccgctgcccagcatcgcgagagagtatcgtaccgcatatcgctagcccgggaaaagatcaaaattcaaaattcgaagtacggtttctactgaatgcgtatcgctttcgcaccatcgtaaagtcgaaaaatcgtaagtcgaaccatcgtaagtcggggaccgtctg

SEQ ID NO: 53 - DNA, 97 bp, Mus musculus
cagatgctcctcgacttacgatggggttacgtcccgataaacccatcgtaaagtcgaaaaatcgtaagtcgaaccatcgtaagtcggggaccgtctg

SEQ ID NO: 54 - DNA, 224 bp, Mus musculus
caggggtcggcaaactacggcccgcgggccaaatccggcccgccgcctgtttttgtaaataaagttttattggaacacagccacgcccattcgtttacgtattgtctatggctgctttcgcgctacaacggcagagttgagtagttgcgacagagaccgtatggcccgcaaagcctaaaatatttactatctggccctttacagaaaaagtttgccgacccctg

SEQ ID NO: 55 - DNA, 341 bp, Mus musculus
caggggtcggcaaactacggcccgcgggccaaatccggcccgccgcctgtttttgtacggcccgcgagctaagaatggtttttacatttttaaatggttgaaaaaaaaatcaaaagaagaataatatttcgtgacacgtgaaaattatatgaaattcaaatttcagtgtccataaataaagttttattggaacacagccacgctcattcgtttacgtattgtctatggctgctttcgcgctacaacggcagagttgagtagttgcgacagagaccgtatggcccgcaaagcctaaaatatttactatctggccctttacagaaaaagtttgccgacccctg

SEQ ID NO: 56 - DNA, 386 bp, Mus musculus
caggggtcggcaaactacggcccgcgggccaaatccggcccgccgcctgtttttgtacggcccgcgagctaagaatggttttaacagatgaacatttgcaatcgatttcgatgatagggaacactaactttgaaccccaattaagcaaaatgttatctccccaaaaagaattccattcttctcattagtagacctgtattacaaaaaattgtactcaattattattattattatattttgaatttcatcaataaaaattttgtggaaatttgttttctctcttgttatataagtacctacataatatcctcgattttgcctcttggcccgcaaagcctaaaatatttactatctggccctttacagaaaaagtttgccgacccctg

SEQ ID NO: 57 - DNA, 263 bp, Mus musculus
cagtgctactcaaagtgtggtccgcggaccggtgccggtccgcgaactgtttgttaccggtccgcgacgagataagtacagaaattgagagtaagcgtttagaaacttttatagcaatttgacattgccgcgacatccaagtacgtgatcatttttctagtaattcatttttattgtattttacaaaagtatcggtctgcgacggattggagaaaacaaaaaaaaaaactggtccttcaccacagatagtttgagaagcactg

SEQ ID NO: 58 - DNA, 865 bp, Mus musculus
cagcaggtcctcgaataacgtcgtttcgttcaacgtcgtttcgttataacgttgatgagaaaaaaaatcgattcccggccggggccactgtctgtgtggagtttgcacgttctccccatgtctgcgtgggttttctccgggtactccggtttcctcccacatcccaaagatgtgcacgttaggttaattggcgtgtctamatggtcccagtctgagtgagtgtgggtgtgtgtgtgagtgcgccctgcgatgggatggcgtcctgtccagggttggttcccgccttgcgccctgagctgccgggataggctccggccacccgcgaccctgaactggaataagcgggttggaaaatgaatgaatgaatgaatacaaattattgtaaaataaaaatttataaagtatacgataatcatacaaatgcacgacaataaatgatgtggtacgaaagtgctcagcgagcccgccatatttgtgattgtttgtttttgaactgcgtggtggtaggaggtgctccttacaattttcgctttgcaaacatttattccttgatttaacccaccaccactacgaccgccgtcactcactgattcaccaaaaattgggtaaataattatcttacttgtttttattaatctttcttaaatgtatgtatagctcacatttatttcaatgtttaatattagaagtgttttggtctttatttagaagtttggtgatgtttttgtgaccagaaatatgccgtaggaacttaactcttgtttatatcaattagcctatggtaaaattggtttcgttatacgtcgtttcgcttaaagtcgcagtttccaagaacctatcgacgacgttaagtgaggacttactg

SEQ ID NO: 59 - DNA, 202 bp, Mus musculus
cagtaagtcctcacttaacgtcgtcgataggttcttggaaactgcgactttaagcgaaacgacatactgtatgccataggaacttaactcttgtttatatcaattagcctatggtaaaattggtttcgttatacagtacgtcgtttcacttaaagtcgcagtttccaagaacctatcgacgacgttaagtgaggacttactg

SEQ ID NO: 60 - DNA, 1205 bp, Mus musculus
cagtggcgtaccaagggcggggcggtgggagcggtccgccccgggtgcaggcaataagggggtgcattgtctgtagagaatttaaaaacaataataaaaccgactaaaagtcggtctgctttttattatcaccatgcgccggcaattctaaacaatgtcagtgataaaatactcctccccgaaaaatcttttgttggtctaagttctaaacaattgctgcggttactgttgagttttaataatatatatgtaagcttcaaattagcacatttttattacttatcctttaataaacattgtattctacatggaagttaattcggagaactcccagttatacagtcggcccccgacacacgcggactcagctacacgcgttcgtttcgagagtaagttcgtaacggttcggaatcgttcgagctcgcttcgggcgcagttcgtgtctccaacccctgtggtactacatattcctgcgtttaaacagtagattcgaaataaacaatgatagcacagtgattgtaaagacgaagaaacagaacttgagttacttcaattctgtcattctatgtgaccacttggagtttttatttgtgtttaaaatttaaaacagtgaaacagagtgcgaactgcgaggtgtaatatttttgtttggtaagtgcaaattttagacttttcatatttgtatatctgttgcttcatgtgaaagaaacttttcgaaattaaaattaataaaaagtgttcttcgatcaactatgagcgaagatagattgacaaatctggctatactgtctattgaacatgaatatgcgaagaagatcaattttgacgaagtcattgacaaatttgcagaagttaaggctcgaaaacagaaactgtaatgttattattcattactgcgacagaccaatatgtaggtataattttttccttttttcaaaaaatacattaatgtaattaaaaagtattaatccattactttttttccttttttgtactgtaatatttattttttattttttatactggcatgattatatatacgaagttcaataaaagaaaattttcactgtctgcgtttcttttctggccattattattattcgtttcatttcatgattattactgaaaataattttgtcgtatagaggaggggggtgttaaaaaatgatccgctccgggtgtcaaatacgctaggtacgccactg

SEQ ID NO: 61 - DNA, 756 bp, Mus musculus
caggtatccctcgctatctgaactctcactatccgaatattcgctataacgacttgcaaaaatttttacccaaaattcactatccgaatcgaaaacctgctataatgaatctgcatgtgcgcgccagcgaaaacgtttaagttgcgcgcgagtccgggcgagaggatgtagagtgcgctgcagtcgtatctcagctgttctcccgataggatcgcgtctcgtgctcgcgttgtttaaacgtgttgtgcattatcgctatcatcttccccaccttttccctgagggtttagcccttcatgggtcccagtgtttgcttctgccaggcgcctgggggcactaccaacccgggtccaatttagatagtatctttaacatattatttcattgtttatttacattacagtacatgttcgttgcagtgtagaaggaaaacgtaattcgtatccgatactgtacagtatcgttgcgtactgcacacaaacatacccactaatgagttcattaagtgttaaataattaggtaattggtgttttaaatgctttatattatgcagaaatccttggtggattgttatataggtgtttaagagtgttttagtgatatttggggaaattggttggggtttttggatgggctgggaacgcattattatttttcccatttaaaataatggaatataggctcccgctatccgaaaattcgctatccaacacgttttcaggaacggattagattcggataacgagggatgcctg

SEQ ID NO: 62 - DNA, 1708 bp, Mus musculus
gggtttggatcataatcccaaaagacacaatcccaaacgccataatcccgaatgttgaaatcccgaaagatcaaaatccctaaagtctaaaatccctaaagtctaaaatcccaaaaattcacacaggatggttgcatcatgttaggcagaactgttattttcttattgtctttatgcagaaaaaatggattttaattgaatccccaaaccataatgacagatttggaattaggtgcgatcaaggcttctaaaagtgaatttcaaggtgttaccaataaagtttgtttttttccattcagcccaatgcatttggtggaaaattcagatgagtggattggccatgcgatacggcaacgacgaaaacttcagtttaaaaatgcgtcatttgcctgcattggcattccttccagctgatgacattccgggagcttttaatgaattaaagccgcatttgcctgaagaagtcagcgaagttactgactggttcgaaaataattatgtgcacggtaggataagaagacacttacacaacggtgttgccgttcgattaccagtattgtttctaccaaatttgtggtctgtatatgagtgcatgcagaatggatttctatatacccaaaacaacatagaagcatggcacagaagatgggaaaatttaatagggaatgctcatgtcggtgtatatcgaatcagaagattcaaaaagagcagcgccacgtagaaaatgaatgtgaacatattctccgaggagagccatgtcctaaaagaaaaaaaaaagcagctattcatcgcgatgcaagacttcaaaatatagttaatgatcgtgaaagtcggccagctcttatggactatctccgtgcaattgcccataatctatccctgtaatatactttttcatatgtcgaattttctttttagttttttttcactattttaaattgtcagcattattttttacaattcgctatgctatgtatttcatcttcgcatcatttccaatactggaggtataaattgtgtaaagacttttagagagttctaattcgttttatgcattttttgcaaatttgactccacgaaagtgcattatcacaacgttgactttgtgtgtaagcattgtgcgtgtacgtaaaaacgttgaaacttcctcaataaatgaagagatgtcctttttgtacatctgcatttgtgaaagataaaatttctcgagatctcggctctttgggcgactgcatatgcagtggtgacccatcgcggtttttgatcgatctcgtcaaaagacttaggttgttcgtcacggtatttcagatgaccgcagttataaagctgggtgcacacaattaccaaccatagtgatatgcgtttatacatttccctttttgacctatttctttatgaatacggttcgtctgctcataactgttatacccgtgcgactgtcattagtatacctgagtgtttatgcttgcaaaaatatgtatgttattattgcctattttattgtgtaaagtggcctatgaagtgttctgtcatgtttttatatgtttctcaaataaatccccttttaaaaatgtaaataaatatcttttaaaaaatttttaaattattttttccagaattatatttttgggattttgatctttcgggatttcaacattcgggattatggcgttcgggattgtgtctttcgggattatgatcggctccc

SEQ ID NO: 63 - DNA, 1181 bp, Mus musculus
  misc_feature (1067)..(1067): n is a, c, g, or t
gggtttggatcataatcccgaaagacacaatcccgaacgccataatcccgaatgttgaaatcccgaaagatcaaaatccctaaagtctaaaatccctaaagtctaaaatccctaacgtctaaaatcccgaaaatcacgaatcatagaagaatttcaaaaagagcagcgccacgtagaaaatgaatgtgaacgtattctccgaggagagccatgtcctaaaagaaaaaaagcagctattcatcgtgatgcaagacttcaaaatatagttaatgatcgtgaaagtcggccagctcttatggactacctccgtgcaattgcccataatctatccctgtaatacactttttcatatgtcgaattttctttttagtttttttcttttcttttttagtttttttcactattttaaattgtcagcattattttttacaattcgctatgctatgtatttcatcttcgcatcatttccaatactggaggtataaattgtgtaaagacttttagagagttctaattcgttttatgcattttttttgcaaatttgactccacgaaagtgcattatcacaacgttgactttgtgtgtaagcattgtgcgtgtacgtaaaaacgttgaaacttcctcaataaatgaagagatgtcctttttgtacatctgcatttgtgaaagataaaatttctcgagatctcggctctttgggcgactgcatatgcggtggtgacccatcgcggtttttgatcgatctcgtcaaaagacttaggttgtccgtcacggtatttcagatgaccgcagttataaagctgggtgcacacaattaccaaccatagtgatatgcatttatacatttcgctttttgacctatttctttatgaatacggttcatctgctcataactgttatacccgtgcgactgtcgttagtatacctgagtgtttatgcttgcaaaaatatgtatgttattattgcctattttattgtgtaaagtggcctatgaagtgttctgtcgtgtttttatatgtttctcaaataaatccccttttaaaaatgtaaataaatgtcttttaaanaattttaaattattttttccagaattatattttcgggattttgatctttcgggatttcaacattcgggattatggcgttcgggattgtgtctttcgggattatggcccaaaccc

SEQ ID NO: 64 - DNA, 202 bp, Mus musculus
  misc_feature (93)..(93): n is a, c, g, or t
ccacgcgtgtccaaccttttgacattgcaacacgacgttgtcatctacaaagttcacgctcgagaaccgcgcaatcgagttacaaaagaaatntcaaaaaaactcataatgttttaagtaagtttatgattttgtgttgggccacattcatagctgtcctcggctgcatgtggcccatgggccgcgggttggacatgcctgg

SEQ ID NO: 65 - DNA, 1647 bp, Mus musculus
ttgattcatcaatgaaattgcgtacggctcattagagcagatatcaccttatccgggatcctcatatggataactgcggaaatactggagctaatacatgcaactataccccaacgcaaggcggggtgcaattattagaacagaccaaacgttttcggacgttgtttgttgactctgaataaagcagtttactgtcagtttcgactgactctatccggaaagggtgtctgccctttcaactagatggtagtttattggactaccatggttgttacgggtaacggagaataagggttcgactccggagagggagccttagaaacggctaccacgtccaaggaaggcagcaggcgcgaaacttatccactgttgagtatgagatagtgactaaaaatataaagactcatccttttggatgagttatttcaatgagttgaatacaaatgattcttcgagtagcaaggagagggcaagtctggtgccagcagccgcggtaattccagctctcctagtgtatctcgttattgctgcggttaaaaagctcgtagttggatctaggttacgtgccgcagttcgcaatttgcgtcaactgtggtcgtgacttctaatttgctggtttgaggttgggttcgcccttcaactgccagcaggtttaccttgaataaatcagagtgctcaatacaagcgcttgcttgaatagctcatcatggaataatgaaacaggacttcggttctttttgttggttctagaactgatttaatggttaagagggacaaaccgggggcattcgtatcattacgcgagaggtgaaattcgtggaccgtagtgagacgcccaacagcgaaagcatttgccaagaatgtcttcattaatcaagaacgaaagtcagaggttcgaaggcgattagataccgccctagttctgaccgtaaacgatgccatctcgcgattcggagggtttttgccctgccgaggagctatccggaaacgaaagtctttcggttccgggggtagtatggttgcaaagctgaaacttaaagaaattgacggaagggcaccacaaggcgtggagcttgcggcttaatttgactcaacacgggaaaactcacccggtccggacaccattaggactgacagattgaaagctctttctcgatttggtggttggtggtgcatggccgttcttagttggtggagtgatttgtctggtttattccgataacgagcgagactctagcctgctaaatagttggcgaatcttcgggttcgtataacttcttagagggataagcggtgtttagccgcacgagattgagcgataacaggtctgtgatgcccttagatgtccggggctgcacgcgtgctacactggtggagtcagcgggtttttcctatgccgaaaggtatcggtaaaccgttgaaattcttccatgtccgggatagggtattgtaattattgcccttaaacgaggaatgcctagtaagtgtgagtcatcagctcacgttgattacgtccctgccctttgtacacaccgcccgtcgctatccgggactgaactgattcgagaagagtggggactgtcgcttcgaggtttaacgacttcgttgttgcggaaaccatttttatcgcattggtttgaaccgggt

SEQ ID NO: 66 - DNA, 1869 bp, Mus musculus
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 67 - DNA, 791 bp, Mus musculus
  misc_feature (569)..(569): n is a, c, g, or t
cagtagacccttggcattcgcggatttaacattcgcggtttcgactattcgcgagcgaccccgaaggtccatgacatgtagtaatttgtaattttgctgaggcacgaatttgaatcgcatgcgctgcgaggctggtgtgcaggagcgagtcacttagctagtgagtgagcctagccgaccgcccagcatccgcatctcaacgcggctttgttgttctctactcatcgtcgcgtacgcagtaactctcgtgaagtgataaaaactttgtttctttgtgaaaaatggccccgaaaagaaagccaactgctagtgctggtgatggaagtgaagagaaagtgaagaggtctaagaaagtgatggttcttagccagaaaatagaagttttggataaattaaagagtggaatgtcgaattcggcggtggctcggatctatgacgtgaacgagtccaccatatgctctatacggaaacaagaaaaagcgattcgtgaaactgtttcagcgagtgctccagccagtgcaaaaattgctcatcaataataggaaacaagaaaaagcgattcgtgaaantgtttcagcgagtgctccagccagaatttattttaatagctttataaatgactttagtcctgtatttatagaatcattaagggtctgaaggggtcacttaaatttttcagttatactttactgcattttatgggggaaattatatgctatagtggtatttgcgaatttggggattcgcgaaggtctcgggacgtatccctcgcgaatgtcaagggtctactg

SEQ ID NO: 68 - DNA, 726 bp, Mus musculus
  misc_feature (392)..(392): n is a, c, g, or t
  misc_feature (500)..(500): n is a, c, g, or t
cagttgacccttgaacaacacgggtttgaactgcgcgggtccacttatacgcagatttttttttttttgagatttgcgacaatttgaaaaaactcgcagatgaaccgcatagcctagaaatatcgaaaaaattaagaaaaagttaggtatgtcatgaatgcataaaatatatgtagataatagtctattttatcatttactaccataaaatatacacaaatctattataaaaagttaaaatttatcaaaacttacgcacacaattacagaccgtacatggcgccattcgcagtcgagagaaatgtaaacaaacgtaaagatgcagtattaaatcataactgcataaaattaactgtagtacatactgtactactgtaataatttcgtagccncctcctgttgctattgcggtgagctcaagtgttgcgagtatccgcttaaaacgccgtgtgacgctgatcatctccgcgtgagcagttcgtctctccagtaaattgcgnatcgcagtaaaaagtgatctctcgcggttctcgcgtatttttcaccgtatataatacatatgacatacaaaatatgtgttaatcgactgtttatgttatcggtaaggcttccggtcaacagcaggctattagtagttaagttttgggggagtcaaaagttatacgcggatttttgactgcgcgggggatcggtgcccctaaccccgcgttgttcaagggtcaactg

SEQ ID NO: 69 - DNA, 366 bp, Mus musculus
cagacggtccccgacttacgatggttcgacttacgatttttcgactttacgatggtgcgaaagcgatacgcattcagtagaaaccgtacttcgagtacccatacaaccattctgtttttcactttcagtacagtattcaataaattacatgagatattcaacactttattataaaataggctttgtgttagatgattttgcccaactgtaggctaatgtaagtgttctgagcacgtttaaggtaggctaggctaagctatgatgttcggtaggttaggtgtattaaatgcattttcgacttacgatattttcaacttacgatgggtttatcgggacgtaaccccatcgtaagtcgaggagcatctg

SEQ ID NO: 70 - DNA, 446 bp, Mus musculus
cagatgctcctcgacttacgatggggttacatcccgataaacccatcgtaagttgaaaatattgtaagtcgaaaatgcatttaatacacctaacctaccgaacatcatagcttagcctagcctaccttaaacatgctcagaacacttacattagcctacagttgggcaaaatcatctaacacaaagcctattttataataaagtgttgaatatctcatgtaatttactgaayayartacactgtagartayyggttgtttaccctcgtgatcgcgcggctgactgggarctgcggytcactgycgctgcccagcatcgcgacagagtattgtaccgcatatcgcyagcctgggaaaagatcagaaattcgaagtacggtttctactgaatgcgtatcgctttcgcaccatcgtaaagttgaaaaatcgtaagttgggaaccatctg

SEQ ID NO: 71 - DNA, 624 bp, Mus musculus
cagtcagttctgctataacgcttgttttgaaaacgcgaatttgttccaacgcgattgatatattagggaacaatttgagcataacgcgaatttcgcgtttgcttatgcgcgatttcgtccgcgagaaacactaggtgaacgcagaaaactgcacccagctgaaccgagccgcgtaggaatacacaaaacgcacacacgcacacacctcaaacatctaccagctacctcagttcaccgcgtgtgttatgagccacacccatccacatctggtgttacaactttccatccgatttcagataaccctccttccaccacttcacaataactcacaagctgcaacccttccgacgcccacttccacaagcaaacttcaggtctttttcaaggtaaagtgccatatttattgtagtatttatgtatttcttaaccatttaacatgtgtaaaactgtgctaccatttttattaggttcctatcttttttttttatgtgtcactgacgaagtttttgagtgttgtgcccctaaccccattttccccataagccctgtggtttttattgcgcgattttgcatagcgcggtgatttttaggaacgcatatgtcgcgttatagcagaactgactg

SEQ ID NO: 72 - DNA, 76 bp, Mus musculus
gaccacgtggcctaatggataaggcgtctgacttcggatcagaagattgagggttcgaatcccttcgtggttacca

SEQ ID NO: 73 - DNA, 76 bp, Mus musculus
ggccgcgtggcctaatggataaggcgtctgattccggatcagaagattgagggttcgagtcccttcgtggtcgcca

SEQ ID NO: 74 - DNA, 75 bp, Mus musculus
tcctcgttagtatagtggtgagtatccccgcctgtcacgcgggagaccggggttcgattccccgacggggagcca

SEQ ID NO: 75 - DNA, 75 bp, Mus musculus
gccatgatcgtatagtggttagtactctgcgctgtggccgcagcaacctcggttcgaatccgagtcacggcacca

SEQ ID NO: 76 - DNA, 77 bp, Mus musculus
gctccagtggcgcaatcggttagcgcgcggtacttataatgccgaggttgtgagttcgagcctcacctggagcacca

SEQ ID NO: 77 - DNA, 77 bp, Mus musculus
ggccggttagctcagttggttagagcgtggtgctaataacgccaaggtcgcgggttcgatccccgtacgggccacca

SEQ ID NO: 78 - DNA, 85 bp, Mus musculus
ggtagcgtggccgagtggtctaaggcgctggatttaggctccagtcatttcgatggcgtgggttcgaatcccaccgctgccacca

SEQ ID NO: 79 - DNA, 86 bp, Mus musculus
gtcaggatggccgagcagtctaaggcgctgcgttcaaatcgcaccctccgctggaggcgtgggttcgaatcccacttttgacacca

SEQ ID NO: 80 - DNA, 76 bp, Mus musculus
gcctcgttagcgcagtaggcagcgcgtcagtctcataatctgaaggtcgtgagttcgagcctcacacggggcacca

SEQ ID NO: 81 - DNA, 75 bp, Mus musculus
ggctcgttggtctaggggtatgattctcgcttcgggtgcgagaggtcccgggttcaaatcccggacgagccccca

SEQ ID NO: 82 - DNA, 85 bp, Mus musculus
gacgaggtggccgagtggttaaggcgatggactgctaatccattgtgctctgcacacgtgggttcgaatcccatcctcgtcgcca

SEQ ID NO: 83 - DNA, 85 bp, Mus musculus
gcagcgatggccgagtggttaaggcgttggacttgaaatccaatggggtctccccgcgcaggttcgaaccctgctcgctgcgcca

SEQ ID NO: 84 - DNA, 85 bp, Mus musculus
gtagtcgtggccgagtggttaaggcgatggacttgaaatccattggggtttccccgcgcaggttcgaatcctgccgactacgcca

SEQ ID NO: 85 - DNA, 85 bp, Mus musculus
gtagtcgtggccgagtggttaaggcgatggactagaaatccattggggtctccccgcgcaggttcgaatcctgccgactacgcca

SEQ ID NO: 86 - DNA, 76 bp, Mus musculus
ccttcgatagctcagctggtagagcggaggactgtagatccttaggtcgctggttcgattccggctcgaaggacca

SEQ ID NO: 87 - DNA, 236 bp, Mus musculus
  misc_feature (7)..(7): n is a, c, g, or t
  misc_feature (45)..(45): n is a, c, g, or t
  misc_feature (48)..(48): n is a, c, g, or t
  misc_feature (59)..(59): n is a, c, g, or t
  misc_feature (207)..(207): n is a, c, g, or t
taggttncgaatatgcgtagctcgtttcgtctctgacaataattncanataccctacgntaggaaacttggcccgcaaattacccacaaaaattcgggccgggtgttcggtacccgaattaattacccgaaaatgactgcctggggttggaccaagtattgtctcatcagcattcagcaccactgccatagcatgaaggagaaaagnaaacacagaaacgcgagaatgaaagaaga

SEQ ID NO: 88 - DNA, 340 bp, Mus musculus
  misc_feature (13)..(13): n is a, c, g, or t
  misc_feature (18)..(18): n is a, c, g, or t
  misc_feature (36)..(36): n is a, c, g, or t
  misc_feature (190)..(190): n is a, c, g, or t
tctcctcttcttnccccntcccgttcttcctttctncaccgctctcatagacttgaacggcgaaagccgtctacagttcatctaaggatcaaacaccttaagctgttgttttcaagttttattaatgttttccaactcatttcctattttcctgctgaaaaccctgccaaaagcactctttggcggatantaaaataattcggatagcagacatccgatccaaattttttgcggataattagcggatcggatatccgcgagaagcgggtaatttttattatccgcggatagttcgctaccgcggatattttactacccgcacatctcaatttctccagag

SEQ ID NO: 89 - DNA, 289 bp, Mus musculus
  misc_feature (14)..(14): n is a, c, g, or t
  misc_feature (24)..(24): n is a, c, g, or t
  misc_feature (33)..(33): n is a, c, g, or t
  misc_feature (37)..(38): n is a, c, g, or t
  misc_feature (77)..(77): n is a, c, g, or t
  misc_feature (256)..(257): n is a, c, g, or t
  misc_feature (273)..(273): n is a, c, g, or t
ttcatacatagtanctttcattantaacatcgncacnnttattacgcatcttcgtttagaagccgccttcgtttagnagccgccctcatttagtagccgcacctttaccatgcaagccgcaggggaaagtaattaaatttaatagaagccgccctcgttttgaagccgccctcgatttaaagccgcagggggaagtaattaaatttaatagaagccgcggcttctaaacgaagatatacggtatttgcagtcatgnntactacgacttttatncaagcgtgcatgtact

SEQ ID NO: 90 - DNA, 330 bp, Mus musculus
  misc_feature (24)..(24): n is a, c, g, or t
  misc_feature (42)..(42): n is a, c, g, or t
  misc_feature (52)..(52): n is a, c, g, or t
  misc_feature (54)..(54): n is a, c, g, or t
  misc_feature (65)..(65): n is a, c, g, or t
  misc_feature (308)..(308): n is a, c, g, or t
cgtagactttagtaataagtttgntgcgctatactgttctgnagctcgaagntnaattcaaatgnttcttgatttacatggaaatatactgtaaaacgcgaaattaacgcgtcaagttaatttcgcgctcccctcgcctcgggctgattagcgcaaattaaatttcacgctaatcagcccgagcagttagcgcgcaatacggaaatccgggatttccgctgattgcggtaaaaatatattcgcgctatttgcgcaatgcgcgaatatcgcgaaaatatttttatagcagcatttaatagttttacagnatttaatcaagacggaaaatta

SEQ ID NO: 91 - DNA, 343 bp, Mus musculus
  misc_feature (36)..(36): n is a, c, g, or t
  misc_feature (58)..(58): n is a, c, g, or t
  misc_feature (60)..(60): n is a, c, g, or t
  misc_feature (62)..(62): n is a, c, g, or t
caaaccatgaacctttatctgaaattcgtaaagttngagaagactggatgattttttntntnttatttttcattttcgcgcgcctctgcacttcctggttccggccgggaccggaagcggaagtgccgaaataccgcgagaaaggctgttctcgcggtatttccggcccgaccggaagcaggaagtaccggaaatctcgcgagaaagcctgttctcgcgagatttccagcccaattttgcagacccaagaggttcggataagcttcagataagcatccgaacttctgggtgcttatccgaactgaaactccgaaccttttgaggttcgcccatcactgataat

SEQ ID NO: 92 - DNA, 1268 bp, Mus musculus
  misc_feature (24)..(24): n is a, c, g, or t
  misc_feature (34)..(34): n is a, c, g, or t
  misc_feature (315)..(315): n is a, c, g, or t
  misc_feature (904)..(905): n is a, c, g, or t
  misc_feature (1033)..(1033): n is a, c, g, or t
  misc_feature (1178)..(1178): n is a, c, g, or t
  misc_feature (1182)..(1182): n is a, c, g, or t
  misc_feature (1216)..(1216): n is a, c, g, or t
  misc_feature (1231)..(1232): n is a, c, g, or t
  misc_feature (1238)..(1238): n is a, c, g, or t
tgtatttatgcaagttcgcacatnttaaactctntattttctaatagacaagcgtttagggctagattttcaaagagtgcattttaagcgcgcaattaggggcaatgacgcgcaaaattgcgcgcgcaaagaaatagaattattttcagaagtgcgattgaagcgcctagtgcaactgaaaataagggatttgtgctgcccaattgcgcgcgcaacctatcatcagattttcaaaaaattcagggaaaagtttctgtgctcgcagttgcacactgatcagcgccctctcgctcattaacatacgcgtcccctccnatttgtgctctcatttgcactgtgtaaacagcttctaacagctcctttcccctcttcgttcaaacaaacaatggcctttgtaaccaagaagaaaattagtagcaggtgaaagcttatttttaatgatgctgagaccaaattgttatttaacttaaaacattaaaacactcctttccctcaaatcccttttaattcctcgcagatattaattatagtttgtcaaacctgtacctctgaaatacttgctcctctgaaagagttcaaaaactaaccctttgcctgtataccctattagcaggataaaaacgccttttctctttcactatttttccaatacatatgatgaaaacatatgagatcgttgggatttatataggcagatttggcagccttttcgtatttacttggactgtgagatataaatcgaatttggggctctctctccaagaataagttatttgttatctgaataagtatgtgttagtggatcagacatggataaggtgtttctattataactgtgtttgttaggtatgatttacattgttctatattgttttataccgtttttcattttcggttacttaannttttttctgttttgttttatgtatttagtcacggttggttggttttttaactttgtaacactttaatgcgaaggaaattaaaacaacaacaaaagaaaacattttctaaatgtgttcgcaatcaaatntatccctcggtaatttgatttgtaggaaacggagagcaaagcattacaaacagagtgcttttcaataattcataattccttaaacgtgcaaatccgtcggctaagtttaggagcgcaatttgcgcctctaactctgttgaaaatncacncgcgtgcttaaatagatggccacgccctcaggcncgcccaccttcgttnnctctcncgcttgggtacgcggtagatttcgcgcttc

SEQ ID NO: 93 - DNA, 182 bp, Mus musculus
tacagtaaaacctcgttaagccgatattggtttattcaaaatagcgcataattcaaagcaactgctattccctagccgactagatgccctattgttctttaataaaaatatcggataatccgaatctagttaattcaaagtccattttttctagtcccttgcatttcgaattaatgagrttt

SEQ ID NO: 94 - DNA, 165 bp, Mus musculus
aattgaaaaaacgcttttactgcacgcagtaattgacattaagtgctgttttagaaggaaaccagtgattttcaattgacattaaatagaaaattatttaatgtcaattgatattaaatgggtaaaattgtactaaattaatataaaatggtttggtggaaaaaa

SEQ ID NO: 95 - DNA, 323 bp, Mus musculus
  misc_feature (13)..(13): n is a, c, g, or t
  misc_feature (28)..(28): n is a, c, g, or t
  misc_feature (49)..(49): n is a, c, g, or t
  misc_feature (93)..(93): n is a, c, g, or t
  misc_feature (172)..(172): n is a, c, g, or t
  misc_feature (219)..(219): n is a, c, g, or t
  misc_feature (271)..(271): n is a, c, g, or t
  misc_feature (283)..(283): n is a, c, g, or t
  misc_feature (299)..(299): n is a, c, g, or t
  misc_feature (304)..(304): n is a, c, g, or t
tggttgtcttttnaactttgtaaaactntaatacaaaggaaattaaaanaagaaacaatgaaaacattttcaaaataatttcaaaatcaaatntatccctcagtaatttgatttgtaggaaacggagaacaaagcattacaaacagagtgcttttaaataaatcataattanttacacgtgcaaatgcatcgactaagtttggacgcgcaattagacgnctaattatgttgaaaatgcaattgcacgcttaaatagatggccacgcccccnggcccgcccagntgtttctgctctcttntgtangcgctaggtttcgtgcttc

SEQ ID NO: 96 - DNA, 2623 bp, Mus musculus
  misc_feature (839)..(839): n is a, c, g, or t
  misc_feature (846)..(846): n is a, c, g, or t
  misc_feature (916)..(916): n is a, c, g, or t
  misc_feature (1006)..(1006): n is a, c, g, or t
  misc_feature (1014)..(1014): n is a, c, g, or t
  misc_feature (1062)..(1062): n is a, c, g, or t
  misc_feature (1064)..(1064): n is a, c, g, or t
  misc_feature (1137)..(1137): n is a, c, g, or t
  misc_feature (1184)..(1184): n is a, c, g, or t
  misc_feature (1313)..(1313): n is a, c, g, or t
cagtggcgtaccaagggcggggcggtgggagcggtccgccccgggtgcaggcaataagggggtgcattgtctgtagagaatttaaaaacaataataaaaccgactaaaagtcggtctgctttttattatcaccatgcgccggcaattctaaacaatgtcagtgataaaatactcctccccgaaaaatcttttgttggtctaagttctaaacaattgctgcggttactgttgagttttaataatatatatgtaagcttcaaattagcacatttttattacttatcctttaataaacattgtattctacatggaagttaattcggagaactcccagttatacagtcggcccccgacacacgcggactcagctacacgcgttcgtttcgagagtaagttcgtaacggttcggaatcgttcgagctcgcttcgggcgcagttcgtgtctccaacccctgtggtactacatattcctgcgtttaaacagtagattcgaaataaacaatgatagcacagtgattgtaaagacgaagaaacagaacttgagttacttcaattctgtcattctatgtgaccacttggagtttttatttgtgtttaaaatttaaaacagtgaaacagagtgcgaactgcgaggtgtaatatttttgtttggtaagtgcaaattttagttcatacatgaaatattttactgaatttgaataatatctttaaaattgaaatttattctttttaaattgttaattgttttaaaactaaagaacgaatcaagaaaataaaatattacatcagtggtacgatttagtagttgcctaaattttaaaagcataatttaggaattntttttgntagcactccgcatgcttcacacacggatcaaacgcgaaaagtgatcaaatatgtctatattgaagatganaaagtcgaaataaaggaattcttcttgggcttctttgatatttctaggaaaactgctgctgagcccacagaaaagatatcgaagcaactngatggtgntggactggacataaacctctgccgtggtcaaggatatgacaatgccgnanctatggccagtactcactgtggtgttcgggcaaaaatcaaagaaattaatcccaaatccttatttgtgccttncgcaaatcattctctgaacctttgcggagttcactcttttggaagtntttcttcatgtgtgacattttttggaactttggaaaaaaattattcattcttttcagtctcacctcatcgatggaaaatgctgcagaatgtaggtataacagtgaaaagactttcccagacgagatggngtgctcattatgaagctgtgcgcgcagtaaagacaaattttgaaaagttaatctcaacctttgaagtactgtgcgatccaaaagaaaatgtggacacaagagaatcagctcagattttgctctctgctgtatgcgatttttcttttctgagttatctttttttctggtgtgaagttttagatgaggttaatcagacacaaaaatatttgcaaacagccagaatcagccttgaacaatgtacagtgaaacaccaagctttaaaattgttccttgaagatcggcgcacagaaattgtggagaaggccattaactatgcaacaacaaaatgtaaggaaatggacatttacatagaaaaaagaatcaaatttcgaagaagaatgccaggagaaacgacaaaagatgctggtcttacattgccagaagaaatcaaaagggcaatgtttgaatgcctcgatcgttttcaccaagaactggacactcgttctaaagcaatggatcaaataatgtcaatgttcgctatcattcagccattttctctgatttttgcagaagaagaaaaacttcggaagtttttaccaaatataatagaaatttatgatgaattttctggtgaagatattttagtggaaatttttcgactgcggagacatttgaaagccgctagaatcgatcccgaagaaacaaagacatggacagtattgcaatttctggaatttattgtgaaatgggatttttatgaatctctgccaaacttatccttatgtttaagacttttcctaactatttgtatatctgttgcttcatgtgaaagaaacttttcgaaattaaaattaataaaaagtgttcttcgatcaactatgagcgaagatagattgacaaatctggctatactgtctattgaacatgaatatgcgaagaagatcaattttgacgaagtcattgacaaatttgcagaagttaaggctcgaaaacagaaactgtaatgttattattcattactgcgacagaccaatatgtaggtataattttttccttttttcaaaaaatacattaatgtaattaaaaagtattaatccattactttttttccttttttgtactgtaatatttattttttattttttatactggcatgattatatatacgaagttcaataaaagaaaattttcactgtctgcgtttcttttctggccattattattattcgtttcatttcatgattattactgaaaataattttgtcgtatagaggaggggggtgttaaaaaatgatccgctccgggtgtcaaatacgctaggtacgccactg

SEQ ID NO: 97 - DNA, 687 bp, Mus musculus
agaggccgttcaccttccagcccgccgagctgttgctgtcgcccttatccttgaagtagggcacgctcttcaccatccactcgtagatctgcgacaggcaggctcaggttgctcataaagtcggtgctgacagcggacgccgaggccaggctcgccgcggcgtcggggttggcggccgcgccgcccgacggcgccggactggaggtggtcgagttggactggttaaactccggcctgggcagcggccaggtacaggagcgctgccggggcagcggctcgaagtccgggtcggtctccaccacctggggcgcttcggccatggtgcccccgcccctcccccaccagcagagaagtaccgggagacgcggccaccgggggcgcggagcgggcgacccgagtgtcgctccgagattggggggccgcggacggcggacgcacgccggagtcaggcgcggcggggcgcagcggacggacgcgcccagaacttaacttcgctgggtcaccggtgtctaaggagcctccgagttcgtgcctaggacggggcaccgaccaggccgcggagcccggcagctcgggcaccgcgctcccgctgacaagggccgcggacgccaaggcagacgggcggacgctgcgggccgctctagctctccgcggccgcgcgacagcggcgctgctgcctgttgaatg

SEQ ID NO: 98 - DNA, 388 bp, Mus musculus
gcagatggcgcgggatgatgcgcgtcttcttgttgtcgcgggccgcgttgcccgccagctccaggatctcggccgttaggtactccagcaccgccgccatgtacaccggcgcgccggcgcccacgcgctccgcgtagttgcccttgcgcagcagccggtgcacacgccccaccgggaactgcagcccggcgcgggacgaccgcgacttcgccttggcgcgggccttgcctccttgtttgccacggccagacatggcggaataagcaatttctgctgctttttccaagggaaggatatggaaaggccccactctgtgatctcctacgagttccttagcaaccgtggaatctaacctcagaagacaccaaagtcccgcatgcataaggac

SEQ ID NO: 99 - DNA, 226 bp, Mus musculus
ggactcgaggcaaccggtgagagtgaacctcgtcgtcgaggagtgagcacgccgcgctttggatggctgcaagacttggcctgatgactgggttgtcggaatcgtgacatcttcgaagaactgttgctgtaactcttggtatatttggaacttatggcatacacaatccacaagcatacgatactgtgaagcagatttttgctttagaataaaaaagctgtgatac

SEQ ID NO: 100 - DNA, 698 bp, Mus musculus
ggccccatcgaccaggaaggacttaaatggagcccgcggagccacaccaacgcgccagcagaccagggactttggcaggcccggtctgtctacagcacaaacgcagggcctgtaggagcgtgtcgtggtgacagcgtaaagtggacagaacgaacgaatcctaagacgaaaactatttaaaagcaatggcctgcggccgacgcgggagaaaagagaggtggacagcctacagctttcccaaagcaagagcgtgtcaataatcgcaccgtctcgggggcgagggcgaagcgcccgaccaaccttggcaaccctgagccgccgccgccgctcccgcttcccgccgccgccgccggcccctgcgccgccgccgccaccatggctccctcctgctcgcccgccgagccgcctaccgccgccgttgccgccgccgccgccccgtcccctccgaactgctcctccgacatagtgctactgccgccgccgaagacgacacccgccgacgccgccgccgccgcgaaccgaaactagcagcaaagtaatccccgccgccgccgcgcgatgcccgctctacctcgcgaggcaccctagacaaggcgcgcgcgtggctgcaagggctcctgcgcctctccccggcctgcccccctcgcctcccactctcgcgcagcgcgctctctcgctctccccggctgcactgaaaa

SEQ ID NO: 101 - DNA, 521 bp, Mus musculus
ggaggggggcggcggagacggctcgtctgtgtccgagtcgtcgtcgtcctcgggcacctgaggcttgagcagcaccgaacggccccgactgcggggccggcgcggcgaacacatgaaaacgtcggcagccatgaggagaacctggtccggggcatcccccgggagccggggcgacgtaaagcggagaagcacggggaacggagggggccgccggcccgacccggcccggccctcgcgcagactgcccgggaacgcgcggcctgagggggagggcgcgggcctcctcgtaacgtcactgtcgtcttggccaatcggatgctcgcctctgcctcttaaaggaaatccagtgagatggttcagtaggtaaataaaagcacttgctgttaagtctgaccttcattccaacccgtgggacccacaagatagaagactcccaacaacttaatctctgaccaccccaaccccactgtggcatctgcgcttctcacgataaataaaggtaaaaataaataaataaaaga

SEQ ID NO: 102 - DNA, 1747 bp, Mus musculus
aaaggcggatgcacggacctcccagacgcggcctcccgtgcctcggagccccggccacgcgtgcgtcccgccgagcagcgctccgtcgctccgcatgggtgacccgcaggccgaggcgcccccacggacacagcctcgctgagccgccgctgaagtttccgagaggacctaagggacaaaagccgccccgcgccccgcccggcccgcgccgctcgggcacacgcggccctttgttgtccggcggcatcggcgacgtcggggcgacccggacccgcgcccggggacccacgcgagctcgggtatcctgagccactggtccctggggccaaacttttgcggagtcccggatgagaaacgcccagaggtccccgccgccgtgcatatgctgcgagttggcgactttcgctgtgctttggaaactggcaagacgccatcaccaggaaatcacattgtcctgctcccgacagccccgacggcgaccgcagcccgcggctcactccgagctggacactgagatgtagcacggtcgcggccctaagcacgtggcttgtcaggtgaagcacacacaacaaaaagggaaaaaaaaaaaaaaaaaaagagttgggtgcctcaagcagcggaccgcgccgccgccttcatccgcactgtttggtgccgcgggccacggccacccctcccgtgtcgcccgcacccacgctacaggtgaccagacggacagcgaactttgggctgcagagaccactcggcaccggccgagtgcaaagaccgcgtgctgcgcgctcattgtcgcagccgcgtccctgcggacttctccaacaacgccacgccgcgtgcgtcgcgccatcccggcgctggccctcaccagctgccttcagcctgtgtggccgccggctccaggaactggcaagttagtttccgagctccggctttgttgttcacccgcgtgcaaacaccgtcgcgagcgccggggtagcgcaacacgcgggtgcacgcggagcccaagcccctggccagagcagagcccacacgggatctgcgaccccgaccctgctctccgggtctgcaggaaacactacgacctagaaaagtcattgtcactttcagcgtggatgcaagtccgccgcagctcgttcgtttccacgctgcactcactttccccacaccctgactttcggtttacctcaaatatcctcctcaattaagaagcgcatctccaacgcctctgtacaaaagaatctggagtccggagagcacgagggcagcgctccccgcaatgccatctccaagacaaaacttcctgtgtgtttgtgtatgggaaggacggaggaggcgggggaggtaaagggaggaaagaaaaaaaaaaaatcacagaaaacaaaacctcggagacagttttgcaccgatgtccggactagaatcactcaccttccacgcgcagcagcgtccccacggtgcaaactgcaccaggccaacctcctgcaagcactccctccgccagcgccgagtccagaaagattgtctccagcagttcttcagttgctaccgcgaacggagggcctgcctggcttgtgagactgctcaactggtactttcaagtctgatcacctgaaggcaataaccaacttctgcaaattcgccccgcgcaccacatgcacacacaaattaaaatttcaattctccaacagagttttcgttctttggggctttgatgataaattgtttaataaagtcgcattttgaaatggtc

SEQ ID NO: 103 - DNA, 1729 bp, Mus musculus
aaaggcggatgcacggacctcccagacgcggcctcccgtgcctcggagccccggccacgcgtgcgtcccgccgagcagcgctccgtcgctccgcatgggtgacccgcaggccgaggcgcccccacggacacagcctcgctgagccgccgctgaagtttccgagaggacctaagggacaaaagccgccccgcgccccgcccggcccgcgccgctcgggcacacgcggccctttgttgtccggcggcatcggcgacgtcggggcgacccggacccgcgcccggggacccacgcgagctcgggtatcctgagccactggtccctggggccaaacttttgcggagtcccggatgagaaacgcccagaggcgccgccgtgcatatgctgcgagttggcgactttcgctgtgctttggaaactggcaagacgccatcaccaggaaatcacattgtcctgctcccgacagccccgacggcgaccgcagcccgcggctcactccgagctggacactgagatgtagcacggtcgcggccctaagcacgtggcttgtcaggaagcacacacaacaaaaagggaaaaaaaaaaaaaaagagttgggtgcctcaagcagcggaccgcgccgccgccttcatccgcactgtttggtgccgcgggccacggccacccctcccgtgtcgcccgcacccacgctacaggtgaccagacggacagcgaactttgggctgcagagaccactcggcaccggccgagtgcaaagaccgcgtgctgcgcgctcattgtcgcagccgcgtccctgcggacttctccaacaacgccacgccgcgtgcgtcgcgccatcccggcgctggccctcaccagctgccttcagcctgtgtggccgccggctccaggaactggcaagttagtttccgagctccggctttgttgttcacccgcgtgcaaacaccgtcgcgagcgccggggtagcgcaacacgcgggtgcacgcggagcccaagcccctggccagagcagagcccacacgggatctgcgaccccgaccctgctctccgggtctgcaggaaacactacgacctagaaaagtcattgtcactttcagcgtggatgcaagtccgccgcagctcgttcgtttccacgctgcactcactttccccacaccctgactttcggtttacctcaaatatcctcctcaattaagaagcgcatctccaacgcctctgtacaaaagaatctggagtccggagagcacgagggcagcgctccccgcaatgccatctccaagacaaaacttcctgtgtgtttgtgtatgggaaggacggaggaggcgggggaggtaaagggaggaaagaaaaaaaaaaaatcacagaaaacaaaacctcggagacagttttgcaccgatgtccggactagaatcactcaccttccacgcgcagcagcgtccccacggtgcaaactgcaccaggccaacctcctgcaagcactccctccgccagcgccgagtccagaaagattgtctccagcagttcttcagttgctaccgcgaacggagggcctgcctggcttgtgagactgctcaactggtactttcaagtctgatcacctgaaggcaataaccaacttctgcaaattcgccccgcgcaccacatgcacacacaaattaaaatttcaattctccaacagagttttcgttctttggggctttgatgataaattgtttaataaagtcgcattttg

SEQ ID NO: 104 - DNA, 395 bp, Mus musculus
acgggagggacgcagcgggccacctggtgcgcgccagcggggccgcaaagccagccggccggtgtgaacttcggggctacaggatccgctgggccgaacctccggccccgcgggcggggtggccaagttccacttgataccaactatattaaaaatgcacaatttaatcagaacaagggctgaccattttgaaggacctttatttccttgatgctcccggtctttgtctccaccgtctatcgtctctcatcgaatggctgagctctgtgtgcgtcacgaaggcatcaaacatgatgacagcgataaccaccaggatggtgatgacgacaacaacgatgacggtggtgatgatgatgacgacgatgacgatgacgacgacgatgacgatgatggta

SEQ ID NO: 105 - DNA, 409 bp, Mus musculus
cggccgcgcgcgatgtgggagctcgcggccggagcgcccggggaggccgggcccacgacgcccgtggctccgctgcggcagcggcggtgctggtgtcgcgcgcggccgggaggcggcttcgcgccgcgggcgggaggctgcggcgggcgacccgtcctggacacgcgaggaagagcgagccgatggcggcaggggccgcgcttcgacccggtaacttagaagatgataattaatgtggttgctgataattctgaataaatacagcttttatcccagaaatgtgaatcctcagatggaatgaaaggcctgcaccatagacatcgaagcatttacaccccgcttgaagagtttgaaatggactttaccactgagaaatcaagatggcagcccattatggggaattgaggaa

SEQ ID NO: 106 - DNA, 409 bp, Mus musculus; identical to SEQ ID NO: 105

SEQ ID NO: 107 - DNA, 310 bp, Mus musculus
gacccgtcctggacacgcgaggaagagcgagccgatggcggcaggggccgcgcttcgacccggtaacttagaagatgataattaatgtggttgctgataattctgaataaatacagcttttatcccaggtgtgccattttgaagactgagaccatagagttctaagaataaaggaaagagcccttgggaaattattatatatagcaagtaagtttttttaattgttatatttgaatatttgccaacatttgggtaggaatatatcattaaagcttgtcaataaaaaaatactgtttgaccgtgtatatat

SEQ ID NO: 108 - DNA, 158 bp, Mus musculus
ctttcttccacttcatgcgacggttctggaaccagattttgatctgacgctcggacaggcaaagcgcgtgggcgatctcgatacgtcgccgccgggtcagacgcctcttcgccctaggctggcctctgcggagattccaggccccacagagaccagga

SEQ ID NO: 109 - DNA, 532 bp, Mus musculus
gactggcgtcccggagctccgggcgcccctcgaccgccaccggcatcacctgggccgctacgccgcagtgcctggcctgctgtccccgagcccggagcttcccgtctgctcccgggcgaaatcggagacaatttcaactctgagaggagcaacccagcaagcgaaataaactgtacaccaccatgcacgtgcgaccaaaatctctcggccctcctccatttcgaatacttctctccttatttccgaaatacgcattgttttaatacttcatctcagttccaatagaatggagatattcttaactgtgaactttgcagacaatcaaaaccttttttaaagccattccaccgtgacaacaaagcgattaagatgttttctatttcgtaaataccatttaaaattgctatattttatgctctgggttctacactgtcttatttgcacactattttgtgtctgatttaatctataaccgaaaacataatgtattaagtgaatacatatttgctcagctgataagctttttttaata

SEQ ID NO: 110 - DNA, 735 bp, Mus musculus
aaatgaaaaagatccacaccacgacttacaagtcacaaaaacgccagatctttccgctaggagtctgaaacgggggttggggggatgtaacgcaacccagcacaaagttttacgaaatcgaagtgcagaatcacaacagacatggcagcggaggccgcgcagaacaacagcagggtcccgagacccagtccccgcagcagccgccgcgcagctccggagcgggagcagagcaccgttcccatttaaggcttccagaagcgtccccggagtggtgcgaaggcgaacaaaagggcaagcgcgggcgggccgggccgcggggccggggggtctctcccgcaggccccgcggaggcgcgtcgcggcctcccctgcgccgccaccgtcgcccccaaacttcgccggcctcccgcggcgcacccgcactccgtcgccacccgccaactcaccgggatcccgagaaactcatcctccggcgctcggcgcgggcgggcacggacagccgcggggcggcggggcccggctcggcctccgccctcggcgtgcagcggcccgggcccggtcgcccctcgcgcggccgtacagtcgtcgtctcgcgcggggccgacgcgcgggagctcgagcctccctccccgcccttccctcctccctccgccctcctcctcctcctcctccgcgcggcgggcaagcagattccaatctctgccgcctcagccgcggaggacgctcgctcgttcgc

SEQ ID NO: 111 - DNA, 344 bp, Mus musculus
ggcgggacaccagccactggcgcggctctcggtcgtagtcgctgcacgcaggggacgtggggcacgtctctacctgcggcgcgcagtgcaatgcatctggccagggggaaagagatggcgatatgctagcgactggcgcacgcggagcgagggcaccgtggaaccgccgacgctcaggatccccggacaatcctgtgcctttggcggtgtgtggagcgctctcgacgttaaagcccagtaaatagacgaaggacttgtttcaatttcaacttcacaaggatccctttctaatcactcctttcttccctgattttttttaattaaaaaatacagatcaaagtaaa

SEQ ID NO: 112 - DNA, 686 bp, Mus musculus
agacacactggagggaaaggtcaaactaaaggcgaacgaacggagagaacgcagcgagcggccgccgcgccgtgcgccatgacggacgcggccgggccggcttcccagcggccgccgcccgcgcccctcgcccgcgcgtgctcgcgaccccctccccggccgcgacgggcgcgcgacgggcgcgaggcgcgcgcgggcgggcgggcgggaggtggactcagccgggcgggcgggcgggcgggcgacgggcggcggcctctcgtcgcgcgtctccggcctcgcgcggtcggtcggcgagagcggccaggaaactaatagaagaaacaaaagcaaaatgtttcggtgttgttttgtaagcaaagccaagcttgctgtcagaaagtctgtgtctgtaaacatccccgactggaagctgtaagccacagccaagctttcagtcagatgtttgctgctactggctcttcgaatgcatcttttggacaatctggcctaaggattggagctgaggatgctgagaaatgcctgggatgcatcagaaatcttgaggagcattcatttactgcttgcttctgctggcagcttctaatgttgggagctgactaaacatgcttcttctggccttagaggatctgtctaaaacaagttttaatactgtttttgccgtgtcagtacaagtttatccttta

SEQ ID NO: 113 - DNA, 183 bp, Mus musculus
gccgaagccgcgccctacaccagccggaaggacgtataaatactcgcgcccgcgctgggccgtcccgcttgtgtccaggaacccgtggcgacgttggcgtgttcgggttcccctggatatggacgttgccgccgcctcccgcaagaattgtcctcttttctcaaataaagtgatttaacctaa

SEQ ID NO: 114 - DNA, 105 bp, Mus musculus
attgtccaaacgcaattctcgagtctctggctccggccgagagttgcgtctggacgtcccgagccgccgcccccaaacctcgagggggagaggccgggcggagcg

SEQ ID NO: 115 - DNA, 869 bp, Mus musculus
gtcccggccccgcggccgtggcggtgtcttgcgcggtcttggagagggctgcgtgcgaggggaaaagtcgctcgtcgacctcccctcctccgtccttccatctctcgcgcaatggcgccgcccgagttcacggtgggttcgtcctccgcctccgcttctcgccgggggctggccgctgtccggtctctcctgcccgacccccgctggcgtggtcttctctcgccggcttcgcggactcctggcttcgcccggagggtcagggggcttcccggttccccgacgttgcgcctcgctgctgtgtgcttgggggggggggccgctgcggcctccgcccgcccgtgagcccctgccgcacccgccggtgtgcggtttagcgccgcggtcagttgggccctggcgttgtgtcgcgtcgggagcgtgtccgcctcgcggcggctagacgcgggtgtcgccgggctccgacgggtggcctatccagggctcgcccccgccgtcccccgcctgcccgtcccggtggtggtcgttggtgtggggagtgaatggtgctaccggtcattccctcccgcgtggtttgactgtctcgccggtgtcgcgcttctctttccgccaacccccacgccaacccaccgccctgtgctccgcgcccggtgcggtcgacgttccggctctcccgatgccgaggggttcgggatttgtgccggggacggaggggagagcggataagagaggtgtcggagagctgtcccggggcaacgctcgggttggctttgccgcgtgcgtgtgctcgcggacgggttttgtcggaccccgacagggtcggtctggccgcatgcactctcccgttccgcgcgagcgcccgcccggctcac

SEQ ID NO: 116 - DNA, 1831 bp, Mus musculus
agggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggagtttcgaagacgatcagataccgttgtagttccaaccataaacgatgccgactggcaatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggccataagcttgcgttgattaagtccctgccctttgtacacaccgcccgtcgctactaccgattggatggtttagtgaggcccacggccctggtggagcgctgagaagacggtcgaacttgactatctagaggaagtaaaagtcgtaacaaggtttccgtaggtgaacctgcggaaggatcattaacgggagactgtggaggagcggcggcgtggctcgctctccccgtcttgtgtgtgtcctcgccgggaggcgcgtgcgtcccgggtcccgtcgcccgcgtgtggagcgaggtgtctggagtgaggtgagagaaggggtgggtggggtcggtctgggtccgtctgggaccgcctccgatttcccctccccctcccctctccctcgtccggctctgacctcgccaccctaccgcggcggcggctgctcgcgggcgtcgtgcctctttcccgtccggctcttccgtgtctacgaggggcggtacgtcgttacgggtttttgacccgtcccgggggcgttcggtcgtcggggcgcgcgctttgctctcccggcacccatccccgccgcggctctggcttttctacgttggctggggcggttgtcgcgtgtggggggatgtgagtgtcgcgtgtgggttcgcccgtcccgatgccacgcttttctggcctcgcgtgtcctccccgctcctgtcccgggtacctagctgtcgcgttccggcgcggaggtttaaagaccccgggggggtcgccctgccgcccccagggtcgggggg

SEQ ID NO: 117 - DNA, 430 bp, Mus musculus
tttctatgctcgcacgcagcgcggagcatggcgtccccgggagctggggcatgggaggcggttgtggcgtgggatctagggtgtctgcagcggactcggcggccgtgtgaggcgctcggcccgccggaccccgcctgaagcgggtcggtgtggaaaccgagcgccgtttggatgtagatcttcgccgtaggcccagaactgggcgggaataaagcgaagacccaggtcacacggtgagtacctgaagcttacggatatttcctgaacgacacgtgggagcacagagtgggttaaagggtgtccttgatgatttggaaatttgatctacagaagcacgagtgatttaaattttctaggggccgtgtcaaaactccatagatgaatgctcatttgtaacaagactatgaataaatgcttcgatgtgctgttc

SEQ ID NO: 118 - DNA, 303 bp, Mus musculus
ggcatgggaggcggttgtggcgtgggatctagggtgtctgcagcggactcggcggccgtgtgaggcgctcggcccgccggaccccgcctgaagcgggtcggtgtggaaaccgagcgccgtttggatgtagatcttcgccgtaggcccagaactgggcgggaataaagcgaagacccaggtcacacgggtgtccttgatgatttggaaatttgatctacagaagcacgagtgatttaaattttctaggggccgtgtcaaaactccatagatgaatgctcatttgtaacaagactatgaataaat

SEQ ID NO: 119 - DNA, 716 bp, Mus musculus
aggagccctcgtcgcggttgcggtacttgtacatggcgtagaggagaatgaggatgcagagcgccgccgccgccacgatgcccaccaccatgcccgtggtgctgctggattcgcggatcacctccactgcacctggcgggccgcgctcccccggacccgtggggaaaagaaagagaagagagaagagagggcgtcagcgagggccagggcgcgggcggccggcacagagagagaaacaacagcctcacacccaaatcggttcaattggctttggcggagactacgcgggccgggcccgcccgccagccggccagcgcctgcgctttaagcgggctgcggctcggatgccctgggcaccccacgcgcgggctctgtgactttgggttttggtctttttgtttcgtcttaagaaaccaaacggaacagaaaggaaaggaaattgaaaagaaacggaattttttttttttctactggtttaaggctttaaaaacatacaacagcagcatttaaacaaacctaacaacaatatcttttagggttttttttatatatatttctttttgcttttttttttttttgcttttgtttttaaaaaagaagatagcataccacggaattcaggcaacttacatcaacaaataggccgtgtgattttagcatgaagaaaaaaattacaaacagagctgtgtaagcgggttctcccgaaaaaaaataag

SEQ ID NO: 120 - DNA, 519 bp, Mus musculus
cggccgccgcggctcgaaggccgtggggaccccaggccccaggggaaaagaaagagaagagagaagagagggcgtcagcgagggccagggcgcgggcggccggcacagagagagaaacaacagcctcacacccaaatcggttcaattggctttggcggagactacgcgggccgggcccgcccgccagccggccagcgcctgcgctttaagcgggctgcggctcggatgccctgggcaccccacgcgcgggctctgtgactttgggttttggtctttttgtttcgtcttaagaaaccaaacggaacagaaaggaaaggaaattgaaaagaaacggaattttttttttttctactggtttaaggctttaaaaacatacaacagcagcatttaaacaaacctaacaacaatatcttttagggttttttttatatatatttctttttgcttttttttttttttgcttttgtttttaaaaaagaagatagcataccacggaattcaggcaacttacatcaacaa

SEQ ID NO: 121 - DNA, 69 bp, Homo sapiens; identical to SEQ ID NO: 1

SEQ ID NO: 122 - DNA, 69 bp, Homo sapiens; identical to SEQ ID NO: 2

SEQ ID NO: 123 - DNA, 69 bp, Homo sapiens; identical to SEQ ID NO: 3

SEQ ID NO: 124 - DNA, 69 bp, Homo sapiens; identical to SEQ ID NO: 4

SEQ ID NO: 125 - DNA, 69 bp, Homo sapiens; identical to SEQ ID NO: 5

SEQ ID NO: 126 - DNA, 69 bp, Homo sapiens; identical to SEQ ID NO: 6

SEQ ID NO: 127 - DNA, 69 bp, Homo sapiens; identical to SEQ ID NO: 7

SEQ ID NO: 128 - DNA, 69 bp, Homo sapiens; identical to SEQ ID NO: 8

SEQ ID NO: 129 - DNA, 69 bp, Homo sapiens; identical to SEQ ID NO: 9

SEQ ID NO: 130 - DNA, 2524 bp, Homo sapiens; identical to SEQ ID NO: 10
  misc_feature (2408)..(2408): n is a, c, g, or t

SEQ ID NO: 131 - DNA, 325 bp, Homo sapiens; identical to SEQ ID NO: 11

SEQ ID NO: 132 - DNA, 1237 bp, Homo sapiens; identical to SEQ ID NO: 12

SEQ ID NO: 133 - DNA, 357 bp, Homo sapiens; identical to SEQ ID NO: 13
  misc_feature (355)..(355): n is a, c, g, or t

SEQ ID NO: 134 - DNA, 277 bp, Homo sapiens; identical to SEQ ID NO: 14
  misc_feature (52)..(52): n is a, c, g, or t
  misc_feature (63)..(63): n is a, c, g, or t
  misc_feature (87)..(87): n is a, c, g, or t
  misc_feature (123)..(123): n is a, c, g, or t
  misc_feature (198)..(198): n is a, c, g, or t
  misc_feature (204)..(204): n is a, c, g, or t
  misc_feature (208)..(208): n is a, c, g, or t
  misc_feature (214)..(214): n is a, c, g, or t
  misc_feature (271)..(271): n is a, c, g, or t

SEQ ID NO: 135 - DNA, 375 bp, Homo sapiens; identical to SEQ ID NO: 15
  misc_feature (21)..(21): n is a, c, g, or t
  misc_feature (24)..(24): n is a, c, g, or t
  misc_feature (46)..(46): n is a, c, g, or t
  misc_feature (334)..(334): n is a, c, g, or t
  misc_feature (351)..(351): n is a, c, g, or t
  misc_feature (354)..(354): n is a, c, g, or t

SEQ ID NO: 136 - DNA, 184 bp, Homo sapiens; identical to SEQ ID NO: 16
  misc_feature (20)..(20): n is a, c, g, or t
  misc_feature (37)..(37): n is a, c, g, or t
  misc_feature (165)..(165): n is a, c, g, or t

SEQ ID NO: 137 - DNA, 168 bp, Homo sapiens; identical to SEQ ID NO: 17
  misc_feature (62)..(62): n is a, c, g, or t

SEQ ID NO: 138 - DNA, 273 bp, Homo sapiens; identical to SEQ ID NO: 18
  misc_feature (244)..(244): n is a, c, g, or t

SEQ ID NO: 139 - DNA, 169 bp, Homo sapiens; identical to SEQ ID NO: 19
  misc_feature (3)..(3): n is a, c, g, or t
  misc_feature (60)..(60): n is a, c, g, or t
  misc_feature (165)..(165): n is a, c, g, or t

SEQ ID NO: 140 - DNA, 424 bp, Homo sapiens; identical to SEQ ID NO: 20
  misc_feature (42)..(42): n is a, c, g, or t
  misc_feature (55)..(55): n is a, c, g, or t
  misc_feature (234)..(234): n is a, c, g, or t

SEQ ID NO: 141 - DNA, 316 bp, Homo sapiens; identical to SEQ ID NO: 21
  misc_feature (82)..(83): n is a, c, g, or t
  misc_feature (171)..(171): n is a, c, g, or t

SEQ ID NO: 142 - DNA, 192 bp, Homo sapiens; identical to SEQ ID NO: 22
  misc_feature (166)..(166): n is a, c, g, or t

SEQ ID NO: 143 - DNA, 248 bp, Homo sapiens; identical to SEQ ID NO: 23
  misc_feature (44)..(44): n is a, c, g, or t
  misc_feature (186)..(186): n is a, c, g, or t

SEQ ID NO: 144 - DNA, 208 bp, Homo sapiens; identical to SEQ ID NO: 24
  misc_feature (37)..(37): n is a, c, g, or t
  misc_feature (83)..(83): n is a, c, g, or t
  misc_feature (182)..(182): n is a, c, g, or t
  misc_feature (187)..(187): n is a, c, g, or t
  misc_feature (194)..(194): n is a, c, g, or t

SEQ ID NO: 145 - DNA, 302 bp, Homo sapiens; identical to SEQ ID NO: 25
  misc_feature (264)..(264): n is a, c, g, or t
  misc_feature (270)..(270): n is a, c, g, or t
  misc_feature (281)..(281): n is a, c, g, or t

SEQ ID NO: 146 - DNA, 292 bp, Homo sapiens; identical to SEQ ID NO: 26
  misc_feature (41)..(41): n is a, c, g, or t
  misc_feature (199)..(199): n is a, c, g, or t
  misc_feature (215)..(215): n is a, c, g, or t
  misc_feature (221)..(221): n is a, c, g, or t
  misc_feature (236)..(236): n is a, c, g, or t
  misc_feature (270)..(270): n is a, c, g, or t

SEQ ID NO: 147 - DNA, 562 bp, Homo sapiens; identical to SEQ ID NO: 27
  misc_feature (7)..(7): n is a, c, g, or t
  misc_feature (209)..(209): n is a, c, g, or t
  misc_feature (279)..(279): n is a, c, g, or t
  misc_feature (504)..(504): n is a, c, g, or t

SEQ ID NO: 148 - DNA, 301 bp, Homo sapiens; identical to SEQ ID NO: 28
  misc_feature (155)..(155): n is a, c, g, or t
  misc_feature (299)..(299): n is a, c, g, or t

SEQ ID NO: 149 - DNA, 240 bp, Homo sapiens; identical to SEQ ID NO: 29
  misc_feature (2)..(2): n is a, c, g, or t
  misc_feature (22)..(22): n is a, c, g, or t
  misc_feature (96)..(96): n is a, c, g, or t
  misc_feature (180)..(181): n is a, c, g, or t
  misc_feature (232)..(232): n is a, c, g, or t
  misc_feature (235)..(235): n is a, c, g, or t
  misc_feature (237)..(237): n is a, c, g, or t

SEQ ID NO: 150 - DNA, 270 bp, Homo sapiens; identical to SEQ ID NO: 30
  misc_feature (30)..(30): n is a, c, g, or t
  misc_feature (68)..(68): n is a, c, g, or t
  misc_feature (130)..(130): n is a, c, g, or t
  misc_feature (137)..(137): n is a, c, g, or t
  misc_feature (142)..(142): n is a, c, g, or t
  misc_feature (144)..(144): n is a, c, g, or t

SEQ ID NO: 151 - DNA, 75 bp, Homo sapiens
ggaatggaatggaatggaatggaatggaatggaatggaatggaatggaatggaatggaatggaatggaatggaat

SEQ ID NO: 152 - DNA, 335 bp, Homo sapiens
cagtcatgcgctgcataacgacgtttcggtcaacgatggaccacatatacgacggtggtcccataagattataataccgtatttttactgtaccttttctatgtttagatacacaaatacttaccattgtgttacaattgcctacagtattcagtacagtaacatgctgtacaggtttgtagcctaggagcaataggctataccayatagcctaggtgtgtagtaggctataccatctaggtttgtgtaagtacactctatgatgttcgcacaacgaaattgcctaatgacgcatttctcagaacgtatccccgtcgttaagcgacgcatgactg

SEQ ID NO: 153 - DNA, 126 bp, Homo sapiens
gtattatgacatcacaatatattatgacatcataattcgtatgtattatgacatcacaatatattatgacatcataattcgtatgtattatgacatcacaatatattatgacatcataattcgtat

SEQ ID NO: 154 - DNA, 170 bp, Homo sapiens
ccattcgattccattcgatgattccattcgattccattcgatgatgattccattcgattccattcgatgattccattcgattccattcgatgatgattccattcgattccattcgatgattccattcgattccattcgatgatgattccattcgattccattcgatgatt

SEQ ID NO: 155 - DNA, 1287 bp, Homo sapiens
ttaggttggtgcaaaagtaattgcggtttttgcattgttggaatttgccgtttgatattggaatacattcttaaataaatgtggttatgttatacatcattttaatgcgcatttctcgctttacgtttttttgctaatgacttattacttgctgtttattttatgtttattttagactatggaaatgatgttagacaaaaagcaaattcgagcgattttcttattcgagttcaaaatgggtcgtaaagcggcggagacaactcgcaacatcaacaacgcatttggcccaggaactgctaacgaacgtacagtgcagtggtggttcaagaagttttgcaaaggagacgagagccttgaagatgaggagcgtagtggccggccatcggaagttgacaacgaccaattgagagcaatcatcgaagctgatcctcttacaactacgcgagaagttgccgaagaactcaacgtcgaccattctacggtcgttcggcatttgaagcaaattggaaaggtgaaaaagctcgataagtgggtgcctcatgagctgagcgaaaatcaaaaaaatcgtcgttttgaagtgtcgtcttctcttattctacgcaacaacaacgaaccatttctcgatcggattgtgacgtgcgacgaaaagtggattttatacgacaaccggcgacgaccagctcagtggttggaccgagaagaagctccaaagcacttcccaaagccaaacttgcaccaaaaaaaggtcatggtcactgtttggtggtctgctgccggtctgatccactacagctttctgaatcccggcgaaaccattacatctgagaagtatgctcagcaaatcgatgagatgcaccgaaaactgcaacgcctgcagccggcattggtcaacagaaagggcccaattcttctccacgacaacgcccgaccgcacgtcgcacaaccaacgcttcaaaagttgaacgaattgggctacgaagttttgcctcatccgccatattcacctgacctctcgccaaccgactaccacttcttcaagcatctcgacaactttttgcagggaaaacgcttccacaaccagcaggatgcagaaaatgctttccaagagttcgtcgaatcccgaagcacggatttttacgctacaggaataaacaaacttatttctcgttggcaaaaatgtgttgattgtaatggttcctattttgattaataaagatgtgtttgagcctagttataatgatttaaaattcacggtccaaaaccgcaattacttttgcaccaacctaa

SEQ ID NO: 156 - DNA, 970 bp, Homo sapiens; identical to SEQ ID NO: 34

SEQ ID NO: 157 - DNA, 3509 bp, Homo sapiens; identical to SEQ ID NO: 35

SEQ ID NO: 158 - DNA, 5035 bp, Homo sapiens; identical to SEQ ID NO: 36

SEQ ID NO: 159 - DNA, 439 bp, Homo sapiens
tgtccggagctgcacgccccggccatagcgaataataattaacgattaaaacgcctgagctctattcatttccaccttctacctcctccctatctttgccttttttcccctgtactaatacctcgttaaagatggcgctcttcctgcttcttcttcactcacttttcccgcgcccgggaaaattgttacttaatagcgcaagcgcaacatgacgtccgaccggagaaaccgaaactaacctggccacgccctcggcaatgagatcatttccgccttagcccaaccccttcccttccaagtgtatataaggcagtgcattaccgccattaaacgagacttgatcagagcactgtcttgtctccatttctcgtgtctcttgttccccaaattcccaccccctcctccagggcctgctctgactatcccgcgggccgggata

SEQ ID NO: 160 - DNA, 437 bp, Homo sapiens
tgtccggagctgcacgccccggccatagcgaataataattaaagattaaacgcctgagctatattcatttccaccccacaccttctccctagatttaccttcttccctgtattaataccgccattaaaagatggcgctcttcccgcttcttcttcattcatttttcccgcgcccgcgaaaagactacctgacagcgcaggcgcaacatgacgtccgaccggagaaaccgaagcctatctggccacgccttccgcaatgaggtcatttccgccttagcccaaccccttcccctccaaatgtatataaggcattgcattaccgccattaaacgagacttgatcagagcactgtcttgtctccatttctcgtgtctcttgttccccaaattcccaccccctcctccagggcctacactgactatcccgcgggccgggata

SEQ ID NO: 161 - DNA, 371 bp, Homo sapiens
tgttaggcaggaatctagacccaacatggcggtatcacccggcatggcaggccctttgttaggacttcccgcccttcacttcctgctaagactctcagcgcgcgaaaaaagcccgcgcccgccaaaaaacccccgctctgcgcaagctcctggacacgtcattcctcagaaatcgaaacctaactcaggaaaaccgaaacctacaaaccccgcctacctcgccctataaaaggcccccgatacccgccccgagcgcgacttcctcggccctcctcctaggggaccggtgaacctcgcccgcgagcccaataaaggctacctctgttctcatctgcctcgtgtcttcttgctcggctccccattacattaca

SEQ ID NO: 162 - DNA, 494 bp, Homo sapiens
tgtttgggtgagggagaaaggacaagatggaggaaggtgaacaagaaggcacaatccatgttgcttccgggttcttcctcaccaactttcccgcgcgcgggaaaatgcagcccgcgcccgggaagatgcagatcaaccgagcatgcgccaggtgacgtcaatccgaagagatcgaaacttacccggccacgcctacggagacgcccctatcacgcccttatcccgcccactgccctcccccttccagtaccaatgcataaaagtccgccgccggcaggagccggcgtgacttcttcggcccccgcattcgtggaccggagaacctcacccgagagcgccggcgcgacttccctggccccccacacctgaggaccggagaacctcgcccgagagtgtgtgcatatttgcaataaaagactgccgctttcttacgtactttggcctcatgtttaattatttagctctcctaaattaagttaaattaaattaagaca

SEQ ID NO: 163 - DNA, 499 bp, Homo sapiens
tgtttgggtgagggagaagggacaagatggaggaaggtgaacaagaaggcaccgcccctgttgcttccgggttcttcatcaccaacttacccgcgcgcgggaaaatgcagcccgcgcccgggaaaatgcagatcaactgagcaggcgccgcgggacgtcaatccgaagagatcgaaacttacccggccacgcctacggagacgcccctatcacgcccttatcccgcccactgccctcccccttccagtaccaatgcataaaagtccgctgccggcaggagccggcgcgacttcctcggcccccgcattcgtggaccggagaacctcgcccgagagcgccggcgcgacttccctggccccccacacctgaggaccggagaacctcgcccgagagtgtgcgcatatttgcaataaaagactgccgctttcttatgtactttggcctcatgtttaattacttagctctcctaaattaagttacattaaattaaattaagaca

SEQ ID NO: 164 - DNA, 468 bp, Homo sapiens
tgtctggacggggggagaggacaaagacgactaagatggcgcatttccgggttcttcatcaccaacttacccgcgcgcgggaaaatgcagcccgcgcccgggaaaaatacagaccaactgcgcaggcgcaacgtggcgtccgatcgaggaaaccgaaacttacctggccgcgcctacggaacgcccccgacacgcccgtgtcccgcctattgccctcccactcccaagccttagacagaaaagccgctcccggcaggcgcgcggcgcgaacttcctcggcccctcctcatatgcggacctaggaacctcgcccgagaacgccggagcgacttcctcggcctccaccgccggagaccggtgaacctcgccctttcttccttcacattggctagctaataaagtttctttttacctcgcctacttgcctcttctctggcgcctgctccggtggtcgcataaaacaaatca

SEQ ID NO: 165 - DNA, 459 bp, Homo sapiens
tgtctggacggagggaggagggaaacaaagaacaaaagggactaagatggcgtatttccgggttcttcatcaccaactttcccgcgcccggggaaagacacaggtcaactgcgcaggcgcaacctgacgtccgaccgaggaaaccgaaacctacctggccgcgcctaccgcacggcccccgacccgcccatgtccggcctactgccctcccactcccaggcccaagacataaagccgctccgggcagacgcgcggcgcgaacttcctcggcccctcctcatatgcggacccaggaacttcgcccgagaacgccggagcgacttcctcggcctccaccgccggagaccggtgaacttcgccctttcttctttcacgttggctagctaataaagtttctttttaccttgcctacttgccttttctctggcgcctgctctggtggtcgcacaaaacaaatca

SEQ ID NO: 166 - DNA, 454 bp, Homo sapiens
tgttcgggtgagggagaaaggacaagatggaagaaggtaaagaaggtaaacaagatggcgcagttccgggttcttcatcagcgactttcccgcgcccgggaaaaacaccgactgtctgcgcctgcgcattgtgacgtcaaaacaaagaaatcgaaacttacccggccacgcctatgaagacgcccttacccccgcccctgtcctgcccacctcaagccccatccataaaaggccgctcccggaagacatcggcgcgaacttcctcggcccctcctcatatgcggacctaggaacctcgcccgagaacgccggagcgacttcctcggcctccaccgccggagaccggtgaacctcgccctttcctccttcacattggctagctaataaagtttttttaccttgcctacttgcctcatctctggcgcctgctccggtggtcgcataaaacaaatca

SEQ ID NO: 167 - DNA, 358 bp, Homo sapiens
tgtagaggactacgtgctcgcaaacagggcgttccccataagtcctgctctcgcaaacgaagcagggcgttcccgacaagtcctgctctcgcaaacgaagcagggcgttcccgataagtcctgctcttgcaaacgaagcagggcgttgggggcctgtttatatgtaaacatcttgaaaatccagaaagtcagggaaaggtcagaaaaacaacgatgtgtcttgtgacttggcaacattccacaaacgactgtataaaataaagcggagcgcgccattcgaggcggccgccatgtttgtcttgtcttgtgttgtcttgtgtgttcattcctttgtttaggaaacacgcggaccccaaca

SEQ ID NO: 168 - DNA, 320 bp, Homo sapiens
tgtagaggactacgtgctcgcaaacggggcgttcccgataagtcctgctctcgcaaacgaagcagggcgttcccgataagtcctgctcttgcaaacgaagcagggcgttgggggcttgtttatgtgtaaacatcttgaaaatccagaaagtcagggaaaggtcagaaaaacaacaatgtgtcttgtgacttggcaacattccacaaacgactgtataaaataaagcagagcgcgccattcgaggcggccgccatgtttgtcttgtcttgtgttgtcttgtgtgttcattcctttgtttaggaaacacgcggaccccaaca

SEQ ID NO: 169 - DNA, 123 bp, Homo sapiens; identical to SEQ ID NO: 37

SEQ ID NO: 170 - DNA, 180 bp, Homo sapiens; identical to SEQ ID NO: 38

SEQ ID NO: 171 - DNA, 729 bp, Homo sapiens; identical to SEQ ID NO: 39
  misc_feature (471)..(471): n is a, c, g, or t
  misc_feature (540)..(540): n is a, c, g, or t
  misc_feature (547)..(547): n is a, c, g, or t
  misc_feature (577)..(577): n is a, c, g, or t

SEQ ID NO: 172 - DNA, 583 bp, Homo sapiens
ccgcggttcccaaactgtgcgccgaggcgccccggggcgccgcagcgaactcacaggggcgccgcgggatattttaaattttcgagggaaacacagcgatactcgacatctgtcggacaccgcgcgaactactagctcgaggtagttcacagtttcaacattagatcgcgctacattcctttcgatgacgtcatatctttgcgaagctgggttttcggcggttgctgtgataaaaagcaagtaccgcgcgaaaatcaatgtggaacaggaaatgagggtggcagtgtccaatctgattccaaggtttgagaagttgtgcagtgcccaacaggcgcacacatcccattagtaagtaattgtggttatttaagaatgaaataaaatattattttttctttcaatttatgtgtattattttttcaaatggctactaagttgttaggacataaatacttattaagttgtttggacctaactacttaataaacggaactgttaggtatttcttttggcctaggggcgccgtgaaaaaattactgagacactaagggcgccgtgaaccgagaaagtttgggaacctctg

SEQ ID NO: 173 - DNA, 399 bp, Homo sapiens; identical to SEQ ID NO: 40

SEQ ID NO: 174 - DNA, 210 bp, Homo sapiens; identical to SEQ ID NO: 41

SEQ ID NO: 175 - DNA, 176 bp, Homo sapiens; identical to SEQ ID NO: 42
  misc_feature (24)..(24): n is a, c, g, or t
  misc_feature (111)..(111): n is a, c, g, or t
  misc_feature (122)..(122): n is a, c, g, or t

SEQ ID NO: 176 - DNA, 364 bp, Homo sapiens; identical to SEQ ID NO: 43
  misc_feature (164)..(164): n is a, c, g, or t
  misc_feature (261)..(261): n is a, c, g, or t
  misc_feature (286)..(286): n is a, c, g, or t
  misc_feature (361)..(361): n is a, c, g, or t

SEQ ID NO: 177 - DNA, 469 bp, Homo sapiens; identical to SEQ ID NO: 44
  misc_feature (50)..(50): n is a, c, g, or t
  misc_feature (71)..(71): n is a, c, g, or t
  misc_feature (356)..(356): n is a, c, g, or t

SEQ ID NO: 178 - DNA, 475 bp, Homo sapiens; identical to SEQ ID NO: 45
  misc_feature (76)..(76): n is a, c, g, or t
  misc_feature (108)..(108): n is a, c, g, or t
  misc_feature (122)..(122): n is a, c, g, or t
  misc_feature (176)..(176): n is a, c, g, or t
  misc_feature (213)..(213): n is a, c, g, or t
  misc_feature (269)..(269): n is a, c, g, or t
  misc_feature (303)..(303): n is a, c, g, or t
  misc_feature (327)..(327): n is a, c, g, or t
  misc_feature (422)..(422): n is a, c, g, or t
  misc_feature (467)..(467): n is a, c, g, or t

SEQ ID NO: 179 - DNA, 174 bp, Homo sapiens; identical to SEQ ID NO: 46

SEQ ID NO: 180 - DNA, 106 bp, Homo sapiens; identical to SEQ ID NO: 47
  misc_feature (3)..(3): n is a, c, g, or t
  misc_feature (96)..(96): n is a, c, g, or t

SEQ ID NO: 181 - DNA, 140 bp, Homo sapiens; identical to SEQ ID NO: 48
  misc_feature (3)..(3): n is a, c, g, or t
  misc_feature (14)..(14): n is a, c, g, or t
  misc_feature (120)..(120): n is a, c, g, or t
  misc_feature (122)..(122): n is a, c, g, or t

SEQ ID NO: 182 - DNA, 213 bp, Homo sapiens; identical to SEQ ID NO: 49

SEQ ID NO: 183 - DNA, 344 bp, Homo sapiens; identical to SEQ ID NO: 50

SEQ ID NO: 184 - DNA, 339 bp, Homo sapiens
cagtagtccccccttatccgcggtttcactttccgcggtttcagttacccgcggtcaaccgcggtccgaaaataggtgagtacagtacaataagatattttgagagagagagaccacattcacataacttttattacagtatattgttataattgttctattttattattagttattgttgttaatctcttactgtgcctaatttataaattaaactttatcataggtatgtatgtataggaaaaaacatagtatatatagggttcggtactatccgcggtttcaggcatccactgggggtcttggaacgtatcccccgcggataaggggggactactg

SEQ ID NO: 185 - DNA, 550 bp, Homo sapiens
cagtagtccccccttatccgcggtttcgctttccgcggtttcagttacccgcggtcaaccgcggtccgaaaatataaatggaaaattccagaaataaacaattcataagttttaaattgcgcgccgttctgagtagcgtgatgaaatctcacgccgtcctgctccgtcccacccgggacgtgaatcatccctttgtccagcgtatccacgctgtatacgctacccgcccgttagtcacttagtagccgtctcggttatcagatcgactgtcgcggtatcgcagtgcttgtgttcaagtaacccttattttacttaataatggccccaaagcgcaagagtagtgatgctggcatattgttataattgttctattttattattagttattgttgttaatctcttactgtgcctaatttataaattaaactttatcataggtatgtatgtataggaaaaaacatagtatatatagggttcggtactatccgcggtttcaggcatccactgggggtcttggaacgtatcccccgcggataaggggggactactg

SEQ ID NO: 186 - DNA, 733 bp, Homo sapiens
cagtagtccccccttatccgcggtttcgctttccgcggtttcagttacccgcggtcaaccgcggtccgaaaatattaaatggaaaattccagaaataaacaattcataagttttaaattgcgcgccgttctgagtagcgtgatgaaatctcgcgccgtcccgctccgtcccgcccgggacgtgaatcatccctttgtccagcgtatccacgctgtatacgctacccgcccgttagtcacttagtagccgtctcggttatcagatcgactgtcgcggtatcgcagtgcttgtgttcaagtaacccttattttacttaataatggccccaaagcgcaagagtagtgatgctggcaattcggatatgccaaagagaagccgtaaagtgcttcctttaagtgaaaaggtgaaagttctcgacttaataaggaaagaaaaaaatcgtatgctgaggttgctaagatctacggtaagaacgaatcttctatccgtgaaattgtgaagaaggaaaaagaaattcgtgctagttttgctgtcgcacctcaaactgcaaaagttacggccacagtgcgtgataagtgcttagttaagatggaaaaggcattaaatttgtgggtggaagacatgaacagaaacgtgttccgattgatggcaatcgggttcggtactatccgcggtttcaggcatccactgggggtcttggaacgtatcccccgcggataaggggggactactg

SEQ ID NO: 187 - DNA, 705 bp, Homo sapiens; identical to SEQ ID NO: 51

SEQ ID NO: 188 - DNA, 1040 bp, Homo sapiens
cagggccggcttcatgggcgtgcgacctgtgcagtcgcacagggccccgcgctcagaagggccccgcgcttggtttaatgctctgctgtcgccgtcttgaaattcttaataattttatctttgaacttgtgttttgtaagtgaagtccgatgggacaatggagcatgcgcgtgagcagaggagatacgcgcaatatgcgtgtccgccgttccttgccgccccatttgcatatagcgttcgcgatgccccatgagcacagaattccggtggacccacgatgcgtgggagttcagcgagactcaaagcgagtacaaggtaagcgtgttacgtctacgactgagtaagcgggggcgctgacagccccgagaggccacgctttccgttcgaaccagaacttgcttcgaacgcagaaagaaggcaatggcattctaagaaacacgaacgaccaaggaaccctatcatatcctttcttactcgtgttacttccctgtattagccaaccacttacgctgaaaatgatgacatagaaggaaagggaaagatagggcaacccatagttccttttcctttcagtccttccttactcatcagtaagccgaaggtagagagtgttggtagaatgtgcgcgtatcaagaagtgaaataaaaacagttgagttagttttgtgcagcgtttccactgttctggtaagaacgaaatacatatgcatgtacgagctacgaaatacgaattgtgtaatttcggtgattccgcatacgagttaaatgctcttatatttgcatttaaaactggcattgcacaatataaagatgaatggtaaaattcatgctaataatttaaaattttaatttttctttacttagaatgacattaaatagcaaatataaaaacaccatgacaagtcgagagagagaccgcggaagaaaggaaaaagctttatattttagtacctttaatggcacttttttcctgctttttgaacaaggggccccacattttcattttgcactgggccccgcaaattatgtagccggccctg

SEQ ID NO: 189 - DNA, 418 bp, Homo sapiens; identical to SEQ ID NO: 52

SEQ ID NO: 190 - DNA, 97 bp, Homo sapiens; identical to SEQ ID NO: 53

SEQ ID NO: 191 - DNA, 432 bp, Homo sapiens
tgttaaagcgaactaaatacggcctgagaaggactccgtacttctatatttgagtccttgtggacgaaccgtaacctagcttaataggcagacaagattgaaaacctaacttaggagtatgcgcctgtaacaatagctgagtcttggccaatcccagcggccatacttcaaccactcatagactgccgagcgttcaaactgtgttcaaataaggcaaacgccgacccgtaaccaatccagccgtttctgtacctcacttccgatttctgtacgtcacttccctttttttgtctataaatttgttctgaccacgaggcatccctggagtctctctgaatctgctgtgattctgggggctgcccgattcgcgaatcgttcattgctcaattaaactcctttaaatttaattcggctgaagtttttcttttaaca

SEQ ID NO: 192 - DNA, 403 bp, Homo sapiens
tgttaaattaagtttagcctaaagctgcctccttacatattttaagttcggcctaaaggtttctccgtacatagtgaaccgtaacctaactggatgtgtaaacagaccgtaacctactcttgtaccaatcaccgagtttcggccaatcacaggcggccaactgttcaaaccgtgttcaaataaggcaaacgccgagctgtaaccaatccggctgtttctgtacctcacttccgttttctgtacgtcgctttcctttttctgtccataaatcttctccgaccacgcggcagccccggagtctctctgaacctattctggttccgggggctgcccgattcgcgaatcgttctttgctcaattaaactctgttaaatttaatttgtctaaagtttttcttttaaca

SEQ ID NO: 193 - DNA, 224 bp, Homo sapiens; identical to SEQ ID NO: 54

SEQ ID NO: 194 - DNA, 341 bp, Homo sapiens; identical to SEQ ID NO: 55

SEQ ID NO: 195 - DNA, 386 bp, Homo sapiens; identical to SEQ ID NO: 56

SEQ ID NO: 196 - DNA, 263 bp, Homo sapiens; identical to SEQ ID NO: 57

SEQ ID NO: 197 - DNA, 865 bp, Homo sapiens; identical to SEQ ID NO: 58

SEQ ID NO: 198 - DNA, 1061 bp, Homo sapiens
  misc_feature (633)..(633): n is a, c, g, or t
  misc_feature (644)..(644): n is a, c, g, or t
  misc_feature (695)..(695): n is a, c, g, or t
  misc_feature (748)..(748): n is a, c, g, or t
  misc_feature (750)..(751): n is a, c, g, or t
cagtggtgtgctggagccggctcataccggctcgcgagagccgattgttaaattttcaggaattttgcgagccggttgttaaacacagccattattaaaaattaaattatataaacttacaattaaataaattatattaaaaacaaaggtaataaatactcaaaactcatcacttcctaattattttactacattttactattatctatgctcttgaggttatttacgtctattgtatctgtatggtggaaatactatataatggtgtgctactgcgcatctcttcccaactccgcgttcagtgacgtcacgttggtagcttgaaatcggccatggtgggagtatttacaccacggaaattggcaaacgctacaaatcagggcttgatttattgttttgttgattgtctagacttaagaaagtgatggagaaaatgttaataatgcagattaaacttaaaagtgtgtcgtgtctgtagccgttacattgtgaatagcacaaaaaattgaggaaatattcttccagtatttgaaaactattatccgattcagcaaagaagtcgctcacatcattgacgaacgagtgaagttccgacatacgtcttcgttgtttcactttcgtcttactttaatnaatataatttntacgaaggtgagaaatagtttaacagtagatcacatcagttattatgaaantaaatttattggaaagagttatagattgggatgcaactccatttgtcaaatcntnntcttactcattaatgtaaacgaaaatatcaaccaacattcatgttggaactacactcgttcgtcaattgcaaccataggttggctacggatacaagagttcggcaaaaatcaataaaagcattctgtgagaatcaattggctatatggaatttacaataaagagtattgtatattttattattatttgtaaattgtgtgctacacatcctttatatcagtaaaatttataataaacttatatatgtatatacatacatacattttttcccccagagagccagttgttaaacatttaccagcacaccactg

SEQ ID NO: 199 - DNA, 605 bp, Homo sapiens
cagcaggtcctcgaataacgtcatttcgttcaacgtcgtttcgttataacgttgatgagaaaaaaaatcgattcccggccggggccactgtctgtgtggagtttgcacgttctccccatgtctgcgtgggttttctccgggtactccggtttcctcccacatcccaaagatgtgcacgttaggtkaattggcgtgtctacatggtcccagtctgagtgagtgtgggtgtgtgtgtgagtgcgccctgcgatgggatggcgtcctgtccagggttggttcccgccttgtgccctgagctgccgggataggctccggccacccgcgaccctgaactggaataattgggtaaataattatcttacttgtttttattaatctttcttaaatgtatgtatagctcacatttatttcaatgtttaatattagaagtgttttggtctttatttagaagtttggtgatgtttttgtgaccagaaatatgccgtaggaacttaactcttgtttatatcaattagcctatggtaaaattggtttcgttatacgtcgtttcgcttaaagtcgcagtttccaagaacctatcgacgacgttaagtgaggacttactg

SEQ ID NO: 200 - DNA, 210 bp, Homo sapiens
cagtaagtcctcacttaacgtcgtcgataggttcttggaaactgcgactttaagcgaaacgacgtacagcaggtcctcgaataacgtcgtttcgttcaacgtcgtttcgttataacgttgatgaggaaaaaattggtttcgttatacatcatttcgcttaaagtcacagtttccaagaacctatcgatgacgttaagtgaggacttactg

SEQ ID NO: 201 - DNA, 202 bp, Homo sapiens; identical to SEQ ID NO: 59

SEQ ID NO: 202 - DNA, 514 bp, Homo sapiens
  misc_feature (86)..(86): n is a, c, g, or t
cccttttcccgtttgccccgagaatactcgccggcggcgcttgcggctgcagcgtttaccccgagataactttgccatgaaatatnttgcttttattattattttcgcatcgttctagtatatcgactttggaaacaaaagacatcgttctatttatagcattctgtttttagtagtggtatttccatttacaaaatatagtaattctcgattgctgaaaatgtcaaatcctagaaaacgtagcattcctacacgtgatgttaacatcgttctcgaacagttgttggccgaagattcatttgatgaatccgatttttccgaaatagacgattctggtgattcagatgattctgatgttagttctgtttagaaataactccaagaacagtttttatattttattttcacattgaaaatcagtcagatttgcttcagcctcaaagagcgtgtttatgtaaaattaaatgagcgctggcagcgagctgcacttttttttttctaaacgggaaaaggg

SEQ ID NO: 203 - DNA, 77 bp, Homo sapiens
aacccatttcccgtttgccccgagaatactgcgctggcagcgagctgcactttttttttctaaacgggaaatgggtt

SEQ ID NO: 204 - DNA, 239 bp, Homo sapiens
  misc_feature (19)..(20): n is a, c, g, or t
  misc_feature (102)..(102): n is a, c, g, or t
cagttgtccctctgtatanncgggggattggttccaggacccytgtgtatacmaaaatccgcgcatactcaagtcccgaagtcggccctgcggaacccacgnatatgaaaagtcggccctccatatatacgggtttcgcatcccgcgaatactgtattttcaatccgcgtttgattgaaaaaaatccgcgtataagtggacccacgcagttcaaacccgtgttgttcaagggtcaactg

SEQ ID NO: 205 - DNA, 894 bp, Homo sapiens
  misc_feature (180)..(181): n is a, c, g, or t
  misc_feature (378)..(378): n is a, c, g, or t
  misc_feature (404)..(404): n is a, c, g, or t
  misc_feature (435)..(435): n is a, c, g, or t
  misc_feature (442)..(442): n is a, c, g, or t
  misc_feature (494)..(494): n is a, c, g, or t
  misc_feature (717)..(717): n is a, c, g, or t
  misc_feature (734)..(734): n is a, c, g, or t
  misc_feature (843)..(843): n is a, c, g, or t
cagtggcgtaccaagggcggggcggtgggagcggtccgccccaggtgcaggcaataagggggtgcattgtctgtagagaatttaaaaacaataataaaactgactaaaagtcggtctgctttttattatcaccatgcgccggcaattctaaacaatgtcagtgataaaatactcctcccnnaaaaatcttttgttggtctaagttctaaacaattgctgcggttactgttgagttttaataatatatatatgtaaacttcaaattagcacatttttattacttatcctttaataaacattgtattctacatggaagttaattcggagaactcccagttatacagtcggcccccgacacacgcggactcagctacacgnattcgtttcgagagtaagttcataanggttcggaatcattcgagctcgcttcgggtncagttcntgtctccaacccctgtggtactacatattcctgcgtttaaacagtagatttnaaataaacaatgatagcacagtgattgtaaagacgaagaaacagaacttgagttacttcaattctgtcattctatgtgaccacttggagtttttatttgtgtttaaaatttaaaacagtgaaacagagtgcgaactgcgaggtgtaatatttttgtttggtaagtgcaaattttagttcatacatgaaatattttactgaatttgaataatatctttaaaatngaaatttattcttcttnaaattgttaattatttgttttaaaactaaagaacaaaatcaaaaaaatgattattactgattattacatgattattactgaaaataattttgtcatatagaggaagggngtgttaaaaaatgatccgctctgggtgtcgaatacgctaggtacgccactg

SEQ ID NO: 206 - DNA, 1205 bp, Homo sapiens; identical to SEQ ID NO: 60

SEQ ID NO: 207 - DNA, 756 bp, Homo sapiens; identical to SEQ ID NO: 61

SEQ ID NO: 208 - DNA, 240 bp, Homo sapiens
ccctttgcactcggatgtcgagtgtgactcgacacggttagcaaaaattatagagattaaaattactctttgaatgtatcaataatttgaaatataaaaaaatccaaataaataagtttgtatgaaaagaaactccagttttttattctactgccacgctttgtaaaatctggggtatttaaaaaattaaatcccgagtagaataaaggaatcgagaaaaaagcaagcgagtgcaaaggg

SEQ ID NO: 209 - DNA, 348 bp, Homo sapiens
ccacttcgggacgagcgtcgactatagtcgacagccacagatgaacgcgcacagcgactttagccgacagccgtgatatgacttttctaatttttcatttatcaaaataaaattgtgaacatttaaaaataacataatgaaaacatatatgtatatgttacctattctgatttacattacaagtaaagctgcctgtaaagtaaaacaagctttcagtgctttaaagctttcctcatcacacaagagcaaaacggattcgtcgtcaatgcacagcacaaactatcgtgcggactgtgagtgccggctgtgggcaaggtttcgcggccggtgagcgccgtaccgaagtgg

SEQ ID NO: 210 - DNA, 1708 bp, Homo sapiens; identical to SEQ ID NO: 62

SEQ ID NO: 211 - DNA, 1181 bp, Homo sapiens; identical to SEQ ID NO: 63
  misc_feature (1067)..(1067): n is a, c, g, or t

SEQ ID NO: 212 - DNA, 1647 bp, Homo sapiens; identical to SEQ ID NO: 65

SEQ ID NO: 213 - DNA, 1869 bp, Homo sapiens; identical to SEQ ID NO: 66

SEQ ID NO: 214 - DNA, 87 bp, Homo sapiens
gcgcctctctgcgcctgcgccggcgcsscgcgcctctctgcgcctgcgccggcgcsscgcgcctctctgcgcctgcgccggcgcssc

SEQ ID NO: 215 - DNA, 174 bp, Homo sapiens
gcgcctctctgcgcctgcgccggcgcsscgcgcctctctgcgcctgcgccggcgcsscgcgcctctctgcgcctgcgccggcgcsscgcgcctctctgcgcctgcgccggcgcsscgcgcctctctgcgcctgcgccggcgcsscgcgcctctctgcgcctgcgccggcgcssc

SEQ ID NO: 216 - DNA, 791 bp, Homo sapiens; identical to SEQ ID NO: 67
  misc_feature (569)..(569): n is a, c, g, or t

SEQ ID NO: 217 - DNA, 1068 bp, Homo sapiens
cagttgacccttgaacaacacgggtttgaactgcgcgggtccacttatacgcggatttttttcaataaatatattggaaaattttttggagatttgcgacaatttgaaaaaactcgcagacgaaccgcgtagcctagaaatatcgaaaaaattaagaaaaagttaggtatgtcatgaatgcataaaatatatgtagatactagtctattttatcatttactaccataaaatatacacaaatctattataaaaagttaaaatttatcaaaacttacgcacacacttacagaccgtacatggcgccattcgcagtcgagagaaatgtaaacaaacgtaaagatgcagtattaaatcataactgcataaaattaactgtagtacatactgtactactgtaataatttcgtagccacctcctgttgctattgcggtgagctcaagtgttgcgagtatccgcttaaaacgccgtgtgacgctaatcatctccgcgtgagcagttcgtctctccagtaaattgcgtatcgcagtaaaaagtgatctctcgcggttctcgcgtatttttcatcgtgtttagtgcaataccgtaaaccttgaataacaccatgggacccatacgaagtgccactagtgatgctggaagtgctcccaagaagcagagaaaagtcatgacattacaagaaaaagttgaattgcttgatatgtaccgtagattgaggtctgcagctgcggttgcccgccatttcagacagatgattcatcttgtaaacagatgacgtaaacttacggtatcgataaatacagtacagtactgtaaatgtattttctcttccttatgattttcttaataacattttcttttctctagcttactttattgtaagaatacagtatataatacatataacatacaaaatatgtgttaatcgactgtttatgttatcggtaaggcttccggtcaacagtaggctattagtagttaagtttttggggagtcaaaagttatacgcggattttcgactgcgcggggggtcggcgcccctaacccccgcgttgttcaagggtcaactg

SEQ ID NO: 218 - DNA, 602 bp, Homo sapiens
cagtcatgcgccacataacgacgtttcggtcaacgacggaccgcatatacgacggtggtcccataagattataatggagctgaaaaattcctatcgcctagtgacgtcgtagccgtcgtaacgtcgtagcgcaattactttatttttaaataaatttagtgtagcctaagtgtacagtgtttataaagtctacagtagtgtacagtaatgtcctaggccttcacattcactcaccactcactcactgactcacccagagcaacttccagtcctgcaagctccattcatggtaagtgccctatacaggtgtaccattttttatcttttataccgtatttttactgtaccttttctatgtttagatatgtttagatacacaaatacttaccattgtgttacaattgcctacagtattcagtacagtaacatgctgtacaggtttgtagcctaggagcaataggctataccatatagcctaggtgtgtagtaggctataccatctaggtttgtgtaagtacactctatgatgttcgcacaacgacgaaatcgcctaacgacgcatttctcagaacgtatccccgtcgttaagcgacgcatgactg

SEQ ID NO: 219 - DNA, 321 bp, Homo sapiens
cagtcatgcgccgcataacgacgtttcggtcaacgacggaccacatatacgacggtggtcccataagattataatggagcatatatagaaacctgatatatggcacttgatattggcattgcagatcaagtaggggaaatgactgatattcagtaatggtgctgggacatttggttttccatatgaaaaaatatatataaataaaaatatatataccatctaggtttgtgtaagtacactctatgatgttcgcacaacgacaaaatcgcctaacgacgcatttctcagaacgtatccccgtcgttaagcgacgcatgactg

SEQ ID NO: 220 - DNA, 236 bp, Homo sapiens
caggttgagcatccctaatccgaaaatccgaaatccgaaatgctccaaaatccgaaactttttgagcgccgacatgacgctcaaaggaaatgctcattggagcatttcggatttcggattttcggattagggatgctcaaccggtaagtataatgcaaatattccaaaatccgaaaaaatccgaaatccgaaacacttctggtcccaagcatttcggataagggatactcaacctg

SEQ ID NO: 221 - DNA, 366 bp, Homo sapiens; identical to SEQ ID NO: 69

SEQ ID NO: 222 - DNA, 446 bp, Homo sapiens; identical to SEQ ID NO: 70

SEQ ID NO: 223 - DNA, 624 bp, Homo sapiens; identical to SEQ ID NO: 71

SEQ ID NO: 224 - DNA, 76 bp, Homo sapiens; identical to SEQ ID NO: 72

SEQ ID NO: 225 - DNA, 76 bp, Homo sapiens; identical to SEQ ID NO: 73

SEQ ID NO: 226 - DNA, 75 bp, Homo sapiens; identical to SEQ ID NO: 74

SEQ ID NO: 227 - DNA, 75 bp, Homo sapiens; identical to SEQ ID NO: 75

SEQ ID NO: 228 - DNA, 77 bp, Homo sapiens; identical to SEQ ID NO: 76

SEQ ID NO: 229 - DNA, 77 bp, Homo sapiens; identical to SEQ ID NO: 77

SEQ ID NO: 230 - DNA, 85 bp, Homo sapiens; identical to SEQ ID NO: 78

SEQ ID NO: 231 - DNA, 86 bp, Homo sapiens; identical to SEQ ID NO: 79

SEQ ID NO: 232 - DNA, 76 bp, Homo sapiens; identical to SEQ ID NO: 80

SEQ ID NO: 233 - DNA, 75 bp, Homo sapiens; identical to SEQ ID NO: 81

SEQ ID NO: 234 - DNA, 85 bp, Homo sapiens; identical to SEQ ID NO: 82

SEQ ID NO: 235 - DNA, 85 bp, Homo sapiens; identical to SEQ ID NO: 83

SEQ ID NO: 236 - DNA, 85 bp, Homo sapiens; identical to SEQ ID NO: 84

SEQ ID NO: 237 - DNA, 85 bp, Homo sapiens; identical to SEQ ID NO: 85

SEQ ID NO: 238 - DNA, 76 bp, Homo sapiens; identical to SEQ ID NO: 86

SEQ ID NO: 239 - DNA, 76 bp, Homo sapiens
ggggaattagctcaaatggtagagcgctcgctttgcttgcgagaggtagcgggatcgatgcccgcattctccacca

SEQ ID NO: 240 - DNA, 77 bp, Homo sapiens
  misc_feature (55)..(55): n is a, c, g, or t
gtctctgtggcgcaatcggttagcgcgttcggctgttaaccgaaaggttggtggntcgagcccacccagggacgcca

SEQ ID NO: 241 - DNA, 75 bp, Homo sapiens
tccctggtggtctagtggdtaggattcggcgctctcaccgccgcggcccgggttcgattcccggtcagggaacca

SEQ ID NO: 242 - DNA, 76 bp, Homo sapiens
gtttccgtagtgtagtggttatcacgttcgcctcacacgcgaaaggtccccggttcgaaaccgggcggaaacacca

SEQ ID NO: 243 - DNA, 145 bp, Homo sapiens
  misc_feature (1)..(1): n is a, c, g, or t
nagctttgcgcagtggcagtatcgtagccaatgaggtttatccgaggcgcgattattgctaattgaaaacttttcccaataccccgccgtgacgacttgcaatatagtcggcattggcaatttttgacagtctctacggagactg

SEQ ID NO: 244 - DNA, 107 bp, Homo sapiens
gtgctcgcttcggcagcacatatactaaaattggaacgatacagagaagattagcatggcccctgcgcaaggatgacacgcaaattcgtgaagcgttccatattttt

SEQ ID NO: 245 - DNA, 236 bp, Homo sapiens; identical to SEQ ID NO: 87
  misc_feature (7)..(7): n is a, c, g, or t
  misc_feature (45)..(45): n is a, c, g, or t
  misc_feature (48)..(48): n is a, c, g, or t
  misc_feature (59)..(59): n is a, c, g, or t
  misc_feature (207)..(207): n is a, c, g, or t

SEQ ID NO: 246 - DNA, 340 bp, Homo sapiens; identical to SEQ ID NO: 88
  misc_feature (13)..(13): n is a, c, g, or t
  misc_feature (18)..(18): n is a, c, g, or t
  misc_feature (36)..(36): n is a, c, g, or t
  misc_feature (190)..(190): n is a, c, g, or t

SEQ ID NO: 247 - DNA, 289 bp, Homo sapiens; identical to SEQ ID NO: 89
  misc_feature (14)..(14): n is a, c, g, or t
  misc_feature (24)..(24): n is a, c, g, or t
  misc_feature (33)..(33): n is a, c, g, or t
  misc_feature (37)..(38): n is a, c, g, or t
  misc_feature (77)..(77): n is a, c, g, or t
  misc_feature (256)..(257): n is a, c, g, or t
  misc_feature (273)..(273): n is a, c, g, or t

SEQ ID NO: 248 - DNA, 330 bp, Homo sapiens; identical to SEQ ID NO: 90
  misc_feature (24)..(24): n is a, c, g, or t
  misc_feature (42)..(42): n is a, c, g, or t
  misc_feature (52)..(52): n is a, c, g, or t
  misc_feature (54)..(54): n is a, c, g, or t
  misc_feature (65)..(65): n is a, c, g, or t
  misc_feature (308)..(308): n is a, c, g, or t

SEQ ID NO: 249 - DNA, 343 bp, Homo sapiens; identical to SEQ ID NO: 91
  misc_feature (36)..(36): n is a, c, g, or t
  misc_feature (58)..(58): n is a, c, g, or t
  misc_feature (60)..(60): n is a, c, g, or t
  misc_feature (62)..(62): n is a, c, g, or t

SEQ ID NO: 250 - DNA, 1268 bp, Homo sapiens; identical to SEQ ID NO: 92
  misc_feature (24)..(24): n is a, c, g, or t
  misc_feature (34)..(34): n is a, c, g, or t
  misc_feature (315)..(315): n is a, c, g, or t
  misc_feature (904)..(905): n is a, c, g, or t
  misc_feature (1033)..(1033): n is a, c, g, or t
  misc_feature (1178)..(1178): n is a, c, g, or t
  misc_feature (1182)..(1182): n is a, c, g, or t
  misc_feature (1216)..(1216): n is a, c, g, or t
  misc_feature (1231)..(1232): n is a, c, g, or t
  misc_feature (1238)..(1238): n is a, c, g, or t

SEQ ID NO: 251 - DNA, 182 bp, Homo sapiens; identical to SEQ ID NO: 93

SEQ ID NO: 252 - DNA, 165 bp, Homo sapiens; identical to SEQ ID NO: 94

SEQ ID NO: 253 - DNA, 323 bp, Homo sapiens; identical to SEQ ID NO: 95
  misc_feature (13)..(13): n is a, c, g, or t
  misc_feature (28)..(28): n is a, c, g, or t
  misc_feature (49)..(49): n is a, c, g, or t
  misc_feature (93)..(93): n is a, c, g, or t
  misc_feature (172)..(172): n is a, c, g, or t
  misc_feature (219)..(219): n is a, c, g, or t
  misc_feature (271)..(271): n is a, c, g, or t
  misc_feature (283)..(283): n is a, c, g, or t
  misc_feature (299)..(299): n is a, c, g, or t
  misc_feature (304)..(304): n is a, c, g, or t

SEQ ID NO: 254 - DNA, 2623 bp, Homo sapiens; identical to SEQ ID NO: 96
  misc_feature (839)..(839): n is a, c, g, or t
  misc_feature (846)..(846): n is a, c, g, or t
  misc_feature (916)..(916): n is a, c, g, or t
  misc_feature (1006)..(1006): n is a, c, g, or t
  misc_feature (1014)..(1014): n is a, c, g, or t
  misc_feature (1062)..(1062): n is a, c, g, or t
  misc_feature (1064)..(1064): n is a, c, g, or t
  misc_feature (1137)..(1137): n is a, c, g, or t
  misc_feature (1184)..(1184): n is a, c, g, or t
  misc_feature (1313)..(1313): n is a, c, g, or t

SEQ ID NO: 255 - DNA, 234 bp, Homo sapiens
caggttgagcatcccaaatccgaaaatccgaaatccgaaatgctccaaaatccgaaactttttgagcgccgacatgacgctcaaaggaaatgctcattggagcgttttggatttcggattttcagatttgggatgctcaaccggtaagtataatgcaaatattccaaaatccaaaaatccgaaatccgaaacacttctggtcccaagcattttggataagggatactcaacctg

SEQ ID NO: 256 - DNA, 131 bp, Homo sapiens
aaaaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 257 - DNA, 134 bp, Homo sapiens
aaaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 258 - DNA, 310 bp, Homo sapiens
atggcagagatagaataaaaacagaaaaatggcgacggtcacgttgtggcgagccttgctgcgtcattagataatcctcatgcaaatagcgggaagaacaaaggaaggggagcccgggacccccgggggcgcaggatccggcgggaggagtctaagaggaggaggcggcggtgccggaggaggaggaggagggagggagaagagaggaagaccggagtccccgcggcggcggcggtccggagagagggcgagccccgcgcggcgccggggaccgggcgctaccacgaggccgggacgctggagtctgggg

SEQ ID NO: 259 - DNA, 209 bp, Homo sapiens
agggattttttaattttaagctatttgtctgttaagtatataataccaaaacgcaggttgtttaaattaggatttccaagtaatttatgtcgtcttcaaaattcctggggtctatcaatcagaaacgccagaaagtttgtgtactagtttcacattgttaagggagtatctataataaaattcaaatgcgttattttaaaataagtaaa

SEQ ID NO: 260 - DNA, 102 bp, Homo sapiens
ggtcctctgactctcttcggtgacgggtattcttgggtggataatacggattacgttgttattgcttaagaatacgcgtagtcgaggagagtaccagcggca

SEQ ID NO: 261 - DNA, 575 bp, Homo sapiens
gtacgacacaggaaaacgtcagagactaagcaaatttgaatagacctctgagtaaatatttcccttttggagttttcaggactttctgtcccgctgttttatggggaaggcgggggaagacacgcagacatcaattcgactaaacaaagtttatcaataatattaaaaataaaaagtaaagcccctttctgaacattatggcttattccttgattatccttcttaatgccacagcgtggctatctcagaacaaaattagagactaacagtaaatttactttaatttttacgaataattctctatcaatgtaaaaatttttcatatacgcgtgtatgaaagaaaaacactactatttttccacattcgtgaaaactgcttaactgggacagtaaccgagcggccagtgaatatttaaaatactgtaaccgacgaatacgtatgcttctaggaccacgtggaatctttcccatggtaggtaagcaaaacaaatgtgcttcccagaaccctccaaactgctcgtgggtatattactacagattacataatgcagcagcgttccgggacgcaagcccag

SEQ ID NO: 262 - DNA, 82 bp, Homo sapiens
ccgatacaatgatgataacatagttcagcagactaacgctgatgagcaatattaagtctttcgctcctatctgatgtatctg

SEQ ID NO: 263 - DNA, 130 bp, Homo sapiens
ggaggtgagttcgcagccggaacgttgcaggcacttgtttcctcagtggatgcctttcggcgcgccgccgcccggcgccgcgggactgggagaccggatggtggaattctggaaacatcctgtgttgaag

SEQ ID NO: 264 - DNA, 216 bp, Homo sapiens
gtttcctcagtggatgcctttcggcgcgccgccgcccggcgccgcgggactgggagaccggagtcaacttttataacactgttactgggaatacttgacttactaagcttttactgaacactttaattttgggagtacaatttctaaactcacgaaataatcttcatgcaacaagatgttattttatcaaattttggtattacatgctcattcttt

SEQ ID NO: 265 - DNA, 233 bp, Homo sapiens
attccttctgcctccccctaacatttctggaccgaggcggagagagctcccggtagaaccgacgcaaccatcccgggatggtgaccaacgcgcggctgctgcgggcagagggactgaagagaaccggtggtcccgacgctggccccgagcccgaggagttaaactaacaaagagctccccgagccgcgaaaccccccagagcggaagcgacgcctccccgccgccgccgccgc

SEQ ID NO: 266 - DNA, 553 bp, Homo sapiens
cttccaccaataaattcaagtttttattcttgtttataaccggcatttccaggtccggccaggtccaaaaaaaaaaaaaaggaattgagggtttgcatcgtttttcaatataactggaagctgttttatcactttgtattttgagaaccgctccctttcccgcacacgcattgctgcctcctctcgggcttggttaatgagtctgtgcgccaagccaggtcgctccgggcagttcgcgcttccgcgcctcggcactcggatgaccgtgtcctattcgtctctctcgtgaatgtcgctggctggtggcgcggggaaaccatggcagcattgcggctccggcgggcgggctctgcgcggggccccaccgagctttccgggagcctctcccgcagccgatgggcatctaggggcgcagaacgaagagtgggcgccgaaacgggtgtaggcgctggaggccgacggggaggcccggggcggtcaggcttctcggtagagagggccgtgcacctcgcggcctgcgggctaaggcggggagccgctcctgcggcggccg

SEQ ID NO: 267 - DNA, 218 bp, Homo sapiens
ccaagcgggcgcgaagagggactggaccagcgggttggcgggcggagggcggaggcgcggcgtgccgcgggagcgcgcgcgcctcgccagctttcaggggaagagggcctttgccgccgttcgcgtcagggcgagttccgggacgtcagtgcggtctccgccaatgtatggatttgacaatgcctttgattgacctcgcagctgctcatcacacaaaa

SEQ ID NO: 268 - DNA, 391 bp, Homo sapiens
taggacgtgcccccactaatcaggtagtcggccatggccaacacaggacaccgccgcgtgtctccgaggaccgcgccgcagagacctctgccgtctgggccgcgccaatataaaccagccgatttcgtcactgcgctgcgcgcgacgctgacgtcagggcgtagtgcgcgcgggcggaacctacccgcctcgctgcggcgggggcggagctggcgctgaagaaacagcgcatgcgtatagcacatagtggccttgtcactgaggagctgccacttcacgtcccaccccacactccccgcaggacctcggccagttcaattaacaggcaagggtttacttctactgtcccgccgggctcctccccaaaataaagagacgttgttcactaaca

SEQ ID NO: 269 - DNA, 500 bp, Homo sapiens
gctttggggccacagaacaatcaggcgcgcatggctttttctccgggagatgccgctgaaaacgcacaagtcgccatctgagctgcaagagtcagccccaaattgtgtcctttcattttagggttcggcaaaaacggggagcaaaataggtgaaagtcgccccggaacttattgctgtgcgggcgaagggcgaagagcagcgagtgcaccgcgggcgcgaggctggggaagggcgagagctcggagctccgcggcggcgactcagctccggcggtccatggccggcgaagctgcccacctcctcgtttggcgcccgggtccgaggggcgggagagcgggccggcgggaggcgggcggtcccgggcacaacggcggcggcggaagggctcgctgggcagctgccgcacggaccccggctctgggcggcggaggcggctccgtggagctcgcagcagatttccacgcgatcctgtgccccgcaaacagactgaccgaccgcg

SEQ ID NO: 270 - DNA, 424 bp, Homo sapiens
ctaactgcccggcaccccgcgactgggttggggtcactcgtctccctcgcgttctcccgggatcttatgtttctctgcgtttcattccgttcaaagaaacgtggggcacgcgcggcgggtgcggctgcagcaggcgaccctccagcgcaccttcgaaggacgtccctgccctctgccttgcctcgtattgtggttcactatttgtcattcacctgaaaggagaaggaaaagtacgaaaggtttctctgcttgggaaaaggtgaatgttgttatatcaagaacgattacacacatggatctcatacatgtttttagaacattgttcttctgattgaagaagtctgatgctcctgaaaaatcttaaaatatctgacttgtattgaagaaaattatttaattaaatttttaaaggctggttgaaaaa

SEQ ID NO: 271 - DNA, 612 bp, Homo sapiens
tccccaccactccagtagcggcgggagcagcagcgttaggccggggtgtggctgcacctctgcgaaggctgtcgtgccccgtgcagctgcggcgttcgggtgggggaggggaggctcacaccctgcggcggcttaagcctggatttacgcactggaggagggaattagccctgagatgtcagccctgtgcccctggcgctgtccacccggacgcccgcggagcgccaggcgccgcagcggaccgcgcgcgcactgattgccgcagctactctccctgccgtcgccgcccgggaccgggtgacattgaggttttcgtctatcgccgctgggccgatagggcttctttttaagatttggctttccaagtaagatttttgtttgcctgcttttctcctggaatttttttgtttttggtcgaagaagcaggtaacttgcatatcttaaggaaaacattgtttttgctttgcttttgttgtttaaagatcctaaaacgtaccgtgattcctttacgttactaataattggaaagtgtgttaaatcaagggttcgcttaatgttctttagcctttgtacatagaatatataaatgtcgtgagtcatctttacgaggtt

SEQ ID NO: 272 - DNA, 327 bp, Homo sapiens
ggaatagcgtcatcagttctataagagagcgtgtgccgaaggcctcggcctttcacattcgggaagcgtcgggattaggtgaaagaagctgagctgaacacattacgatggatgatggaaacataagactatcaagaaatccaagtggtaatgggcgaagtttattcagcatccggcaatggacttatcgtagttggggaaacgggtgttccgaataatatcctggaagttatcaggacacctattttaaatataggcctgaattttgtaaagtaatatttaaggtggtccgtgataattaaataaaatgcttaattcatgtggcta

SEQ ID NO: 273 - DNA, 189 bp, Homo sapiens
cgtagtccgactagcagcagccgctgctccccggtggttcagagccgcgcgagctgagcgtttcgcctacaaaagcatcttcggatcggtttcccaccgccgaatgttgcttgagccaatgtgactggctctaggaggaaataaatcacatctgtcaaaagagcctcgagtcgaaggataggagaaacc

SEQ ID NO: 274 - DNA, 481 bp, Homo sapiens
acaagtcccggaggcacagaaagggcaaccattctaaatccttggtaagggctccgtgcagtagttaaccccccaaatccgactaaggagagcaggacggcggaacccgggtgccctaatcaaatatgggcaccaaggaaggaaaaaagcgtccgcatctgctcgctggtcaccgccgacgacagattcgtcacctgaatcaccgacgtgggggtgaggccgaagcctaagcccaaaccgaagccgccgccgctgttcatcccgatcccgctgccgttgccttcccgctccccaacgtctacgacgcgtcagcgacggagccgggaaagcggagaacgcgcggccgcgagcgcgctcccgctggcgaattcacagcgccgggcagatctagccgcccatttcacaactcccctgctcccggggctgcgcgcgccagccggaagcgtcgccccggcaacccggcggttctcgcgcggctgcg

SEQ ID NO: 275 - DNA, 411 bp, Homo sapiens
agccacagaactcggccgagggttcagcggactgcggctgcgcggcgccggcgacagcgtcaactgcttttgtgtcaaaaggaaaccaacagccggctccatagctcagggggggagacgatggcactaccgcgccccttcccgccatgcgtttgtttgcggcgttccccgacgcgcgcgctgaggctctggtctgcggctctgcgcttggcggctgcgcgccctcggccttcgggtccccgccgcctccgtgctgcaaggtctgatttcttcctgtggagttaacacggaaaagcgcagacggccacattcatgacccgggagtaacggctctacctgtcacattcgctttcagcctaaaacaaaattttagtttattttagacaaaatatataactaaggtatgctggc

SEQ ID NO: 276 - DNA, 850 bp, Homo sapiens
cggccgccccggcctccacgcaagcacccgagcgttactttcgtttccgcggcaaacgtctggaggcccgggtgagggaagaagcggcggcggagggttggggattactccgacgccgcggcagccttgcccgggcgtgctgggggagggcgcgcgaaccccgaccaggggggtcaccgggactgagcccggcggcctccggaatgttccccgcgcggtgccagtccgctcaccgcgcccttctccgtggccgcgccgtccagccaagtcctgccgcgctccgtcggccccgcccggatggccgcgctctggcgccccagctgtgggtcccctggagtcgccgcgctccggcagctggcccgccgtccgcgtcgagcgcgcccggctggagggcgtggactgttcgcgctgcctcctccccctgccccggcccttgccggggaggaggaccgcgagagcgccgccccgccgctcgtgggacctcgcgcccggggtctgagtcccggagctctcttgcgggctagaggatcgcggactttgttttcaagcgagtggggcggcctacgtaaaagaggaaaccgaaacctccgcgcgcacgagagggagaagggtctggagggaagcactgggtggattacagcgggggcgctatccggaggattcagagctctgcgggttagcataggcaagttagcattcctctaccttcatctgtaaagtggggctgttgaggaggaaatgtgattattttgcagccctttaaaatggtaattgtgaagtctcgagcaaagttggaactgcatgctattcatgtatattaaaaatcaaataataaaattatatgcacatc

SEQ ID NO: 277 - DNA, 455 bp, Homo sapiens
gcggagagggggcgcgctgacgtctcgccggcttaacctgttgctctcgagactgcgtcggagcccgcaggcagggccgcgggaagccggccgggccgccgccgccatgctcggccgtgtgcgcgccgccgccagccacgggcttgcagccggacgcccggactcggagacacgatccgctgaacgcccgcaataagaaagaaaacccaccccacgctgctctccacctcaacgaggtccggaatgggaggcggggtgcggttctcgtccaagttctaccccatttgctttgtggctgcatccgaaacggacgtgccccgggaaggcgtccacgctccaagacaaggttgcctcagtgcagtttctacagcccgagtttaaactagtcctttgcattctacccagccctctgcaatgcggatgtattttaattcttaaattctttatggaacagc

SEQ ID NO: 278 - DNA, 374 bp, Homo sapiens
aggcccccggcgccggcattccgggcgtcgagcacatcttgacggcgcggcgcggcccccacttccggaaattaagggccgcgcctcagggcctcgaaatcctccgcgccgcctgaaggcgagtcccagccaaactaggttcaaaggacccagtggaggggaagaatgccggcgaggccttttctcggcaactcccccgtcgagggtcctccgggactcccgccacggactgggagaacatactatttcatatcaaatcagattctccacgttacctactcaagtcataagcgtctgaagaaagtgctctgtaaattctcagcagaatatgcttttaacaattctttaaaaacaataaagtcataataaaaatc

SEQ ID NO: 279 - DNA, 75 bp, Homo sapiens
tatctgtgatgatcttatcccgaacctgaacttctgttgaaaaaaaaaaacttttacggatctggcttctgagat

SEQ ID NO: 280 - DNA, 70 bp, Homo sapiens
aatcaatgatgaaacctatcccgaagctgataacctgaagaaaaataagtacggattcggcttctgagat

SEQ ID NO: 281 - DNA, 495 bp, Homo sapiens
tcgtccgggcaccggcacggcccgctgcagttctggccgacggccggctgcagggaggacagggcggtcagcggcgctcggtcggcgcgcacgccctccccggccggccccgagtccctccctggcagccgccggccgcagcgggggctcccggcgcttacccggctgcagagggcgaggaggaccacgaaggcgacgcggacggggcccatactggcggcggtcatggttggcactgcgggcggagcggagggcgcggtggcggcgagcggggagcggcggggcctggagcgctggcggtggtcggaggtatttcctatatcttcctgtcttttccatggccaaaaatgatcaagtaaagaacattaaagaagaaataaattactacatattaaaaaatacatactgtaagatgttctttgtacaaaaatgtgacaacttcaataagagtaacttggacaacctgaggcattaattgaggatcaatatgatgac

SEQ ID NO: 282 - DNA, 2025 bp, Homo sapiens
gagtctacaagggaaggggagccggcaacacgctcgagcggaaccctgcggagctgcaaccccgggaggagtcgccggacagactcagacgcggtcccctgggacccagggatgcaccgcgcccctcccgccccagcctgcagagactggatgcttccctccccacctggcgggaagcagaggccctcgggcccgaccgagttcgggccaggacccgccccgctttctgcgagcgaccgactgcggccgccacactcacctcgccgccaagcagcactcgggttctgactcggtctcgggttccggtggcggtggtggcggcggcggcggtggcggcggctcctccatggcggcggcaggcgtgacggcaaaggcgggaggagggaccagcgcggcagcggcttctctcatccgtgcacggagccccgcatggccgcggcgggcagtaagctgcagcctggcgcggggcaccggagccccgaagtgcgggagtgactcgactccgcctccgcctctaccccgccgcaggagccgtgcgcgccgcggccatcttggaaacggcgaccggccgagcgccggggctacggtgggcggcgagggacaagcctcccagatcggcggcggtggcggcggcggcggcgggcgccggaatgcgaccggacgggggcggagacggcggcagggaggggcggggagcgccgtcaagtgtcggcgcgattcggtcctggagccgggtctcgccagctcgcccggtgtcgccccggccggtggcagtgggggactgtggagcggagccgggctgtgcagcgggctgggagctcgaggcttcccaggcggtcgtcggtctgggagggacgctgggtagggccgatgagcggggcgggcgcgacgctatgagcccggccggagtcgccgcccagggacctgccacccggatcccaccttccaggaggacggacgtcacccagacgctcgcaactccggtctcttgacagcgcagccttccacaggctcgaatttttaagacttgacataaagtctccggaattccagattattaaagtgaccccctcatttcaacccggcaaaacttctcaccccagtgtcataagattcgatagcgatctcccagaaacttggactggaatgctcctacactgaggggatggggggaagaagaaatgctaaagattatttcagagttacagaaatcctgttgaaattccaatgcatcgtttactcttagcgctggggcttcacagtttttttttaagttggaaatatttacaaaatttcccatagggtgaaatccagagttaatccaaaaccagaatcttttaaaattaagtacattcaaatgtgcagatttcttaaacctcatttagttgagctttgataggtgaaacgcacgttttgaacaactctggtaacaaagaaaaacattaaattgattataggtatgttatatttaaagaccagttgtgaaattagctatattgtgctcaaagtatgttactgacatttctccgtcgttaaaaggaactgctccattctagttttttataatagtctgtattatgtatatagcctgatagtgctgaatttctgaattgaccttcatcttatttactcaataatattcatttgacaaatacttattaagtgcatatatgtgtcaggaactgtactagatgctgaggatatagcagtaagcgcaacagaccaagctaacagcttagtaaagggtgaggtaaaacaaaacaaaaaagtattcaaaaaaataaactaatttttatcttaatttaaaaaattacaaatttttaaaaatcacaaattggtatccatgtataattcatttccgtgcattttcttgtgtgaagaaagctcagtaaaagtatttcttaggtttctgtaattctagttctctactcgattttcttctgcaattttctgagccagaacccttcttagaaggcatattagagactttaaaagttaaataataaaataaatctcttctttaaa

SEQ ID NO: 283 - DNA, 896 bp, Homo sapiens
ccccggagcgcgcctgcgtggggcgggggcggcagccgactaggggctgggtctggccgtttagggccgggtcttggcccgtcgcccacggtgcggagggctggtgggctttccttggccgtcgggcccgccacggcgcgggtcttggctgcggggcggaggtggggcgggagagccgaggataagagtttgaggcttttcgaggcgcgtgccgcggcgtccgcctctgcgggactctgcgccgggcgccctcggccggcgcgcccggctcccgctttgtcgccgagggaagcacgcgcgacgcccctcccgtcgccgccgtggcttctttcggtgttcgtgatttgctgagaggctggaaagcagcacggcggagaggagccttgcactcgccaggcgggaagcctgcgcggacacgcgtgcgcacccacggggcggcgggcgggcgtggggggtccgggccacgcgggcgacgcgcctctagggaagcgatcttgttgcaccttcccgttattctgaaagcaaatcgtagccagacccgagcgcagcggcttagcaaataataaggggagcgtcagtcgtgctcgaaatgcttccttcgcgatggcgtcagtgttccgtgagggaatgaagccgcagtaggaaataaagaggctgtgcgcgtagtctgaaaagcagaagtcaacatttttacagatgaagaaagaatacggaggcaagaggtctttctctgcagtttggtggatttccaacatttagacttgtttggaagaatttcctcagctgcaccaatgaagtccttgatctatagaagtcggcagtccctaaatctacgtctgcattttgttgcaaatcctttataacattccattaaaataatgcagagttatttaata

SEQ ID NO: 284 - DNA, 313 bp, Homo sapiens
ggaatgcggtcggagcgtgggcgcacgggccgcgcgctccgtgagtagggggcctctcccgcggcgcctgagagccgggtccgcgcgcagaggccctgggggcgcgcgtgccgggtgcgcgctggccagctgtgactgccgctgagggagctgcgcggcgcctctgggagccgagcgccgggcgcctggagggcaggtcgggcctcttgccgctaaggtcagcggctgcggtttgagaagactgcagctgcttcctgtgttacgctgtcgcagacaagcagaagttaaaaactagtaaatgacttaaaggtga

SEQ ID NO: 285 - DNA, 540 bp, Homo sapiens
gcgatgaggacgcgctcgggacactcacgttttctgcaccggcttccgccgcttgcgactggcgtaggtgatgttggcgctgctgctgttcatggtgcccagccgacggcggcggctactccccgggcactggcggccgggtccccggaacccggtgtaggctgggaccactgcggcggccggcgccgggttccgcgaagccgcgcccgctccacctgcgtcctctcgggacagtggcgtgggcgccgcgcctcaacgcctcggctcacgcaggcttccgcagcccgcgccagtacagtgttgtgaaccacttgtcatgggataaccaaaattaaacctggaatcgcagaagaaacaaattttttgaagaaattggatgtacttttttaaaatttttcattcaactaaaactttgttaactgtttcttggttgtggtcttctctaaattgtaatctttggcattcacttccaaagtatccaccactaaacttcataacatttattgtgtctcttcatcattaaactttgatgaatttaca

SEQ ID NO: 286 - DNA, 143 bp, Homo sapiens
gtcctcaggccaaaggagcgactccgtttccagtttcggaaggggtttctccagaataccaaacaccagtactcgtccaccgccgccgcgccgccgagaggcaacgtcaaccgctccattgccggtggggaagccaaaggctt

SEQ ID NO: 287 - DNA, 708 bp, Homo sapiens
acatcgcggctccgcgccgcggaggagacttaaatatcccagcgtgcaccgcgccgcgcgtcgcgtcatcgcgcgccctctacgtcatgcgcgcgcgactccgggagacgctaccgcccggcagcgcccgagacccaactggtttggaaaatgccgcctgcctttaacgcctgtttctgactcttgaggttttccgcgtggctcccagcgccacatccatccggaggcgatcccgtggaaggatcgcgtccaagataaaagggcccgagacctatgcttacgtaattcctaatccgtgatcttttatgggggctgaattcccctccagcacagcctgttaccaggtacttttcacaaacgctactagcggttgtgagtccctcccccctccatttctatgttacaaattgaaaacgtgggacgattacctaccaaatttcacatcatggcccagatatcgggtggattttgtggctctgtgatcgacttatacctatgctaaagttaacggtctgagaaggtagattagtggttgcctagggctagggagtgggtttgggggtggggaatagaaagtgactggtcatggttataggcttttttgtagcaccataaagatagtttaaaattagattgtggtggtgattgcaccaccctatgatttgcatggtatgtgaattatatcttaataaagctgtttaaaaagtc

SEQ ID NO: 288 - DNA, 48 bp, Homo sapiens
aatggcggcggcggcggcggcggccgcggcggcggcggcggcggcggc

SEQ ID NO: 289 - DNA, 510 bp, Homo sapiens
acccggggcggctccttccgaaggctccactccagcggctcgctgcgggagaccaaggccagccgccgtcccaggggacggccctcggggaacctgaatgacgtgcgaggccgtacctcggggcgctggactccgctcgggcgctcccgctctccgctcccgctctcgggtcccaatcccgacgctccgcgcgctcccggactaagtgcggacgcgccccacgccccccggcgccagcccgggacagcgctgcacctgttgccacgtgcgacgcggcgctcagcggctgccactgccgccggcgcacctggctgcaccgcgcgtcccgggaccacgctcgtccccgcaggagcccggggctccgccgcacccagcgctagggcccaggccgggccgcgtcagtcaacgaacattcgagatcctgtcatgtgtcagacactgtgcggagagctggagataaaactgtcaacaagatgaatgcagcctctaaaataaagagtttctaatcca

SEQ ID NO: 290 - DNA, 710 bp, Homo sapiens
acggcgcacggaaacgcaggggagcctgcaccggctgacggcggcggaaaagggcgaacgaagcgcctgagggccctggcagcactcaccagggtcgttccagcccagggcagggcccgcggtcagcagaaacaaatgcaagccgaggagcaagcagtacccggccgctcccggcccgcaggctgcggccatggcgctcgatgaagatggcgccgggctgccagacgcctacgggccgaacctgggtgcggtagcgcgcgcgacgctgcgcagctacaccgctacccctggcggcggcgaaggaacggcccgactgcagagctgcagccgcaacggatccgtgcgccaaccctacgtcactgccgctgcggcccgtggggctcagtgagtggccgccgtgggcccccgcccctggagcacagcgcggcttggggcggggcgaggcggtgtgggcggggccaagggcctggggaggcacggctggaggcgggttggaggcgtggcgccgcggtgtgggcggggccgagggcctgtcgccgccgcgcccgcgggcggggcgagggcagcgcgcagctttgttgtgtctcgtctgagattcgggaagcttctgtgcgtaggcgatgcagtttgagaggctgaaaacgtcacgacgcgaaatctaagaaatgtagtcaaattatgtattattaaaacctcaataattgtaccaa

SEQ ID NO: 291 - DNA, 302 bp, Homo sapiens
ctttcccgcgcgaccggcgagggaggaagaagcgcgaagagccgttagtcatgccggtgtggtggcggcggcggagactgcgggcccgtagctgggctctgcgagcatataggttgctgtagatgaatgttcttagctgtcatgtttaaaaatacttctgcttcgttacctcaagtgtggcatgcagcattttggaaggaaaattgaagacgtgttcaagaaaacatgaacagaagcaaatgatgaaaatgagcattttacttgatgttgataacatcacaataaattatggagaaaaatac

SEQ ID NO: 292 - DNA, 340 bp, Homo sapiens
tccagcaggcgcagcgagcggcggtggcgcgcgcacgaggccgccagcaccgtgtgcgcccgcaactccctcagcgccccgagcaccccgcagcgggtccaagtccgagggcagcacgcggcccgggaagcgctgctcagctagcaggtcacggacgcgcggctgcagcaggcgcgcgcgcagcacggcgcacggaaggcgcaccaggtaccagacgtccagcagcgcaaagacagcgagccccagggccagcaacgccaccagcagccccagcatagcggcggcggcgggcgcggggtcctgcggagtccgtggcgccggtgccggtgtccgcggcgct

SEQ ID NO: 293 - DNA, 302 bp, Homo sapiens
ggcgctcccggggaaagcagtttgtgctcagtgcgttctgcagcataaggaatcctaacgcgaatccgtgaacagtctattccgacggcgcagagaaatctcacgcggggatccccacgatgtgccggcgcttaacgcagacgcccgaacccaggggaaggctgcgactccgcgatgagtgacatgcgggcttgagacgttctaaaggaacgtggaccgagttagggaacgtaactgaagctcattttgtcttagttatggactatgagtccttaataaatgtatgtcattatgtttaaaga

SEQ ID NO: 294 - DNA, 362 bp, Homo sapiens
agagtgagatcctgtctctaaagaaaaaaaaattaattaattatacaagtaatatatgattttattcccatgctaaaaatagtcaaacaacaaagatcatgtaacaacccatgtaatcagtttggttttttttcctctggttcttactcaacgcatttacatatatgtctagtgtatacagtatacgcgtgtgcggcgtacgcagtatacgcgtgtccggcatacgcagaatatgcgtgtctggtgtacgcagtatatgcgtgtctggtgtatgcagtatatgcatgtgtacatatgtatgtacacatatatatctatatatatatcacattgtacaatttacattatacttttttcctctg

SEQ ID NO: 295 - DNA, 738 bp, Homo sapiens
ggcggcgagcgcgcgcggcaaccgccaaacgcccgcctttgtagccccggccccgcgtccttaccctgccgacggctgctcggctccggcccgcggcggcggccccagagccgaggaccggggtgggctcgcgctcttcgtcactggagaagtccgggggccccttgctgttggtgccggcggggagcggcggccggttgcgagccgtgaggtgctgcaggaccgaggggtcttccaggaactccggcatctcggggatctgcgaagccccctccccacagcctttgccgccgccgctcacgccggcgcgcttgctcctggccggggaccgcgctgccgcctccgagcctcacggctcctgcccgggaagagcgctgcggagcggaacaaaaactcgcggacacaaagccaagccagacccggacacaaaagaccccagagccgaactacgaaccaactgcggccaagctggaagctcgcaccaaccccagcccacacactacaggcaggaggcgagcagcctgcttcgcccacgccccaagaacgcgcgccgccattggcgggcgacgggaggaagcgcggcggtgattggcgagacggctcgcgcgggttccattagccgccgcgctgagcgagaggagacgccgataagggacaggtcgtactgcttttgtgcgccgtttcctctccctcccccagcttgagaagcagctttcgtctctggggtcgatcgcctac

SEQ ID NO: 296 - DNA, 331 bp, Homo sapiens
gcaacagccacaaaacccgccgcctcgggccggggccgcactcgggaacggggcgtcgccgctacagccgctgccttgggagccggtccacactgccgttatgagctgtcgcactcacgtgcgcgaaagtctgcagcttcattcctgaagccagcgagaccacgaacccaccgtgaagaacaagcaactccagacgcgtggcatcaagagctggaacactcaccgcgaaggtctgccacttcactcctgagccagcgagaccacgaacccaccagaaggaagaaactccgaacatatcagagcgaacaaattccagacacgccgcctttaa

SEQ ID NO: 297 - DNA, 136 bp, Homo sapiens
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 298 - DNA, 92 bp, Homo sapiens
ggatcgatgatgacttccatatgtacattccttggaaagctgaacaaaatgagtgaaaactctataccgtcatcctcgtcgaactgaggtcc

SEQ ID NO: 299 - DNA, 92 bp, Homo sapiens
ggatcgatgatgacttccacatatacattccttggaaagctgaacaaaatgagtgaaaactctataccgtcatcctcgtcgaactgaggtcc

SEQ ID NO: 300 - DNA, 577 bp, Homo sapiens
gccccggcgccgggcttccccccgccttaggaagctcgcccttccagccagcggccccgactgtactggctccgcggcagccttggctcaggcggccagcgctggacagcggccggggaatcccgtgctgcgcggcgcggccgaggggcgggcctggcccggagagtaccgcggcttcggccctctgccgtgcgcctcgcgcgttttgcatcgctgagcgaagcagagacgcccccccttttcggtctccttaactggttcgtcttcgtagttggaattcctttgcatgaggctatggaatatacactaccttctctggacgttaactattagctacagcttgaggactcccaaatttatgcctttagcaacacgatccgagccctgcaacttttcgacctcattttgagccaacattattttgctcccttattttgttgaagtactttctccagttatttacttaaaatgttcacatgagagacatactatttgattccttgcttgtactaagatgtaatgattgtaacgacttcattttcctagacccagataaaagtctcgcttcattcatgaa

SEQ ID NO: 301 - DNA, 732 bp, Homo sapiens
gccgccgcagcgaagaggccatgttgtgtggtgtgtggggcggggggaggaggaggagggagtaaagcctagagaggagacgggagagagacaccacacacacggcacaagatggccggagaggcagcagcaccccgagctgtcaggcgttccgccgcggccgcgaggcccgccggccggcggggagctacgcccggacggccagcaggcccgcgggagtggggctgccgcggctgaggcgaggcgggccgcgcgcgtcggcgtcacagcccgcggcagaggcgcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggttctccaacggaggagctttttaacctctttccggtgaggtgggaactcatcttcatgatcgaatttaaaagaacaatggaaccctgactacgtttcaacaaaaataaaacttgtttttttccctcctattgggtgttggcttttaactctttcaaagccgattttgaaacggctgcagtgatacatgcgaaggtacttgctgcttattaaacctttatgacgttaaggttgtctttgaaaaatgcaagttggaggagtgcttctggtatttcaga

SEQ ID NO: 302 - DNA, 578 bp, Homo sapiens
tcaggcgttccgccgcggccgcgaggcccgccggccggcggggagctacgcccggacggccagcaggcccgcgggagtggggctgccgcggctgaggcgaggcgggccgcgcgcgtcggcgtcacagcccgcggcagaggcgcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggttctccaacggaggagctttttaacctctttccggtgaggtgggaactcatcttcatgatcgaatttaaaagaacaatggaaccctgactacgtttcaacaaaaataaaacttgtttttttccctcctattgggtgttggcttttaactctttcaaagccgattttgaaacggctgcagtgatacatgcgaaggtgacttaagagattaaaattaatttggttgctgttggttctgaacaaataatgagttcttttatttgagg

SEQ ID NO: 303 - DNA, 556 bp, Homo sapiens
gcggccgcgaggcccgccggccggcggggagctacgcccggacggccagcaggcccgcgggagtggggctgccgcggctgaggcgaggcgggccgcgcgcgtcggcgtcacagcccgcggcagaggcgcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggttctccaacggaggagctttttaacctctttccgccgattttgaaacggctgcagtgatacatgcgaaggtgacttaagagattaaaattaatttggttgctgttggttctgaacaaataatgagttcttttatttgaggtatgccattttgaagactgagacgttggagttttatcctagaggataaaggaaatctttgggaaagtcagtattttatatagcaaaaatatgaacctcaaactgaatcctctaaag

SEQ ID NO: 304 - DNA, 490 bp, Homo sapiens
gcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggttctccaacggaggagctttttaacctctttccggtgaggtgggaactcatcttcatgatcgaatttaaaagaacaatggaaccctgactacgtttcaacaaaaataaaacttgtttttttccctcctattgggtgttggcttttaactctttcaaagccgattttgaaacggctgcagtgatacatgcgaaggtgacttaagagattaaaattaatttggttgctgttggttctgaacaaataatgagttcttttatttgaggtatgccattttgaagactgagacgttggagttttatcctagaggataaaggaa

SEQ ID NO: 305 - DNA, 275 bp, Homo sapiens
ccgggctctgagtgctcttgcccgtccggccccagccgcggcccgggaatctacgtcacccgaaaagcgactataaacgccggcgcctccgtccccagccgcggctcgggaatccacccgaagagtggctataaacgtccgcgcctccattgcgctctcctcttcacttaggacactggtcctcccacgcctgacaccgacgtcgccaggaccgcggggttgggggaacttggctgtcccacgtctttcaaataaagctgttttgtctaactcac

SEQ ID NO: 306 - DNA, 735 bp, Homo sapiens
gctgcagcggcggcggggaccgtggggccgaggtggctgccagccggccaatgtctaagcgaggcggagcggcccaggcggcccgagcctgggggagcgcgcagccggccagtggcggcctcgccggcggcctcttcccgggctcgcagtaggcccgagtcgtcgccgggagctcctgggagcagcgtccccgccctgctcccctcgctcccgcctcttgcggccccacggcccctcagcgcccgcccccggctccgcccgccgcagccgcagcccctggcgctaacggtcggtaacggcccgcgcgcgccgcccgccgggggctcgcgccagccacgagggagcgtccgcggcccgcgcgcccgcgcggcggaggagaggtgttaagtgtgatgcttccataatacatttggatgctgtcagctaagttcacttctgaactaaggggttcctccaaatgttggctgaaattcatcccaaggctggtctgcaagtgagtgtctgcacacagtttgcttgtatgtggagtcgatccaaaatagcatcaatgttggttttaccaaagtatttattattgataatagaggctaagtacaaaatgtagagaatgtcagctacttgaggcctttgattattaaaaattttattaatgcattaaacaagagtacagtaaatagataaattttaggttcatgaaataaaactgaataatttatttttacttactatttatca

SEQ ID NO: 307 - DNA, 327 bp, Homo sapiens
tggtaggaacagcgtctttaatgggccccgccccccacgcccgccaaaccgccccccttgcgggccactttggcctcctccaccgcggcacggagggcccgggccgggtccccgcggagccgggccaattctccgagcagcgcagcggagtcgtcccccgtacggagctgtcggccgttcagaaaatagtgaggcaacgtcccggcctcgagcactcggccgagctcgtctagcagcccgaggcagcaggcgactcggacgctgaaacgtcactaggcgatttttccaaagacaccggcgcctcgtgctcagtgacgtagctgtgcg

SEQ ID NO: 308 - DNA, 72 bp, Homo sapiens
tgtcctgatgatacttgtaataggaagtgccgtcagaagcgataactgacgacgtctaatgtctatctgacc

SEQ ID NO: 309 - DNA, 71 bp, Homo sapiens
tgctctgatgaaatcactaataggaagtgccgtcagaagcgataactgacgaagactactcctgtctgatt

SEQ ID NO: 310 - DNA, 428 bp, Homo sapiens
caggcagagtgcgtgggcgatctcgatgcgccgtcgccgggtcagccgtttcctctccctcgccggcctcggcggagattccaggccctatagaaaccaggacgtcccttagcgccaccgcctcacatgccagtgctgccgggaacccagcgatatccgcaccagcggagaaggttccaggctgccggcggcggcgcagagagcgggaagagaggctcggaggaagccccgggcgtggcgtggtcaggctccgagagcggccgggatgcggccacaccggcctggtaaactcgcacctcttaggatcttgctcccggactcattcccttccccaccccctattttaaagttttatttgggtcgtctgtatcaatttagaacgagataaattaagacaaagaaagtaaaataaatcgaaataaaatata

SEQ ID NO: 311 - DNA, 522 bp, Homo sapiens
cctcggcactgagctgagacgcgctgagcagtttgctctcctttttccacttcatgcgtcggttctggaaccatatcttgatctgcctctccgtcaggcacagggcgtgcgcgatctcgatgcgccgccgccgcgtcagccgtttcctctccctcgccggcctcggcggagattccaggccctatagaaaccaggacgtcccttagcgccaccgcctcacatgccagtgctgccgggaacccagcgatatccgcaccagcggagaaggttccaggctgccggcggcggcgcagagagcgggaagagaggctcggaggaagccccgggcgtggcgtggtcaggctccgagagcggccgggatgcggccacaccggcctggtaaactcgcacctcttaggatcttgctcccggactcattcccttccccaccccctattttaaagttttatttgggtcgtctgtatcaatttagaacgagataaattaagacaaagaaagtaaaataaatcgaaataaaatata

SEQ ID NO: 312 - DNA, 133 bp, Homo sapiens
gcgctgtctttgagcccccgccgagcttcctcgtggcgccgggggtcaatctgcagcgctagagcatgtgcttgcgcataactggggccgcctggcctcccgcgggcggcctttttaaccgcgagcgacaaga

SEQ ID NO: 313 - DNA, 249 bp, Homo sapiens
tattttaataattctttttattgtttttttaatggagtgttgatttccttacataaatttaattgtaaacatgtacataggaccgtgattttgaattaaaaaaatactttgggccttcggccttcttagtagttttttttgttttgttttgttttcgtttttgttttgatacggcgtcttgctctgtcgcccagaaaataaacaaataaaagtcttcctcagcgacaaaagcaaacgcaagcccgagct

SEQ ID NO: 314 - DNA, 93 bp, Homo sapiens
ccttccggcgtcccaggcggggcgccgcgggaccgccctcgtgtctgtggcggtgggatcccgcggccgtgttttcctggtggcccggccatg

SEQ ID NO: 315 - DNA, 132 bp, Homo sapiens
tcgaaagagcatgaaacggaggagacgttacagcaacgtgtcagctgaaatgatgggcgtagacgcacgtcagcggcggaaatggtttctatcaaaatgaaagtgtttagagattttcctcaagtttcaaat

SEQ ID NO: 316 - DNA, 651 bp, Homo sapiens
tcgctaacgaggccgccgacaagggcatcgccaacgaggacgccgcccacggcatcgccaacgaggacgccgcccacggcatcgccagcgaggacgccgcccacggaatcgccagcgaggacgccgcccacggcatcgccagcgaggacgccgcccagggcatcgccaacgaggacgccgcccagggcatcgccaaggaggacgccgtccacggcatcgccaacgaggacgccgcccagggcatcgccaaggaggacgccgcccagggcatcgccaacgaggacgccgcccagggcatcgccaaagaggacgccgcccagggcatcgccaaggaggacgccgcccagggcatcgccaacgagggcgccgcccagggcatcgccaaagaggacgccgcccatggcatcgccaacgaggacgccgcccagggcatcgccaacgaggacgccgcccacggaatcgccagcgaggacgccgcccacggcatcgccagcgaggacgccgtccagggcatcgccaaggaggatgccgcccagggcatcgccaacgaggacgccgcccagggcatcgccaacgaggacgccgcccagggcatcgccaaagaggacgccgcccacggcatcgccaacgagctgtatacgacatcgctaa

SEQ ID NO: 317 - DNA, 516 bp, Homo sapiens
ggcgtggcagcggcaatccctggccaaaggtacttggggtcattttccgcggggggttacgtgcgggagcgtgtcctccacaaacggattttccccccttaagcggacttatttccatccggagtgacagaatttaattccaaaccgagagctttccagactgacgaatttttaccgggactaacagagaattacctcagcctgacaacattttatctcgtgcgccacttcgggatccgagtggagccgaaagtcgagatagagcgcagttgggccttgggttcgaaaagactggacagaggtttcagtgagccgagatcgcgccactgcaccccggcctgggcgacagagcgagactccgtctcaaaaaaagaaaaagaaaaaagcctggaaagaaattctgcggaatagaagtctttaagaaatgagatcgtgccctttaaaactaagtttaacaatagtgagggcttaataaacgataggtgttatctctgggttggaggggaataccttcaa

SEQ ID NO: 318 - DNA, 150 bp, Homo sapiens
atgccttaaacttatgagtaaggaaaataacgattcggggtgacgcccgaatcctcactgctaatgtgagacgaatttttgagcgggtaaaggtcgccctcaaggtgacccgcctactttgcgggatgcctgggagttgcgatctgcccg

SEQ ID NO: 319 - DNA, 204 bp, Homo sapiens
gcggcggcagccctgtctggcttggcggttcggctgtcgcgctcggcggcggcccgaggctcatacggcgccttctgcaaggggctcacgcgcacgctgctcaccttcttcgacctggcctggcggctgcgcatgaacttcccctacttctacatcgtggcctcggtgatgctcaacgtccgcctgcaagtgcggatcgagtga

SEQ ID NO: 320 - DNA, 27 bp, Artificial
  Primer
gcgcgtaatacgactcactataggcga

SEQ ID NO: 321 - DNA, 27 bp, Artificial
  Primer
cgcaarraaccctcactaaagggaaca

SEQ ID NO: 322 - DNA, 22 bp, Artificial
  Primer
gaaattaatacgactcaatagg

SEQ ID NO: 323 - DNA, 29 bp, Artificial
  Primer
tctagcatttaggtgacactatagaatag

(19) United States
(12) Patent Application Publication
(10) Pub. No.: US 2020/0268786 A1
(43) Pub. Date: Aug. 27, 2020

(54) RNA CONTAINING COMPOSITIONS AND METHODS OF THEIR USE

(71) Applicants: Icahn School of Medicine at Mount Sinai, New York, NY (US); Institute for Advanced Study-Louis Bamberger & Mrs. Felix Fuld Foundation, Princeton, NJ (US); ECOLE NORMALE SUPERIEURE, Paris (FR); Centre national de la recherche scientifique, Paris (FR); Université Paris-Diderot, Paris (FR); Sorbonne Université, Paris (FR)

(72) Inventors: Benjamin D. Greenbaum, New York, NY (US); Nina Bhardwaj, New York, NY (US); Arnold Levine, Princeton, NJ (US); Remi Monasson, Paris (FR); Simona Cocco, Paris (FR)

(21) Appl. No.: 16/786,709

(22) Filed: Feb. 10, 2020

Related U.S. Application Data
(62) Division of application No. 15/550,548, filed on Aug. 11, 2017.
(60) Provisional application No. 62/116,298, filed on Feb. 13, 2015.

(51) Int. Cl. A61K 31/7105; G01N 33/50; A61K 39/39; A61K 45/06; C12Q 1/68; C12N 15/00; G01N 33/566

(52) U.S. Cl. CPC A61K 31/7105; G01N 33/5011; A61K 39/39; A61K 2039/55561; C12Q 1/68; C12N 15/00; G01N 33/566; A61K 45/06

(57) ABSTRACT

The present invention relates to a composition comprising an isolated, single stranded RNA molecule having a nucleotide sequence comprising 20 or more bases and a pattern of CpG dinucleotides defined by a strength of statistical bias greater than or equal to zero, and a pharmaceutically acceptable carrier suitable for injection. The present invention also relates to a kit comprising a cancer vaccine and the composition of the present invention as an adjuvant to the cancer vaccine. The present invention further relates to a method of treating a subject for a tumor and a method of stimulating an immune response.

This application is a divisional of U.S. patent application Ser. no. 15/550,548, filed Aug. 11, 2017, which is a national stage application under 35 U.S.C. § 371 of PCT Application No. PCT/US2016/018001, filed Feb. 15, 2016, which claims the benefit of U.S. Provisional Patent Application Ser. No. 62/116,298, filed Feb. 13, 2015, each of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 10, 2016, is named 29539_0571_ST25.txt and is 252 kilobytes in size.

### FIELD OF THE INVENTION

The present invention relates to RNA containing compositions and methods of their use.

### BACKGROUND OF THE INVENTION

The recent development of total RNA sequencing has allowed a better appreciation of the complexity and breadth of the entire transcriptome (Djebali et al., “Landscape of Transcription in Human Cells,” Nature 48:101-108 (2012); ENCODE Project Consortium, “An Integrated Encyclopedia of DNA Elements in the Human Genome,” Nature 489:57-74 (2012); Harrow et al., “GENCODE: The Reference Human Genome Annotation for the ENCODE Project,” Genome Res. 22:1760-1774 (2012), and Martin et al., “Next-Generation Transcriptome Assembly,” Nature Rev. Genet. 12:671-682 (2011)). Analysis by the Encyclopedia of DNA Elements (“ENCODE”) consortium unexpectedly showed that far more of the mammalian genome than previously appreciated is transcribed into non-coding RNA (“ncRNA”). Several short ncRNA have conserved metabolic and regulatory functions and some anti-viral properties have been assigned to novel classes of ncRNA such as eukaryotic small-interfering RNA, piwi interacting RNA, and prokaryotic CRISPR RNA (Rinn et al., “Genome Regulation by Long Noncoding RNAs,” Ann. Rev. Biochem. 81:145-66 (2012)). In eukaryotes, long non-coding RNA (“lncRNA”), such as long-intergenic non-coding RNA, have been associated with transcriptional, post-transcriptional, and epigenetic regulation (Atianand et al., “Molecular Basis of DNA Recognition in the Immune System,” J. Immunol. 190:1911-1918 (2013) and Zhang et al., “The Ways of Action of Long Non-Coding RNAs in Cytoplasm and Nucleus,” Gene 547:1-9 (2014)).

It is now evident that germ line and cancer cells can have atypical ncRNA transcription, including repetitive elements from regions usually silenced in steady state (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013) and Ting et al., “Aberrant Overexpression of Satellite Repeats in Pancreatic and Other Epithelial Cancers,” Science 331:593-596 (2011)). In eukaryotes, transcription of endogenous retroviruses and mobile elements is mostly repressed epigenetically through processes such as histone modification and DNA methylation, preventing disruptive or deregulatory effects due to integration into coding regions. In mammals, DNA methylation targets the cytidine in CpG motifs to form 5-methyl cytosine contributing to down-regulation of transcription for methylated sequences (Jones et al., “The Role of DNA Methylation in Mammalian Epigenetics,” Science 293:1068-1070 (2001)). Epigenetic regulation is strongly associated with developmental process whereas its deregulation, such as by disruption of DNA methylation, can be associated with de-differentiation and carcinogenic processes (Feinberg et al., “The History of Cancer Epigenetics,” Nature Rev. Cancer 4:143-153 (2004) and Yi et al., “Multiple Roles of p53-Related Pathways in Somatic Cell Reprogramming and Stem Cell Differentiation,” Cancer Res. 72:5635-5645 (2012)).

When expressed, endogenous retroviral RNA can activate the innate immune response via several pathways (Zeng et al., “MAVS cGAS and Endogenous Retroviruses in T-independent B Cell Responses,” Science 346:1486-1492 (2014)). In cancers, such as those driven by p53 mutations and epigenetic alterations, ncRNA associated with repetitive elements can be induced (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013) and Ting et al., “Aberrant Overexpression of Satellite Repeats in Pancreatic and Other Epithelial Cancers,” Science 331:593-596 (2011)). In a study of mouse and human epithelial malignancies (Ting et al., “Aberrant Overexpression of Satellite Repeats in Pancreatic and Other Epithelial Cancers,” Science 331:593-596 (2011)), several repetitive elements emanating from genomic dark matter and often repressed in steady state conditions, particularly pericentromeric repeats such as GSAT (major satellite) in mouse and HSATII in humans, were only transcribed in cancer cells. A strong induction of repetitive elements from the mouse genome (particularly GSAT, B1, and B2) along with several other ncRNAs in cells bearing p53 oncogenic mutations and exposed to epigenome altering demethylating agents has been demonstrated (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013)). Anomalous expression of the murine repetitive element GSAT was shown to trigger transcription of repeat-dependent activated interferon response (TRAIN), which can regulate apoptosis related cell death. The mechanism is that the double strands form immediately via bi-directional transcription. That is, as GSAT is being transcribed in the positive sense by one polymerase (pol II) its complementary DNA strand is also being transcribed by pol-III at the same time. In this model, there is never single stranded GSAT transcribed; the double stranded RNA is formed during RNA transcription. There has been no indication in Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013) or elsewhere that single stranded RNA GSAT would be immunostimulatory.

The present invention is directed to overcoming these and other deficiencies in the art.

### SUMMARY OF THE INVENTION

One aspect of the present invention relates to a composition comprising an isolated, single-stranded RNA molecule having a nucleotide sequence comprising 20 or more bases and a pattern of CpG dinucleotides defined by a strength of statistical bias greater than or equal to zero, and a pharmaceutically acceptable carrier suitable for injection.

Another aspect of the present invention relates to a kit comprising a cancer vaccine and the composition of the present invention as an adjuvant to the cancer vaccine.

A further aspect of the present invention relates to a method of treating a subject for a tumor. This method involves administering to a subject the composition of the present invention (i.e., a composition comprising an isolated, single stranded RNA molecule having a nucleotide sequence comprising 20 or more bases and a pattern of CpG dinucleotides defined by a strength of statistical bias greater than or equal to zero, and a pharmaceutically acceptable carrier suitable for injection) under conditions effective to treat the subject for the tumor.

Another aspect of the present invention relates to a method of stimulating an immune response. This method involves providing the composition of the present invention (i.e., a composition comprising an isolated, single-stranded RNA molecule having a nucleotide sequence comprising 20 or more bases and a pattern of CpG dinucleotides defined by a strength of statistical bias greater than or equal to zero, and a pharmaceutically acceptable carrier suitable for injection) and contacting a cell or tissue with the composition under conditions effective to induce or increase an immune response against cancer in the cell or tissue.

A set of novel mathematical tools originally developed to analyze potentially immunostimulatory motif usage in viral and host genome coding sequences was used here. These methods were recently recast in the language of statistical physics and are extended here to analyze ncRNA motif usage (Greenbaum et al., “Patterns of Evolution and Host Gene Mimicry in Influenza and Other RNA Viruses,” PLoS Path. 4:e1000079 (2008) and Greenbaum et al., “Quantitative Theory of Entropic Forces Acting on Constrained Nucleotide Sequences Applied to Viruses,” Proc. Natl. Acad. Sci. 111:5054-5059 (2014)). For the first time, large-scale patterns of motif usage in human and murine transcriptomes, which are used to find anomalies ncRNA expressed in cancer transcriptomes (Rinn et al., “Genome Regulation by Long Noncoding RNAs,” Ann. Rev. Biochem. 81:145-66 (2012) and Ulitsky et al., “lincRNAs: Genomics Evolution and Mechanisms,” Cell 154:26-46 (2013)), were analyzed. As a result, features of ncRNA over-expressed in cancerous cells relative to normal cells were characterized (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013); Ting et al., “Aberrant Overexpression of Satellite Repeats in Pancreatic and Other Epithelial Cancers,” Science 331:593-596 (2011); Levine et al., “The maintenance of epigenetic states by p53: the guardian of the epigenome,” Oncotarget 3:1503-1504 (2012)). This analysis includes several large datasets of functionally characterized ncRNA, in addition to pseudogenes and repetitive elements such as satellite DNA, endogenous retroviruses, and long and short interspersed elements. It is demonstrated that many ncRNAs preferentially expressed in cancerous cells display anomalous motif usage patterns compared to the vast majority of ncRNAs whose patterns of motif usage are shown to be consistent with those in coding regions. Based on their unusual pattern of motif usage and differential expression in cancerous versus normal cells, it is predicted that the ncRNA HSATII (human) and the nRNA GSAT (murine) incorporate immunostimulatory motifs in humans and mice respectively. Remarkably, the prediction demonstrating that both directly stimulate antigen-presenting cells and accordingly label them immunostimulatory ncRNAs (“i-ncRNAs”) is validated.

Other features and advantages of the invention will be apparent from the following detailed description and claims.

### BRIEF DESCRIPTION OF DRAWINGS

FIGS. 1A-B demonstrate that ncRNA expressed in cancer differ from general lncRNA motif usage patterns. FIG. 1A shows the fraction of GENCODE human lncRNA sequences where a motif occurs the expected number of times as defined by corresponding to a probability p greater than 0.05 (EQUATION 5). FIG. 1B is a graph showing the fraction of GENCODE lncRNA sequences in humans and mice where the occurrence of CpG motifs occurs the expected number of times compared to those expressed in human cancerous cells and mouse cancer cell lines.

FIGS. 2A-B are graphs demonstrating that CpG and UpA are generally under-represented in ncRNA. FIG. 2A shows the histogram of forces (i.e., strength of statistical bias) on CpG, and FIG. 2B shows the histogram of forces (i.e., strength of statistical bias) on UpA, both for lncRNA from the GENCODE human transcript database. These forces (i.e., strengths of statistical bias) are consistent with those observed in mice and those from coding regions.

FIGS. 3A-B demonstrate that forces (i.e., strengths of statistical bias) on CpG and UpA dinucleotides are independent. FIG. 3A is a graph showing the least principal components for all significant forces (i.e., strengths of statistical bias) on motifs for human GENCODE ncRNA, and FIG. 3B shows the least principal components for all significant forces (i.e., strengths of statistical bias) on motifs for mouse GENCODE ncRNA. In both cases, CpG and UpA dominantly project onto the two least axes of variation.

FIGS. 4A-B demonstrate that GSAT is expressed in mouse testicular teratoma and liposarcoma by showing the study results of the relative levels of expression of GSAT RNA by a custom TAQMAN qPCR assay in normal murine tissue versus murine tumor tissue samples. FIG. 4A is a graph showing results from the testicular teratoma tumor mouse models. FIG. 4B is a graph showing results from the liposarcoma induced tumor in p53KO background. In all instances, GSAT levels were increased in the tumor samples as compared to normal samples, to varying degrees.

FIGS. 5A-D demonstrate that ncRNA from cancer cells contain outliers from normal motif usage. The distribution of the strength (force) of statistical bias is shown for UpA and CpG (FIGS. 5A-B) and CAG and CUG (FIGS. 5C-D) in lncRNA taken from human tumors (FIG. 5A and FIG. 5C) and murine cell lines (FIG. 5B and FIG. 5D), (dark data points), plotted against lncRNA from GENCODE (light grey data points). Each ellipse indicates one standard deviation from the mean value in the GENCODE dataset.

FIGS. 6A-C demonstrate that ncRNA require transfection to induce cellular innate immune responses. 2 ug/ml of the various ncRNA (HSATII, HSATII-sc; GSAT; GSAT-sc) were used to stimulate human DCs in 96 well plates with (DOTAP) or without (NT) the use of DOTAP as a gentle liposomal transfection reagent. In absence of transfection reagent, the ncRNA were not sensed by the DCs whereas transfected immunogenic ncRNA HSATII and GSAT, in addition to Poly-IC and R848, were properly sensed and induced a cellular inflammatory response in TNFalpha (FIG. 6A), IL-12 (FIG. 6B), and IL-6 (FIG. 6C).

FIG. 7 is a schematic illustration showing the innate immune pathways involved in the sensing of nucleic acids which were investigated in the work described herein. MYD88 and UNC93b were directly implicated in i-ncRNA sensing.

FIGS. 8A-B demonstrate that i-ncRNA stimulates human moDC cytokine production. Quantification of inflammatory cytokine production upon liposomal transfection of human in human i-ncRNA (HSATII) and murine i-ncRNA (GSAT) versus their scrambled and endogenous controls is shown for human moDCs in FIG. 8A and murine imBM in FIG. 8B. Each point represents the mean value of the experimental replicates for each individual condition; the bar represents the median. The significance of i-ncRNA stimulation is analyzed by the non-parametric Mann-Whitney test to compare their effect versus their scrambled and endogenous controls.

FIGS. 9A-C demonstrate that human moDCs and mouse imBM cells respond to common PAMPs and DAMPs. Quantification of inflammatory cytokine production in human moDCs is shown in the graphs of FIG. 9A, and in murine imBM in the graph of FIG. 9B, upon stimulation with common PAMPs or DAMPs known to activate PRR innate immune pathways, which are listed in the Examples infra. Each point represents the mean value of the experimental replicates for each individual condition; the bar represents the median. FIG. 9C is a heat map showing the inflammatory response related to type I IFN pathway induction in imBM upon stimulation of the PRR related innate immune pathways analyzed by qRT-PCR. The heat-map represents the log of the relative expression of each gene based on relative quantification analysis using the ddCT bi-dimensional normalization method (housekeeping genes and non-stimulated cells).

FIGS. 10A-C demonstrate that MYD88 and UNC93b control GSAT i-ncRNA stimulation. FIGS. 10A-C are graphs showing the results of genetic screening of the innate immune pathway related to i-ncRNA function in murine imBM. imBM cells of different genotype (WT (FIG. 10A), MYD88 KO (FIG. 10B), and UNC93b3d/3d MUT (FIG. 10C)) have been stimulated by liposomal transfection of the murine i-ncRNA (GSAT). TNFa production in the supernatant has been quantified, and each point represents the mean value of the experimental replicates for each individual condition; the bar represents the median.

FIGS. 11A-B show that the genetic screen of innate immune pathways related to i-ncRNA function in murine imBM. FIG. 11A is a series of graphs showing imBM cells of different knockout genotypes related to TLR PRRs (TLR2-4 dbKO, TLR3 KO, TLR4 KO, TLR7 KO, TLR9 KO). FIG. 11B is a series of graphs showing imBM cells of different knockout genotypes related to STING, inflammasome, and MAV dependent helicases pathways (STING KO, MAV KO, ICE KO); and common innate immune signaling (TRIF KO, TRAM KO, IRF3/IRF7 dbKO). Cells have been stimulated by liposomal transfection of the murine i-ncRNA (GSAT). The TNFa production in the supernatant has been quantified and each point represents the mean value of the experimental replicates for each individual condition; the bar represents the median.

FIGS. 12A-B show the stimulation of KO and mutant imBM with common PAMPs and DAMPs. Quantification of inflammatory cytokine production in PRR KO imBM (FIG. 12A) and innate immune signaling related KO and mutant (FIG. 12B) upon stimulation with common PAMPs or DAMPs known to activate PRR innate immune pathways is shown. Each point represents the mean value of the experimental replicates for each individual condition; the bar represents the median.

FIG. 13 demonstrates that motif usage in HSATII and GSAT clusters with foreign RNA. A comparison of the forces (i.e., strengths of statistical bias) on CpG dinucleotides is plotted against the distribution of forces (i.e., strengths of statistical bias) on all GENCODE lncRNA relative to a sequences nucleotide bias. The force on CpG dinucleotides for HSATII and GSAT are shown on the distribution, along with the average values for the longest gene (PB2) in human influenza B and avian H5N1 and all E. coli coding regions.

FIGS. 14A-S show mouse repeat RNA sequences from the Repbase database with anomalous CpG motif usage.

FIGS. 15A-F show mouse ncRNA sequences from the ENCODE database with anomalous CpG motif usage.

FIGS. 16A-Y show human repeat RNA sequences from the Repbase database with anomalous CpG motif usage.

FIGS. 17A-L show human ncRNA repeat sequences from the ENCODE database with anomalous CpG motif usage.

### DETAILED DESCRIPTION OF THE INVENTION

The invention described herein relates to RNA-containing compositions and methods of their use.

In a first aspect, the present invention relates to a composition comprising an isolated, single stranded RNA molecule having a nucleotide sequence comprising 20 or more bases and a pattern of CpG dinucleotides defined by a strength of statistical bias greater than or equal to zero, and a pharmaceutically acceptable carrier suitable for injection.

The composition of the present invention may be a pharmaceutical composition in the form of a vaccine, or a pharmaceutical composition intended to be co-administered with a vaccine, e.g., as an adjuvant.

In one embodiment, the RNA molecule in the composition of the present invention is an isolated RNA molecule. The term “isolated RNA molecule” includes RNA molecules which are separated from other nucleic acid molecules which are present in the natural source of the RNA. An “isolated” nucleic acid molecule is free of sequences which naturally flank the nucleic acid (i.e., sequences located at the 5′ and 3′ ends of the nucleic acid molecule). For example, in various embodiments, the isolated RNA molecule contains a defined number of bases. Moreover, an “isolated” nucleic acid molecule is substantially free of other cellular material, or culture medium, when produced by recombinant techniques, or substantially free of chemical precursors or other chemicals when chemically synthesized.

In one embodiment, the RNA molecule is a single-stranded RNA molecule.

In another embodiment, the composition comprises an isolated RNA molecule having a nucleotide sequence comprising 20 or more bases and a pattern of CpG dinucleotides defined by a strength of statistical bias greater than or equal to zero, with the proviso that the RNA molecule is not GSAT.

Suitable RNA molecules in the composition of the present invention include, without limitation, an RNA molecule having the nucleotide sequence of SEQ ID NOs:1-319, or a fragment thereof. Such RNA molecules can be isolated using standard molecular biology techniques and the sequence information provided herein. In one embodiment, using all or a portion of the nucleic acid sequence of SEQ ID NOs:1-319 as a hybridization probe, RNA molecules can be isolated using standard hybridization and cloning techniques (e.g., as described in Sambrook, J. et al. Molecular Cloning: A Laboratory Manual, 2nd, ed., Cold Spring Harbor Laboratory, Cold Spring Harbor Laboratory Press, Cold Spring Harbor, N.Y., 1989, which is hereby incorporated by reference in its entirety).

Moreover, an RNA molecule in the composition of the present invention can be isolated by the polymerase chain reaction (PCR) using synthetic oligonucleotide primers. In one embodiment, the primers are designed based upon the sequence (or a portion thereof) of any one or more of SEQ ID NOs:1-319.

The RNA molecule in the composition is an RNA molecule of about 20 or more bases in length. The length of the RNA molecule (i.e. , the total number of bases) may vary depending on the pattern of CpG dinucleotides and the strength of statistical bias. In one embodiment, the RNA molecule has about 20-1200 bases, about 20-1100 bases, about 20-1000 bases, about 20-900 bases, about 20-800 bases, about 20-700 bases, about 20-600 bases, about 20-500 bases, about 20-450 bases, about 20-400 bases, about 20-350 bases, about 20-300 bases, about 20-250 bases, about 20-200 bases, about 20-190 bases, about 20-185 bases, about 20-180 bases, about 20-175 bases, about 20-170 bases, about 20-165 bases, about 20-160 bases, about 20-155 bases, about 20-150 bases, about 20-145 bases, about 20-140 bases, about 20-135 bases, about 20-130 bases, about 20-125 bases, about 20-120 bases, about 20-115 bases, about 20-110 bases, about 20-105 bases, about 20-100 bases, about 20-95, about 20-90, about 20-85, about 20-80 bases, about 20-75 bases about 20-70 bases, about 20-65 bases, about 20-60 bases about 20-55 bases, about 20-55 bases, about 20-50 bases, about 20-45 bases, about 20-40 bases, about 20-35 bases, or about 20-30 bases.

The RNA molecule of the composition has a pattern of CpG dinucleotides defined by a strength of statistical bias greater than or equal to zero. A physical system can be defined by the various states in which it can exist, and all the parameters involved in known constraints. When no assumption is made about the particular state the system is in, the system can be defined by the probability distribution of each of the states being occupied.

An RNA molecule with a pattern of motifs (e.g., CpG dinucleotides) can be defined by its length, nucleotide frequencies (i.e., the proportion of each nucleotide present in the sequence), and the number of times the motif is observed in the sequence. An RNA molecule of length L can take 4̂^{L} different states, with each of those states being characterized by a number of motifs.

When considering the probability of a number of motifs (e.g., CpG dinucleotides) observed in a particular sequence, a random-nucleotide model can be used to define the probability distribution of observing a given number of motifs in all 4̂^{L} possible sequences of length L, and with nucleotide frequencies according to the proportion observed in the given sequence. The random model gives rise to a distribution of states for such a sequence, each state having a number of motifs.

To quantify deviation of the particular observed sequence (i.e., state) from the random expectation, an additional parameter, referred to here as selective force, or simply force (e.g., force on CpG or force on UpA) may be added to the model. This additional parameter introduces a statistical bias in the probability distribution towards observing a particular state (i.e., a particular number of observed motifs). In the absence of this statistical bias, the probability of a given state (i.e., the number of observed motifs in a particular sequence) simplifies to the product of its nucleotide frequencies, whereas positive force shifts the distribution towards a larger number of observed motifs than what one would expect under the purely random model. Given a particular sequence, the “strength of statistical bias” is defined herein as the value of the force that maximizes the probability of the observed sequence. That is, the strength of statistical bias is the value for the force that results in a probability distribution of the number of motifs for a given sequence with length L and nucleotide frequencies such that the mean of the probability distribution is equal to the observed number of motifs in the sequence, as demonstrated in Example 5 (infra).

The larger the deviation of the number of the motifs observed in a given sequence is from random, the larger the force required to generate a distribution in which the number of observed motifs in the sequence is equal to the mean of the distribution.

The strength of statistical bias can be used as a parameter for identifying anomalous (i.e., outlier) states in a system, including anomalous use of motifs (e.g., CpG dinucleotides and other dinucleotide or trinucleotide repeats) in nucleotide sequences. In order to identify outliers, one must identify a threshold for which any strength of statistical bias that meets or exceeds the threshold will be considered anomalous. In order to identify a threshold, one may generate the distribution of observed strengths of statistical bias against a collection of samples chosen to represent the system (i.e., a reference set or panel). For example, a reference set for nucleotide sequences may include a set of biologically similar sequences, such as non-coding RNAs drawn from a database, such as the ENCODE database, as described in the Examples (infra). After the distribution of observed strengths of statistical bias is generated, it may be fit to a Gaussian distribution, characterized by a mean and standard deviation, and utilized as a null hypothesis (i.e., null distribution) against which to test the strength of statistical bias on any single sample. Once a statistical threshold is set, the identification of anomalous states may be carried out based only on the strength of statistical bias for the particular state in question, without the use of a reference set.

The present invention, as demonstrated in Example 6 (infra), has defined the statistical threshold for identifying sequences with anomalous patterns of CpG dinucleotides as those sequences having a strength of statistical bias greater than or equal to zero.

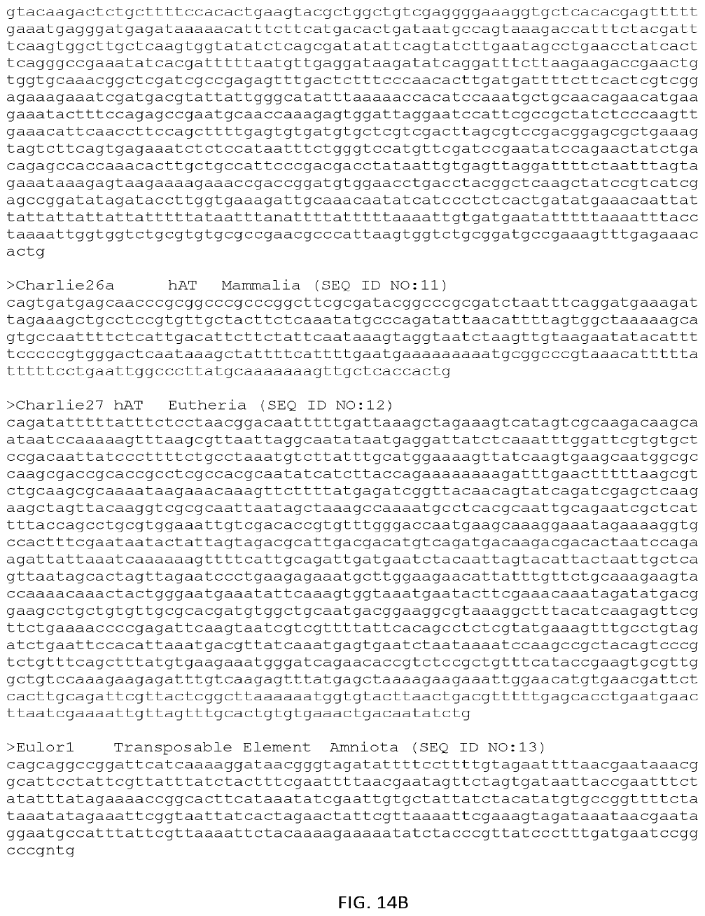

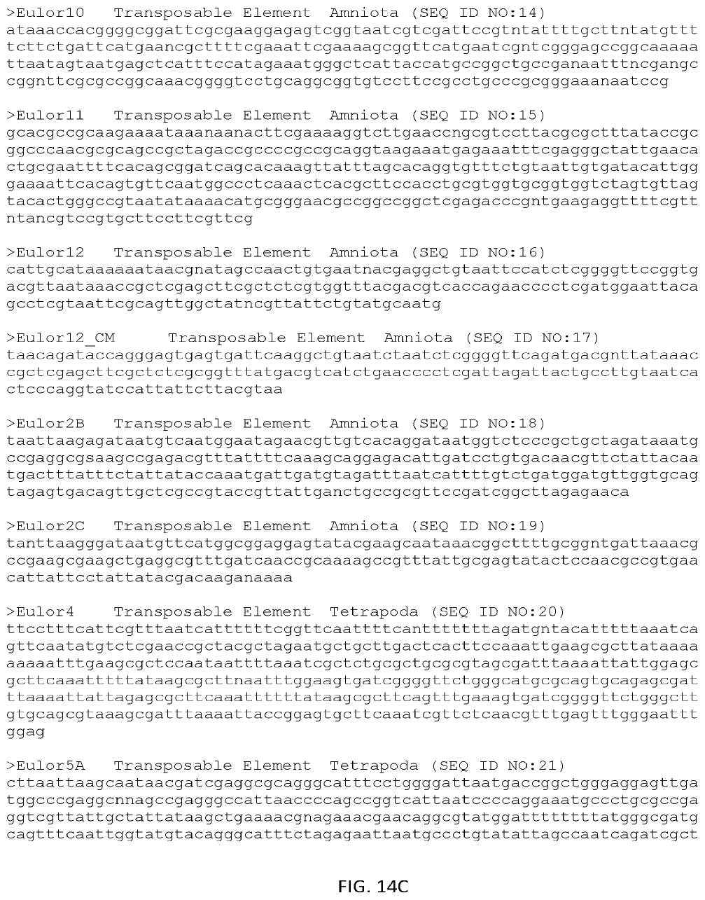

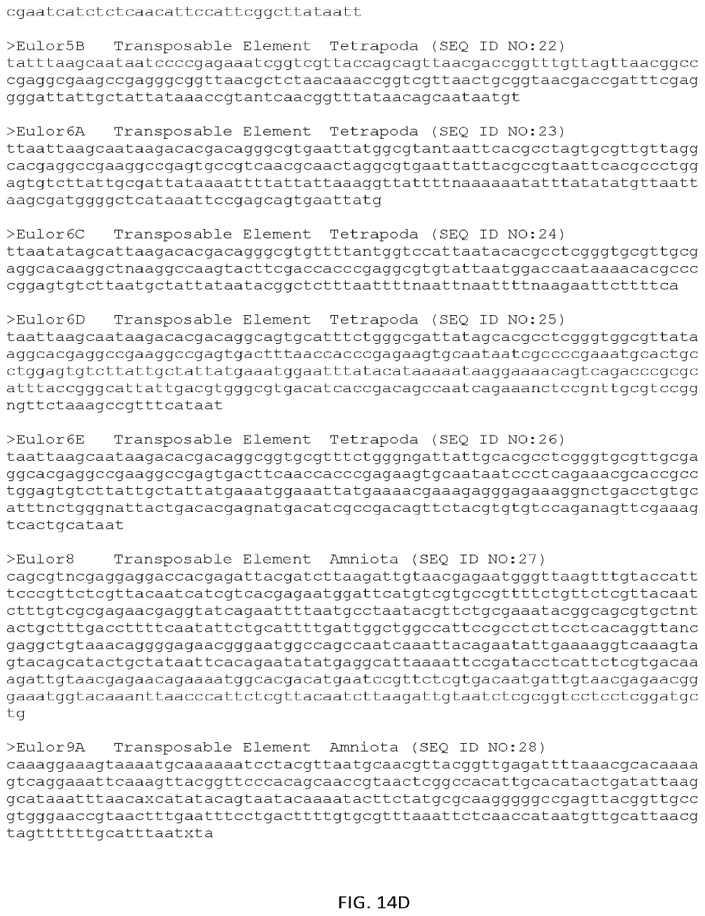

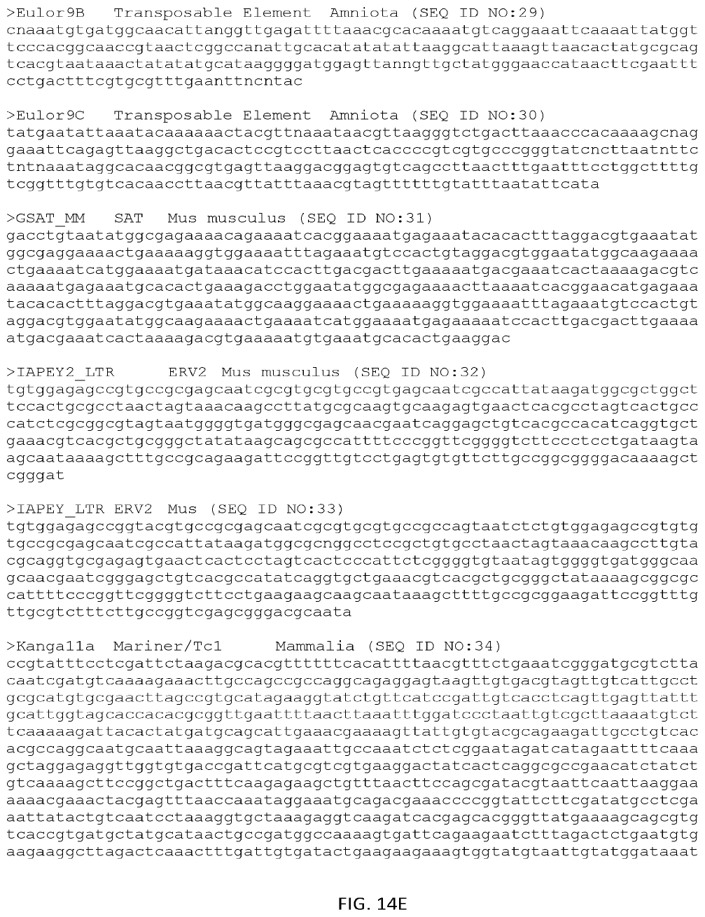

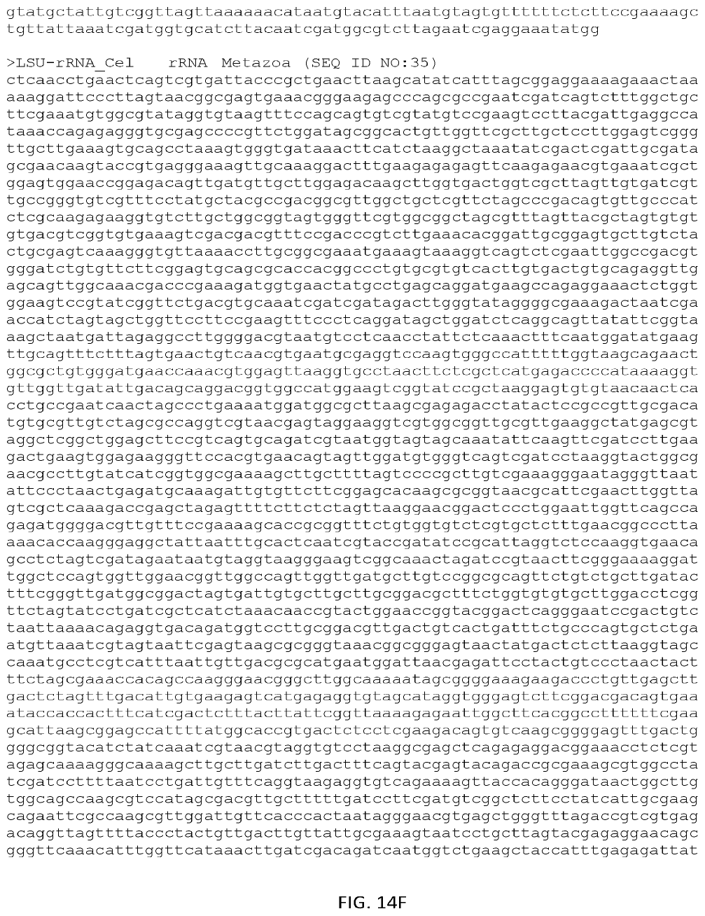

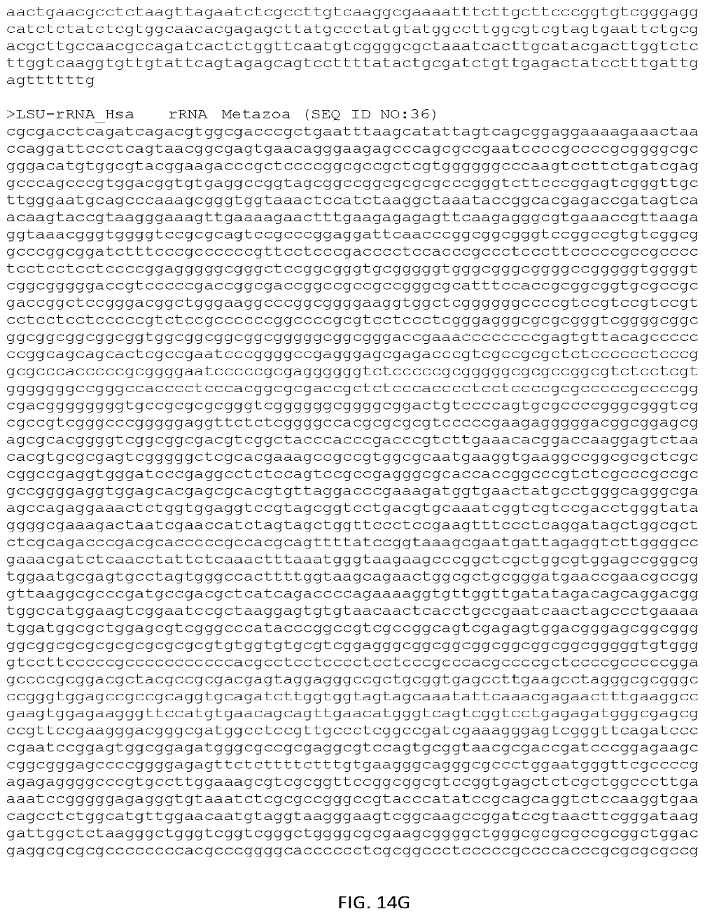

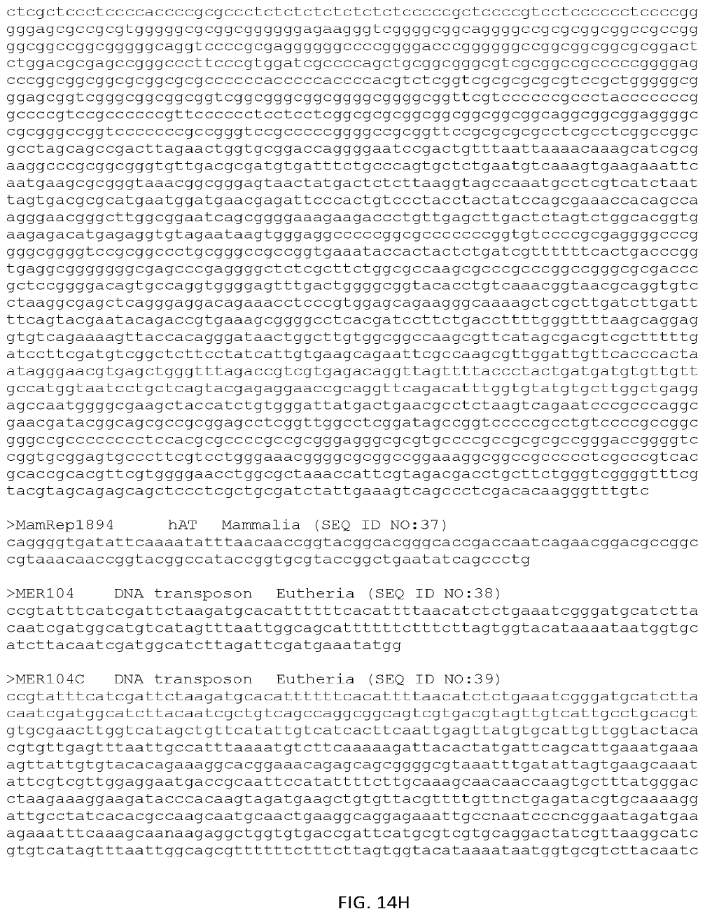

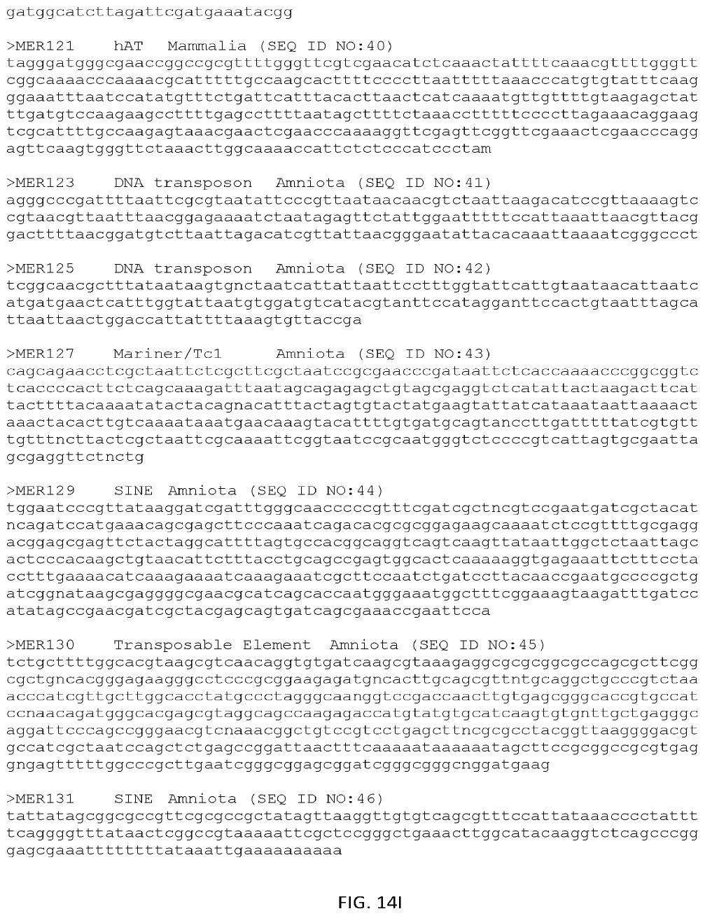

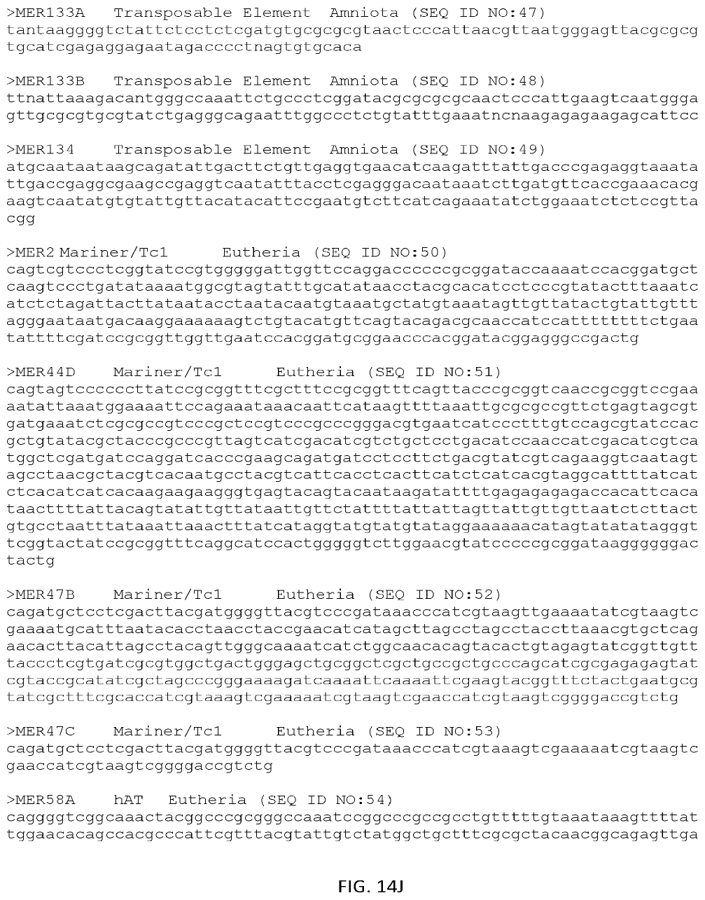

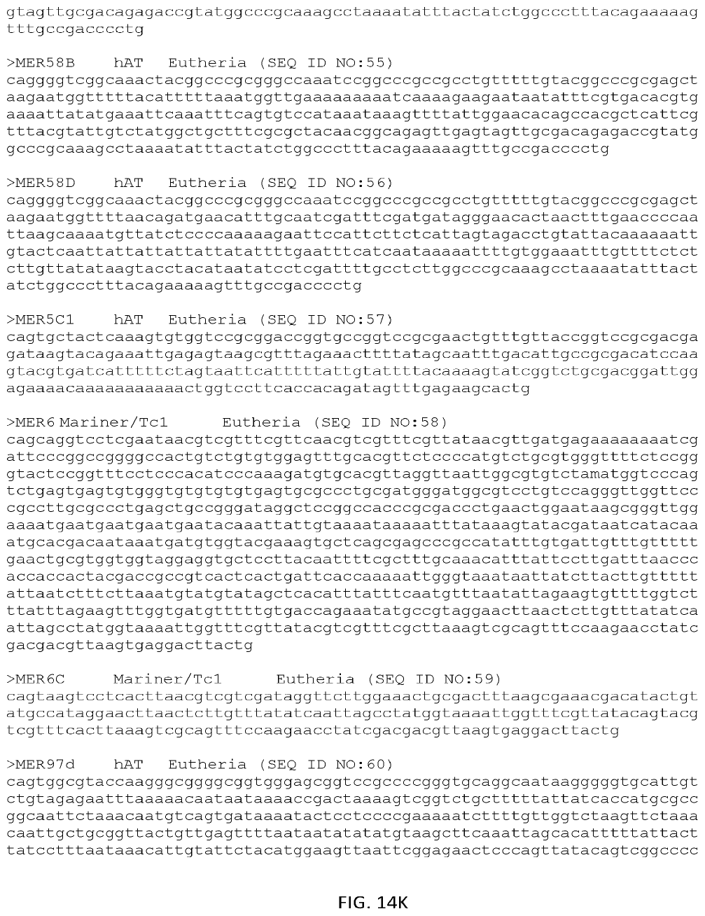

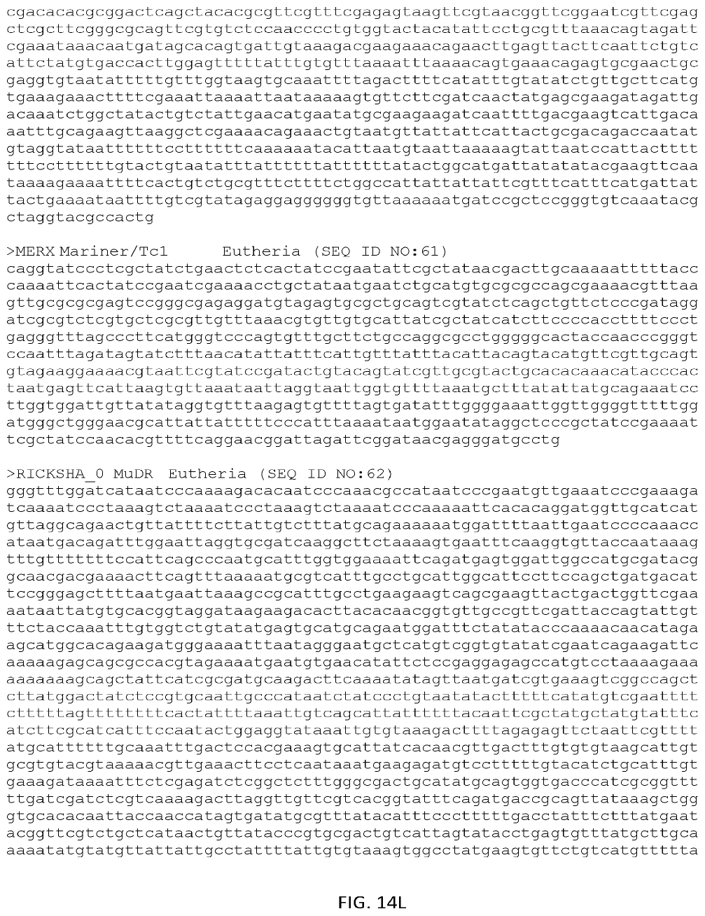

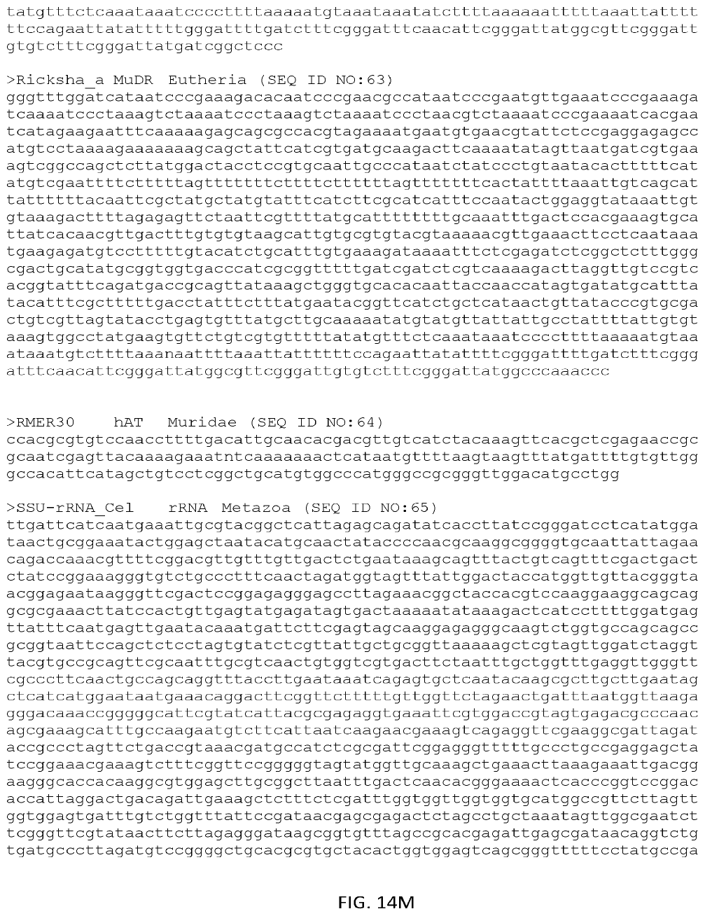

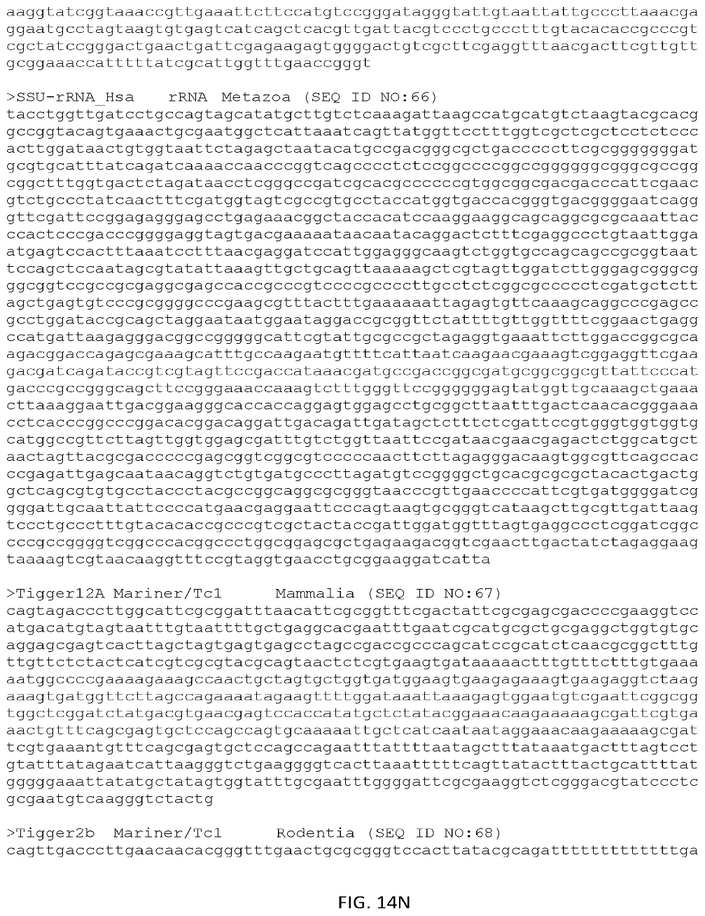

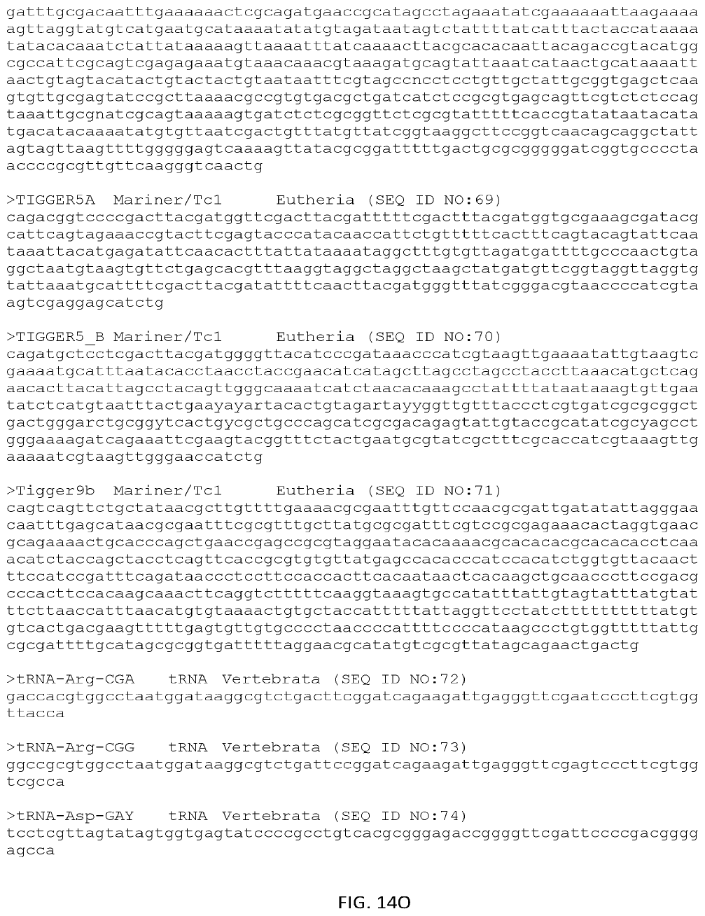

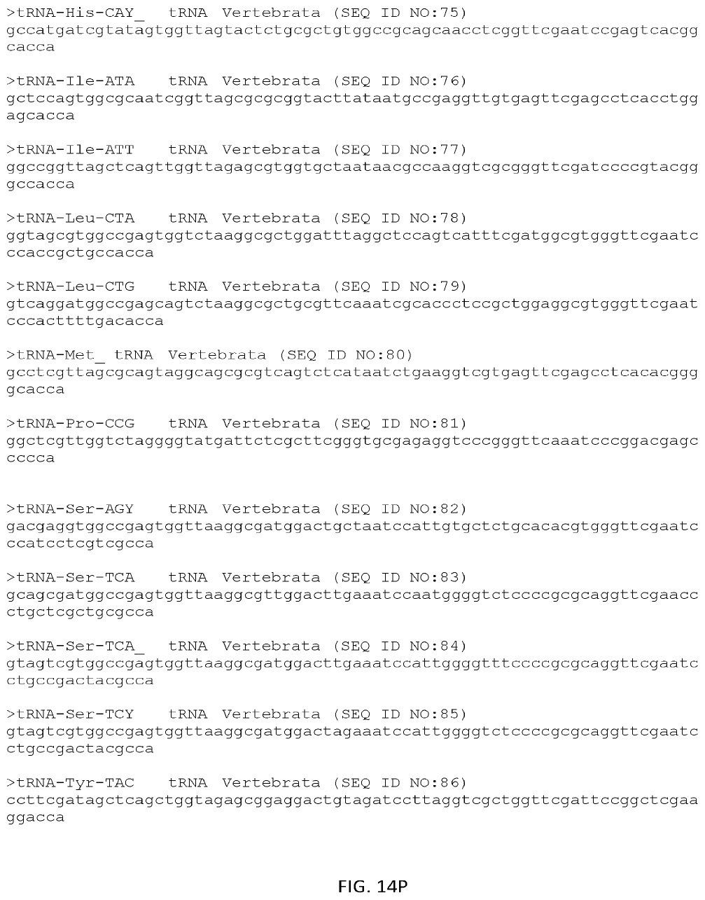

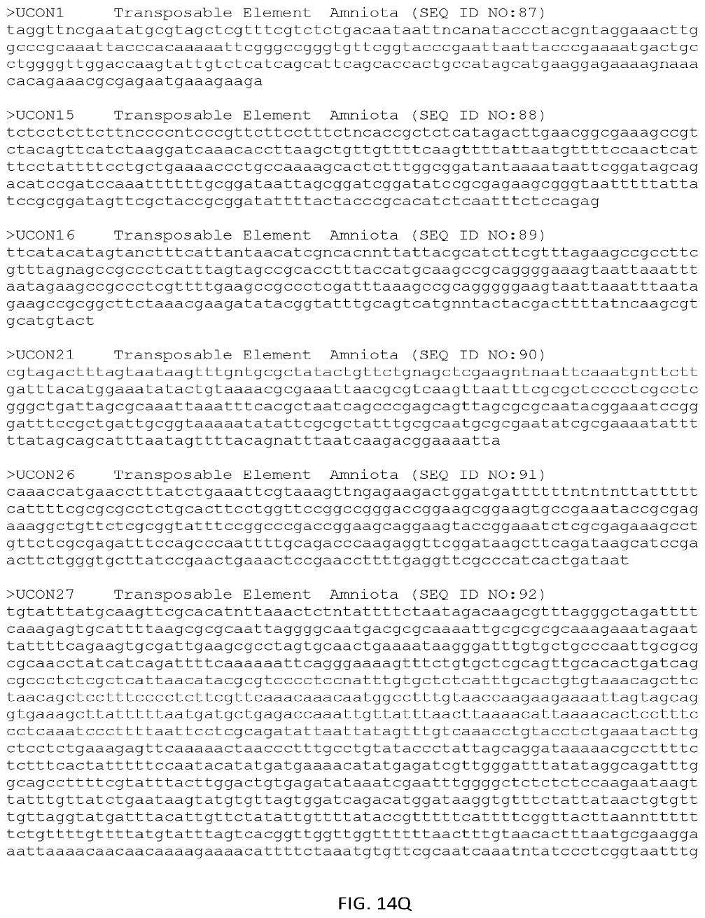

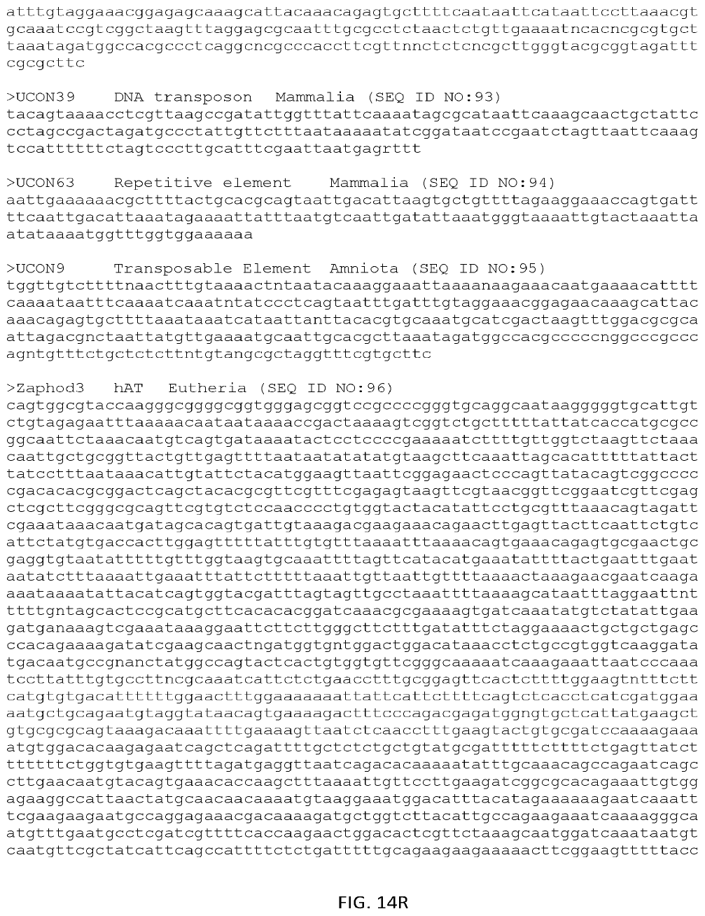

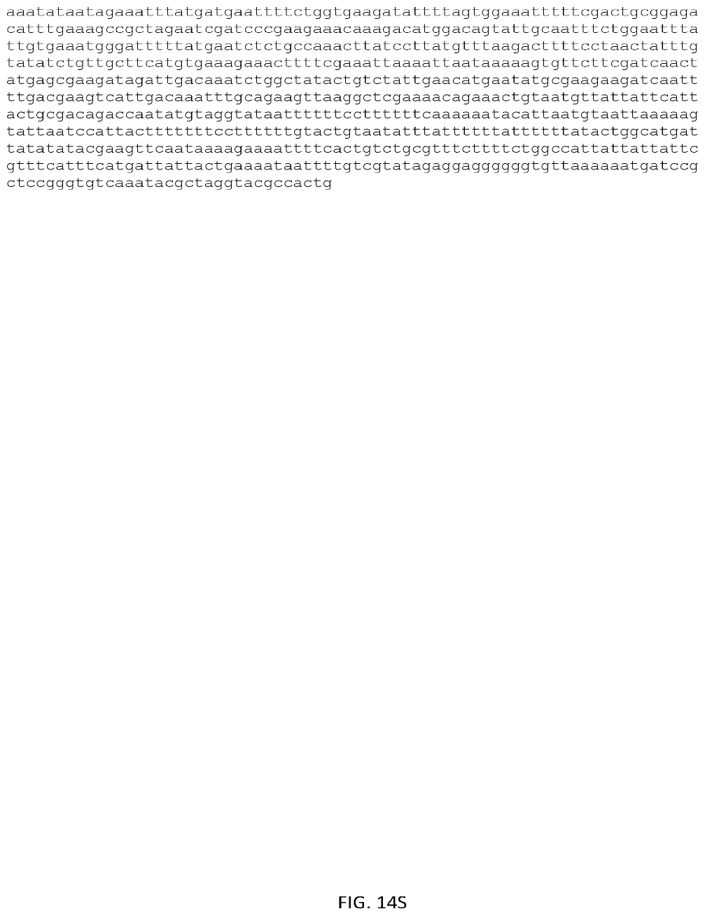

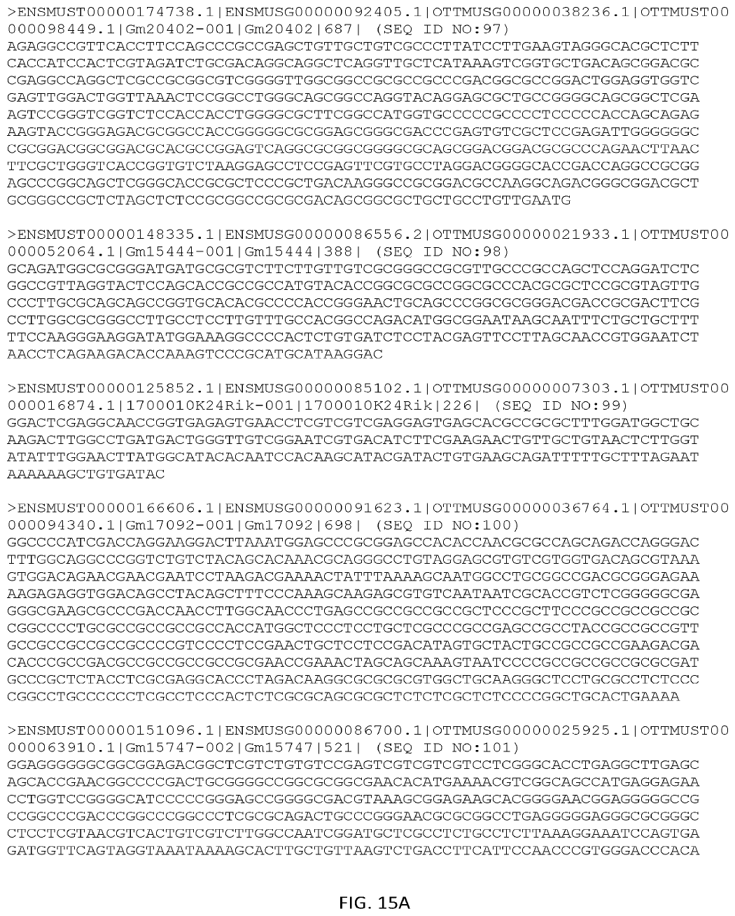

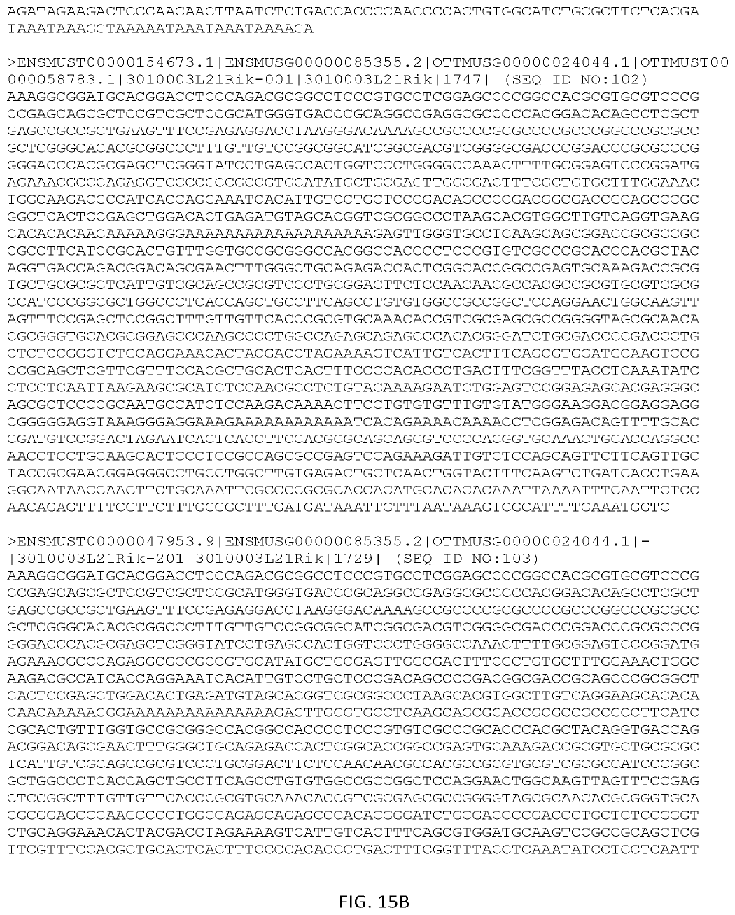

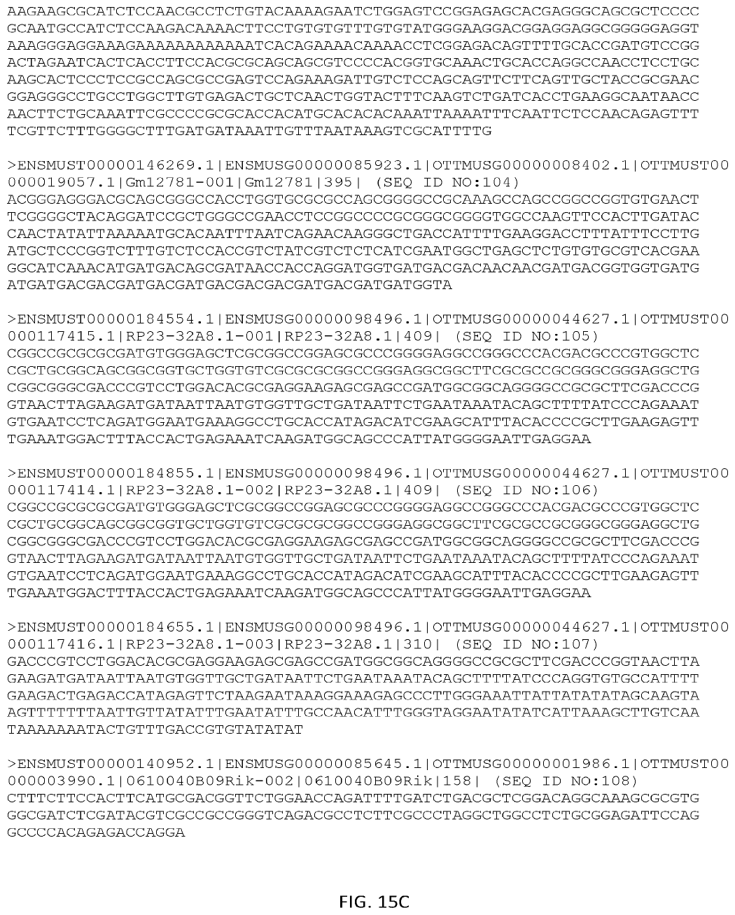

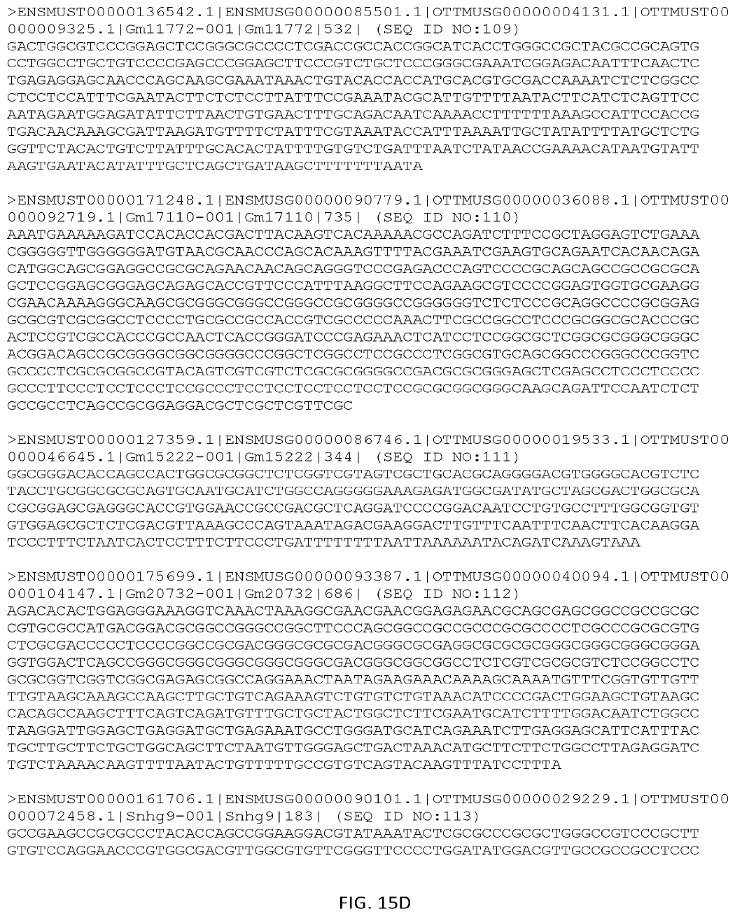

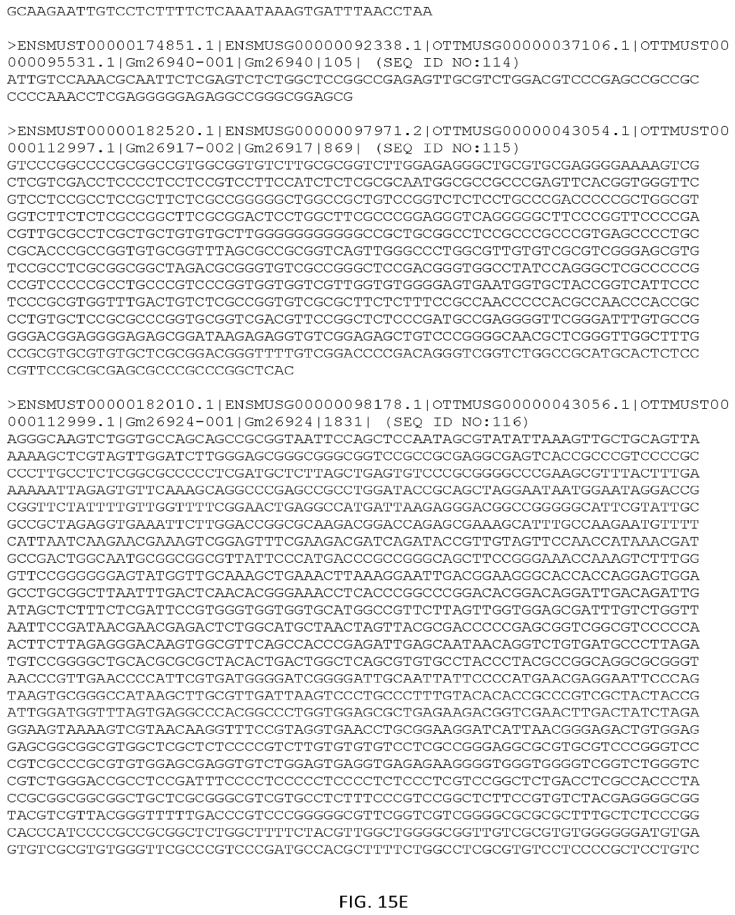

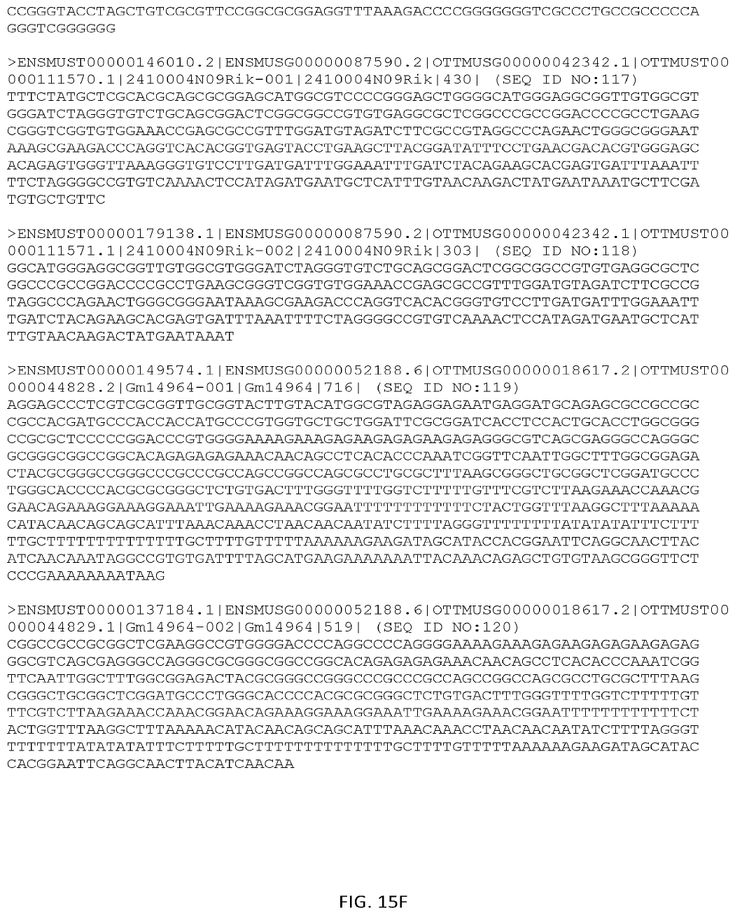

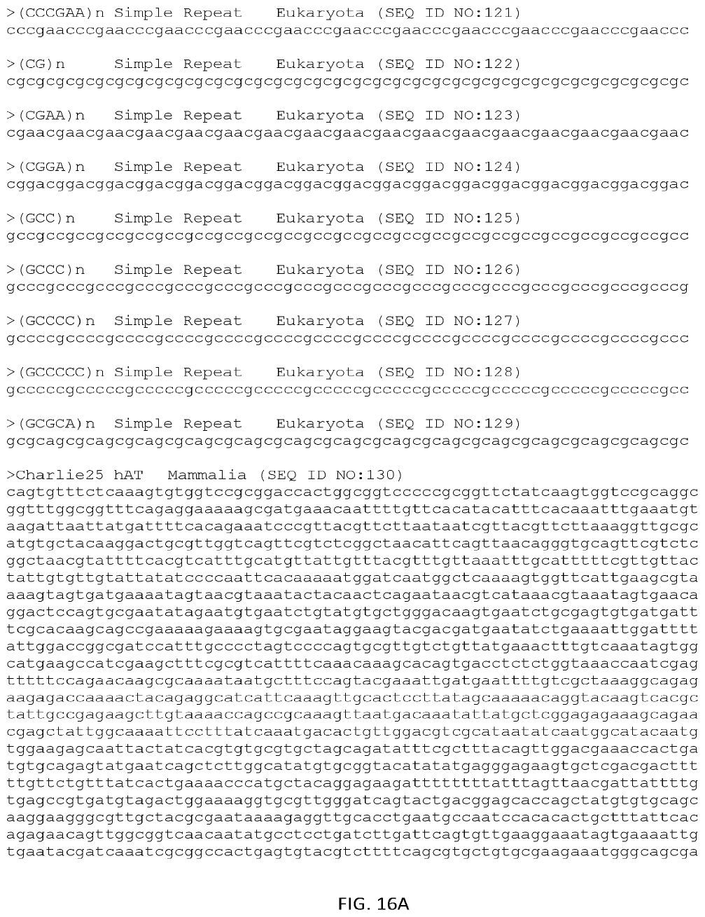

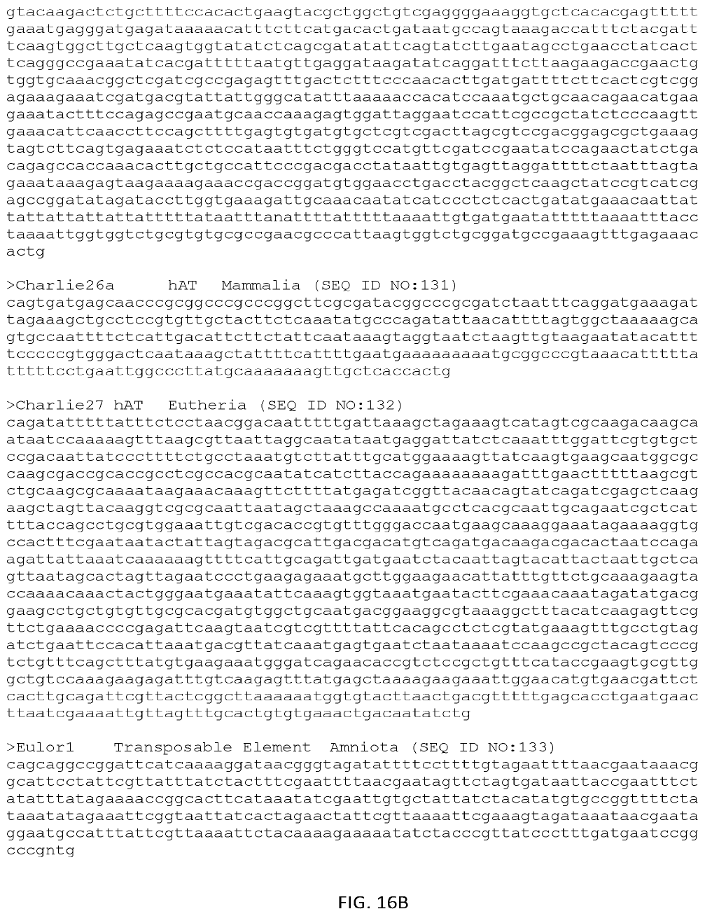

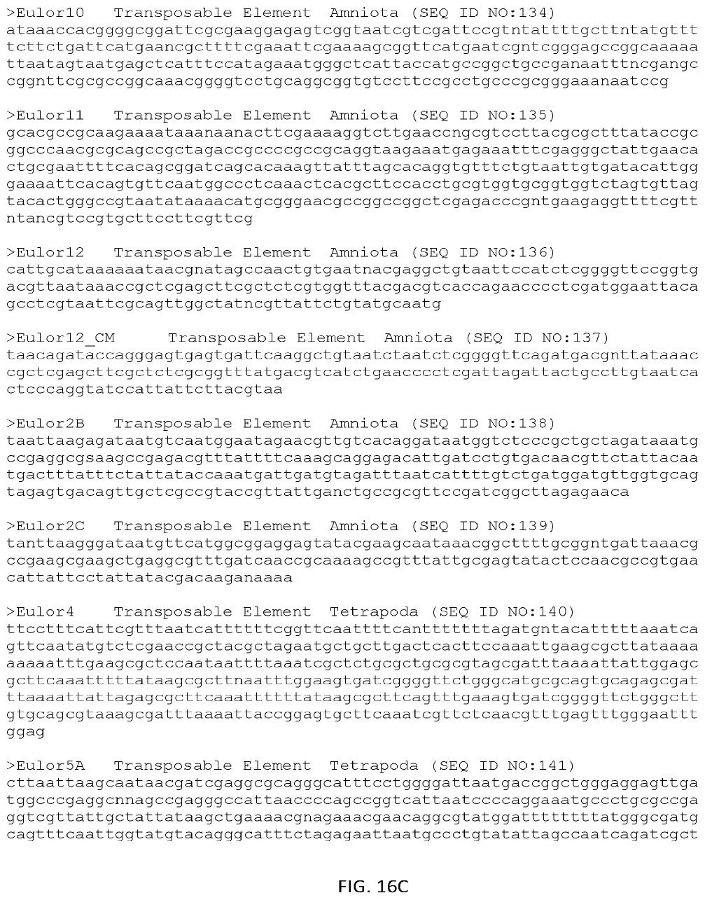

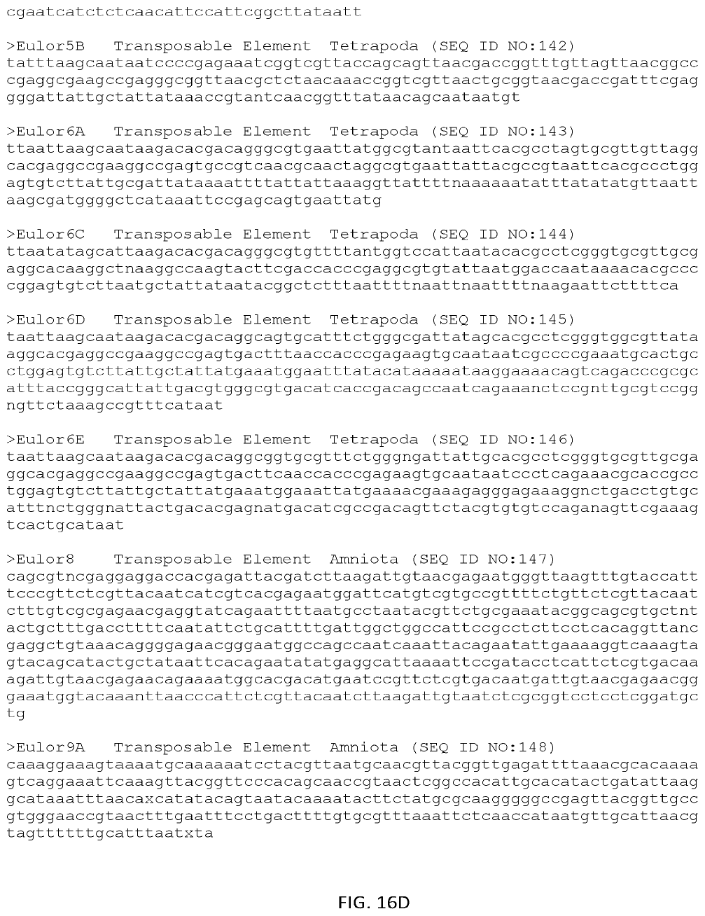

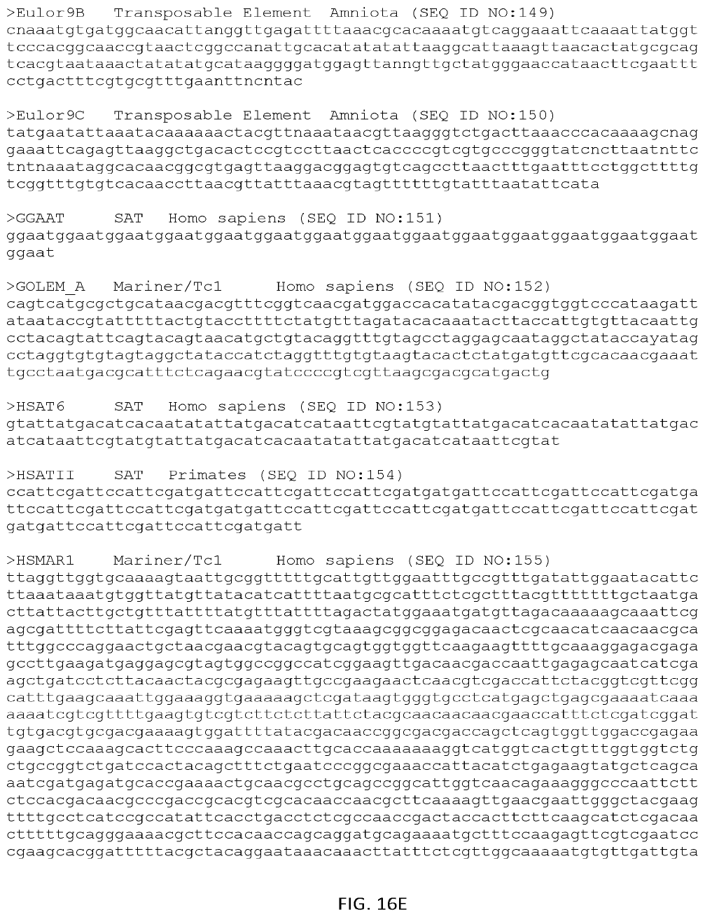

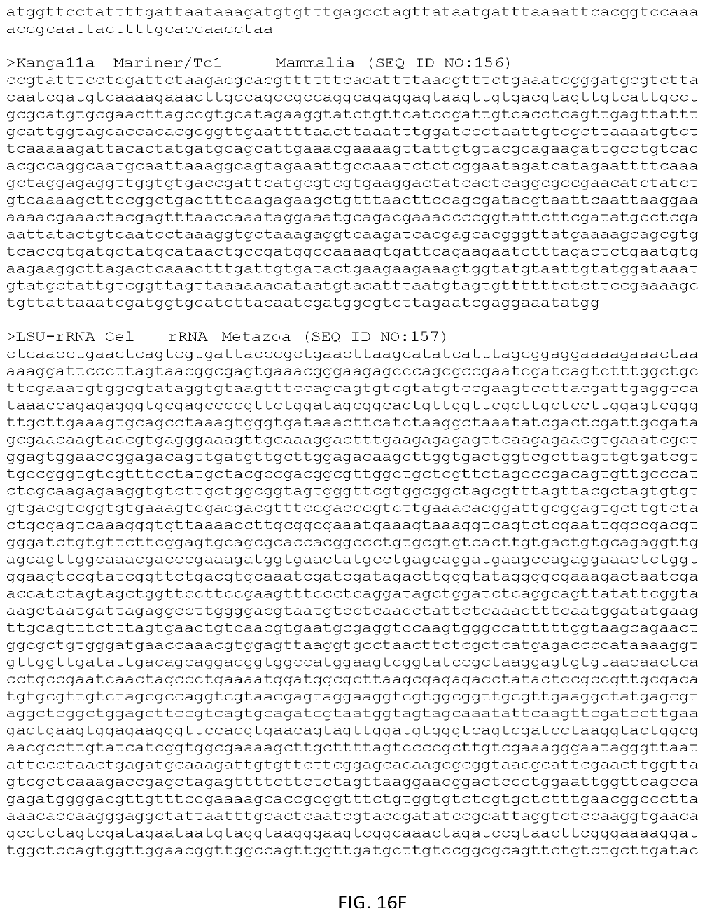

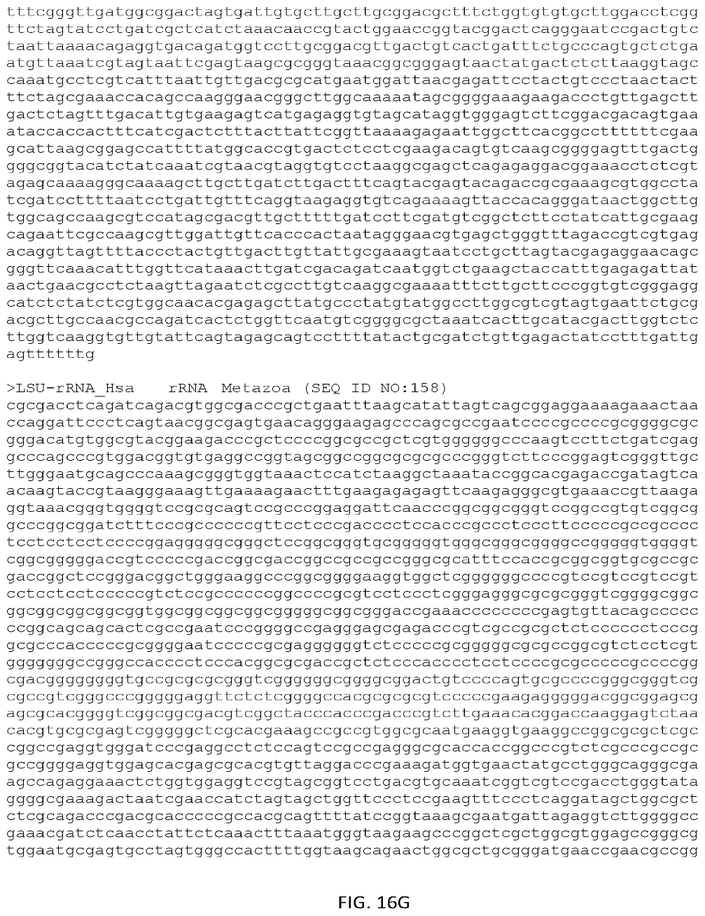

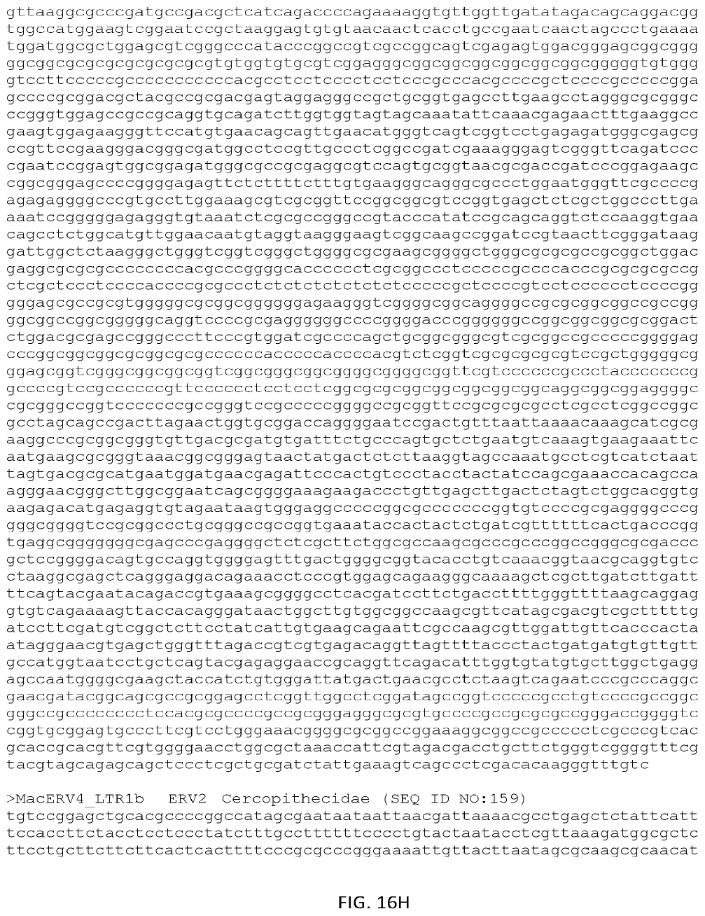

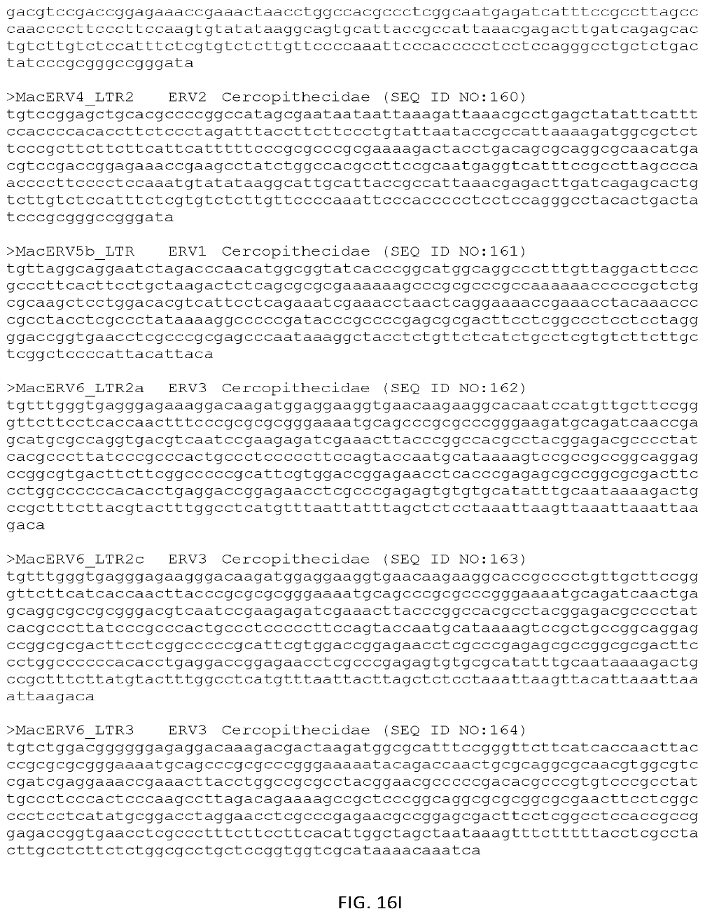

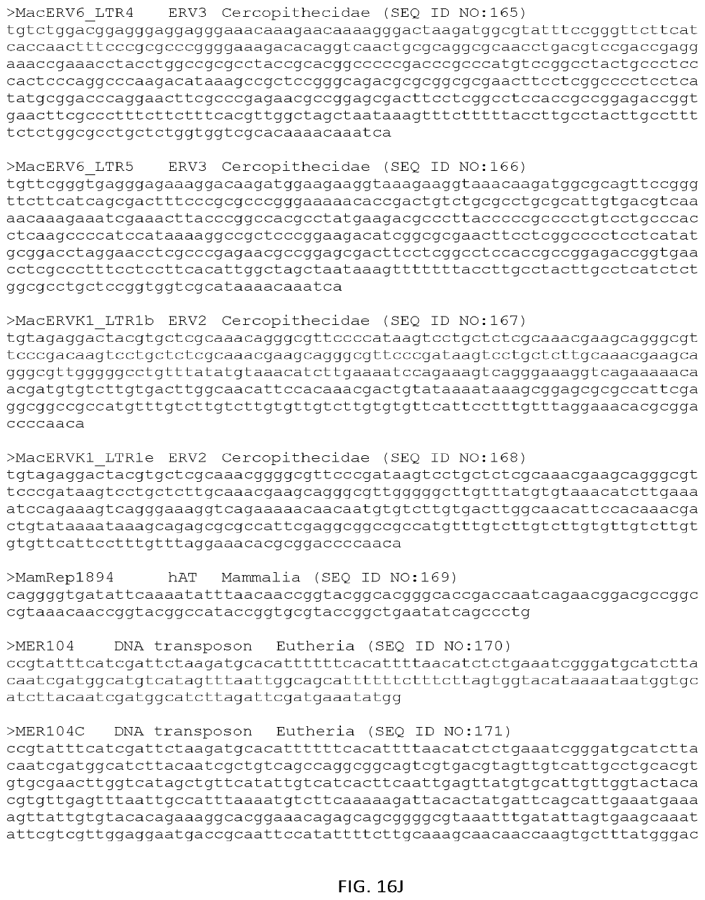

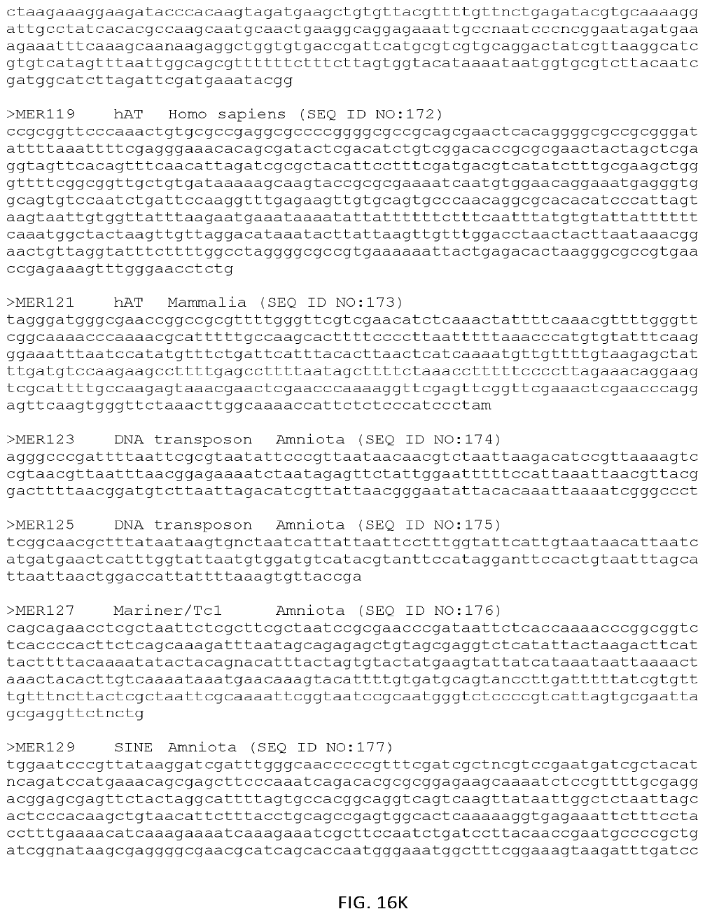

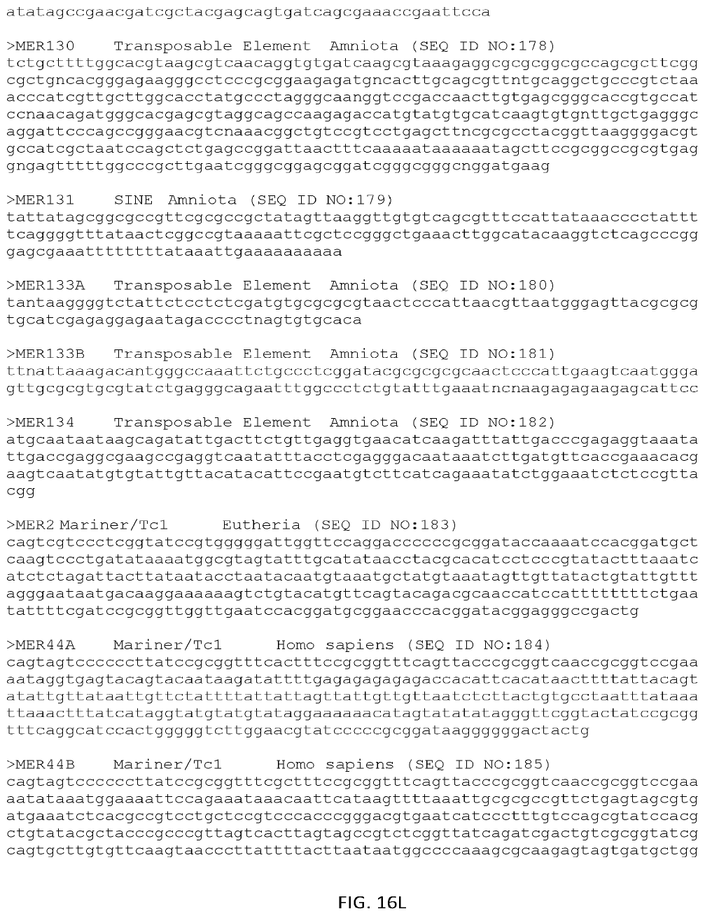

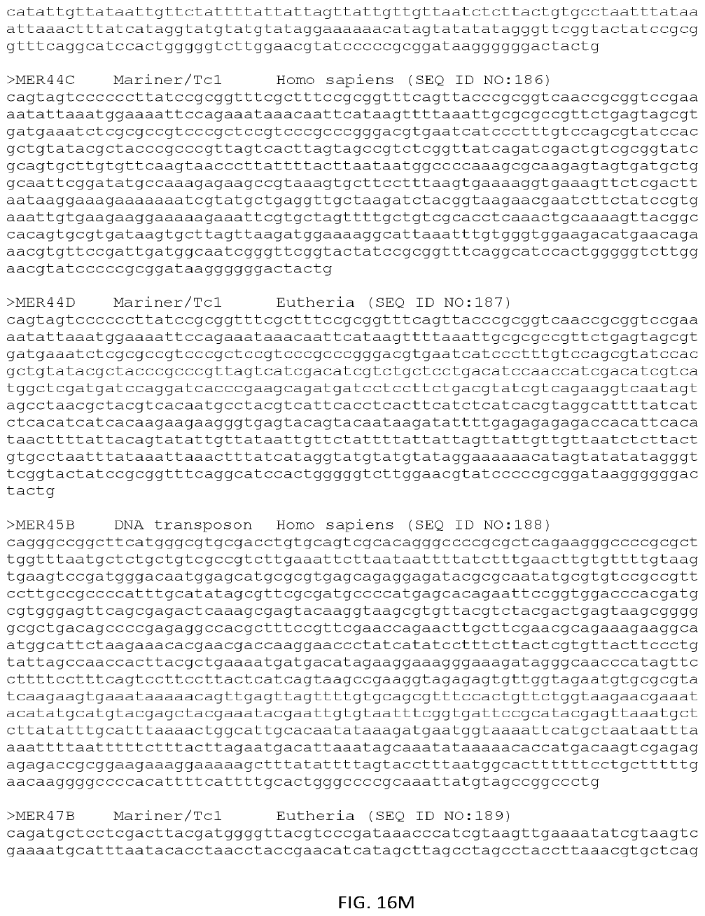

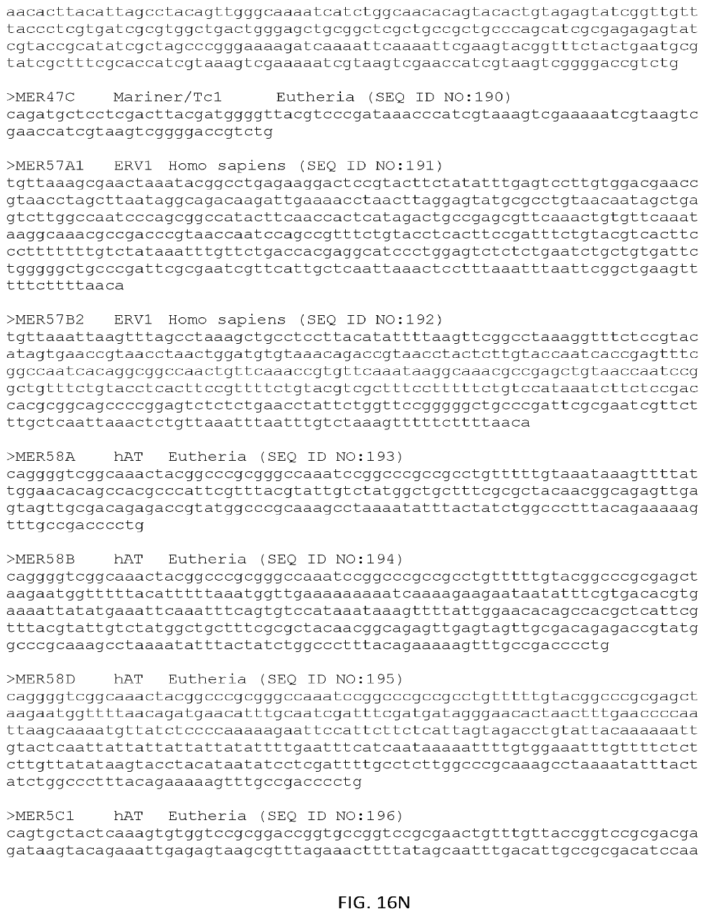

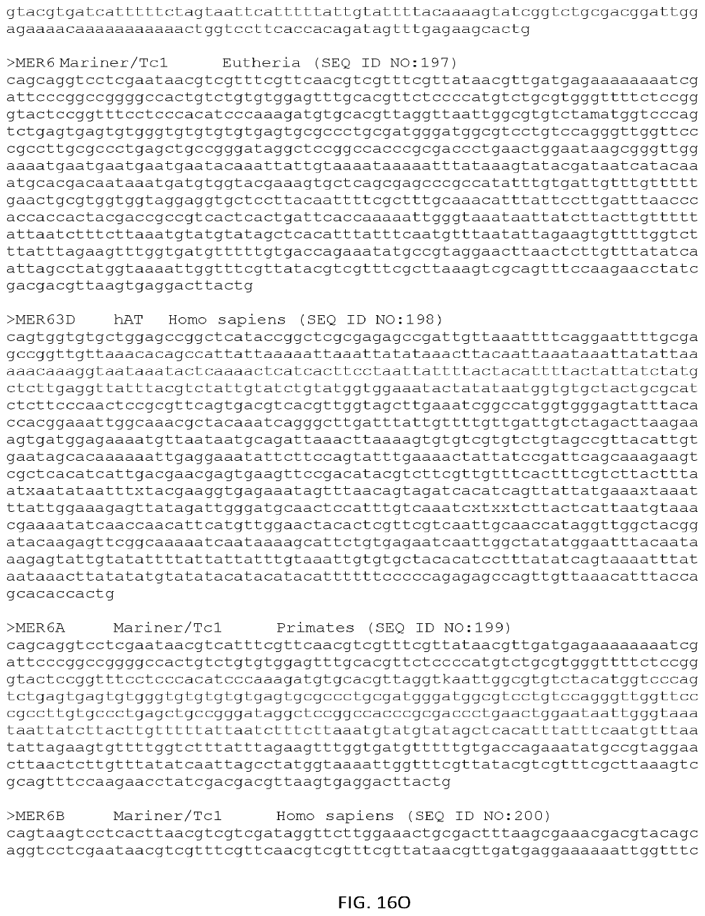

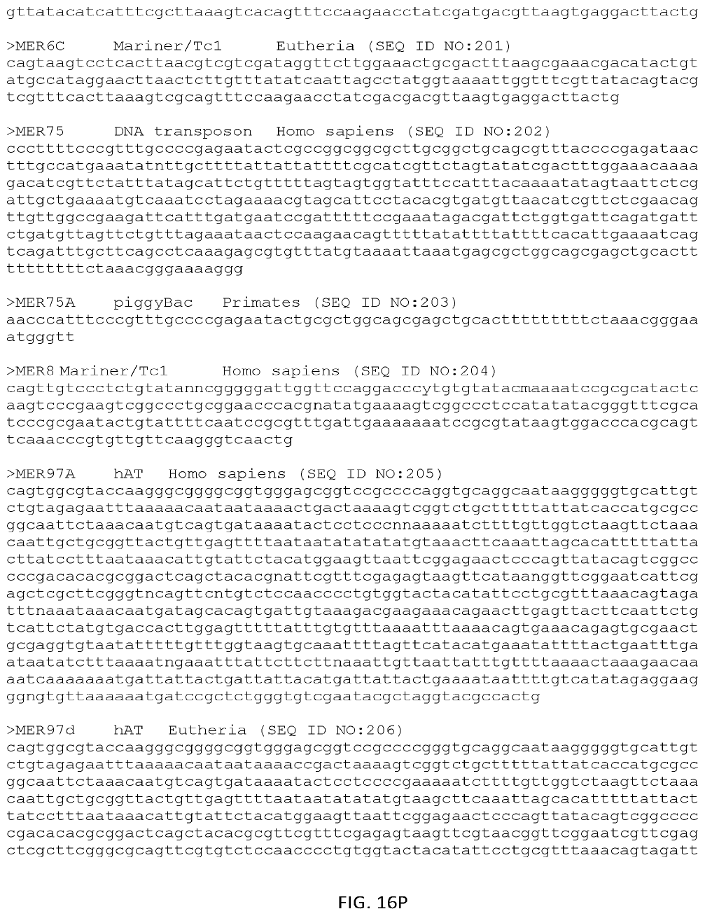

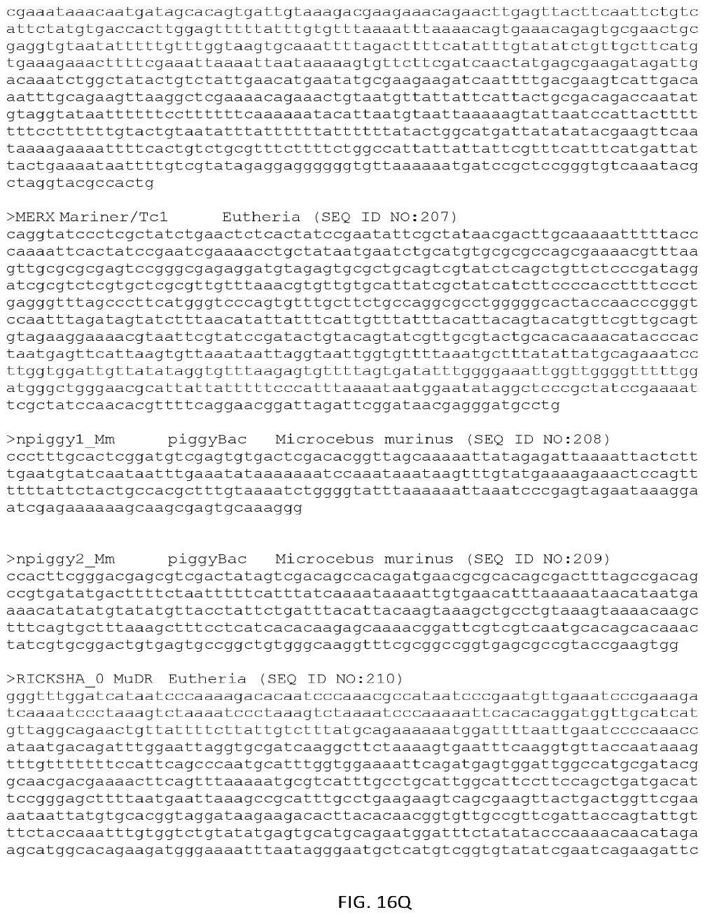

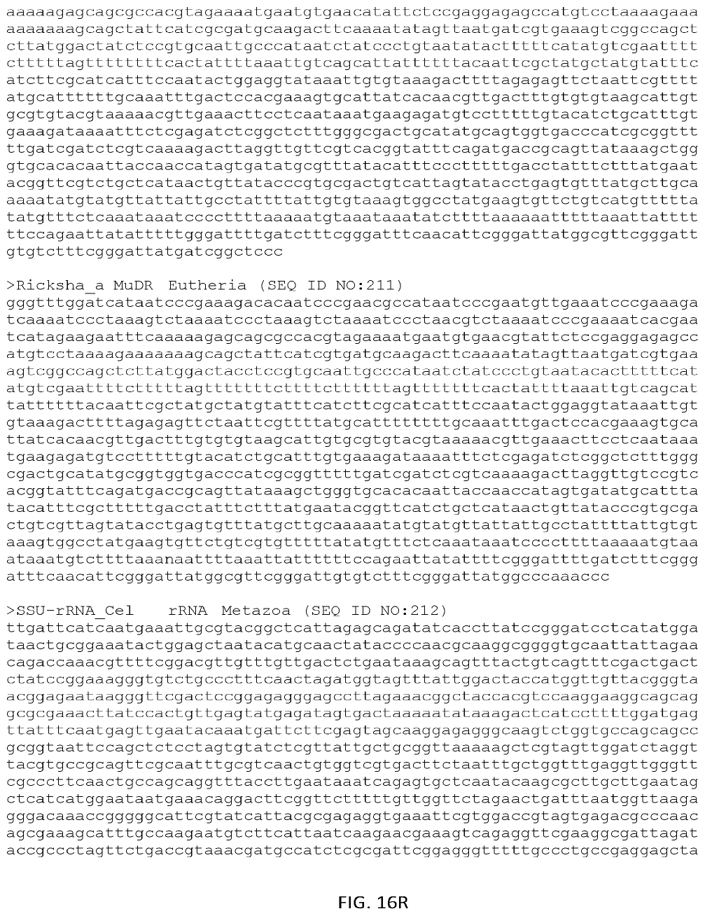

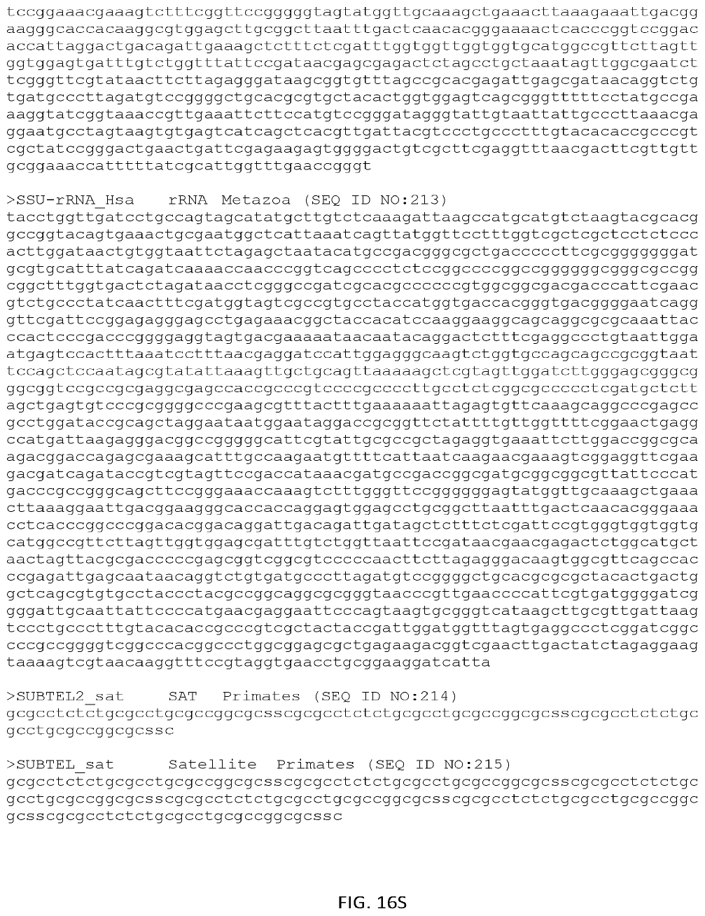

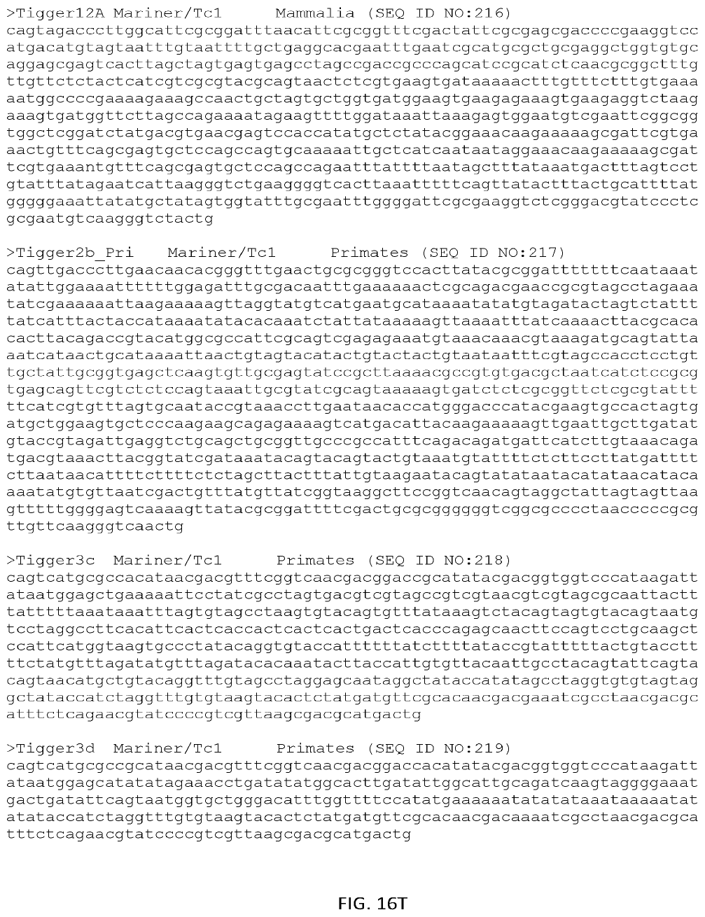

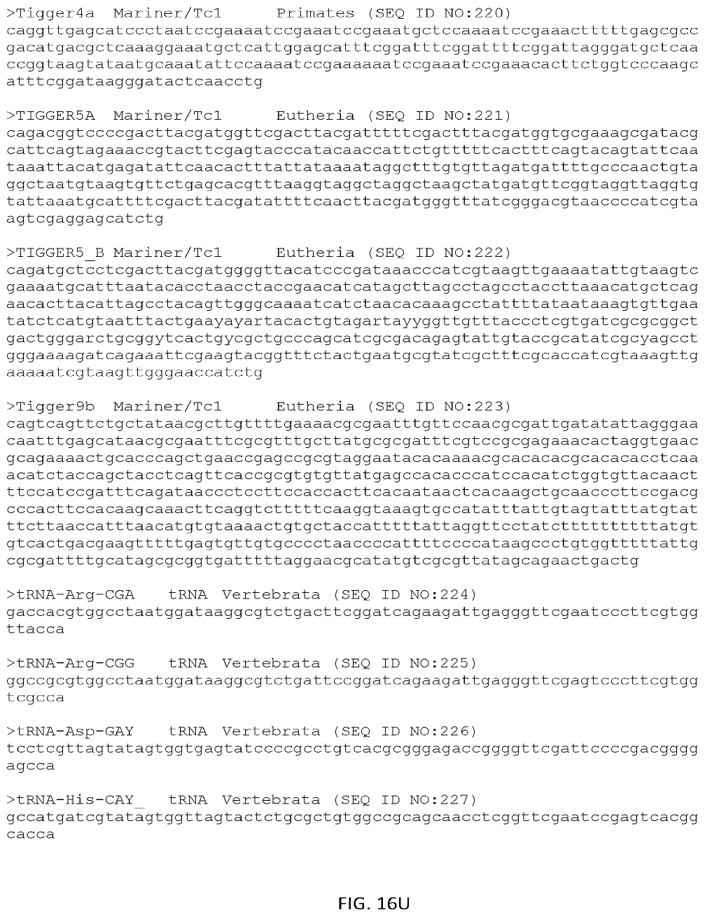

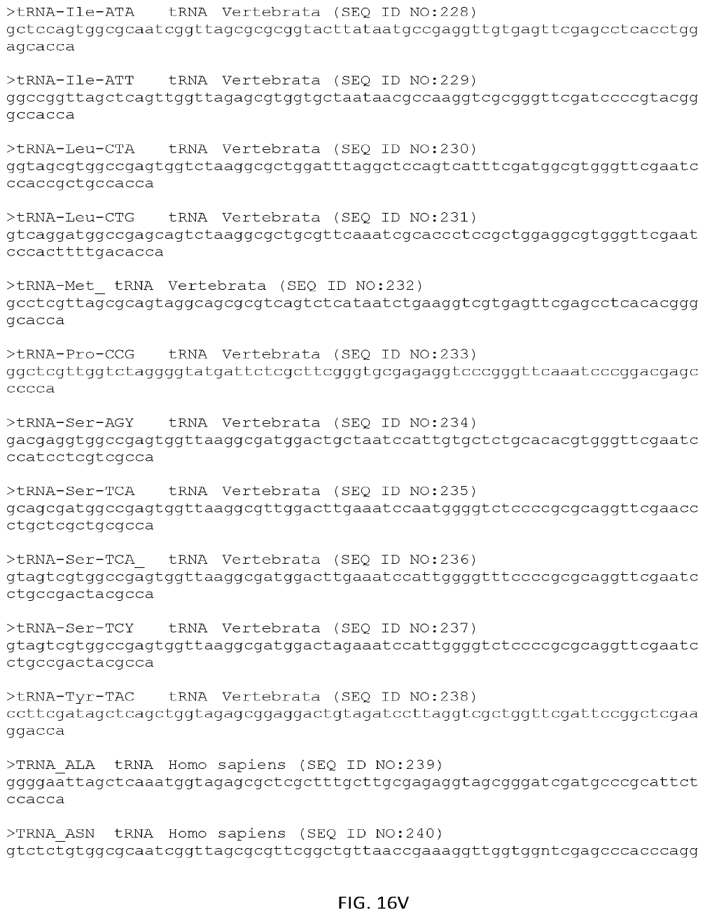

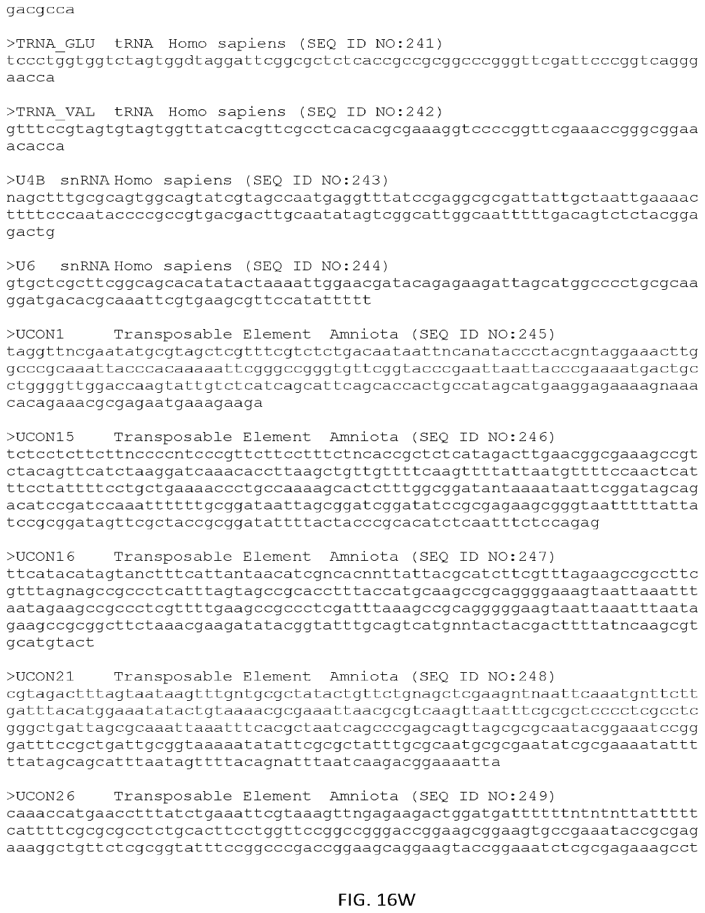

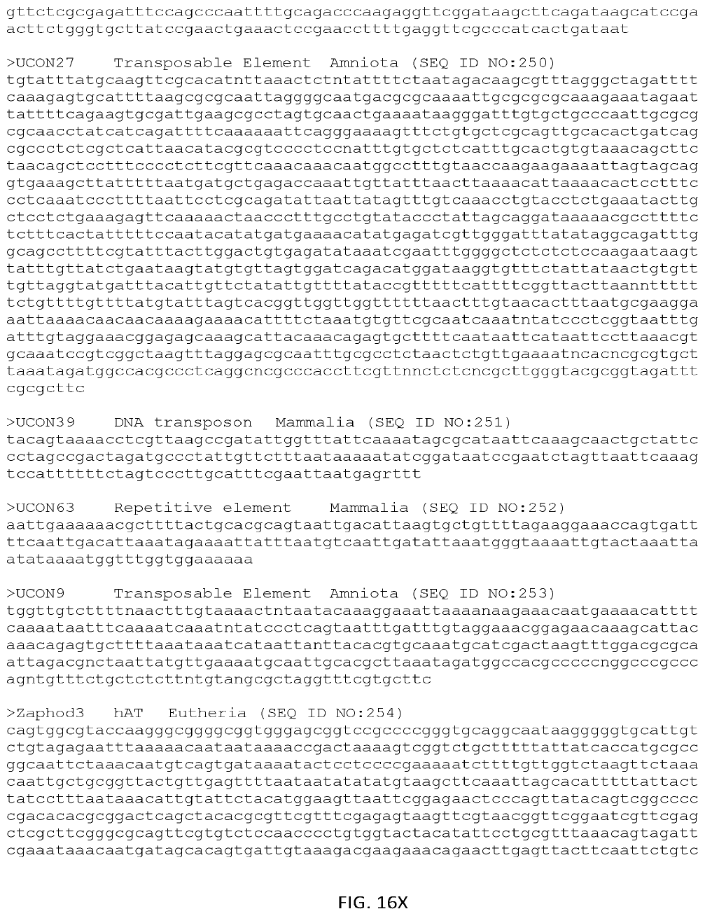

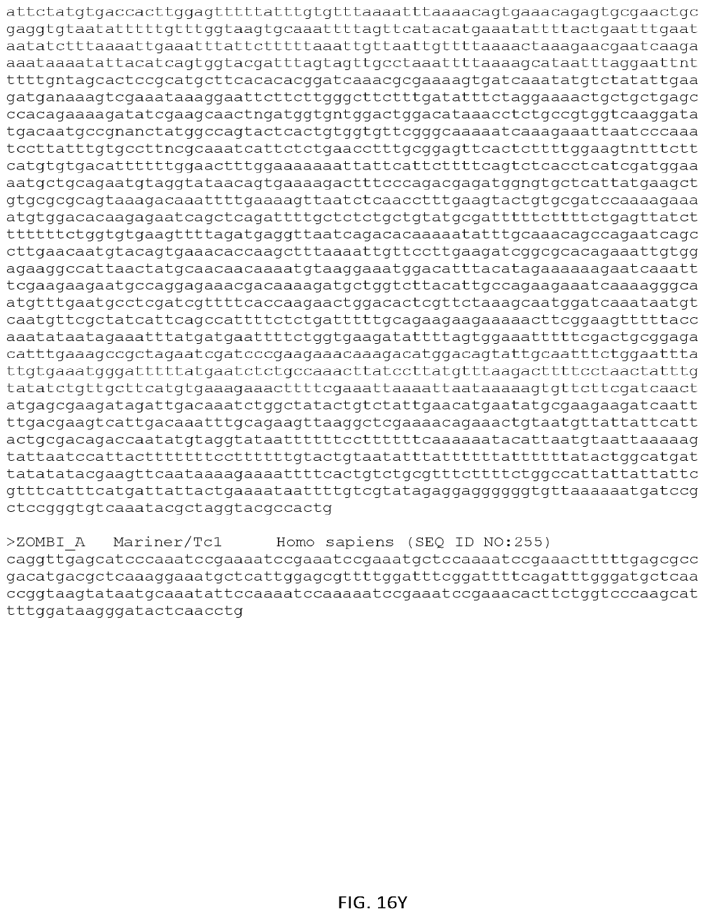

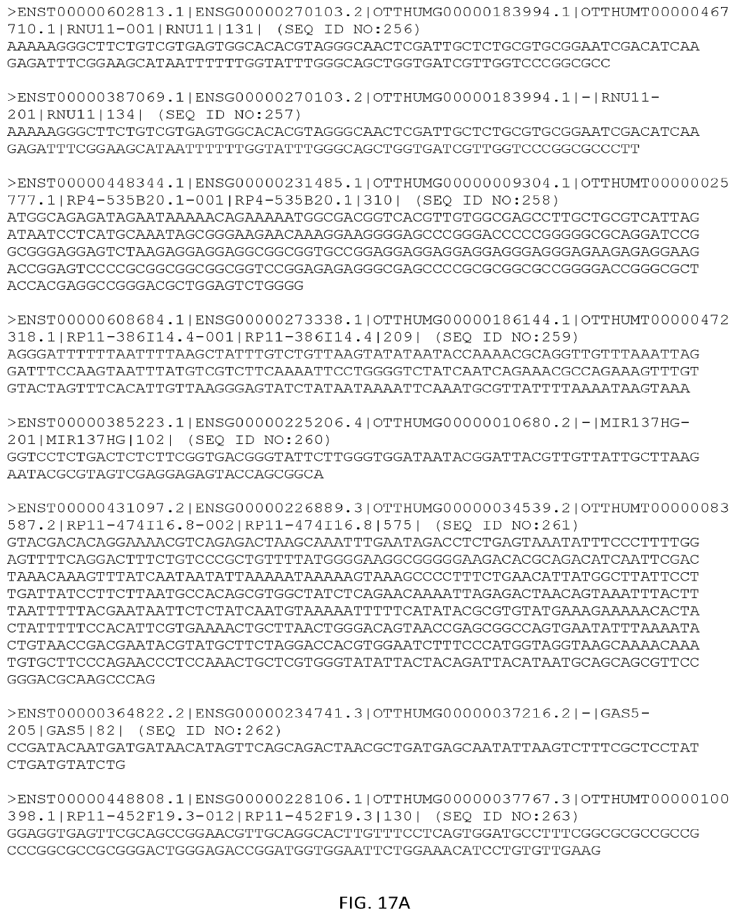

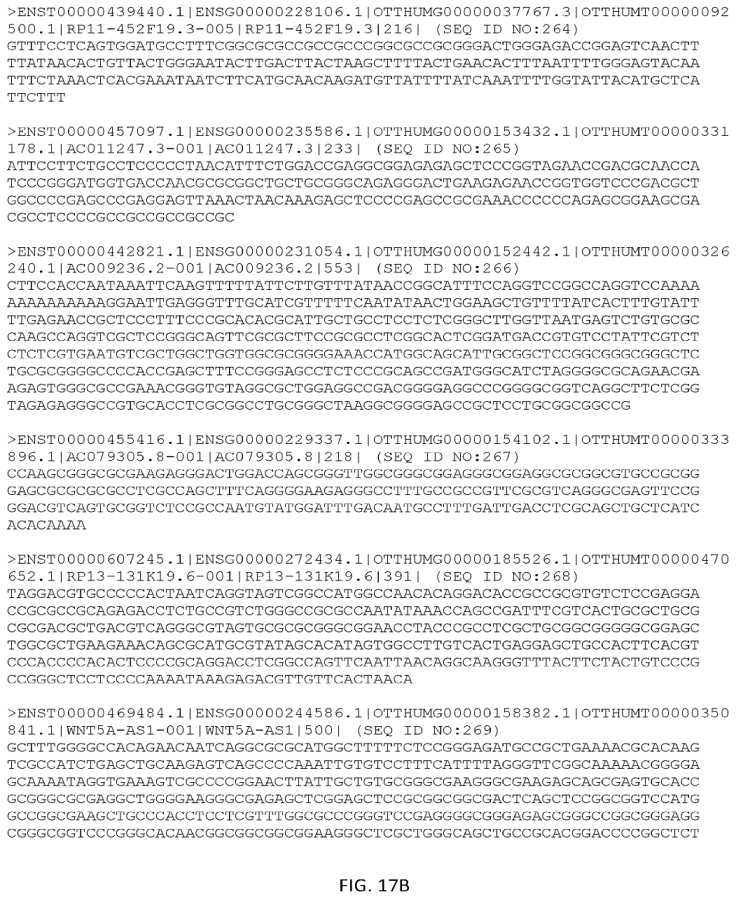

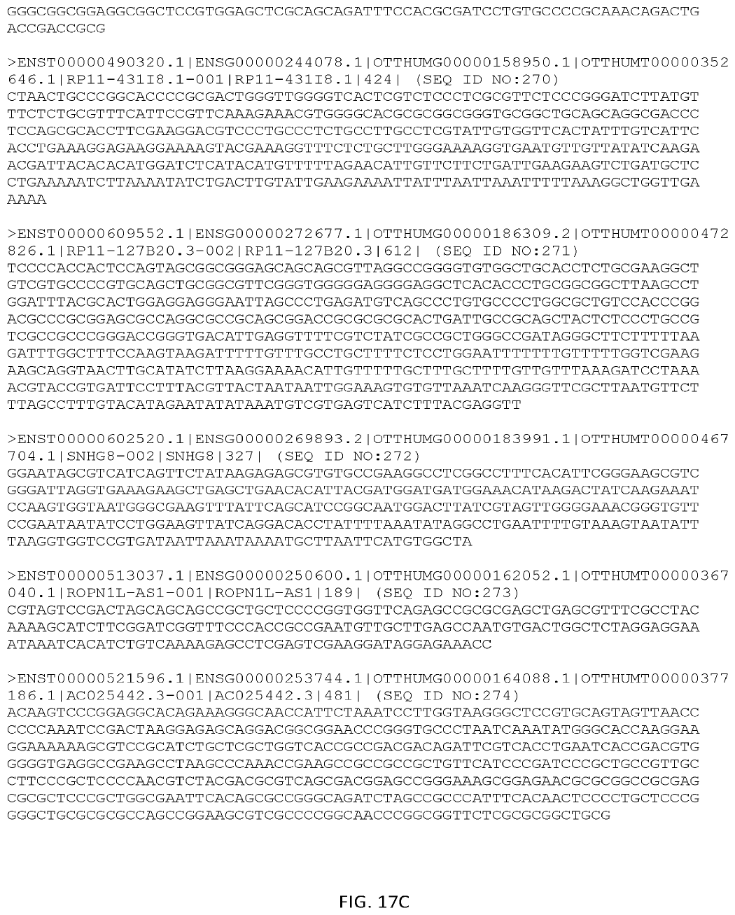

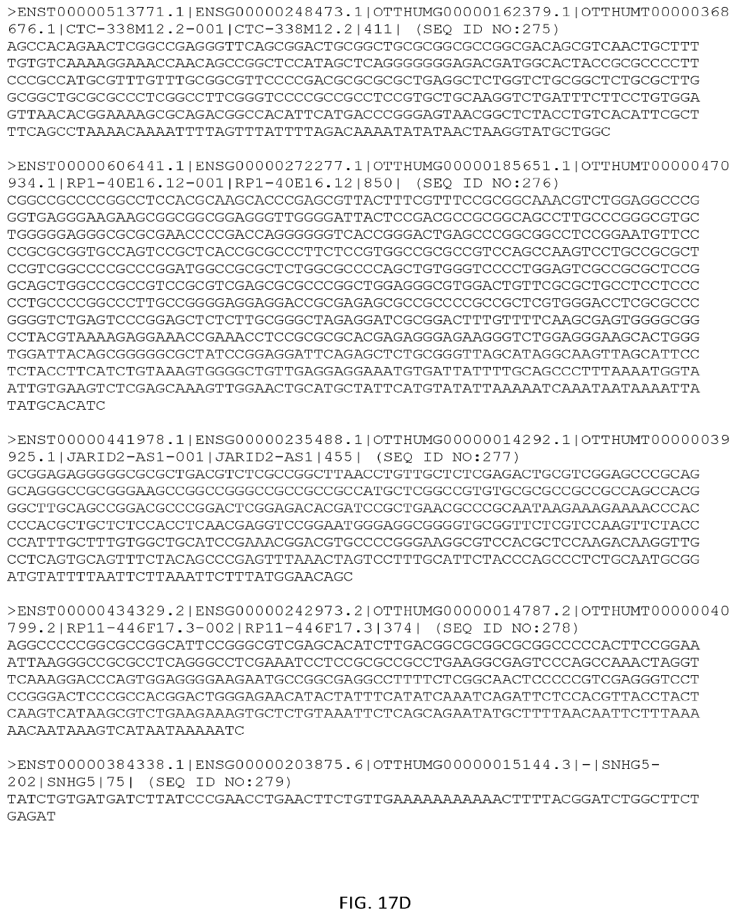

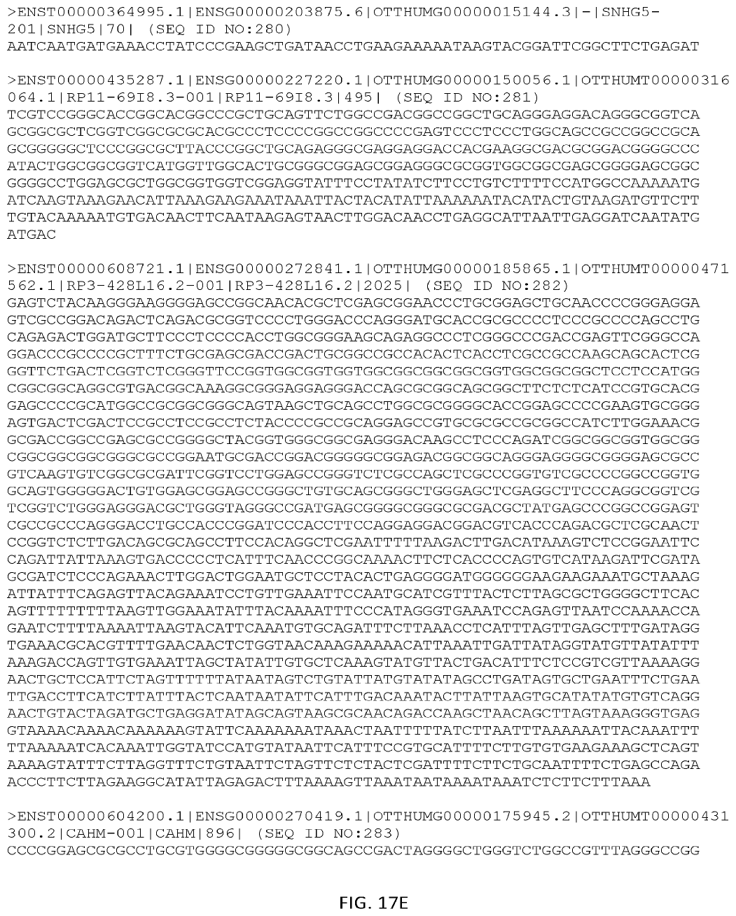

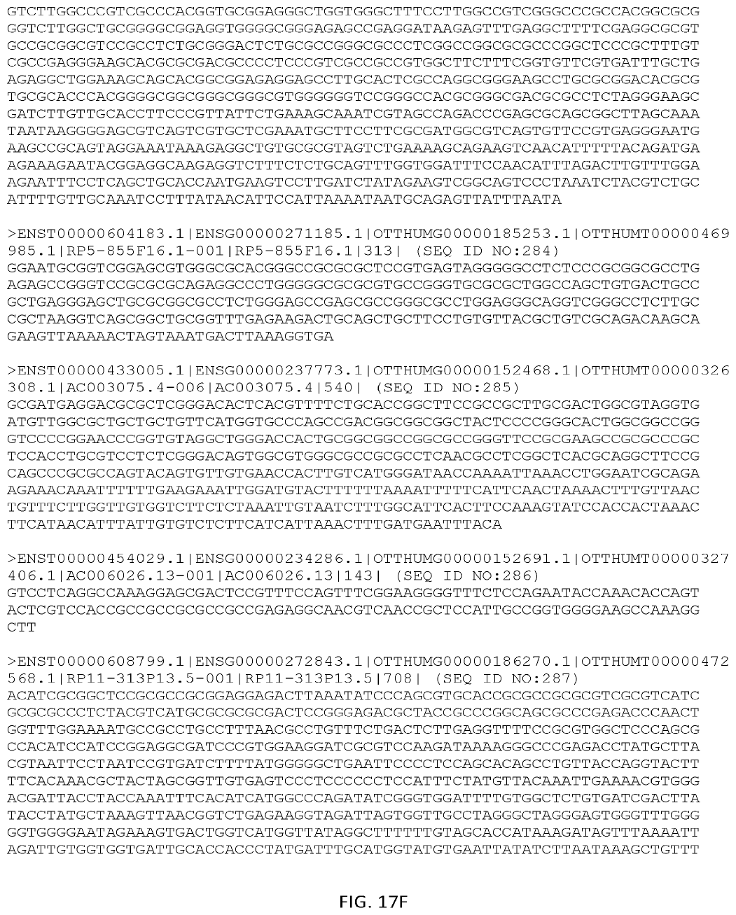

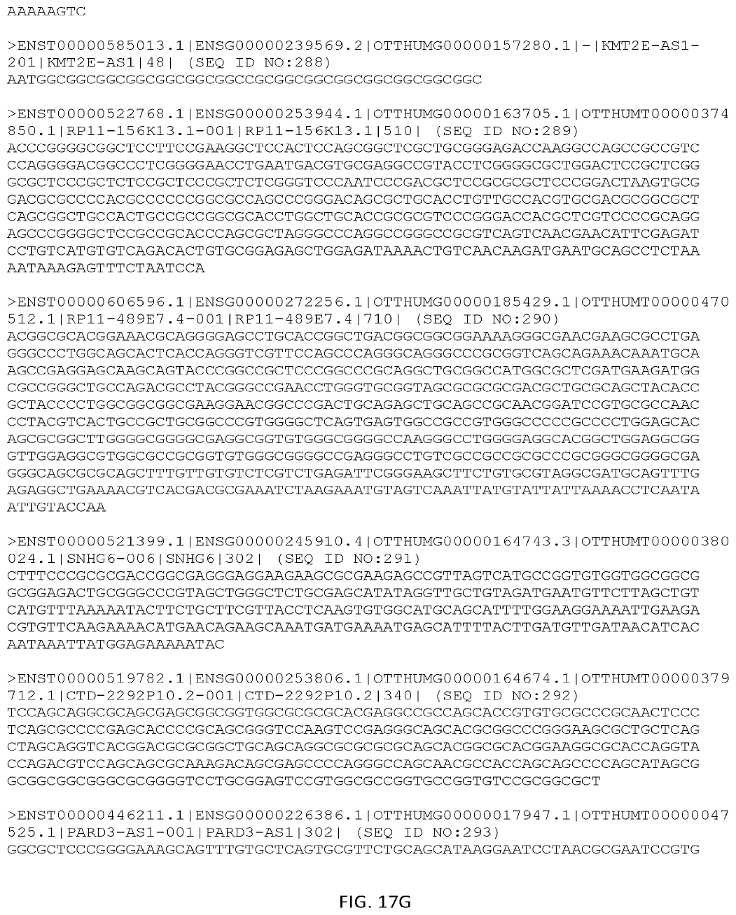

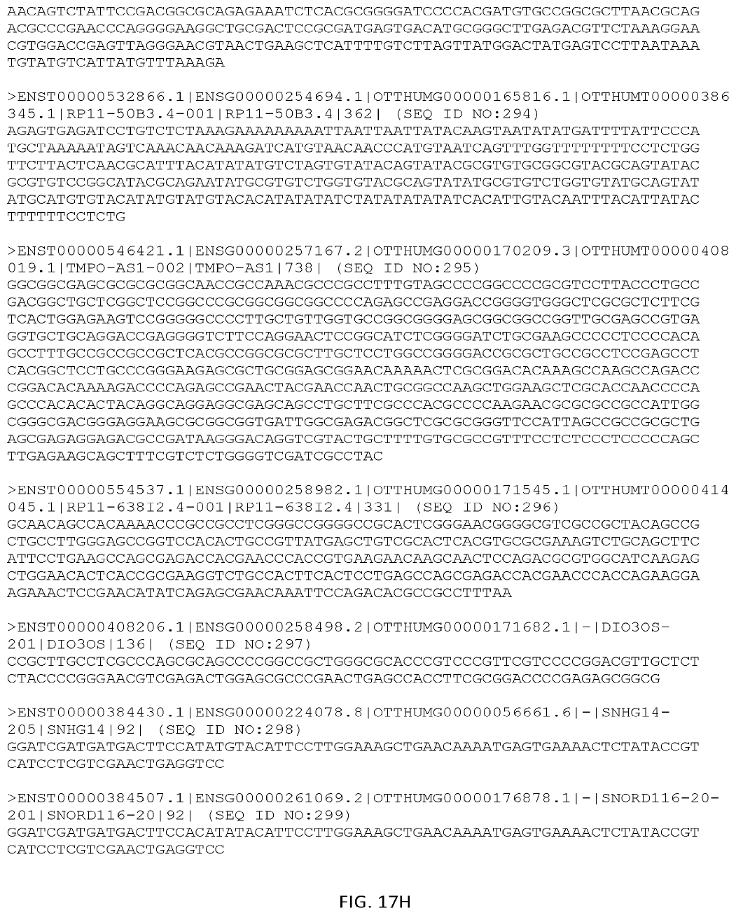

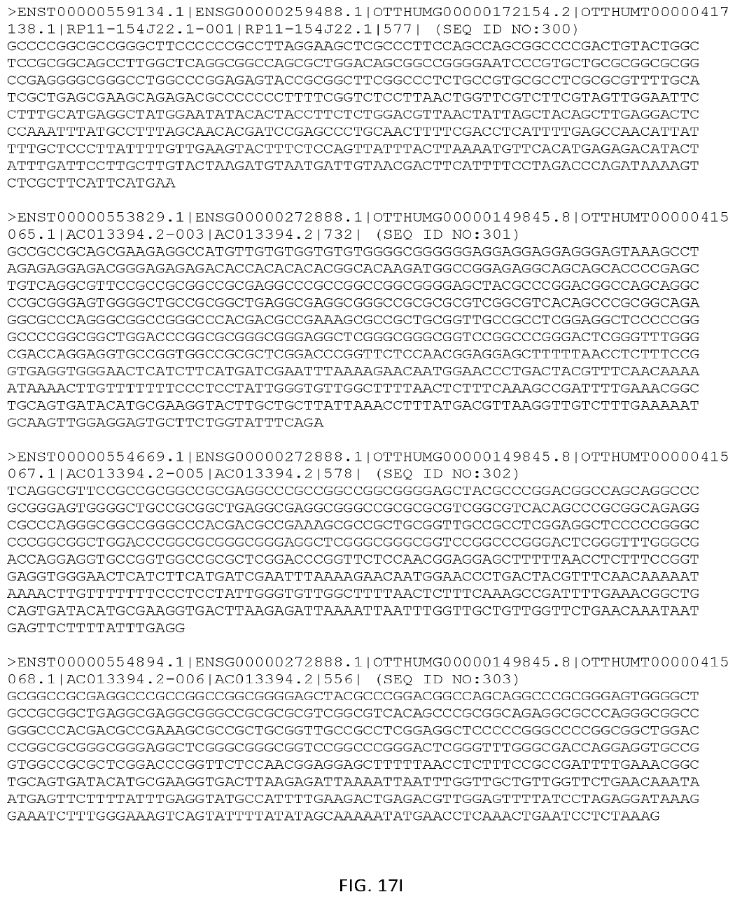

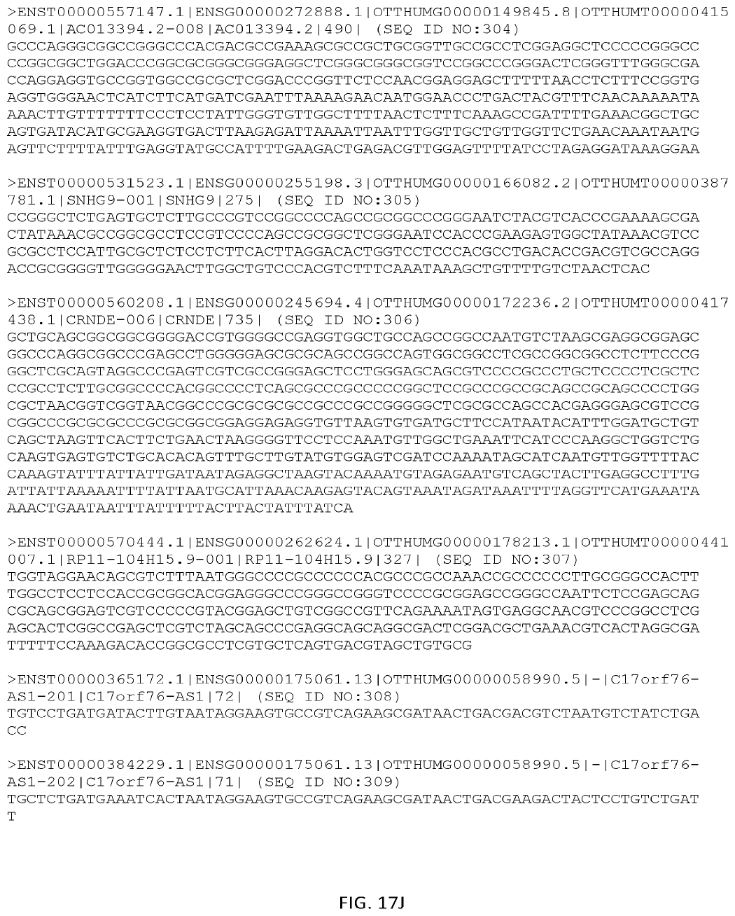

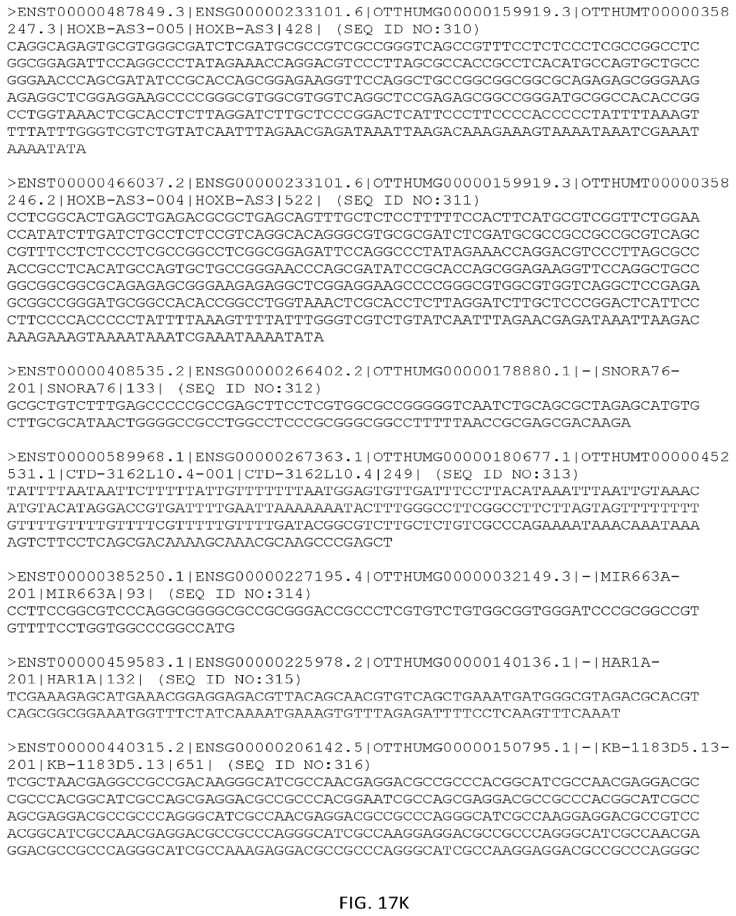

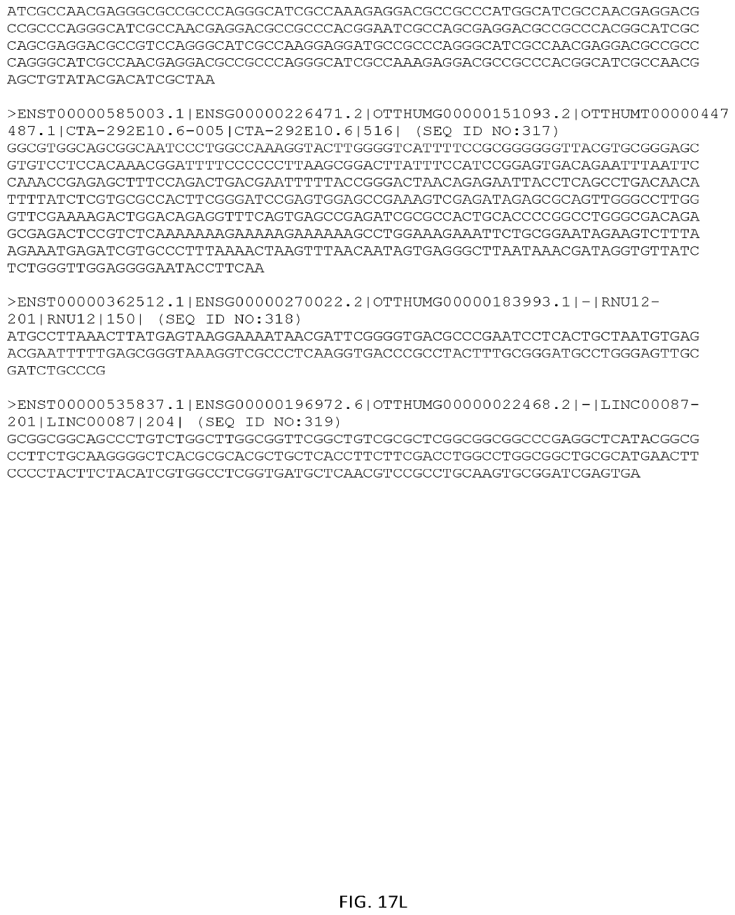

Specific exemplary RNA molecules of the composition include, without limitation, SEQ ID NOs:1-96 (FIGS. 14A-S), SEQ ID NOs:97-120 (FIGS. 15A-F), SEQ ID NOs:121-255 (FIGS. 16A-Y), SEQ ID NOs:256-319 (FIGS. 17A-L), and immunostimulating fragments thereof.

The RNA molecule in the composition of the present invention has an immunostimulating effect on cells, including tumor cells. As used herein, the term “immunostimulating effect” or “stimulating an immune response” includes eliciting an immune response, e.g., inducing or increasing T cell-mediated and/or B cell-mediated immune responses that are influenced by modulation of T cell costimulation. Exemplary immune responses include B cell responses (e.g., antibody production), T cell responses (e.g., cytokine production, and cellular cytotoxicity), and activation of cytokine responsive cells, e.g., macrophages. Eliciting an immune response includes an increase in any one or more immune responses. It will be understood that upmodulation of one type of immune response may lead to a corresponding downmodulation in another type of immune response. For example, upmodulation of the production of certain cytokines (e.g., IL-10) can lead to downmodulation of cellular immune responses. The RNA molecule elicits an immunostimulating effect on immune cells. As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes. The term “T cell” includes CD4+ T cells and CD8+ T cells. The term T cell also includes both T helper 1 type T cells and T helper 2 type T cells.

In formulating the RNA-containing composition of the present invention, the amount of RNA molecule included in the composition will vary depending on the choice of RNA molecule, its immunostimulating activity, and its intended treatment and subject.

In the composition of the present invention, the RNA molecule is incorporated into pharmaceutical compositions suitable for administration (e.g., by injection). Such compositions typically comprise the RNA molecule and a carrier, e.g., a pharmaceutically acceptable carrier. The pharmaceutically acceptable carrier suitable for injection is, according to one embodiment, a carrier for the RNA molecule. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Supplementary active compounds can also be incorporated into the compositions.

The pharmaceutically acceptable carrier may be a stabilizer, an emulsion, liposome, microsphere, immune stimulating complex, nanospheres, montanide, squalene, cyclic dinucleotides, complementary immune modulators, or any combination thereof. The carrier should be suitable for the desired mode of delivery of the composition (i.e., suitable for injection). Exemplary modes of delivery include, without limitation, intravenous injection, intra-arterial injection, intramuscular injection, intracavitary injection, subcutaneously, intradermally, transcutaneously, intrapleurally, intraperitoneally, intraventricularly, intra-articularly, intraocularly, intratumorally, or intraspinally.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., RNA molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound (i.e., RNA molecule) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the methods of the invention (described infra), the therapeutically effective dose can be estimated initially from cell culture assays. A dose may be formulated in animal models to achieve a circulating plasma concentration range that includes the IC₅₀ (i.e., the concentration of the test compound which achieves a half-maximal activity) as determined in cell culture. Such information can be used to more accurately determine useful doses in humans. Levels in plasma may be measured, for example, by high performance liquid chromatography.

As defined herein, a therapeutically effective amount of an RNA molecule (i.e., an effective dosage) ranges from about 0.001 to 30 mg/kg body weight, or about 0.01 to 25 mg/kg body weight, or about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to, the severity of the disease or disorder, previous treatments, the general health and/or age of the subject, and other diseases present. Moreover, treatment of a subject with a therapeutically effective amount of an agent can include a single treatment or, preferably, can include a series of treatments.

In one embodiment, a subject is treated with the composition of the present invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of composition used for treatment may increase or decrease over the course of a particular treatment. Changes in dosage may result and become apparent from the results of diagnostic assays.

In one embodiment, nucleic acid molecules can be inserted into vectors and used as gene therapy vectors. Gene therapy vectors can be delivered to a subject by, for example, intravenous injection, local administration (U.S. Pat. No. 5,328,470, which is hereby incorporated by reference in its entirety) or by stereotactic injection (Chen et al., “Regression of Experimental Gliomas by Adenovirus-Mediated Gene Transfer In Vivo,” Proc. Natl. Acad. Sci. USA 91:3054-3057 (1994), which is hereby incorporated by reference in its entirety). The pharmaceutical preparation of the gene therapy vector can include the gene therapy vector in an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. The pharmaceutical compositions can be included in a container, pack, or dispenser together with instructions for administration.

The composition of the present invention can also include an effective amount of an additional adjuvant or mitogen.

Suitable additional adjuvants include, without limitation, Freund's complete or incomplete, mineral gels such as aluminum hydroxide, surface active substances such as lysolecithin, pluronic polyols, polyanions, peptides, oil emulsions, dinitrophenol, Bacille Calmette-Guerin, Carynebacterium parvum, non-toxic Cholera toxin, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanme-2-(r-2′-dipalmitoyl-s-n-glycero-3-hydroxyphosphoryloxy)-ethylamine (CGP 19835 A, referred to as MTP-PE), and RIBI, which contains three components extracted from bacteria, monophosphoryl lipid A, trehalose dimycolate, and cell wall skeleton (MPL+TDM+CWS) in a 2% squalene/TWEEN® 80 emulsion.

As used herein, “mitogen” refers to any agent that stimulates lymphocytes to proliferate independently of an antigen. The mitogen, in combination with the RNA molecule in the composition of the present invention helps to promote an immunostimulating effect on tumor cells. Exemplary mitogen include, without limitation, CpG oligodeoxynucleotides that stimulate immune activation as described in U.S. Pat. Nos. 6,194,388; 6,207,646; 6,214,806; 6,218,371; 6,239,116; 6,339,068; 6,406,705; and 6,429,199, each of which is hereby incorporated by reference in its entirety. Any suitable dosage of mitogen can be used to promote an immunostimulating effect on tumor cells. For example, a suitable dosage of mitogen comprises about 50 ng up to about 100 μg per ml, about 100 ng up to about 25 μg per ml, or about 500 ng up to about 5 μg per ml.

The composition may also include an antigen or an antigen-encoding RNA molecule. As used herein, “antigen” refers to any agent that induces an immune response, i.e., a protective immune response, against the antigen, and thereby affords protection against a pathogen or disease (e.g., cancer). The antigen can take any suitable form including, without limitation, whole virus or bacteria; virus-like particle; anti-idiotype antibody; bacterial, viral, or parasite subunit vaccine or recombinant vaccine; and bacterial outer membrane (“OM”) bleb formations containing one or more of bacterial OM proteins.

The antigen can be present in the compositions in any suitable amount that is sufficient to generate an immunologically desired response. The amount of antigen or antigen-encoding RNA molecule to be included in the composition will depend on the immunogenicity of the antigen itself and the efficacy of any adjuvants co-administered therewith. In general, an immunologically or prophylactically effective dose comprises about 1 μg to about 1,000 μg of the antigen, about 5 μg to about 500 μg, or about 10 μg to about 200 μg.

According to another embodiment, the composition (i.e., a first pharmaceutical composition) may further include a cancer vaccine (i.e., as a second pharmaceutical composition) that includes an antigen or a nucleic acid molecule encoding the antigen, and a pharmaceutically suitable carrier. According to this embodiment, the first pharmaceutical composition is intended to be co-administered with the second pharmaceutical composition for purposes of enhancing the efficacy of the vaccine. The first pharmaceutical composition is formulated for and/or administered in a manner that achieves an immunostimulating effect on tumor cells.

Cancer vaccines are known, and include, for example, sipuleucel-T (Provenge®, manufactured by Dendreon), which is approved for use in some men with metastatic prostate cancer. This vaccine is designed to stimulate an immune response to prostatic acid phosphatase (“PAP”), an antigen that is found on most prostate cancer cells. Sipuleucel-T is customized to each patient. The vaccine is created by isolating immune system cells called antigen-presenting cells (“APCs”) from a patient's blood through a procedure called leukapheresis. The APCs are sent to Dendreon, where they are cultured with a protein called PAP-GM-CSF. This protein consists of PAP linked to another protein called granulocyte-macrophage colony-stimulating factor (GM-CSF). The latter protein stimulates the immune system and enhances antigen presentation. APC cells cultured with PAP-GM-CSF constitute the active component of sipuleucel-T. Each patient's cells are returned to the patient's treating physician and infused into the patient. Patients receive three treatments, usually 2 weeks apart, with each round of treatment requiring the same manufacturing process. Although the precise mechanism of action of sipuleucel-T is not known, it appears that the APCs that have taken up PAP-GM-CSF stimulate T cells of the immune system to kill tumor cells that express PAP.

Vaccines to prevent HPV infection and to treat several types of cancer are being studied in clinical trials. Active clinical trials of cancer treatment vaccines include vaccines for bladder cancer, brain tumors, breast cancer, cervical cancer, Hodgkin lymphoma, kidney cancer, leukemia, lung cancer, melanoma, multiple myeloma, non-Hodgkin lymphoma, pancreatic cancer, prostate cancer, and solid tumors. Active clinical trials of cancer preventive vaccines include those for cervical cancer and solid tumors. Cancer vaccines approved from these and other trials may be suitable cancer vaccines for use in combination with the composition of the present invention.

Another aspect of the present invention relates to a kit comprising a cancer vaccine and the composition of the present invention, as well as instructions and a suitable delivery device, which can optionally be pre-filled with the vaccine formulation (i.e., the composition of the present invention and the cancer vaccine). An exemplary delivery device includes, without limitation, a syringe comprising an injectable dose.

A further aspect of the present invention relates to a method of treating a subject for a tumor. This method involves administering to a subject the composition of the present invention under conditions effective to treat the subject for the tumor.

In one embodiment of this and other methods described herein, the subject is a mammal including, without limitation, humans, non-human primates, dogs, cats, rodents, horses, cattle, sheep, and pigs. Both juvenile and adult mammals can be treated. The subject to be treated in accordance with the present invention can be a healthy subject, a subject with a tumor, a subject with cancer, a subject being treated for cancer, a subject in cancer remission, or a subject that has an immune deficiency or is immunosuppressed. Although otherwise healthy, the elderly and the very young may have a less effective (or less developed) immune system and they may benefit greatly from the enhanced immune response.

Tumors include, without limitation, sarcoma, melanoma, lymphoma, leukemia, neuroblastoma, or carcinoma cell tumors.

In carrying out this and the other methods described herein, administering may be carried out as described supra, including, for example, intratumorally or systemically using a pharmaceutical composition as described supra, and amounts, dosages, and administration frequencies described supra.

A further aspect of the present invention relates to a method of stimulating an immune response against cancer in a cell or tissue. This method involves providing the composition of the present invention and contacting a cell or tissue with the composition under conditions effective to stimulate an immune response against cancer in the cell or tissue.

Cancers suitable for treatment in carrying out this aspect of the present invention include, for example and without limitation, those that are incident to pathogen infection, e.g., cervical cancer, vaginal cancer, vulvar cancer, oropharyngeal cancers, anal cancer, penile cancer, and squamous cell carcinoma of the skin caused by papillomavirus infection (D'Souza et al, “Case-Control Study of Human Papillomavirus and Oropharyngeal Cancer,” NEJM 356(19):1944-1956 (2007); Harper et al., “Sustained Immunogenicity and High Efficacy Against HPV 16/18 Related Cervical Neoplasia: Long-term Follow up Through 6.4 Years in Women Vaccinated with Cervarix (GSK's HPV-16/18 AS04 candidate vaccine),” Gynecol. Oncol. 109:158-159 (2008), each of which is hereby incorporated by reference in its entirety) and liver cancer caused by Hepatitis B virus infection (Chang et al., “Decreased Incidence of Hepatocellular Carcinoma in Hepatitis B Vaccines: A 20-Year Follow-up Study,” J. Natl. Cancer Inst. 101:1348-1355 (2009), which is hereby incorporated by reference in its entirety) and Hepatitis C virus infection, Burkitt lymphoma, non-Hodgkin lymphoma, Hodgkin lymphoma, nasopharyngeal carcinoma caused by the Epstein-Barr virus, Kaposi sarcoma caused by the Kaposi sarcoma-associated herpesvirus, adult T-cell leukemia/lymphoma, caused by the human T-cell lymphotropic virus type 1, stomach cancer, mucosa-associated lymphoid tissue lymphoma caused by the bacterium Helicobacter pylori, bladder cancer caused by the parasite Schistosoma hematobium, and cholangiocarcinoma caused by the parasite Opisthorchis viverrini. An enhanced immune response achieved by the methods of treatment and compositions of the present invention may enhance the preventative efficacy of such vaccines for the prevention of cancers.

In one embodiment, this and other methods of the present invention are carried out to treat cancers that have already developed in a subject. Thus, the methods and compositions of the present invention are intended to delay or stop cancer cell growth; to cause tumor shrinkage; to prevent cancer from coming back; or to eliminate cancer cells that have not been killed bye other forms of treatment.

According to one embodiment, a composition to be administered includes the antigen that is intended to generate the desired immune response as well as the RNA molecule having a pattern of CpG dinucleotides defined by a strength of statistical bias greater than or equal to zero. Thus, the antigen and the RNA molecule are co-administered simultaneously. The composition may be administered as a vaccine in a single dose or in multiple doses, which can be the same or different.

This embodiment may optionally include further administration of a composition of the present invention that includes the RNA molecule but not the antigen. This composition can be administered once or twice daily within several days preceding vaccine administration and for a period of time following vaccine administration. By way of example, post-vaccine administration can be carried out for up to about six weeks following each vaccine administration, preferably at least about two to three weeks, or at least about 3 to 10 days following each vaccine administration.

According to a second embodiment, a vaccine composition to be administered includes the antigen that is intended to generate the desired immune response but not the RNA molecule. However, the RNA molecule can be co-administered at about the same time. For instance, the dosage of the vaccine can be administered interperitoneally or intransally, and a dosage of the RNA molecule can be administered orally at about the same time (same day). The dosage containing the RNA molecule can also be once or twice administered daily for up to about six weeks following the vaccine administration.

In carrying out this method of the present invention, contacting the cell or tissue with the composition may be carried out in vitro or in vivo.

According to another aspect of the present invention, the RNA-containing composition has an immunostimulating effect that primes (e.g., stimulates, induces, enhances, alters, or modulates) the anti-pathogen response of a subject's innate immune system in non-tumor cells. Such a response may find use, e.g., as an adjuvant to a vaccine, a vaccine supplement, or under conditions where such an immunostimulating effect is desirable.

Yet a further aspect of the present invention relates to a method for identifying RNA molecules with immunostimulating patterns of CpG dinucleotides. This method involves providing an RNA molecule, determining the length and frequency of nucleotides in the RNA molecule, determining the number of CpG dinucleotides present in the RNA molecule, calculating the strength of statistical bias on CpG dinucleotides for the RNA molecule, defining a threshold of statistical bias, determining if the strength of statistical bias on CpG dinucleotides for the RNA molecule meets or exceeds the threshold, and characterizing the RNA molecule sequence as possessing an immunostimulating pattern if it meets or exceeds the threshold of statistical bias.

In carrying out this method of the present invention, nucleotide frequencies are calculated by counting the number of times that a nucleotide occurs and dividing that number by the total length of the sequence, L (which may also occur as ambiguously defined bases that cannot be assigned as A, C, G, U, or T). For example, f⁰(A), the frequency of A nucleotides, would be the number of occurrences of the base, A, in S₀ divided by L, the length of S₀, even when ambiguous bases are included.

In a further embodiment, the strength of statistical bias on CpG dinucleotides for the RNA molecule sequence (x(S₀)) is determined by maximizing the probability of a sequence (S₀) over x, where

$$\begin{matrix}P ( S \vert x , m ) = \frac{1}{{Z}_{m} ( x )} {\prod }_{i = 1}^{L} {f}^{0} ( {s}_{i} ) exp ( x {N}_{m} ( S ) ) & [ EQUATION 1 ] \\ {Z}_{m} ( x ) = {\sum }_{sequences S} {\prod }_{i = 1}^{L} {f}^{0} ( {s}_{i} ) exp ( x {N}_{m} ( S ) ) & [ EQUATION 2 ]\end{matrix}$$

Zₘ is the normalization constant,

P(S|x,m) is the probability of the sequence given the force (x) and motif m,

x is the force on the motif m that introduces a statistical bias over P,

Nₘ(S) is the number of observed motifs, and

f⁰ (sₗ is the nucleotide frequencies.

Defining a threshold of statistical bias can be carried out by providing a reference set comprising a plurality of RNA molecule sequences, calculating the strength of statistical bias on CpG dinucleotides for each RNA molecule sequence in the reference set, generating a distribution of the strengths of statistical bias on CpG dinucleotides for the RNA molecule sequences in the reference set to define a null distribution, setting a statistical significance level, and determining the value of the strength of statistical bias that meets or exceeds the statistical significance value.

The present invention may be further illustrated by reference to the following examples, which should not be construed as limiting.

### EXAMPLES

### Example 1

### General Motif Usage Patterns in lncRNAs

Using a novel approach from statistical physics, the experiments described herein quantify global transcriptome-wide motif usage for the first time in human and murine ncRNAs determining that most have motif usage consistent with the coding genome. However, an outlier subset of tumor-associated ncRNAs typically of recent evolutionary origin has motif usage that is often indicative of pathogen-associated RNA. For instance, as demonstrated in these examples, the tumor associated human repeat HSATII is enriched in motifs containing CpG dinucleotides in AU-rich contexts which most of the human genome and human adapted viruses have evolved to avoid. It is further demonstrated that a key subset of these ncRNAs function as immunostimulatory “self-agonists” and directly activate cells of the mononuclear phagocytic system to produce pro-inflammatory cytokines. These ncRNAs arise from endogenous repetitive elements that are normally silenced, yet are often very highly expressed in cancers. The innate response in tumors may partially originate from direct interaction of immunogenic ncRNAs expressed in cancer cells with innate pattern recognition receptors and thereby assign a new danger-associated function to a set of dark matter repetitive elements. These findings potentially reconcile several observations concerning the role of ncRNA expression in cancers and their relationship to the tumor microenvironment.

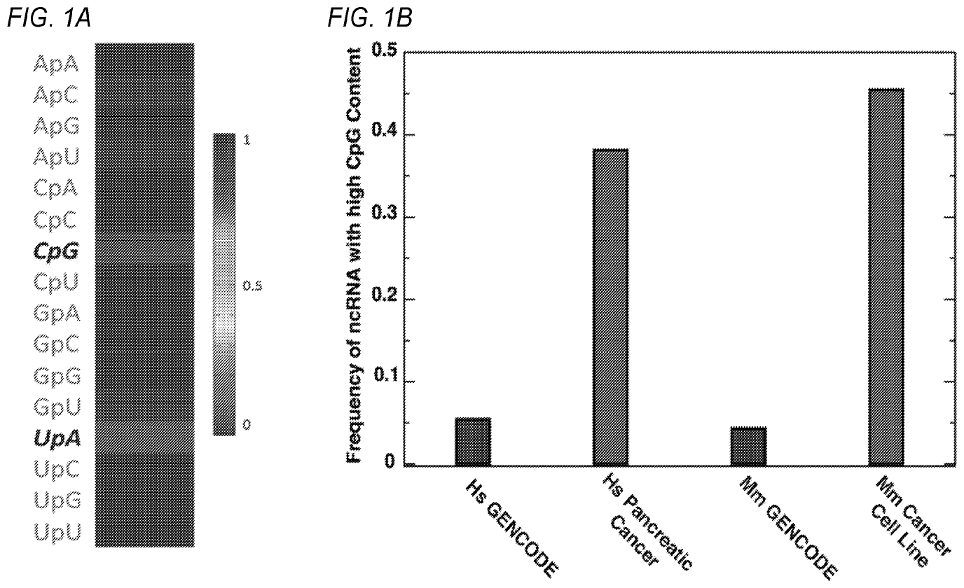

Employing the GENCODE database of long non-coding RNA transcripts from humans and mice (Versions 19 and 2 for human and mouse, respectively) the strength of statistical bias (referred to as a force) on sequence motif usage for all contained lncRNAs was calculated as described in Example 5 (infra). GENCODE lncRNA established a baseline of sequence motif usage expressed in a broad array of cells and tissues so that these patterns of motif usage could be compared with those of ncRNAs expressed in certain cancers. For each sequence, the force (i.e. strength of statistical bias) on all two and three nucleotide motifs was calculated using EQUATION 5 (infra) to calculate the probability of observing a sequence with that number of motifs. The number of sequences in GENCODE for which a given dinucleotide is aberrantly expressed is illustrated in FIG. 1A. CpG dinucleotides are vastly underrepresented, as indicated by their negative forces (i.e. strengths of statistical bias) in Table 1. UpA dinucleotides are often underrepresented though to a lesser extent. These patterns cannot be explained by nucleotide frequencies, such as GC content, which are accounted and normalized for with this method.

TABLE 1
Average Forces on Motifs are Similar between Humans and Mice

| | Human | Mouse |
|---|---|---|
| CG | −1.419 | −1.3750 |
| UA | −0.6040 | −0.5480 |
| ACG | −1.7586 | −1.6216 |
| CAG | 0.5534 | 0.5612 |
| CCG | −1.5095 | −1.3287 |
| CGA | −1.8995 | −1.7082 |
| CGC | −1.7304 | −1.5525 |
| CGG | −1.5110 | −1.2629 |
| CGU | −1.7833 | −1.6463 |
| CUG | 0.6690 | 0.6748 |
| GCG | −1.7480 | −1.5592 |
| GUA | −0.8632 | −0.7451 |
| UAC | −0.7368 | −0.6298 |
| UAG | −0.7330 | −0.5920 |
| UCG | −1.9391 | −1.7049 |

Average force (i.e. strength of statistical bias) on a given motif in the Human and Mouse GENCODE dataset, for lncRNAs with length greater than 500 nucleotides. The forces (i.e. strengths of statistical bias) are listed for the significant motifs in humans. The force is a measure of the strength of statistical bias to enhance or suppress a motif versus what is expected from that sequence's nucleotide content.

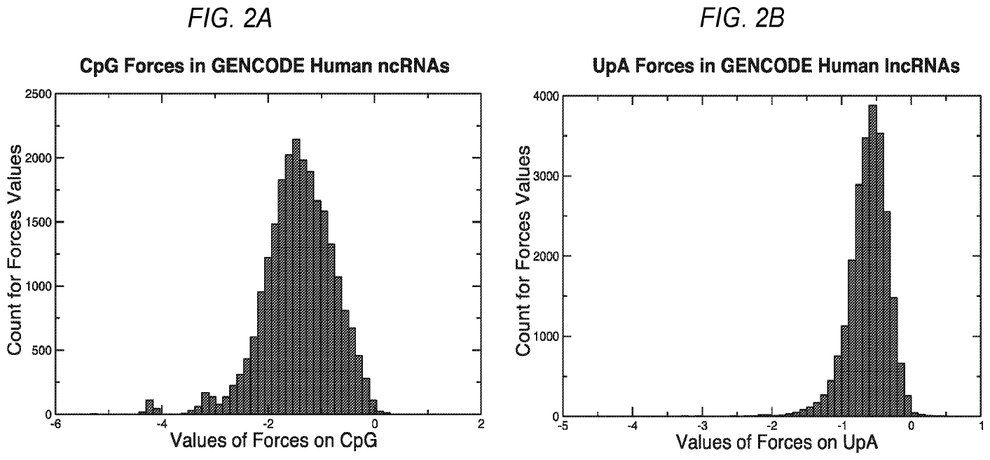

These dinucleotide motif usage patterns are similar in human and mouse genomes across the wide array of cells and cell lines contained in GENCODE (Djebali et al., “Landscape of Transcription in Human Cells,” Nature 48:101-108 (2012) and Harrow et al., “GENCODE: The Reference Human Genome Annotation for the ENCODE Project,” Genome Res. 22:1760-1774 (2012), which are hereby incorporated by reference in their entirety). Strikingly, avoidance of the CpG and UpA dinucleotide motifs in this dataset is stronger than in coding regions (FIGS. 2A-B). One can conclude that the patterns previously observed in virus and host coding genes are not due to effects from coding regions, such as codon usage patterns (Coleman et al., “Virus Attenuation by Genome-Scale Changes in Codon Pair Bias,” Science 320:1784-1787 (2008); Mueller et al., “Live Attenuated Influenza Virus Vaccines by Computer-Aided Rational Design,” Nature Biotech. 28:723-726 (2010); Mueller et al., “Reduction of The Rate of Poliovirus Protein Synthesis Through Large-Scale Codon Deoptimization Causes Attenuation of Viral Virulence by Lowering Specific Infectivity,” J. Virol. 80:9687-9696 (2006), which are hereby incorporated by reference in their entirety). Rather, such constraints in coding regions likely weaken the strength of a statistical bias that comes from the same underlying mechanisms. This suggests selective restrictions on dinucleotide frequencies observed in ncRNAs preserving a function or avoiding a detrimental consequence such as a chronic autoinflammatory response that could result from presenting danger-associated molecular patterns (DAMPs). Adaptation of dinucleotide motif usage in these elements over time is analogous to the viral mimicry of host patterns of sequence motif usage (Greenbaum et al., “Patterns of Evolution and Host Gene Mimicry in Influenza and Other RNA Viruses,” PLoS Path 4:e1000079 (2008) and Karlin et al, “Why is CpG Suppressed in the Genomes of Virtually all Small eukaryotic Viruses but not in those of Large Eukaryotic Viruses?” J. Virol. 68:2889-2897 (1994), which are hereby incorporated by reference in their entirety). When an avian influenza virus enters the human population, one can observe adaptation to analogous patterns emerging over time (Greenbaum et al, “Patterns of Evolution and Host Gene Mimicry in Influenza and Other RNA Viruses,” PLoS Path. 4:e1000079 (2008); Greenbaum et al., “Quantitative Theory of Entropic Forces Acting on Constrained Nucleotide Sequences Applied to Viruses,” Proc. Natl. Acad. Sci. 111:5054-5059 (2014); Greenbaum et al, “Patterns of Oligonucleotide Sequences in Viral and Host cell RNA Identify Mediators of the Host Innate Immune System,” PLoS One 4:e5969 (2009); Jimenez-Baranda et al., “Oligonucleotide Motifs that Disappear During the Evolution of Influenza Virus in Humans Increase Alpha Interferon Secretion by Plasmacytoid Dendritic Cells,” J. Virol 85:3893-3904 (2011), which are hereby incorporated by reference in their entirety). In that case, mutation rates in influenza are very high so one can follow these evolutionary adaptations over far shorter time periods.

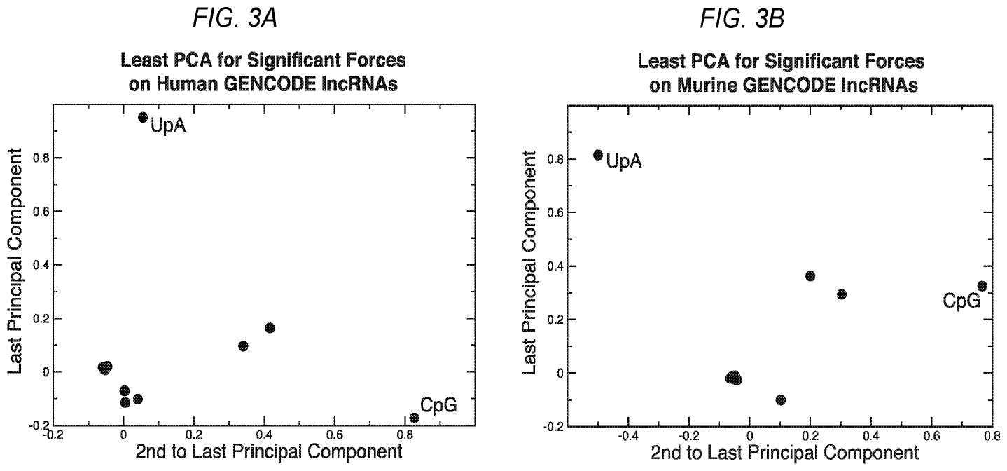

Trinucleotide motifs with significant forces are listed in Table 1, along with dinucleotide motifs. Trinucleotide motifs with significant forces (i.e. strengths of statistical bias) acting on them are conserved between humans and mice, as was the case for dinucleotides, with the exception of UAC and UAG (which are significant in humans but less so in mice). Except for UAG (chain termination codons used in coding RNAs), whenever a trinucleotide motif is significantly enhanced or avoided in humans its reverse complement is also significantly enhanced or avoided suggesting avoidance of complementary motifs. The strongest forces (i.e. strengths of statistical bias) suppress CpG and CpG-containing trinucleotides, particularly when an A or U is next to the core CpG motif. This is consistent with the avoidance of CpGs in AU contexts observed in influenza viruses replicating in humans (Greenbaum et al, “Quantitative Theory of Entropic Forces Acting on Constrained Nucleotide Sequences Applied to Viruses,” Proc. Natl. Acad. Sci. 111:5054-5059 (2014); Greenbaum et al, “Patterns of Olignonculeotide Sequences in Viral and Host Cell RNA Identify Mediators of the Host Innate Immune System,” PLoS One 4:e5969 (2009); Jimenez-Baranda et al., “Oligonucleotide Motifs that Disappear During the Evolution of Influenza Virus in Humans Increase Alpha Interferon Secretion by Plasmacytoid Dendritic Cells,” J. Virol. 85:3893-3904 (2011), which are hereby incorporated by reference in their entirety). Given the apparent bias against CpG and UpA, it was further determined if these were linked. Pearson correlation between these forces across all GENCODE ncRNA in humans and mice showed no correlation between CpG and UpA biases (r=0.0006; FIGS. 3A-B). Therefore, the forces on CpG and UpA are likely independent. Moreover, every significant trimer across GENCODE is correlated to CpG, UpA, or both. As a result, all significant trimers can be explained by their CpG or UpA motif usage.

### Example 2

### Cancer Enriched Non-coding Repeat RNA may have Anomalous Motif Usage

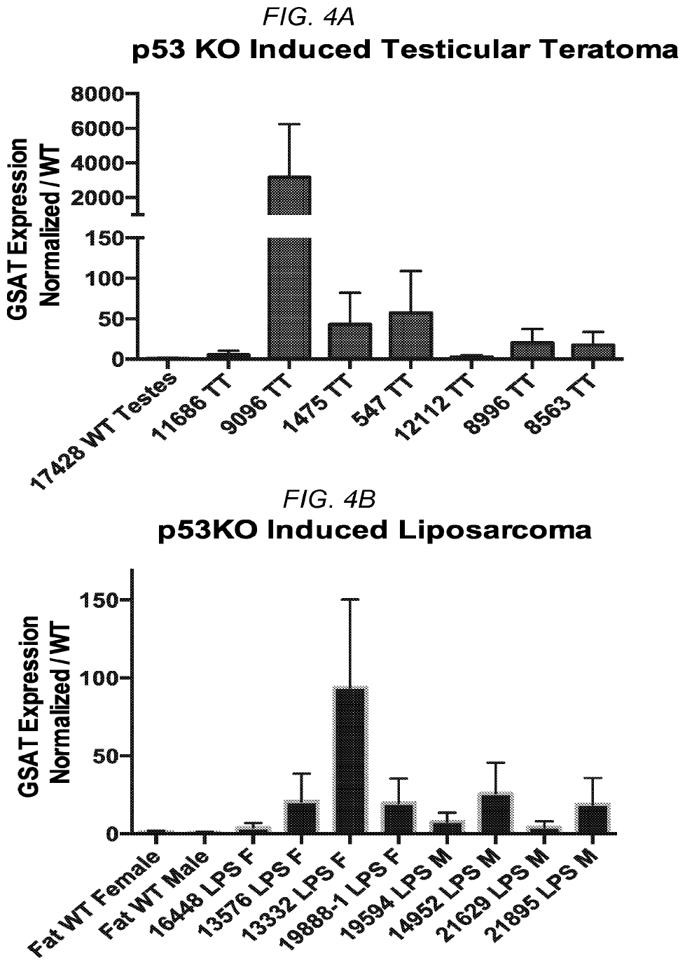

Prior work revealed aberrant expression of non-coding RNA across a spectrum of mouse and human cancers (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013) and Ting et al., “Aberrant Overexpression of Satellite Repeats in Pancreatic and Other Epithelial Cancers,” Science 331:593-596 (2011), which are hereby incorporated by reference in their entirety). These sequences were found in the Repbase database of human and murine repetitive elements and the FANTOM database of murine non-coding elements (currently NONCODE) (Jurka et al., “Repbase Update A Database of Eukaryotic Repetitive Elements,” Cytogenetic and Genome Res. 110:462-467 (2005) and Xie et al., “NONCODEv4: Exploring the World of Long Non-Coding RNA Genes,” Nucleic Acids Res. 42:D98-D103 (2014), which are hereby incorporated by reference in their entirety). A high induction of GSAT in a murine testicular teratoma and liposarcoma tumor model was also found (FIGS. 4A-B) (Leonova et al., “P53 Cooperates with DNA Methylation and a Suicidal Interferon Response to Maintain Epigenetic Silencing of repeats and Noncoding RNAs,” Proc. Natl. Acad. Sci. 110:E89-E98 (2013) and Ting et al., “Aberrant Overexpression of Satellite Repeats in Pancreatic and Other Epithelial Cancers,” Science 331:593-596 (2011), which are hereby incorporated by reference in their entirety). Focusing on these cancer expressed repeats, a surprisingly significant enrichment of anomalous motif usage patterns was found, as compared to other ncRNAs. In Repbase, it was tested whether the bias on di- and tri-nucleotide motifs observed in repetitive element sequences fell outside the distribution obtained from GENCODE lncRNA. Remarkably, hundreds of sequences falling outside of this distribution were found. Many have high usage of CpG dinucleotides including a set of endogenous viruses (Table 2) recently implicated in the innate immune response in tumors (Zeng et al., “MAVS cGAS and Endogenous Retroviruses in T-independent B Cell Responses,” Science 346:1486-1492 (2014), which is hereby incorporated by reference in its entirety). It was concluded that while the portion of the noncoding regions typically expressed as lncRNAs have similar motif usage patterns as RNA from coding regions, there are many genomic regions with atypical motif usage that are not transcribed in normal cells or tissues.

TABLE 2
Many Repetitive Elements Have High CpG Forces

| | | | CpG Force |
|---|---|---|---|
| | | | (Strength of |
| | | Level of | Statistical |
| ncRNA | Class | Conservation | Bias) |
| MER123 | DNA_transposon | Amniota | 1.1039 |
| HSATII | SAT | Primates | 1.0360 |
| UCON21 | Transposable_Element | Amniota | 0.9465 |
| MER6B | Mariner/Tc1 | Homo_spaiens | 0.9230 |
| Eulor1 | Transposable_Element | Amniota | 0.8481 |
| Eulor5B | Transposable_Element | Tetrapoda | 0.8474 |
| Eulor2C | Transposable_Element | Amniota | 0.7676 |
| Eulor6A | Transposable_Element | Tetrapoda | 0.7466 |
| MER131 | SINE | Amniota | 0.6223 |
| Eulor4 | Transposable_Element | Tetrapoda | 0.6067 |
| Eulor10 | Transposable_Element | Amniota | 0.6064 |
| MER6C | Mariner/Tc1 | Eutheria | 0.5667 |
| Eulor12 | Transposable_Element | Amniota | 0.5295 |
| MER5C1 | hAT | Eutheria | 0.4582 |
| MER47B | Mariner/Tc1 | Eutheria | 0.4518 |
| UCON39 | DNA_transposon | Mammalia | 0.4443 |
| UCON16 | Transposable_Element | Amniota | 0.4436 |
| Tigger3d | Mariner/Tc1 | Primates | 0.4374 |
| TIGGER5A | Mariner/Tc1 | Eutheria | 0.4212 |
| MER75 | DNA_transposon | Homo_sapiens | 0.4134 |
| Tigger4a | Mariner/Tc1 | Primates | 0.3815 |
| npiggy2_Mm | piggyBac | Microcebus_murinus | 0.3725 |
| MER58B | hAT | Eutheria | 0.3657 |
| Eulor6C | Transposable_Element | Tetrapoda | 0.3571 |
| Eulor11 | Transposable_Element | Amniota | 0.3561 |
| UCON15 | Transposable_Element | Amniota | 0.3560 |
| Tigger2b_Pri | Mariner/Tc1 | Primates | 0.3548 |
| MER44B | Mariner/Tc1 | Homo_sapiens | 0.3536 |
| SUBTEL_sat | Satellite | Primates | 0.3527 |
| Eulor9A | Transposable_Element | Amniota | 0.3465 |
| MER44C | Mariner/Tc1 | Homo_sapiens | 0.3439 |
| Eulor8 | Transposable_Element | Amniota | 0.3416 |
| MER44D | Mariner/Tc1 | Eutheria | 0.3211 |
| npiggy1_Mm | piggyback | Microcebus_murinus | 0.3131 |
| UCON26 | Transposable_Element | Amniota | 0.2985 |
| MER127 | Mariner/Tc1 | Amniota | 0.2984 |
| MER97d | hAT | Eutheria | 0.2939 |
| Eulor6D | Transposable_Element | Tetrapoda | 0.2866 |
| Eulor2B | Transposable_Element | Amniota | 0.2852 |
| MER119 | hAT | Homo_sapiens | 0.2794 |
| MER134 | Transposable_Element | Amniota | 0.2786 |
| Eulor9C | Transposable_Element | Amniota | 0.2751 |
| MER8 | Mariner/Tc1 | Homo_sapiens | 0.2669 |
| Ricksha_a | MuDR | Eutheria | 0.2607 |
| MER129 | SINE | Amniota | 0.2444 |
| MacERV6_LTR3 | ERV3 | Cercopithecidae | 0.2404 |
| MER57B2 | ERV1 | Homo_sapiens | 0.2403 |
| HSMAR1 | Mariner/Tc1 | Homo_sapiens | 0.2397 |
| Eulor12_CM | Transposable_Element | Amniota | 0.2269 |
| MERX | Mariner/Tc1 | Eutheria | 0.2207 |
| Tigger12A | Mariner/Tc1 | Mammalia | 0.2170 |
| MER58A | hAT | Eutheria | 0.2006 |

Listed above are the repetitive elements from Repbase with a significantly high CpG force. These elements are typically not found to be expressed in normal tissue, yet some may be expressed in cancer cells and cell lines.

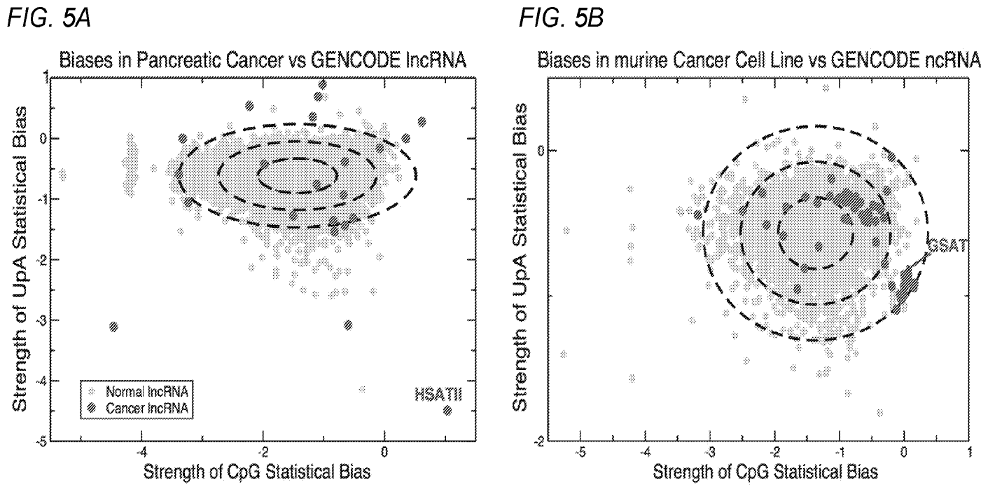

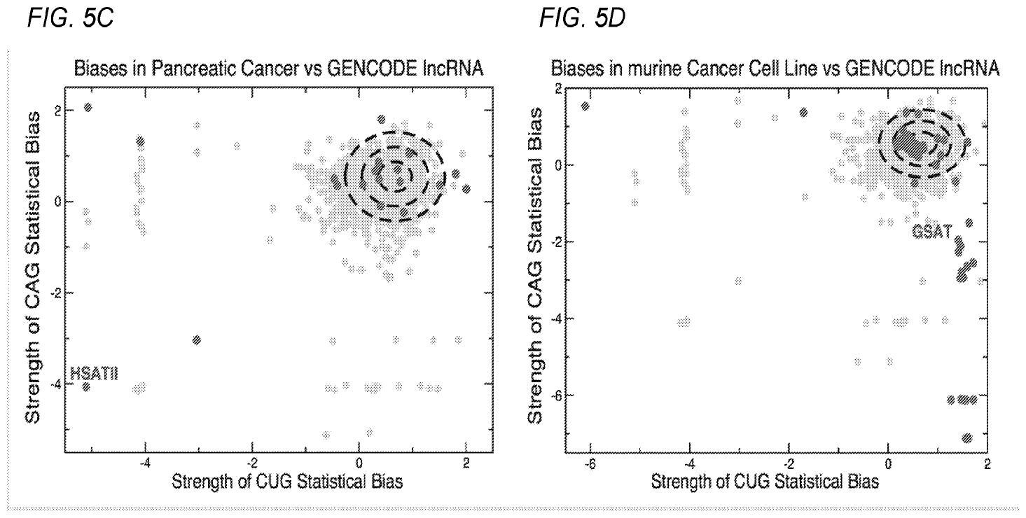

The forces which quantify the strength of the statistical bias on the often underrepresented CpG and UpA dinucleotides were used to differentiate between ncRNAs found preferentially in cancerous cells and the total lncRNA referenced in GENCODE for humans and mice, as these two dinucleotides essentially account for all significant trinucleotide motifs in this set. The distribution of forces (i.e. strengths of statistical bias) on CpG and UpA were used to define a null hypothesis, which was approximate by a Gaussian distribution (FIGS. 5A-D). Many ncRNAs from cancerous cells are clearly outside the distribution—often to a large extent. In particular, HSATII, the main ncRNA upregulated in human pancreatic cancers, is far outside the human distribution, and GSAT, the main murine ncRNA implicated in murine tumoral cell lines, is well outside of the mouse distribution. Within the null hypothesis, the p-values for all ncRNAs considered here are less than 10⁻⁶¹ for human pancreatic cancer data and less than 10⁻² for murine cell line data.

Many of the ncRNAs from Leonova et al., “P53 Cooperates with DNA Methylation and a Suicidal Interferon Response to Maintain Epigenetic Silencing of repeats and Noncoding RNAs,” Proc. Natl. Acad. Sci. 110:E89-E98 (2013) and Ting et al., “Aberrant Overexpression of Satellite Repeats in Pancreatic and Other Epithelial Cancers,” Science 331:593-596 (2011), which are hereby incorporated by reference in their entirety are outliers of at least three standard deviations with respect to at least one of the significant motifs implicated in the previous section, accounting for 70.46% of the modulated Repbase RNA expression induced in pancreatic cancer along with even higher percentages (74.86% and 85.30%, respectively) in the smaller sets of prostate and lung cancers. HSATII is the most differentially expressed (by a considerable margin) in the pancreatic cancer data and HSATII and BSR are the highest in prostate and lung. In p53 knockout murine cell lines treated with demethylation agents, around 68 ncRNAs are significantly modulated (Leonova et al., “P53 Cooperates with DNA Methylation and a Suicidal Interferon Response to Maintain Epigenetic Silencing of Repeats and Noncoding RNAs,” Proc. Natl. Acad. Sci. 110:E89-E98 (2013), which is hereby incorporated by reference in its entirety). Among those, 78.96% of the total expression comes from outliers as defined above, with the vast majority coming from GSAT and B2. Overall, it was observed that repetitive sequences containing unusual motif usage had varying degrees of conservation. However, the subset preferentially expressed in cancerous cells and tissues are encoded by sequences of more recent evolutionary origin. HSATII and GSAT are only conserved back to primates and mouse, respectively, and 21 of the 22 ncRNAs from Ting et al., “Aberrant Overexpression of Satellite Repeats in Pancreatic and Other Epithelial Cancers,” Science 331:593-596 (2011), hereby incorporated by reference in its entirety, are conserved in humans and primates but no further back in evolution. Any function is likely to be species specific.

### Example 3

### ncRNAs with Unusual Motif Usage Highly Expressed in Cancers are Immunostimulatory

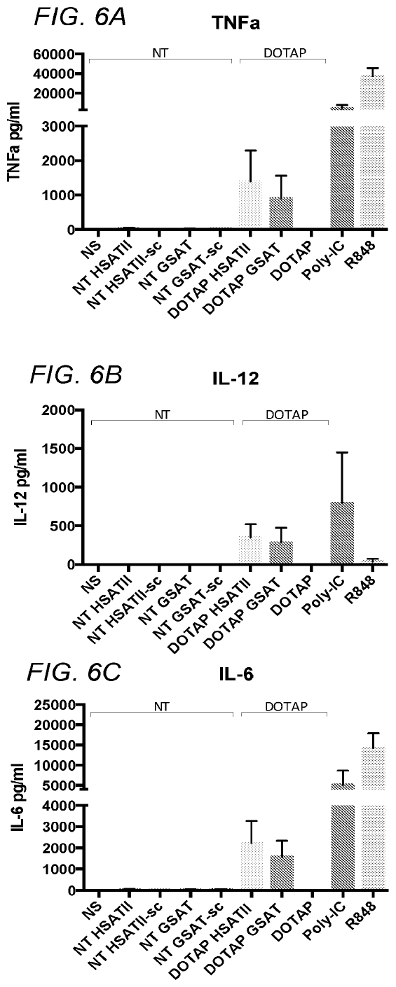

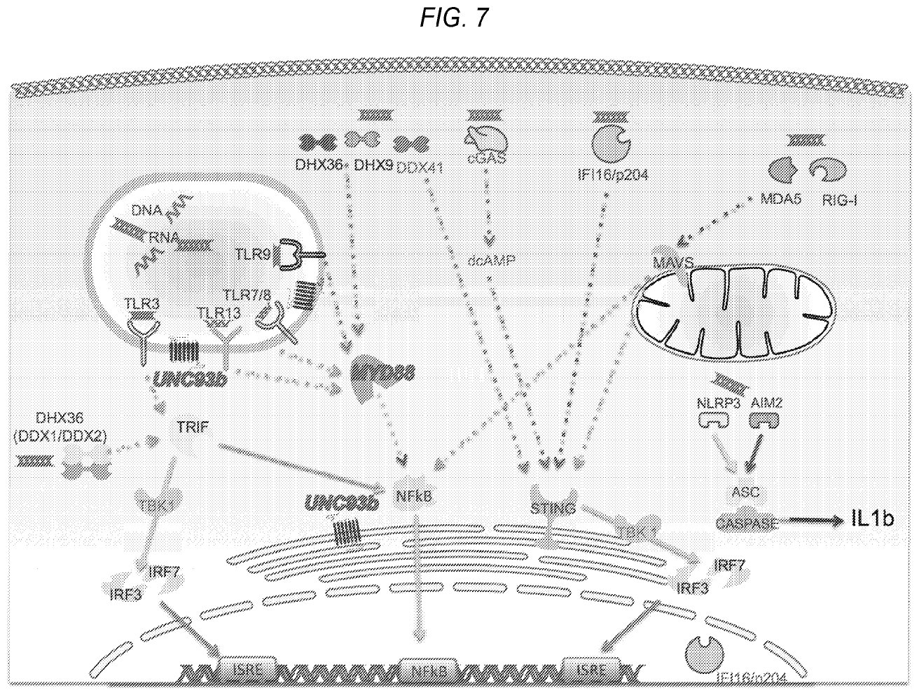

This analysis highlights that many ncRNAs upregulated in cancer display abnormal nucleotide motif usage that had previously been related to immunogenic properties in viruses. The innate immune system contains several effector cells that react to immunogenic nucleic acids such as exogenous viral and bacterial nucleic acids as well as endogenous nucleic acids which can be released upon cell death (Atianand et al., “Molecular basis of DNA Recognition in the Immune System,” J. Immunol. 190:1911-1918 (2013), which is hereby incorporated by reference in its entirety). Among those effectors, the mononuclear phagocytic system (macrophages, monocytes, and dendritic cells (“DC”s)) contains key regulators of innate immune activation and adaptive immunity (Guilliams et al., “Dendritic Cells Monocytes and Macrophages: A Unified Nomenclature Based on Ontogeny,” Nature Rev. Immunol. 14:571-578; Kroemer et al., “Immunogenic Cell Death in Cancer Therapy,” Ann. Rev. Immunol. 31:51-72 (2013); Sabado et al., “Dendritic Cell Immunotherapy,” Ann. New York Acad. Sci. 1284:31-45 (2013), which are hereby incorporated by reference in their entirety). DCs efficiently sense and sample their environment to integrate information and mount a proper response which may be tolerogenic or immunogenic. To test whether ncRNA with highly unusual motif usage could be recognized as a danger-associated molecular pattern (“DAMP”) by some nucleic acid sensing pattern recognition receptors (“PRRs”), the effect of human HSATII and murine GSAT following transfection in human monocyte derived DCs (“moDCs”) and murine bone marrow derived macrophages was studied. Liposomal transfection was required for stimulation, whereas naked RNA had no effect; implying recognition is consistent with activation via an endosomal or intracellular sensor (FIGS. 6A-C). The general sets of recognition pathways tested are indicated in FIG. 7.

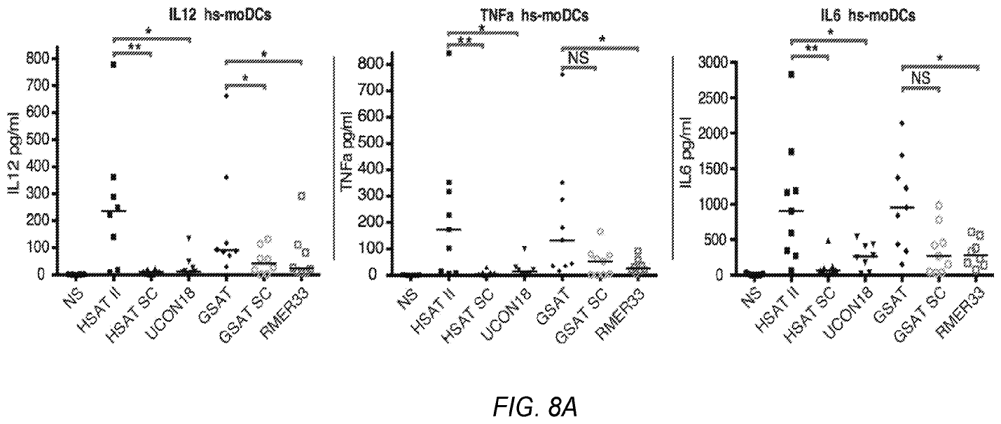

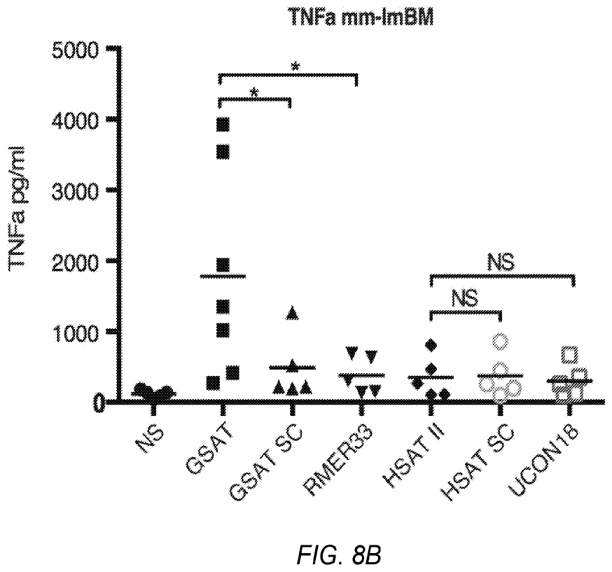

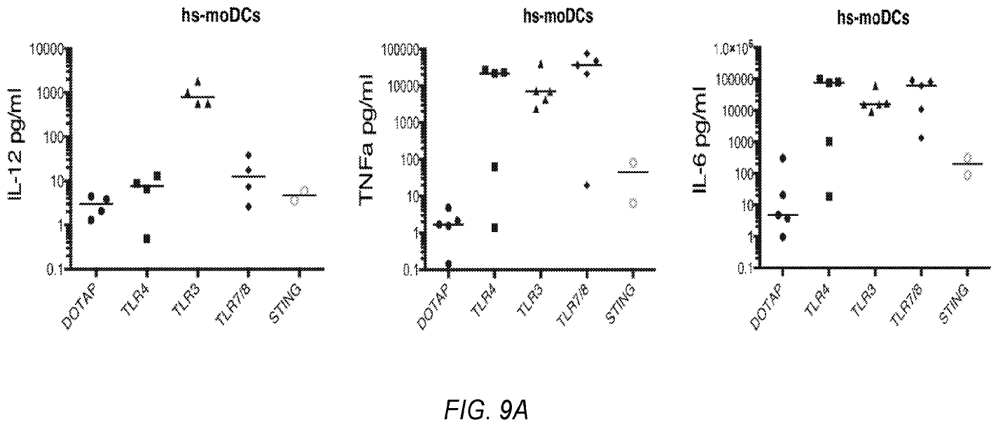

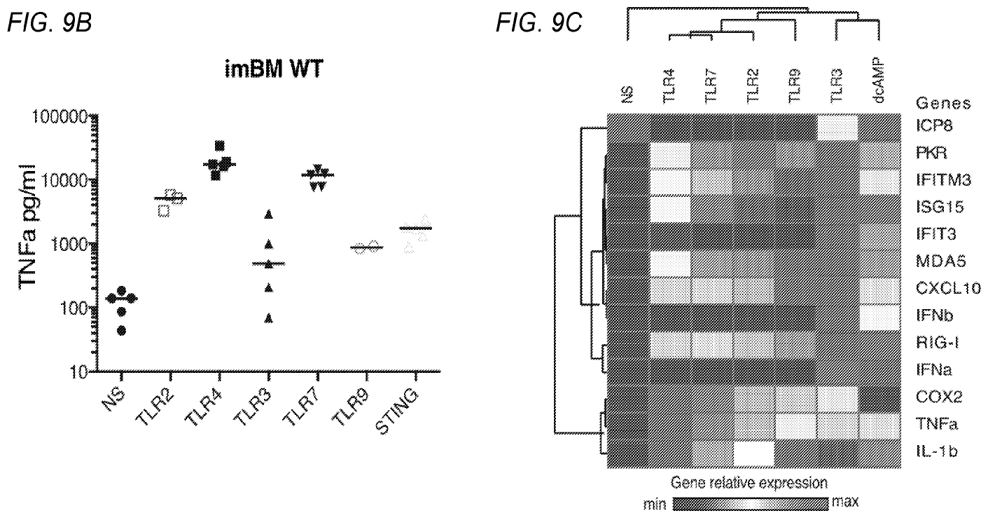

Different ncRNA were generated by in vitro transcription using minigenes coding for the two main candidate outliers computationally predicted to have immunogenic motif usage (HSATII and GSAT). RNA from minigenes was derived as controls, encoding scrambled versions with the same nucleotide content but normal motif usage (labeled “HSATII-sc” and “GSAT-sc”) and repetitive elements of comparable length, but which have normal motif usage patterns (RMER33 and UCON18), as described below. In human moDCs liposomal transfection of HSATII induced significant production of interleukin 6 and 12 (IL-6 and IL-12), and TNFalpha relative to both endogenous controls and their scrambled versions (FIGS. 8A-B). A similar profile of cytokines was elicited by moDCs in response to selected Toll-like receptor (TLR) agonists (FIG. 9A). The candidate murine immunogenic ncRNA GSAT had less pronounced immunogenic properties but still induced IL-12 (FIG. 8A). Upon liposomal transfection of the same ncRNA into immortalized murine bone marrow derived macrophages (“imBMs”), the immunogenic properties of HSATII were strongly attenuated, whereas the murine GSAT induced high levels of TNFalpha (FIG. 8B) and MCP-1 but not interferon gamma, IL-6, or IL-12. imBM almost exclusively regulates TNFalpha in response to pattern recognition receptor agonists (FIG. 9B).

HSATII and GSAT ncRNA induced IL-12 in human moDCs similarly to the TLR3 ligand poly-IC (a synthetic dsRNA mimic; FIG. 7). The absence of an effect by ncRNA with normal motif usage, i.e., the scramble forms (FIGS. 8A-B), suggest specific sequence patterns within the RNA, such as CpG and UpA motifs, regulate immunostimulatory activity. Such motif usage could also influence secondary conformation that may contribute to immunogenic properties, though it was checked that the scrambled sequences did not lower the RNA minimum folding energy. Based upon these observations, HSATII and GSAT are referred to as immunogenic-ncRNA or “i-ncRNA.” Interestingly, this study corroborates previous findings by Leonova et al., “P53 Cooperates with DNA Methylation and a Suicidal Interferon Response to Maintain Epigenetic Silencing of repeats and Noncoding RNAs,” Proc. Natl. Acad. Sci. 110:E89-E98 (2013) that ncRNA such as GSAT can induce an innate response, although in those studies the type I interferon pathway was also activated. The initial investigations into this pathway were inconclusive (FIG. 9C).

### Example 4

### Dissection of the Immunostimulatory Properties of i-ncRNA

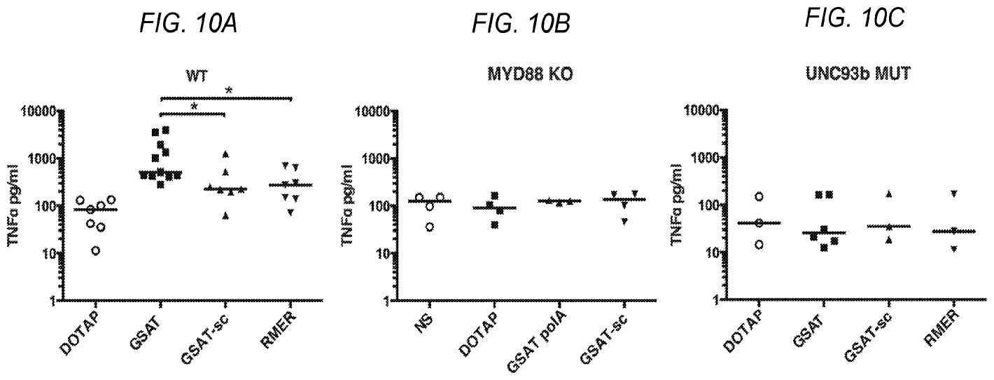

Pathogen-associated molecular patterns (“PAMPs”) and danger-associated molecular patterns (DAMPs) activate innate immune cells through pattern recognition receptors (PRRs). To better characterize the mechanisms involved in sensing i-ncRNA, the immunomodulatory properties of HSATII and GSAT on a panel of imBMs that lack specific PRRs or effector molecules in their downstream signaling pathways was studied (FIG. 7). Whereas GSAT induced a TNFalpha response, HSATII did not induce differential cytokine expression in these immortalized cells, indicating that either there is a species-specific effect, as the cells are murine, or cell type specific effect, as these cells are macrophages. This is perhaps unsurprising as different species and cell types express different pattern recognition receptors, and HSATII and GSAT have different sequence compositions. Significantly, the absence of two key adaptor and regulatory proteins MYD88 and UNC93B1:UNC93B3d (UNC93b), respectively, eliminated the differential response to GSAT in imBMs (FIGS. 10A-C).

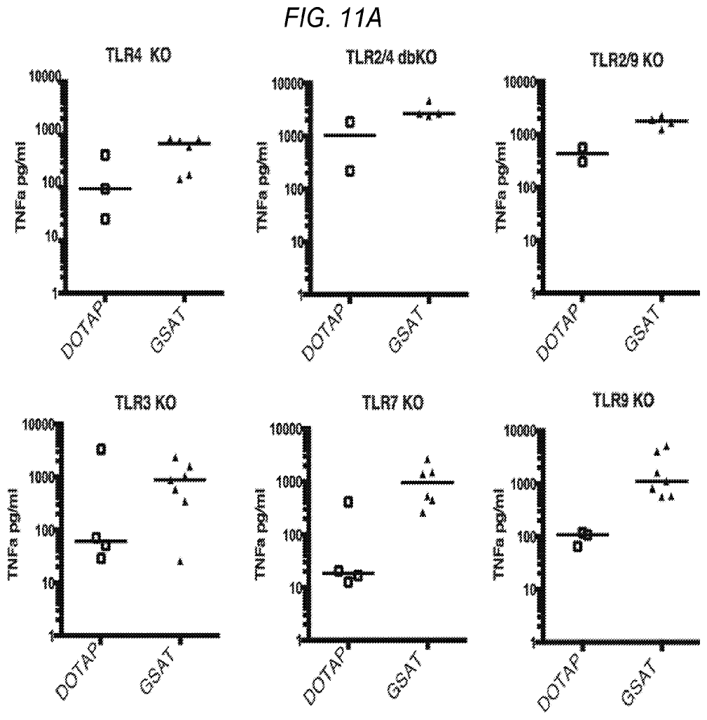

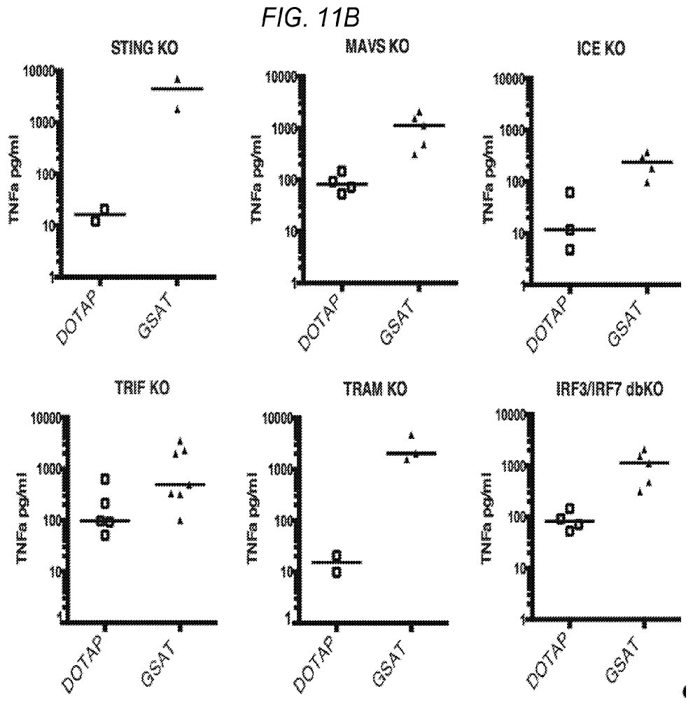

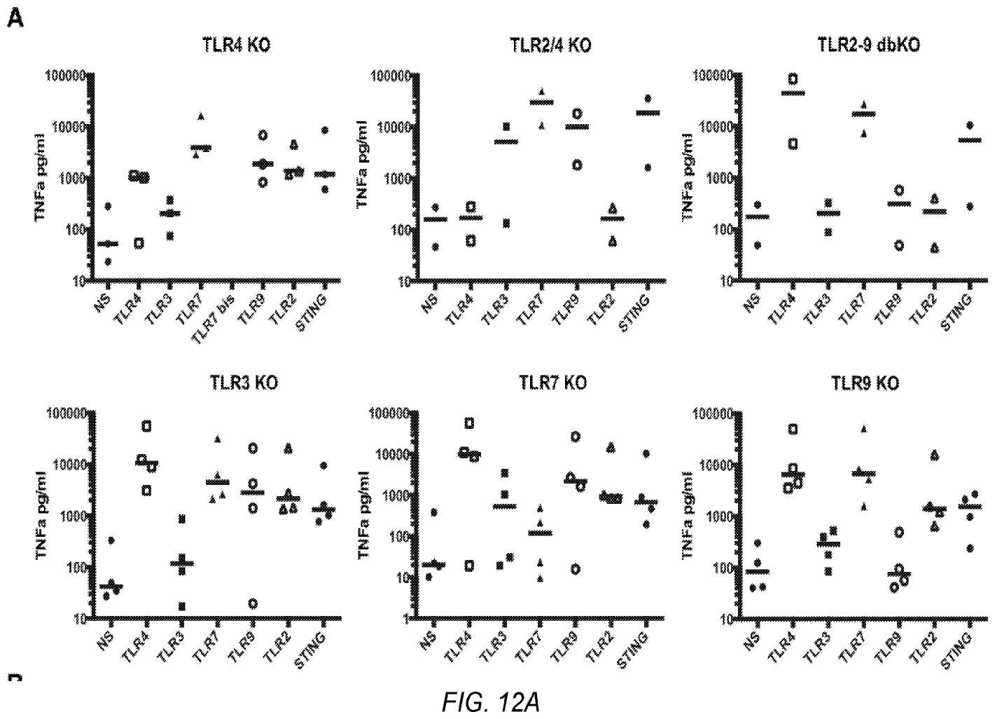

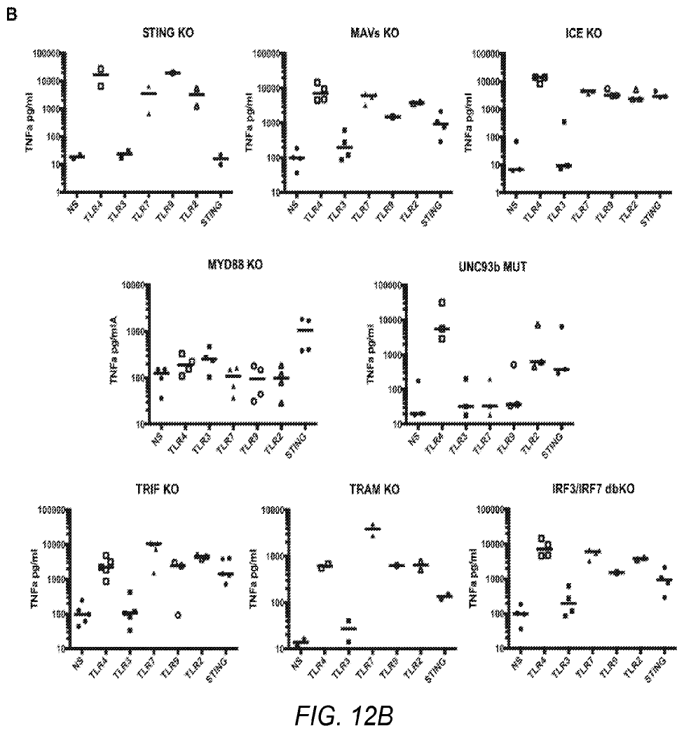

MYD88 is a key cytosolic adaptor protein that is used by all TLRs except TLR3 to activate the transcription factor NFkB. Similarly, the mutated form of UNC93b essentially eliminated inflammatory responses in imBMs. While less well characterized than MYD88, this protein is known to interact with several endosomal Toll-like receptors (TLR3, 7, and 9), and has been implicated in TLR trafficking between the endoplasmic reticulum and endosomes, and their resultant maturation (Casrouge et al, “Herpes Simplex Virus Encephalities in Human UNC-93B Deficiency,” Science 314:308-312 (2006); Lee et al., “UNC93B1 Mediates Differential Trafficking of Endosomal TLRs,” eLife 2:e00291; Tabeta et al., “The Unc93B1 Mutation 3d Disrupts Exogenous Antigen Presentation and Signaling via Toll-like Receptors 3 7 and 9,” Nature Immunol. 7:156-164 (2006), which are hereby incorporated by reference in their entirety). The requirement for TLR3, TLR7, and TLR9, which are known to recognize double-stranded RNA, single-stranded RNA, and CpG DNA respectively, was tested (FIGS. 11A-B, FIGS. 12A-B) (O'Neill et al., “The History of Toll-Like Receptors-Redefining Innate Immunity,” Nature Rev. Imm. 13:453-60 (2013); Broz et al., “Newly Described Pattern Recognition Receptors Team Up Against Intracellular Pathogens,” Nature Rev. Immunol. 13:551-565 (2013); Gajewski et al., “Innate and Adaptive Immune Cells in the Tumor Microenvironment,” Nature Immunol. 14:1014-1022 (2013), which are hereby incorporated by reference in their entirety). None of these receptors were required for GSAT to activate TNFalpha production from imBM. Additional pathways investigated, including the STING and inflammasome pathways, are discussed below and did not contribute to i-ncrNA stimulatory activity. Altogether, the data are consistent with a requirement for i-ncRNA activation through signaling pathways that rely upon MYD88 and UNC93b. The precise receptor involved in initial recognition remains to be determined.

There is a surprising similarity to be drawn between foreign viral nucleotide sequences and select ncRNAs silent in normal cells, yet transcribed in cancer cells, activating innate immunity (Jimenez-Baranda et al., “Olignonucleotide Motifs That Disappear During the Evolution of Influenza Virus in Humans Increase Alpha Interferon Secretion by Plasmacytoid Dendritic Cells,” J. Virol. 85:3893-3904 (2011); Casrouge et al., “Herpes Simplex Virus Encephalitis in Human UNC-93B Deficiency,” Science 314:308-312 (2006); Bogunovic et al., “Immune Profile and Mitotic Index of Metastatic Melanoma Lesions Enhance Clinical Staging in Predicting Patient Survival,” Proc. Natl. Acad. Sci. 106:20429-20434 (2009); Cosset et al., “Comprehensive Metagenomic Analysis of Glioblastoma Reveals Absence of Known Virus Despite Antiviral-Like Type I Interferon Gene Response,” International J. Cancer 135:1381-1389 (2014), which are hereby incorporated by reference in their entirety). It was determined that ncRNAs expressed predominantly in normal cells from humans and mice reflect patterns of nucleotide sequence motif avoidance, such as underrepresentation of CpG containing sequences and reduced UpA, similar to protein coding RNA. This often includes a many-fold underrepresentation of CpG containing sequences and reduced UpA motif usage when compared to expected levels. However, the genome also harbors repetitive elements, which often have abnormal usage of CpG and UpA motifs than that observed in RNA expressed in normal cells and tissues. Sets of these ncRNA, typically newer genome entries over evolutionary time scales, can be expressed in very high levels in cancerous cells and tumors. This is why human and mouse elements expressed in cancer cells can have different sequences but can share high CpG content and are not generally observed in the human or mouse transcriptome in normal cells.

It was previously proposed that immunostimulatory and proinflammatory properties of highly inflammatory influenza and other RNA viruses derive in part from RNA containing CpGs in AU-rich contexts, which are avoided in RNA viruses circulating in humans. Experimental evidence has supported this hypothesis (Jimenez-Baranda et al., “Olignonucleotide Motifs That Disappear During the Evolution of Influenza Virus in Humans Increase Alpha Interferon Secretion by Plasmacytoid Dendritic Cells,” J. Virol. 85:3893-3904 (2011); Atkinson et al., “The Influence of CpG and UpA Dinocleotide Frequencies on RNA Virus Replication and Characterization of the Innate Cellular Pathways Underlying Virus Attenuation and Enhanced Replication,” Nucleic Acids Res. 42:4527-4545 (2014) and Vabret et al., “The Biased Nucleotide Composition of HIV-1 Triggers Type I Interferon Response and Correlates with Subtype D Increased Pathogenicity,” PLoS One 7:e33501 (2012), which are hereby incorporated by reference in their entirety). The analysis was recently recast in the language of statistical physics in a way that is theoretically insightful and computationally efficient (Greenbaum et al., “Quantitative Theory of Entropic Forces Acting on Constrained Nucleotide Sequences Applied to Virus,” Proc. Natl. Acad. Sci. 111:5054-5059 (2014), which is hereby incorporated by reference in its entirety). In this language, the evolution and optimization of nucleotide sequence motifs is driven by the interplay between selective and entropic forces. The latter randomize motif frequencies in a genome under constraints while the former are largely Darwinian, optimizing for functions enhancing viral replication and spreading. However, ncRNAs mostly transcribed in cancerous cells would not be exposed to the same selective and entropic forces as coding and ncRNA transcribed in normal cells. Based on motif usage patterns, it is predicted that many ncRNA may have immunogenic properties, presenting danger-associated molecular patterns.

HSATII and murine GSAT were focused on experimentally, as they are preferentially and highly expressed in carcinogenic processes and exhibit abnormal patterns of motif usage. In particular, human HSATII is enriched in CpG motifs in AU-rich contexts avoided in genomes of humans and human adapted viruses. It is demonstrated that their computationally predicted immunogenic properties lead to the induction of inflammatory cytokines in human and murine innate cells (FIGS. 8A-B). These observations, together with previous work by Leonova et al., “P53 Cooperates with DNA Methylation and a Suicidal Interferon Response to Maintain Epigenetic Silencing of repeats and Noncoding RNAs,” Proc. Natl. Acad. Sci. 110:E89-E98 (2013), which is hereby incorporated by reference in its entirety, strongly suggest that these endogenous i-ncRNA are recognized as DAMPs by cellular nucleic acid pattern recognition receptors.

A key role for MYD88 and UNC93b as regulators of GSAT immunogenicity was identified, but without evidence for the common endosomal nucleic acid sensors typically regulated by UNC93b or associated with the MYD88 adaptor (TLRs 2, 4, 7, and 9). These results indicate that in the murine imBM background there is potent induction of TNFalpha. Further studies will be required to elucidate whether TLR13, identified in murine cells and which recognizes ribosomal bacterial and viral RNA, is involved or whether there exist intracellular sensors of i-ncRNA associated with MYD88 (Li et al., Sequence Specific Detection of Bacterial 23S Ribosomal RNA by TLR13,” eLife 1:e00102 (2012); Oldenburg et al., “TLR13 Recognizes Bacterial 23S rRNA Devoid of Erythromycin Resistance-Forming Modification,” Science 337:1111-1115 (2012); Shi et al., “A novel Toll-like Receptor That Recognizes Vesicular Stomatitis Virus,” J. Biol. Chem. 286:4517-4524 (2012), which are hereby incorporated by reference in their entirety), as there are for dsDNA (DHX-9 or -36) (Kim et al., “Aspartate-Glutamate-Alanine-Histidine Box Motif (DEAH)/RNA Helicase A Helicases Sense Microbial DNA in Human Plasmacytoid Dendritic Cells,” Proc. Natl. Acad. Sci. 107:15181-15186 (2010), which is hereby incorporated by reference in its entirety). Interestingly, it is found that alignment of GSAT contains a subsequence conserved in immunogenic RNA isolated from bacterial ribosomal RNA, which specifically activates murine TLR13 (Oldenburg et al., “TLR13 Recognizes Bacterial 23S rRNA Devoid of Erythromycin Resistance-Forming Modification,” Science 337:1111-1115 (2012), which is hereby incorporated by reference in its entirety).

Activation of innate immune signaling can contribute either to carcinogenesis or antitumoral immunity. Toll-like receptor signaling and MYD88 have been associated with tumor development (Wang et al., “Toll-like Receptors and Cancer: MYD88 Mutation and Inflammation,” Frontiers in Immunology 5(367):1-10 (2014), which is hereby incorporated by reference in its entirety). Given that HSATII and GSAT expression has been found to be pervasive in many tumor types and induces responses that differ by species or cell type, the role of i-ncRNA in tumorigenesis is likely dependent on the particular RNA expressed and other properties of the tumor microenvironment. For instance, HSATII activates macrophages and monocytes in this study, suggesting it may be a mechanism for attraction and retention of tumor associated macrophages. These macrophages have consistently been shown to be a poor prognostic in cancer leading to increased tumorigenesis, metastasis, and immunoevasion (Noy et al., “Tumor-Associated Macrophages: From Mechanisms to Therapy,” Immunity 41:49-61 (2014), which is hereby incorporated by reference in its entirety). Under this hypothesis, HSATII is used by the tumor to keep macrophages in the tumor microenvironment while driving out T cells. Interestingly, the viral like behavior of HSATII transcripts is not only found in the immune response to these elements, but also their ability to reverse transcribe in cancer cells akin to retroviruses (Bersani et al., “Pericentromeric Satellite Repeat Expansions Through RNA-Derived DNA Intermediates in Cancer,” Proc. Natl.. Acad. Sci. 112(49):15148-15153 (2015), which is hereby incorporated by reference in its entirety).

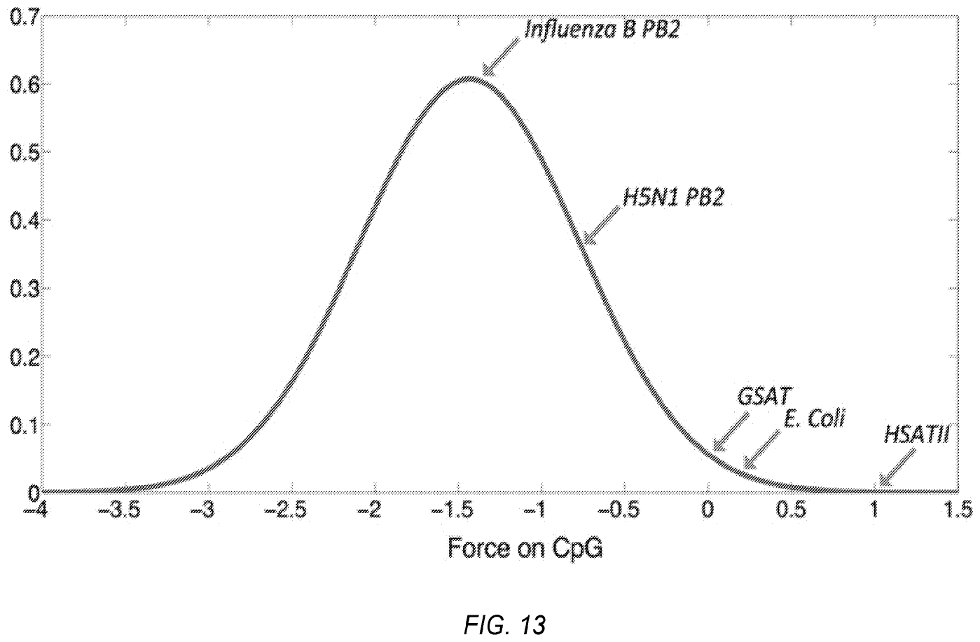

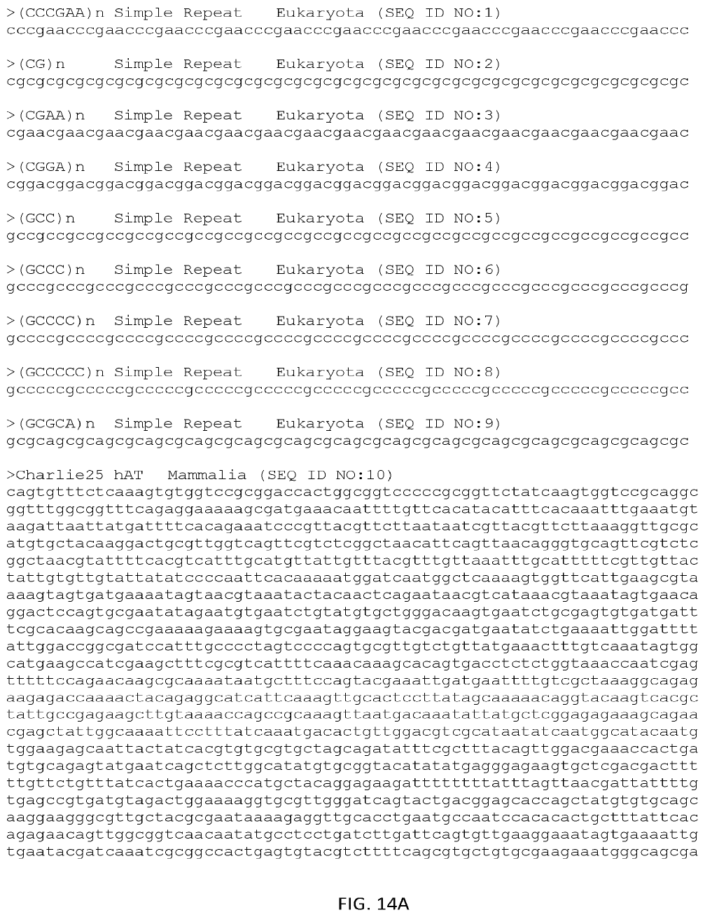

i-ncRNA, not subject to the same forces as ncRNA transcribed in steady state, may retain or evolve to mimic features of foreign RNA, as seen by comparing HSATII and GSAT to typical human ncRNA and foreign genomic material in FIG. 13 (Greenbaum et al., “Quantiative Theory of Entropic Forces Acting on Constrained Nucleotide Sequences Applied to Viruses,” Proc. Natl. Acad. Sci. 111:5054-5059 (2014) and Kent et al., “The Human Genome Browser at UCSC,” Genome Res. 12:996-1006 (2002), which are hereby incorporated by reference in their entirety). Indeed, HSATII and GSAT cluster more closely in terms of motif usage patterns, with bacterial rather than human RNA. Such RNA may have been selected for to identify and eliminate cells when their epigenetic state is disrupted. Essentially self “junk” RNA may have been maintained or evolved to mimic non-self pathogen associated patterns to create a danger signal. Such a mechanism would be a new aspect of “genetic mimicry” where the host is for all practical purposes mimicking pathogen-associated nucleic acid patterns. HSATII and GSAT emanate from the pericentromeres, which harbor new repetitive elements with no known function (Maumus et al., “Ancestral Repeats Have Shaped Epigenomic and Genome Composition for Millions of Years in Arabidopsis thaliana,” Nature Comm. 5:4014 (2014), which is hereby incorporated by reference in its entirety). This region, unlike centromeres or regions critical for structure or regulation, may dynamically produce unusual repetitive elements that can adapt to a particular organism's pattern recognition receptors. These studies indicate that under the “extraordinary” circumstances when these repetitive elements are expressed, they could play a critical role in the regulation of immune responses against cancer.

### Example 5

### Entropy of Nucleotide Sequences for a Given Motif

An RNA sequence of length L, hereafter called S₀, and a motif m (a series of contiguous nucleotides, e.g., CpG) is considered. L is the total sequence length, comprising the nucleotides A, C, G, and U, along with nucleotide bases that are not clearly defined. The objective is to define a probabilistic model over the set of the 4^{L} sequences, S=(s₁ s₂ . . . s_{L}), such that the average value of the number, Nₘ(S), of occurrences of the motif m in S coincides with the number, Nₘ(S₀), of occurrences that motif in S₀. To do so, a random-nucleotide model is considered, where nucleotides are independently distributed according to the frequencies f⁰(s), where s=A, C, G, U, found in S₀ (or where s=A, C, G, T when S₀ is represented as an un-transcribed DNA sequence). The frequency of a nucleotide is calculated by counting the number of times that nucleotide occurs and dividing that number by the total length of the sequence, L (which may also occur for ambiguously defined bases that cannot be assigned as A, C, G, U, or T). For example, f⁰(A), the frequency of A nucleotides, would be the number of occurrences of the base, A, in S₀ divided by L, the length of S₀, even when ambiguous bases are included.

The probability of a sequence Sin this least-constrained, maximum entropy model is

$$\begin{matrix}P ( S \vert x , m ) = \frac{1}{{Z}_{m} ( x )} {\prod }_{i = 1}^{L} {f}^{0} ( {s}_{i} ) exp ( x {N}_{m} ( S ) ) \\ where & [ EQUATION 1 ] \\ {Z}_{m} ( x ) = {\sum }_{sequences S} {\prod }_{i = 1}^{L} {f}^{0} ( {s}_{i} ) exp ( x {N}_{m} ( S ) ) & [ EQUATION 2 ]\end{matrix}$$

ensures the probability is correctly normalized. Parameter x, referred to as a selective force (or just force) on the motif m, introduces a statistical bias over P (Greenbaum et al., “Quantiative Theory of Entropic Forces Acting on Constrained Nucleotide Sequences Applied to Viruses,” Proc. Natl. Acad. Sci. 111:5054-5059 (2014), which is hereby incorporated by reference in its entirety). The force quantifies the strength of statistical bias, which may be due to selection on a motif. In the absence of bias (x=0) the probability of S simplifies to the product its nucleotide frequencies, and the number of motifs is what one would expect in a typical sequence with nucleotide frequencies given by f⁰(s). Positive values for x push the distribution towards sequences with Nₘ(S) larger than what one would expect while negative x favor sequences with a smaller Nₘ(S) than expected.

The value of the force, x(S₀), is computed by maximizing the probability

P(S₀|x,m)

of the sequence S₀ over x. This is equivalent to finding the value of x such that the average number of motifs

$${N}_{m}^{av} ( x ) = {\sum }_{sequences S} P ( S \vert x , m ) {N}_{m} ( S ) = \frac{\partial log {Z}_{m}}{\partial x} ( x ) \qquad [ EQUATION 3 ]$$

equals Nₘ(S₀). By scanning the sequences S₀ in the GENCODE database, the forces x(S₀) shown in FIGS. 5A-D are obtained.

The logarithm of the number of sequences having Nₘ(S) repetitions of m is bounded from above by the entropy of the random-nucleotide model; the equality is reached in the absence of bias only (x=0). The difference between those entropies is the entropy cost corresponding to the constraint on the average number of occurrences of m, and is denoted by σₘ. It is the Legendre transform of log Zₘ(x), see EQUATION 2 and EQUATION 3 (supra).

σₘ=x(S₀)Nₘ(S₀)−logZₘ(x(S₀)) (4)

Efficient computational techniques allow calculation of the sum over the 4^{L} sequences in EQUATION 2 in a time growing only linearly with L.

The aim is to find anomalous motif usage in a sequence where the number of motif occurrences is different from what is expected by chance in the random-nucleotide model, that is, associated to a significant nonzero force. The likelihood of observing the natural sequence S₀ with a given motif count is expressed as

$$P ( {S}^{0} \vert m ) = {max}_{x} [ P ( {S}^{0} \vert x , m ) ] = {e}^{{σ}_{m}} {Π}_{i} {f}^{0} ( {s}_{i}^{0} ) . \qquad [ EQUATION 5 ]$$

This likelihood is therefore directly related to the entropic cost: The larger the cost, the more likely is the motif to be statistically significant.

### Example 6

### Outlier Detection

GSAT and HSATII were demonstrated to be immunogenic, and were outliers relative to the distribution of strengths of statistical bias on CpG and UpA dinucleotides. Since GSAT was less of an outlier than HSATII, GSAT is used to define a minimal threshold of the strength of statistical bias for an immunogenic non-coding RNA. In the mouse GENCODE dataset, version 2 (which is hereby incorporated by reference in its entirety), of long non-coding RNA transcripts, the mean value of the strength of statistical bias on CpG dinucleotides is −1.3678 with a standard deviation of 0.5788, and the mean value of the strength of statistical bias on UpA dinucleotides is −0.5691 with a standard deviation of 0.2455. In the human GENCODE dataset, version 19 (which is hereby incorporated by reference in its entirety), of long-noncoding RNA transcripts, the mean value of the strength of statistical bias on CpG dinucleotides is −1.4341 with a standard deviation of 0.6505, and the mean value of the strength of statistical bias on UpA dinucleotides is −0.6152 with a standard deviation of 0.2834. The strength of statistical bias on GSAT is 0 for CpG dinucleotides and −0.8566 for UpA dinucleotides. This is 2.3629 standard deviations away from the mean of the mouse GENCODE distribution of strengths of statistical bias on CpG dinucleotides and 0.8831 standard deviations away from the mean for UpA dinucleotides. The strength of statistical bias on UpA dinucleotides was therefore not deemed necessary to define GSAT as an outlier as the strength of statistical bias of UpA dinucleotides is not significant for GSAT.

The CpG strength of statistical bias on GSAT is 2.3629 standard deviations from the mean of the distribution of strengths of statistical bias on CpG for the mouse GENCODE dataset and 2.2046 standard deviations away from the mean for the human GENCODE dataset. Therefore, an outlier in the human dataset was defined as a sequence whose strength of statistical bias on CpG dinucleotides has a Z-score (the strength of statistical bias on CpG minus the mean strength of statistical bias divided by the standard deviation) as greater than 2.2046 and for the mouse distribution as having a Z-score greater than 2.3629. This insures that the sequence is both an outlier and that CpG is over-represented relative to the GENCODE distribution.

Mouse repetitive elements meeting this threshold from mouse repeat sequences from the Repbase database are found in Table 3, and their corresponding nucleotide sequences are displayed in FIGS. 14A-S. For calculated values contained herein and throughout the present application, four significant digits are presented.

TABLE 3
Outlier Sequences from the Mouse Repeat
Dataset Showing Anomalous CpG Motif Usage

| | | | Strength of |
|---|---|---|---|
| | | | Statistical |
| Repeat Name | Repeat Class | Conservation | Bias on CpG |
| (CCCGAA)n | Simple Repeat | Eukaryota | 1.0173 |
| (CG)n | Simple Repeat | Eukaryota | 7.4253 |
| (CGAA)n | Simple Repeat | Eukaryota | 2.2781 |
| (CGGA)n | Simple Repeat | Eukaryota | 1.3857 |
| (GCC)n | Simple Repeat | Eukaryota | 1.3414 |
| (GCCC)n | Simple Repeat | Eukaryota | 0.6942 |
| (GCCCC)n | Simple Repeat | Eukaryota | 0.3504 |
| (GCCCCC)n | Simple Repeat | Eukaryota | 0.2198 |
| (GCGCA)n | Simple Repeat | Eukaryota | 0.4899 |
| Charlie25 | hAT | Mammalia | 0.0738 |
| Charlie26a | hAT | Mammalia | 0.0000 |
| Charlie27 | hAT | Eutheria | 0.0860 |
| Eulor1 | Transposable | Amniota | 0.8481 |
| | Element | | |
| Eulor10 | Transposable | Amniota | 0.6064 |
| | Element | | |
| Eulor11 | Transposable | Amniota | 0.3561 |
| | Element | | |
| Eulor12 | Transposable | Amniota | 0.5295 |
| | Element | | |
| Eulor12_CM | Transposable | Amniota | 0.2269 |
| | Element | | |
| Eulor2B | Transposable | Amniota | 0.2852 |
| | Element | | |
| Eulor2C | Transposable | Amniota | 0.7676 |
| | Element | | |
| Eulor4 | Transposable | Tetrapoda | 0.6067 |
| | Element | | |
| Eulor5A | Transposable | Tetrapoda | 0.0000 |
| | Element | | |
| Eulor5B | Transposable | Tetrapoda | 0.8474 |
| | Element | | |
| Eulor6A | Transposable | Tetrapoda | 0.7466 |
| | Element | | |
| Eulor6C | Transposable | Tetrapoda | 0.3571 |
| | Element | | |
| Eulor6D | Transposable | Tetrapoda | 0.2866 |
| | Element | | |
| Eulor6E | Transposable | Tetrapoda | 0.1268 |
| | Element | | |
| Eulor8 | Transposable | Amniota | 0.3416 |
| | Element | | |
| Eulor9A | Transposable | Amniota | 0.3465 |
| | Element | | |
| Eulor9B | Transposable | Amniota | 0.0000 |
| | Element | | |
| Eulor9C | Transposable | Amniota | 0.2751 |
| | Element | | |
| GSAT_MM | SAT | Mus musculus | 0.0000 |
| IAPEY2_LTR | ERV2 | Mus musculus | 0.0783 |
| IAPEY_LTR | ERV2 | Mus | 0.1998 |
| Kanga11a | Mariner/Tc1 | Mammalia | 0.1891 |
| LSU-rRNA_Cel | rRNA | Metazoa | 0.0186 |
| LSU-rRNA_Hsa | rRNA | Metazoa | 0.0330 |
| MamRep1894 | hAT | Mammalia | 0.4662 |
| MER104 | DNA transposon | Eutheria | 0.1428 |
| MER104C | DNA transposon | Eutheria | 0.0370 |
| MER121 | hAT | Mammalia | 0.0000 |
| MER123 | DNA transposon | Amniota | 1.1039 |
| MER125 | DNA transposon | Amniota | 0.0000 |
| MER127 | Mariner/Tc1 | Amniota | 0.2984 |
| MER129 | SINE | Amniota | 0.2444 |
| MER130 | Transposable | Amniota | 0.0000 |
| | Element | | |
| MER131 | SINE | Amniota | 0.6223 |
| MER133A | Transposable | Amniota | 0.4020 |
| | Element | | |
| MER133B | Transposable | Amniota | 0.0000 |
| | Element | | |
| MER134 | Transposable | Amniota | 0.2786 |
| | Element | | |
| MER2 | Mariner/Tc1 | Eutheria | 0.1577 |
| MER44D | Mariner/Tc1 | Eutheria | 0.3211 |
| MER47B | Mariner/Tc1 | Eutheria | 0.4518 |
| MER47C | Mariner/Tc1 | Eutheria | 0.7929 |
| MER58A | hAT | Eutheria | 0.2006 |
| MER58B | hAT | Eutheria | 0.3657 |
| MER58D | hAT | Eutheria | 0.0802 |
| MER5C1 | hAT | Eutheria | 0.4582 |
| MER6 | Mariner/Tc1 | Eutheria | 0.1783 |
| MER6C | Mariner/Tc1 | Eutheria | 0.5667 |
| MER97d | hAT | Eutheria | 0.2939 |
| MERX | Mariner/Tc1 | Eutheria | 0.2207 |
| RICKSHA_0 | MuDR | Eutheria | 0.0000 |
| Ricksha_a | MuDR | Eutheria | 0.2607 |
| RMER30 | hAT | Muridae | 0.1104 |
| SSU-rRNA_Cel | rRNA | Metazoa | 0.0830 |
| SSU-rRNA_Hsa | rRNA | Metazoa | 0.0464 |
| Tigger12A | Mariner/Tc1 | Mammalia | 0.2170 |
| Tigger2b | Mariner/Tc1 | Rodentia | 0.4588 |
| TIGGER5A | Mariner/Tc1 | Eutheria | 0.4212 |
| TIGGER5_B | Mariner/Tc1 | Eutheria | 0.1648 |
| Tigger9b | Mariner/Tc1 | Eutheria | 0.1869 |
| tRNA-Arg-CGA | tRNA | Vertebrata | 0.0000 |
| tRNA-Arg-CGG | tRNA | Vertebrata | 0.2001 |
| tRNA-Asp-GAY | tRNA | Vertebrata | 0.1489 |
| tRNA-His-CAY | tRNA | Vertebrata | 0.2007 |
| tRNA-Ile-ATA | tRNA | Vertebrata | 0.1118 |
| tRNA-Ile-ATT | tRNA | Vertebrata | 0.1970 |
| tRNA-Leu-CTA | tRNA | Vertebrata | 0.0000 |
| tRNA-Leu-CTG | tRNA | Vertebrata | 0.0000 |
| tRNA-Met_{—} | tRNA | Vertebrata | 0.0000 |
| tRNA-Pro-CCG | tRNA | Vertebrata | 0.0000 |
| tRNA-Ser-AGY | tRNA | Vertebrata | 0.0000 |
| tRNA-Ser-TCA | tRNA | Vertebrata | 0.0000 |
| tRNA-Ser-TCA_{—} | tRNA | Vertebrata | 0.2097 |
| tRNA-Ser-TCY | tRNA | Vertebrata | 0.1452 |
| tRNA-Tyr-TAC | tRNA | Vertebrata | 0.0000 |
| UCON1 | Transposable | Amniota | 0.0841 |
| | Element | | |
| UCON15 | Transposable | Amniota | 0.3560 |
| | Element | | |
| UCON16 | Transposable | Amniota | 0.4436 |
| | Element | | |
| UCON21 | Transposable | Amniota | 0.9465 |
| | Element | | |
| UCON26 | Transposable | Amniota | 0.2985 |
| | Element | | |
| UCON27 | Transposable | Amniota | 0.0400 |
| | Element | | |
| UCON39 | DNA transposon | Mammalia | 0.4443 |
| UCON63 | Repetitive element | Mammalia | 0.0000 |
| UCON9 | Transposable | Amniota | 0.0979 |
| | Element | | |
| Zaphod3 | hAT | Eutheria | 0.0077 |

lncRNAs meeting this threshold from the Mouse ENCODE dataset are found in Table 4 and their corresponding nucleotide sequences are displayed in FIGS. 15A-F.

TABLE 4
Outlier Sequences from the Mouse ENCODE Dataset Showing Anomalous CpG Motif Usage

| | Force on |
|---|---|
| IncRNA Identifier | CpG |
| ENSMUST00000174738.1\|ENSMUSG00000092405.1\|OTTMUSG00000038236.1\|OTTMUST00000098449.1\|Gm20402-001\|Gm20402\|687\| | 0.0410 |
| ENSMUST00000148335.1\|ENSMUSG00000086556.2\|OTTMUSG00000021933.1\|OTTMUST00000052064.1\|Gm15444-001\|Gm15444\|388\| | 0.0614 |
| ENSMUST00000125852.1\|ENSMUSG00000085102.1\|OTTMUSG00000007303.1\|OTTMUST00000016874.1\|1700010K24Rik- | 0.0000 |
| 001\|1700010K24Rik\|226\| | |
| ENSMUST00000166606.1\|ENSMUSG00000091623.1\|OTTMUSG00000036764.1\|OTTMUST00000094340.1\|Gm17092-001\|Gm17092\|698\| | 0.1875 |
| ENSMUST00000151096.1\|ENSMUSG00000086700.1\|OTTMUSG00000025925.1\|OTTMUST00000063910.1\|Gm15747-002\|Gm15747\|521\| | 0.0000 |
| ENSMUST00000154673.1\|ENSMUSG00000085355.2\|OTTMUSG00000024044.1\|OTTMUST00000058783.1\|3010003L21Rik- | 0.0000 |
| 001\|3010003L21Rik\|1747\| | |
| ENSMUST00000047953.9\|ENSMUSG00000085355.2\|OTTMUSG00000024044.1\|—\|13010003L21Rik-201\|3010003L21Rik\|1729\| | 0.0058 |
| ENSMUST00000146269.1\|ENSMUSG00000085923.1\|OTTMUSG00000008402.1\|OTTMUST00000019057.1\|Gm12781-001\|Gm12781\|395\| | 0.1098 |
| ENSMUST00000184554.1\|ENSMUSG00000098496.1\|OTTMUSG00000044627.1\|OTTMUST00000117415.1\|RP23-32A8.1-001\|RP23- | 0.2466 |
| 32A8.1\|409\| | |
| ENSMUST00000184855.1\|ENSMUSG00000098496.1\|OTTMUSG00000044627.1\|OTTMUST00000117414.1\|RP23-32A8.1-002\|RP23- | 0.2466 |
| 32A8.1\|409\| | |
| ENSMUST00000184655.1\|ENSMUSG00000098496.1 \|OTTMUSG00000044627.1\|OTTMUST00000117416.1\|RP23-32A8.1-003\|RP23- | 0.0000 |
| 32A8.1\|310\| | |
| ENSMUST00000140952.1\|ENSMUSG00000085645.1\|OTTMUSG00000001986.1\|OTTMUST00000003990.1\|0610040B09Rik- | 0.0541 |
| 002\|0610040B09Rik\|158\| | |
| ENSMUST00000136542.1\|ENSMUSG00000085501.1\|OTTMUSG00000004131.1\|OTTMUST00000009325.1\|Gm11772-001\|Gm11772\|532\| | 0.0779 |
| ENSMUST00000171248.1\|ENSMUSG00000090779.1\|OTTMUSG00000036088.1\|OTTMUST00000092719.1\|Gm17110-001\|Gm17110\|735\| | 0.1405 |
| ENSMUST00000127359.1\|ENSMUSG00000086746.1\|OTTMUSG00000019533.1\|OTTMUST00000046645.1\|Gm15222-001\|Gm15222\|344\| | 0.0926 |
| ENSMUST00000175699.1\|ENSMUSG00000093387.1\|OTTMUSG00000040094.1\|OTTMUST00000104147.1\|Gm20732-001\|Gm20732\|686\| | 0.1916 |
| ENSMUST00000161706.1\|ENSMUSG00000090101.1\|OTTMUSG00000029229.1\|OTTMUST00000072458.1\|Snhg9-001\|Snhg9\|183\| | 0.3679 |
| ENSMUST00000174851.1\|ENSMUSG00000092338.1\|OTTMUSG00000037106.1\|OTTMUST00000095531.1\|Gm26940-001\|Gm26940\|105\| | 0.1422 |
| ENSMUST00000182520.1\|ENSMUSG00000097971.2\|OTTMUSG00000043054.1\|OTTMUST00000112997.1\|Gm26917-002\|Gm26917\|869\| | 0.0677 |
| ENSMUST00000182010.1\|ENSMUSG00000098178.1\|OTTMUSG00000043056.1\|OTTMUST00000112999.1\|Gm26924- | 0.0667 |
| 001\|Gm26924\|1831\| | |
| ENSMUST00000146010.2\|ENSMUSG00000087590.2\|OTTMUSG00000042342.1\|OTTMUST00000111570.1\|2410004N09Rik- | 0.0556 |
| 001\|2410004N09Rik\|430\| | |
| ENSMUST00000179138.1\|ENSMUSG00000087590.2\|OTTMUSG00000042342.1\|OTTMUST00000111571.1\|2410004N09Rik- | 0.0757 |
| 002\|2410004N09Rik\|303\| | |
| ENSMUST00000149574.1\|ENSMUSG00000052188.6\|OTTMUSG00000018617.2\|OTTMUST00000044828.2\|Gm14964-001\|Gm14964\|716\| | 0.0609 |
| ENSMUST00000137184.1\|ENSMUSG00000052188.6\|OTTMUSG00000018617.2\|OTTMUST00000044829.1\|Gm14964-002\|Gm14964\|519\| | 0.0344 |

Human Repetitive elements meeting this threshold from the human repeat sequences from the Repbase database are found in Table 5 and their corresponding nucleotide sequences are displayed in FIGS. 16A-Y.

TABLE 5
Outlier Sequences from the Human Repeat
Dataset Showing Anomalous CpG Motif Usage

| | | | Force on |
|---|---|---|---|
| Repeat Name | Repeat Class | Conservation | CpG |
| (CCCGAA)n | Simple Repeat | Eukaryota | 1.0173 |
| (CG)n | Simple Repeat | Eukaryota | 7.4253 |
| (CGAA)n | Simple Repeat | Eukaryota | 2.2781 |
| (CGGA)n | Simple Repeat | Eukaryota | 1.3857 |
| (GCC)n | Simple Repeat | Eukaryota | 1.3414 |
| (GCCC)n | Simple Repeat | Eukaryota | 0.6942 |
| (GCCCC)n | Simple Repeat | Eukaryota | 0.3504 |
| (GCCCCC)n | Simple Repeat | Eukaryota | 0.2198 |
| (GCGCA)n | Simple Repeat | Eukaryota | 0.4899 |
| Charlie25 | hAT | Mammalia | 0.0738 |
| Charlie26a | hAT | Mammalia | 0.0000 |
| Charlie27 | hAT | Eutheria | 0.0860 |
| Eulor1 | Transposable | Amniota | 0.8481 |
| | Element | | |
| Eulor10 | Transposable | Amniota | 0.6064 |
| | Element | | |
| Eulor11 | Transposable | Amniota | 0.3561 |
| | Element | | |
| Eulor12 | Transposable | Amniota | 0.5295 |
| | Element | | |
| Eulor12_CM | Transposable | Amniota | 0.2269 |
| | Element | | |
| Eulor2B | Transposable | Amniota | 0.2852 |
| | Element | | |
| Eulor2C | Transposable | Amniota | 0.7676 |
| | Element | | |
| Eulor4 | Transposable | Tetrapoda | 0.6067 |
| | Element | | |
| Eulor5A | Transposable | Tetrapoda | 0.0000 |
| | Element | | |
| Eulor5B | Transposable | Tetrapoda | 0.8474 |
| | Element | | |
| Eulor6A | Transposable | Tetrapoda | 0.7466 |
| | Element | | |
| Eulor6C | Transposable | Tetrapoda | 0.3571 |
| | Element | | |
| Eulor6D | Transposable | Tetrapoda | 0.2866 |
| | Element | | |
| Eulor6E | Transposable | Tetrapoda | 0.1268 |
| | Element | | |
| Eulor8 | Transposable | Amniota | 0.3416 |
| | Element | | |
| Eulor9A | Transposable | Amniota | 0.3465 |
| | Element | | |
| Eulor9B | Transposable | Amniota | 0.0000 |
| | Element | | |
| Eulor9C | Transposable | Amniota | 0.2751 |
| | Element | | |
| GGAAT | SAT | Homo sapiens | 0.0000 |
| GOLEM_A | Mariner/Tc1 | Homo sapiens | 0.1066 |
| HSAT6 | SAT | Homo sapiens | 0.6156 |
| HSATII | SAT | Primates | 1.0360 |
| HSMAR1 | Mariner/Tc1 | Homo sapiens | 0.2397 |
| Kanga11a | Mariner/Tc1 | Mammalia | 0.1891 |
| LSU-rRNA_Cel | rRNA | Metazoa | 0.0186 |
| LSU-rRNA_Hsa | rRNA | Metazoa | 0.0330 |
| MacERV4_LTR1b | ERV2 | Cercopithecidae | 0.0000 |
| MacERV4_LTR2 | ERV2 | Cercopithecidae | 0.0455 |
| MacERV5b_LTR | ERV1 | Cercopithecidae | 0.0000 |
| MacERV6_LTR2a | ERV3 | Cercopithecidae | 0.0000 |
| MacERV6_LTR2c | ERV3 | Cercopithecidae | 0.0307 |
| MacERV6_LTR3 | ERV3 | Cercopithecidae | 0.2404 |
| MacERV6_LTR4 | ERV3 | Cercopithecidae | 0.0373 |
| MacERV6_LTR5 | ERV3 | Cercopithecidae | 0.0305 |
| MacERVKlLTR1b | ERV2 | Cercopithecidae | 0.0000 |
| MacERVKILTR1e | ERV2 | Cercopithecidae | 0.0000 |
| MamRep1894 | hAT | Mammalia | 0.4662 |
| MER104 | DNA transposon | Eutheria | 0.1428 |
| MER104C | DNA transposon | Eutheria | 0.0370 |
| MER119 | hAT | Homo sapiens | 0.2794 |
| MER121 | hAT | Mammalia | 0.0000 |
| MER123 | DNA transposon | Amniota | 1.1039 |
| MER125 | DNA transposon | Amniota | 0.0000 |
| MER127 | Mariner/Tc1 | Amniota | 0.2984 |
| MER129 | SINE | Amniota | 0.2444 |
| MER130 | Transposable | Amniota | 0.0000 |
| | Element | | |
| MER131 | SINE | Amniota | 0.6223 |
| MER133A | Transposable | Amniota | 0.4020 |
| | Element | | |
| MER133B | Transposable | Amniota | 0.0000 |
| | Element | | |
| MER134 | Transposable | Amniota | 0.2786 |
| | Element | | |
| MER2 | Mariner/Tc1 | Eutheria | 0.1577 |
| MER44A | Mariner/Tc1 | Homo sapiens | 0.1388 |
| MER44B | Mariner/Tc1 | Homo sapiens | 0.3536 |
| MER44C | Mariner/Tc1 | Homo sapiens | 0.3439 |
| MER44D | Mariner/Tc1 | Eutheria | 0.3211 |
| MER45B | DNA transposon | Homo sapiens | 0.1120 |
| MER47B | Mariner/Tc1 | Eutheria | 0.4518 |
| MER47C | Mariner/Tc1 | Eutheria | 0.7929 |
| MER57A1 | ERV1 | Homo sapiens | 0.0000 |
| MER57B2 | ERV1 | Homo sapiens | 0.2403 |
| MER58A | hAT | Eutheria | 0.2006 |
| MER58B | hAT | Eutheria | 0.3657 |
| MER58D | hAT | Eutheria | 0.0802 |
| MER5C1 | hAT | Eutheria | 0.4582 |
| MER6 | Mariner/Tc1 | Eutheria | 0.1783 |
| MER63D | hAT | Homo sapiens | 0.0665 |
| MER6A | Mariner/Tc1 | Primates | 0.0913 |
| MER6B | Mariner/Tc1 | Homo sapiens | 0.9230 |
| MER6C | Mariner/Tc1 | Eutheria | 0.5667 |
| MER75 | DNA transposon | Homo sapiens | 0.4134 |
| MER75A | piggyBac | Primates | 0.0000 |
| MER8 | Mariner/Tc1 | Homo sapiens | 0.2669 |
| MER97A | hAT | Homo sapiens | 0.0315 |
| MER97d | hAT | Eutheria | 0.2939 |
| MERX | Mariner/Tc1 | Eutheria | 0.2207 |
| npiggy1_Mm | piggyBac | Microcebus murinus | 0.3131 |
| npiggy2_Mm | piggyBac | Microcebus murinus | 0.3725 |
| RICKSHA_0 | MuDR | Eutheria | 0.0000 |
| Ricksha_a | MuDR | Eutheria | 0.2607 |
| SSU-rRNA_Cel | rRNA | Metazoa | 0.0830 |
| SSU-rRNA_Hsa | rRNA | Metazoa | 0.0464 |
| SUBTEL2_sat | SAT | Primates | 0.2960 |
| SUBTEL_sat | Satellite | Primates | 0.3527 |
| Tigger12A | Mariner/Tc1 | Mammalia | 0.2170 |
| Tigger2b_Pri | Mariner/Tc1 | Primates | 0.3548 |
| Tigger3c | Mariner/Tc1 | Primates | 0.1192 |
| Tigger3d | Mariner/Tc1 | Primates | 0.4374 |
| Tigger4a | Mariner/Tc1 | Primates | 0.3815 |
| TIGGER5A | Mariner/Tc1 | Eutheria | 0.4212 |
| TIGGER5 B | Mariner/Tc1 | Eutheria | 0.1648 |
| Tigger9b | Mariner/Tc1 | Eutheria | 0.1869 |
| tRNA-Arg-CGA | tRNA | Vertebrata | 0.0000 |
| tRNA-Arg-CGG | tRNA | Vertebrata | 0.2001 |
| tRNA-Asp-GAY | tRNA | Vertebrata | 0.1489 |
| tRNA-His-CAY_{—} | tRNA | Vertebrata | 0.2007 |
| tRNA-Ile-ATA | tRNA | Vertebrata | 0.1118 |
| tRNA-Ile-ATT | tRNA | Vertebrata | 0.1970 |
| tRNA-Leu-CTA | tRNA | Vertebrata | 0.0000 |
| tRNA-Leu-CTG | tRNA | Vertebrata | 0.0000 |
| tRNA-Met_{—} | tRNA | Vertebrata | 0.0000 |
| tRNA-Pro-CCG | tRNA | Vertebrata | 0.0000 |
| tRNA-Ser-AGY | tRNA | Vertebrata | 0.0000 |
| tRNA-Ser-TCA | tRNA | Vertebrata | 0.0000 |
| tRNA-Ser-TCA_{—} | tRNA | Vertebrata | 0.2097 |
| tRNA-Ser-TCY | tRNA | Vertebrata | 0.1452 |
| tRNA-Tyr-TAC | tRNA | Vertebrata | 0.0000 |
| TRNA_ALA | tRNA | Homo sapiens | 0.0000 |
| TRNA_ASN | tRNA | Homo sapiens | 0.1580 |
| TRNA_GLU | tRNA | Homo sapiens | 0.0000 |
| TRNA_VAL | tRNA | Homo sapiens | 0.5721 |
| U4B | snRNA | Homo sapiens | 0.2960 |
| U6 | snRNA | Homo sapiens | 0.3083 |
| UCON1 | Transposable | Amniota | 0.0841 |
| | Element | | |
| UCON15 | Transposable | Amniota | 0.3560 |
| | Element | | |
| UCON16 | Transposable | Amniota | 0.4436 |
| | Element | | |
| UCON21 | Transposable | Amniota | 0.9465 |
| | Element | | |
| UCON26 | Transposable | Amniota | 0.2985 |
| | Element | | |
| UCON27 | Transposable | Amniota | 0.0400 |
| | Element | | |
| UCON39 | DNA transposon | Mammalia | 0.4443 |
| UCON63 | Repetitive | Mammalia | 0.0000 |
| | element | | |
| UCON9 | Transposable | Amniota | 0.0979 |
| | Element | | |
| Zaphod3 | hAT | Eutheria | 0.0077 |
| ZOMBI_A | Mariner/Tc1 | Homo sapiens | 0.1808 |

Human ENCODE elements meeting this threshold from the Human ENCODE dataset are found in Table 6 and their corresponding nucleotide sequences are displayed in FIG. 17A-L.

TABLE 6
Outlier Sequences from the Human ENCODE Dataset Showing Anomalous CpG Motif Usage

| | Force on |
|---|---|
| IncRNA Identifier | CpG |
| ENST00000602813.1\|ENSG00000270103.2\|OTTHUMG00000183994.1\|OTTHUMT00000467710.1\|RNU11-0011\|RNU11\|131\| | 0.2384 |
| ENST00000387069.1\|ENSG00000270103.2\|OTTHUMG00000183994.1\|—\|RNU11-201\|RNU11\|134\| | 0.2175 |
| ENST00000448344.1\|ENSG00000231485.1\|OTTHUMG00000009304.1\|OTTHUMT00000025777.1\|RP4-535B20.1-001\|RP4- | 0.0753 |
| 535B20.1\|310\| | |
| ENST00000608684.1\|ENSG00000273338.1\|OTTHUMG00000186144.1\|OTTHUMT00000472318.1\|RP11-386I14.4-001\|RP11- | 0.0000 |
| 386I14.4\|209\| | |
| ENST00000385223.1\|ENSG00000225206.4\|OTTHUMG00000010680.2\|—\|MIR137HG-201\|MIR137HG\|102\| | 0.4801 |
| ENST00000431097.2\|ENSG00000226889.3\|OTTHUMG00000034539.2\|OTTHUMT00000083587.2\|RP11-474I16.8-002\|RP11- | 0.0000 |
| 474I16.8\|575\| | |
| ENST00000364822.2\|ENSG00000234741.3\|OTTHUMG00000037216.2\|—\|GAS5-205\|GAS5\|82\| | 0.0000 |
| ENST00000448808.1\|ENSG00000228106.1\|OTTHUMG00000037767.3\|OTTHUMT00000100398.1\|RP11-452F19.3-012\|RP11- | 0.0612 |
| 452F19.3\|130\| | |
| ENST00000439440.1\|ENSG00000228106.1\|OTTHUMG00000037767.3\|OTTHUMT00000092500.1\|RP11-452F19.3-005\|RP11- | 0.1804 |
| 452F19.3\|216\| | |
| ENST00000457097.1\|ENSG00000235586.1\|OTTHUMG00000153432.1\|OTTHUMT00000331178.1\|AC011247.3-001\|AC011247.3\|233\| | 0.0000 |
| ENST00000442821.1\|ENSG00000231054.1\|OTTHUMG00000152442.1\|OTTHUMT00000326240.1\|AC009236.2-001\|AC009236.2\|553\| | 0.0415 |
| ENST00000455416.1\|ENSG00000229337.1\|OTTHUMG00000154102.1\|OTTHUMT00000333896.1\|AC079305.8-001\|AC079305.8\|218\| | 0.2205 |
| ENST00000607245.1\|ENSG00000272434.1\|OTTHUMG00000185526.1\|OTTHUMT00000470652.1\|RP13-131K.19.6-001\|RP13- | 0.0523 |
| 131K.19.6\|391\| | |
| ENST00000469484.1\|ENSG00000244586.1\|OTTHUMG00000158382.1\|OTTHUMT00000350841.1\|WNT5 A-AS1-001\|WNT5A-AS1\|500\| | 0.0460 |
| ENST00000490320.1\|ENSG00000244078.1\|OTTHUMG00000158950.1\|OTTHUMT00000352646.1\|RP11-431I8.1-001\|RP11- | 0.0000 |
| 431I8.1\|424\| | |
| ENST00000609552.1\|ENSG00000272677.1\|OTTHUMG00000186309.2\|OTTHUMT00000472826.1\|RP11-127B20.3-002\|RP11- | 0.0000 |
| 127B20.3\|612\| | |
| ENST00000602520.1\|ENSG00000269893.2\|OTTHUMG00000183991.1\|OTTHUMT00000467704.1\|SNHG8-002\|SNHG8\|327\| | 0.0817 |
| ENST00000513037.1\|ENSG00000250600.1\|OTTHUMG00000162052.1\|OTTHUMT00000367040.1\|ROPN1L-AS1-001\|ROPN1L-AS1\|189\| | 0.0698 |
| ENST00000521596.1\|ENSG00000253744.1\|OTTHUMG00000164088.1\|OTTHUMT00000377186.1\|AC025442.3-001\|AC025442.3\|481\| | 0.2300 |
| ENST00000513771.1\|ENSG00000248473.1\|OTTHUMG00000162379.1\|OTTHUMT00000368676.1\|CTC-338M12.2-001\|CTC- | 0.1332 |
| 338M12.2\|411\| | |
| ENST00000606441.1\|ENSG00000272277.1\|OTTHUMG00000185651.1\|OTTHUMT00000470934.1\|RP1-40E16.12-001\|RP1- | 0.0220 |
| 40E16.12\|850\| | |
| ENST00000441978.1\|ENSG00000235488.1\|OTTHUMG00000014292.1\|OTTHUMT00000039925.1\|JARID2-AS1-001\|JARID2-AS1\|455\| | 0.0711 |
| ENST00000434329.2\|ENSG00000242973.2\|OTTHUMG00000014787.2\|OTTHUMT00000040799.2\|RP11-446F17.3-002\|RP11- | 0.0857 |
| 446F17.3\|374\| | |
| ENST00000384338.1\|ENSG00000203875.6\|OTTHUMG00000015144.3\|—\|SNHG5-202\|SNHG5\|75\| | 0.0000 |
| ENST00000364995.1\|ENSG00000203875.6\|OTTHUMG00000015144.3\|—\|SNHG5-201\|SNHG5\|70\| | 0.0000 |
| ENST00000435287.1\|ENSG00000227220.1\|OTTHUMG00000150056.1\|OTTHUMT00000316064.1\|RP11-69I8.3-001\|RP11-69I8.3\|495\| | 0.0681 |
| ENST00000608721.1\|ENSG00000272841.1\|OTTHUMG00000185865.1\|OTTHUMT00000471562.1\|RP3-428L16.2-001\|RP3- | 0.0099 |
| 428L16.2\|2025\| | |
| ENST00000604200.1\|ENSG00000270419.1\|OTTHUMG00000175945.2\|OTTHUMT00000431300.2\|CAHM-001\|CAHM\|896\| | 0.1563 |
| ENST00000604183.1\|ENSG00000271185.1\|OTTHUMG00000185253.1\|OTTHUMT00000469985.1\|RP5-855F16.1-001\|RP5-855F16.1\|313\| | 0.0000 |
| ENST00000433005.1\|ENSG00000237773.1\|OTTHUMG00000152468.1\|OTTHUMT00000326308.1\|AC003075.4-006\|AC003075.4\|540\| | 0.1390 |
| ENST00000454029.1\|ENSG00000234286.1\|OTTHUMG00000152691.1\|OTTHUMT00000327406.1\|AC006026.13-001\|AC006026.13\|143\| | 0.0000 |
| ENST00000608799.1\|ENSG00000272843.1\|OTTHUMG00000186270.1\|OTTHUMT00000472568.1\|RP11-313P13.5-001\|RP11- | 0.0414 |
| 313P13.5\|708\| | |
| ENST00000585013.1\|ENSG00000239569.2\|OTTHUMG00000157280.1\|—\|KMT2E-AS1-201\|KMT2E-AS1\|48\| | 1.1847 |
| ENST00000522768.1\|ENSG00000253944.1\|OTTHUMG00000163705.1\|OTTHUMT00000374850.1\|RP11-156K13.1-001\|RP11- | 0.0279 |
| 156K13.1\|510\| | |
| ENST00000606596.1\|ENSG00000272256.1\|OTTHUMG00000185429.1\|OTTHUMT00000470512.1\|RP11-489E7.4-001\|RP11-489E7.4\|710\| | 0.0212 |
| ENST00000521399.1\|ENSG00000245910.4\|OTTHUMG00000164743.3\|OTTHUMT00000380024.1\|SNHG6-006\|SNHG6\|302\| | 0.1288 |
| ENST00000519782.1\|ENSG00000253806.1\|OTTHUMG00000164674.1\|OTTHUMT00000379712.1\|CTD-2292P10.2-001\|CTD- | 0.0655 |
| 2292P10.2\|340\| | |
| ENST00000446211.1\|ENSG00000226386.1\|OTTHUMG00000017947.1\|OTTHUMT00000047525.1\|PARD3-AS1-001\|PARD3-AS1\|302\| | 0.3048 |
| ENST00000532866.1\|ENSG00000254694.1\|OTTHUMG00000165816.1\|OTTHUMT00000386345.1\|RP11-50B3.4-001\|RP11-50B3.4\|362\| | 0.2496 |
| ENST00000546421.1\|ENSG00000257167.2\|OTTHUMG00000170209.3\|OTTHUMT00000408019.1\|TMPO-AS1-002\|TMPO-AS1\|738\| | 0.0132 |
| ENST00000554537.1\|ENSG00000258982.1\|OTTHUMG00000171545.1\|OTTHUMT00000414045.1\|RP11-63812.4-001\|RP11-63812.4\|331\| | 0.0258 |
| ENST00000408206.1\|ENSG00000258498.2\|OTTHUMG00000171682.1\|—\|DIO3OS-201\|DIO3OS\|136\| | 0.2684 |
| ENST00000384430.1\|ENSG00000224078.8\|OTTHUMG00000056661.6\|—\|SNHG14-205\|SNHG14\|92\| | 0.0000 |
| ENST00000384507.1\|ENSG00000261069.2\|OITHUMG00000176878.1\|—\|SNORD116-20-201\|SNORD116-201\|92\| | 0.0000 |
| ENST00000559134.1\|ENSG00000259488.1\|OTTHUMG00000172154.2\|OTTHUMT00000417138.1\|RP11-154J22.1-001\|RP11- | 0.0000 |
| 154J22.1\|577\| | |
| ENST00000553829.1\|ENSG00000272888.1\|OTTHUMG00000149845.8\|OTTHUMT00000415065.1\|AC013394.2-003\|AC013394.2\|732\| | 0.0191 |
| ENST00000554669.1\|ENSG00000272888.1\|OTTHUMG00000149845.8\|OTTHUMT00000415067.1\|AC013394.2-005\|AC013394.2\|578\| | 0.2085 |
| ENST00000554894.1\|ENSG00000272888.1\|OTTHUMG00000149845.8\|OTTHUMT00000415068.1\|AC013394.2-006\|AC013394.2\|556\| | 0.1990 |
| ENST00000557147.1\|ENSG00000272888.1\|OTTHUMG00000149845.8\|OTTHUMT00000415069.1\|AC013394.2-008\|AC013394.2\|490\| | 0.0831 |
| ENST00000531523.1\|ENSG00000255198.3\|OTTHUMG00000166082.2\|OTTHUMT00000387781.1\|SNHG9-001\|SNHG9\|275\| | 0.1085 |
| ENST00000560208.1\|ENSG00000245694.4\|0TTHUMG00000172236.2\|OTTHUMT00000417438.1\|CRNDE-006\|CRNDE\|735\| | 0.0000 |
| ENST00000570444.1\|ENSG00000262624.1\|OTTHUMG00000178213.1\|OTTHUMT00000441007.1\|RP11-104H15.9-001\|RP11- | 0.0686 |
| 104H15.9\|327\| | |
| ENST00000365172.1\|ENSG00000175061.13\|OTTHUMG00000058990.5\|—\|C17orf76-AS1-201\|C17orf76-AS1\|72\| | 0.1702 |
| ENST00000384229.1\|ENSG00000175061.13\|OTTHUMG00000058990.5\|—\|C17orf76-AS1-202\|C17orf76-AS1\|71\| | 0.0000 |
| ENST00000487849.3\|ENSG00000233101.6\|OTTHUMG00000159919.3\|OTTHUMT00000358247.3\|HOXB-AS3-005\|HOXB-AS3\|428\| | 0.0586 |
| ENST00000466037.2\|ENSG00000233101.6\|OTTHUMG00000159919.3\|OTTHUMT00000358246.2\|HOXB-AS3-004\|HOXB-AS3\|522\| | 0.0699 |
| ENST00000408535.2\|ENSG00000266402.2\|OTTHUMG00000178880.1\|—\|SNORA76-2011\|SNORA76\|133\| | 0.0000 |
| ENST00000589968.1\|ENSG00000267363.1\|OTTHUMG00000180677.1\|OTTHUMT00000452531.1\|CTD-3162L10.4-001\|CTD- | 0.3777 |
| 3162L10.4\|249\| | |
| ENST00000385250.1\|ENSG00000227195.4\|OTTHUMG00000032149.3\|—\|MIR663A-201\|MIR663A\|93\| | 0.0000 |
| ENST00000459583.1\|ENSG00000225978.2\|OTTHUMG00000140136.1\|—\|HAR1A-201\|HAR1A\|132\| | 0.4985 |
| ENST00000440315.2\|ENSG00000206142.5\|OTTHUMG00000150795.1\|—\|KB-1183D5.13-201\|KB-1183D5.13\|651\| | 0.2327 |
| ENST00000585003.1\|ENSG00000226471.2\|OTTHUMG00000151093.2\|OTTHUMT00000447487.1\|CTA-292E10.6-005\|CTA- | 0.0000 |
| 292E10.6\|516\| | |
| ENST00000362512.1\|ENSG00000270022.2\|OTTHUMG00000183993.1\|—\|RNU12-201\|RNU12\|150\| | 0.1296 |
| ENST00000535837.1\|ENSG00000196972.6\|OTTHUMG00000022468.2\|—\|LINC00087-201\|LINC00087\|204\| | 0.0753 |

### Example 7

### Design of Experimental Controls

For HSATII and GSAT, negative controls were designed in two ways and both negative controls were compared to HSATII and GSAT for all experiments. First, full RNA sequences of both satellites were randomly permuted until scrambled sequences were generated that fell within one half of a standard deviation from the mean value of the strength of statistical bias against CpG and UpA dinucleotides for humans and mice, respectively. These sequences are denoted as HSATII-sc and GSAT-sc. In other words, these sequences had the same length and nucleotide content as HSATII and GSAT but fell within the inner ellipse in FIG. 5A (HSATII-sc) and FIG. 5B (GSAT-sc). In addition, it was checked that in both cases the minimum RNA folding energy was not lowered during the scrambling process so that the permutations did not seem to produce more RNA secondary structure thereby creating the possibility of innate immune stimulation via TLR3. The free energy was calculated using the MATLAB RNAfold routine (Matthews et al., “Expanded Sequence Dependence of Thermodynamic Parameters Improves Prediction of RNA Secondary Structure,” J. Mol. Biol. 288:911-940 (1999) and Wuchty et al., “Complete Suboptimal Folding of RNA and the Stability of Secondary Structures,” Biopolymers 49:145-165 (1999), which are hereby incorporated by reference in their entirety). Endogenous negative controls were created by searching Repbase for the repetitive elements that fell within one standard deviation of the mean strength of statistical bias against CpG and UpA in humans and mice but were also closest in length to HSATII and GSAT. These were UCON38 for HSATII and RMER16A3 for GSAT.

### Example 8

### GSAT RNA Expression Level Detection

GSAT RNA expression levels were investigated by a custom TAQMAN qPCR Assay in normal mouse tissue versus mouse tumor tissue samples (FIGS. 4A-B). The tumor mouse models that were investigated were a model of testicular teratoma (p53−/−129/SvSL) and a model of liposarcoma (p53LoxP/LoxP;PtenLoxP/LoxP). In all instances, GSAT levels were increased in the tumor samples as compared to normal samples but to varying degrees. There was no significant difference in GSAT levels between tumors arising in females versus those arising in males in the liposarcoma model. Also, there was no difference in GSAT levels in p53−/−129/SvSL that developed teratomas at a young age (˜1 month old) versus at an older age (˜3-4 months old) (Harvey et al., “Genetic Background Alters the Spectrum of Tumors that Develop in p53-Deficient Mice,” The FASEB Journal 7:938-943 (1993) and Muller et al., “A Male Germ Cell Tumor Susceptibility Determining Locus pgct1 Identified on Murine Chromosome 13,” Proc. Natl. Acad. Sci. 97:8421-8426 (2000), which are hereby incorporated by reference in their entirety).

### Example 9

### i-ncRNA Generation

Sequences encoding for murine GSAT and human HSATII were generated by custom gene synthesis (Genscript) and cloned into a pCDNA3 backbone (EcoRI/EcoRV) that carries a T7 promoter on the + strand and a SP6 promoter on the − strand (Invitrogen). Sequences encoding for GSAT-sc, HSATII-sc, UCON38, and RMER16A3 were generated as minigenes and sub-cloned in a pIDT-blue backbone with a T7 promoter on the +strand and a T3 promoter on the − strand surrounding the sequence of interest (IDT). To produce high quality RNA, plasmids were digested by the restriction enzymes NotI/NdeI (pCDNA3) and ApaLI (pIDT blue) to isolate the fragment containing the sequence of interest by gel purification (Qiagen). Then the sequences of interest containing the T7 promoter were amplified by PCR (Accuprime-PFX Invitrogen) using the following primer pairs:

pIDT blue
Forward:
(SEQ ID NO: 320)
GCGCGTAATACGACTCACTATAGGCGA;
Reverse:
(SEQ ID NO: 321)
CGCAARRAACCCTCACTAAAGGGAACA
and
pCDNA.3
Forward:
(SEQ ID NO: 322)
GAAATTAATACGACTCAATAGG;
Reverse:
(SEQ ID NO: 323)
TCTAGCATTTAGGTGACACTATAGAATAG.

PCR products were purified by PCR-Cleanup (Qiagen) and controlled by electrophoresis (0.8% Agarose gel). RNAs were generated by in vitro transcription using the mMESSAGE mMACHINE T7 ultra kit (Ambion) followed by a capping and short polyA reaction. RNAs were then purified using RNA-cleanup (Qiagen), quantified using a nanodrop, and checked by electrophoresis after denaturation at 65° C. for 10 minutes (1 5% Agarose gel).

### Example 10

### Cell Stimulation

MoDCs and imBM were both stimulated by i-ncRNA in the same way. The culturing of these cells is described below. Briefly, cells were plated in 96 flat well plates at 200,000 cells per well for primary cells (MoDCs) and 100,000 cells per well for lines (IMBM). i-ncRNA were transfected via liposomes formed using DOTAP (Roche Life Science) at a ratio of 1 μg DNA per 6 μl DOTAP diluted in HBS following the user-guide recommendations. The cells were stimulated using 2 μg/ml of purified i-ncRNA versus 10 μg/ml total RNA. To stimulate the TLR4 pathway, 100 ng/ml Ultrapure LPS (Invivogen) was used for TLR2: 500 ng/ml Pam2CSK4 (Invivogen) for TLR3: 2 μg/ml HMW PolyIC (Invivogen) TLR7/8: 1 μg/ml CLO97 (Invivogen) and 100 ng/ml R848 (Invivogen) TLR9: CpG B-ODN 1826 3 μM or STING CDN 5 μg/ml (Aduro).

### Example 11

### Cell Culture

Human moDCs: Human monocyte derived DCs were differentiated as previously described (Frleta et al., “HIV-1 Infection-Induced Apoptotic Microparticles Inhibit Human DCs via CD44,” J. Clinical Invest. 122:4685 (2012), which is hereby incorporated by reference in its entirety). Briefly, PBMCs were prepared by centrifugation over Ficoll-Hypaque gradients (BioWhittaker) from healthy donor buffy coats (New York Blood Center). Monocytes were isolated from PBMCs by adherence and then treated with 100 U/ml GM-CSF (Leukine Sanofi Oncology) and 300 U/ml IL-4 (RandD) in RPMI plus 5% human AB serum (Gemini Bio Products). Differentiation media was renewed on day 2 and day 4 of culture. Mature moDCs were harvested for use on days 5 to 7. For all experiments, harvested DCs were washed and equilibrated in serum-free X-Vivo 15 media (Lonza).

Murine imBMs: Immortalized macrophages were immortalized by infecting bone marrow progenitors with oncogenic v-myc/vraf expressing J2 retrovirus as previously described (Blasi et al., “Selective Immortalization of Murine Macrophages from Fresh Bone Marrow by a raf/myc Recombinant Murine Retrovirus,” Nature 318:667-670 (1985), which is hereby incorporated by reference in its entirety) and differentiated in macrophage differentiated media containing MCSF. ImBM were maintained in 10% FCS PSN DMEM (Gibco). ImBM lines were provided by several collaborators and also obtained from the BEI resource: ICE (Casp1/Casp11), MAVs, IFN-R, IRF3-7, STING and their rescues, Unc93b1 3d/3d, TLR 3, 4, 7, 9, 2-9, 2-4, MYD88, TRIF, TRAM, and TRIF-TRAM.

### Example 12

### Investigation of Type I Interferon Pathway

To characterize whether this pathway could be modulated in the models, production of type I interferon in response to stimulation by the i-ncRNA using human and murine interferon stimulated response element (ISRE) reporter cell lines was evaluated and transcriptome regulation of a panel of immune genes related to the interferon pathway was monitored. Whereas the effect on the inflammatory response is significant in terms of TNFalpha, IL-6, or IL-12 production, the effect on the type I interferon pathway was less prominent.

### Example 13

### Additional Pathways Investigated

TLR2 or TLR4 were not required, indicating the observed effect was independent of contamination from bacterial products such as lipoproteins and endotoxins (FIGS. 12A-B). TRIF, TRIF/TRAM, and IRF3/IRF7, which participate downstream in the signaling of TLR3, TLR4, and TLR7, were also not obligatory (FIG. 13). A role for candidate molecules for sensing murine GSAT, such sensors related to cGAS-STING signaling or DEAD box RNA helicases such as RIG-I and MDAS (Atianand et al., “Molecular Basis of DNA Recognition in the Immune System,” J. Immunol. 190:1911-1918 (2013); Lee et al., “UNC93B1 Mediates Differential Trafficking of Endosomal TLRs,” eLife 2:e00291 (2013); Burdette et al., “STING and the Innate Immune Response to Nucleic Acids in the Cytosol,” Nature Immunol. 14:19-26 (2013); Vanaja et al., “Mechanisms of Inflammasome Activation: Recent Advance and Novel Insights,” Trends Cell Biol. 25(5):308-15 (2015), which are hereby incorporated by reference in their entirety) was not identified. Inflammatory responses to GSAT did not depend upon the stimulator of interferon genes (STING), which induces type I interferon production when cells are infected with intracellular pathogens. RIG-I (retinoic acid-inducible gene 1) is a dsRNA helicase enzyme that senses RNA viruses through activation of the mitochondrial antiviral-signaling protein (MAVS) (Zeng et al., “MAVS cGAS and Endogenous Retroviruses in T-independent B cell Responses,” Science 346:1486-1492 (2014); Broz et al., “Newly Described Pattern Recognition Receptors Team up Against Intracellular Pathogens,” Nature Rev. Immunol. 13:551-565 (2103); Gajewski et al., “Innate and Adaptive Immune Cells in the Tumor Microenvironment,” Nature Immunol. 14:1014-1022 (2013), which are hereby incorporated by reference in their entirety). MAVS deficient imBMs failed to respond to GSAT stimulation ruling out a contribution of RIG-I in the i-ncRNA signaling (FIG. 11B). Finally, a role for inflammasome related pathways was ruled out using ICE-KO imBM that are essentially a knockout for Caspase 1 and which carry an inactive mutation for Caspase 11.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

The present invention relates to a composition comprising an isolated, single stranded RNA molecule having a nucleotide sequence comprising 20 or more bases and a pattern of CpG dinucleotides defined by a strength of statistical bias greater than or equal to zero, and a pharmaceutically acceptable carrier suitable for injection. The present invention also relates to a kit comprising a cancer vaccine and the composition of the present invention as an adjuvant to the cancer vaccine. The present invention further relates to a method of treating a subject for a tumor and a method of stimulating an immune response.

1. A method of making a composition comprising:
isolating a single stranded RNA molecule of mammalian origin having a nucleotide sequence comprising 20 or more bases and a pattern of CpG dinucleotides defined by a strength of statistical bias greater than or equal to zero,
wherein the strength of the statistical bias for CpG dinucleotides is determined for an RNA molecule that has a nucleotide sequence (x(S₀)) by maximizing the probability of a sequence (S₀) over x, where
$$\begin{matrix}P ( S \vert x , m ) = \frac{1}{{Z}_{m} ( x )} {\prod }_{i = 1}^{L} {f}^{0} ( {s}_{i} ) exp ( x {N}_{m} ( S ) ) & [ EQUATION 1 ] \\ {Z}_{m} ( x ) = {\sum }_{sequences S} {\prod }_{i = 1}^{L} {f}^{0} ( {s}_{i} ) exp ( x {N}_{m} ( S ) ) & [ EQUATION 2 ]\end{matrix}$$
Zₘ(x) is the normalization constant,
P(S|x,m) is the probability of the sequence given the force (x) and motif m,
x is the force on the motif that introduces a statistical bias over P,
Nm(S) is the number of observed motifs, and
f⁰(s) is the number of observed motifs,
modifying at least one nucleic acid in the isolated RNA molecule, and
including a pharmaceutically acceptable carrier suitable for injection.

2. (canceled)

3. The method according to claim 1, wherein the RNA molecule is at least 95% homologous to SEQ ID NO. 154, or an immunostimulating fragment thereof

4. The method according to claim 1, wherein the pharmaceutically acceptable carrier is selected from the group consisting of an emulsion, liposome, microspheres, immune stimulating complex, nanospheres, montanide, squalene, cyclic dinucleotides, complementary immune modulators, and combinations thereof.

5. The method according to claim 1, wherein the RNA molecule is capable of stimulating an immune response to tumor cells.

6. The method according to claim 1 further comprising:
an antigen-encoding RNA molecule.

7. (canceled)

8. The method according to claim 1 further comprising:
a cancer vaccine, wherein the composition is an adjuvant to the cancer vaccine.

9-23. (canceled)
